(19) United States
(12) Patent Application Publication
(10) Pub. No.: US 2023/0125976 A1
(43) Pub. Date: Apr. 27, 2023

(54) MICROBIAL CONSORTIA FOR THE TREATMENT OF DISEASE

(71) Applicant: Federation Bio Inc., South San Francisco, CA (US)

(72) Inventors: Lee Swem, South San Francisco, CA (US); Dante Ricci, Richmond, CA (US); Ariel R. Brumbaugh, Redwood City, CA (US); John Cremin, South San Francisco, CA (US); Joshua J. Hamilton, South San Francisco, CA (US); Shital Tripathi, South San Francisco, CA (US); Lauren Wong, South San Francisco, CA (US); Heather Romasko, South San Francisco, CA (US); Racquel Bracken, South San Francisco, CA (US); Emily Drabant Conley, South San Francisco, CA (US); Anthony Rush, Oakland, CA (US)

(21) Appl. No.: 17/906,060

(22) PCT Filed: Mar. 10, 2021
(86) PCT No.: PCT/US2021/021790

§ 371 Date: Sep. 9, 2022

Related U.S. Application Data
(60) Provisional application No. 62/987,757, filed on Mar. 10, 2020.

(51) Int. Cl. A61K 35/741; A61P 3/00

(52) U.S. Cl. CPC A61K 35/741; A61P 3/00

(57) ABSTRACT

The present invention provides microbial consortia capable of stable engraftment in the gastrointestinal tract of a subject, and degradation of a disease-associated metabolic substrate, and methods of using the same.

### CROSS-REFERENCE TO RELATED APPLICATIONS

This application claims the benefit of and priority to U.S. Provisional Patent Application No. 62/987,757, filed Mar. 10, 2020, the disclosure of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Mar. 10, 2021, is named FBI-002WO_SL.txt and is 878,406 bytes in size.

### FIELD OF THE INVENTION

The invention generally relates to microbial consortia for administration to an animal for degradation of a disease-associated metabolic substrate.

### BACKGROUND

The gastrointestinal tract comprises various biological niches along its longitudinal length having different physical, chemical, and nutrient compositions. As a consequence of these diverse conditions, specific microbial communities are established within a particular biological niche. The microbial species comprising a specific microbial community are highly responsive to their local environment and produce an array of bioactive molecules that facilitate host engraftment, inter-microbial communication, nutrient metabolism, and inclusion or exclusion of competing microbial species. Adding further complexity, there is substantial diversity of microbial species and strains in the human GI tract between individuals, which is attributed to a number of factors including genetics, diet, antibiotic and antifungal use, surgical intervention (e.g., gastric by-pass/bowel resection), presence of inflammatory bowel disease and/or irritable bowel syndrome, and other environmental influences. However, despite this interindividual diversity, the functional attributes of the varying human gut microbiota are relatively consistent among healthy adults and comprise core metabolic pathways involved in carbohydrate metabolism, amino acid metabolism, fermentation, and oxidative phosphorylation.

Modulation of microbial species in the GI tract through the use of antibiotics, antifungals, and more recently, fecal microbial transplantation (“FMT”), have been approaches clinically investigated for the treatment and/or prevention of certain diseases and disorders. For example, Dodd et al. (Nature, 2007, 551: 648-652) have proposed FMT as a therapeutic to modulate the levels of aromatic amino acid metabolites in the serum of gnotobiotic mice, which affect intestinal permeability and systemic immunity. In further examples, administration of bacterial compositions has also been proposed as a method for treating Clostridium difficile infection, ulcerative colitis, cholestatic disease, and hyperoxaluria (see e.g., US 2018/0353554, WO 2019/036510, U.S. RE39,585).

As a modality for treating various diseases and/or conditions, there is a need for microbial compositions comprising a plurality of microbial species having improved therapeutic efficacy and an ability to efficiently engraft in a host, grow, and metabolize pathogenic substrates to non-pathogenic metabolic products within the various biological niches of the GI tract and within the diverse GI environments of different individuals.

### SUMMARY OF THE INVENTION

Disclosed here is a microbial consortium for administration to an animal comprising a plurality of active microbes and an effective amount of a supportive community of microbes. In some embodiments, the plurality of active microbes metabolize a first metabolic substrate to produce one or more than one metabolite, wherein the first metabolic substrate causes or contributes to disease in an animal.

In some embodiments, the supportive community of microbes comprises between 1 and 300 microbial strains and meets one, two, three, or four of the following conditions:
1) the supportive community of microbes metabolizes one or more than one metabolite produced by the plurality of active microbes, wherein the one or more than one metabolite inhibits metabolism of the first metabolic substrate by one or more of the plurality of active microbes,
2) the supportive community of microbes increases the flux of a precursor of the first metabolic substrate into a biochemical pathway that converts said precursor into a metabolite that is not the first metabolic substrate,
3) the supportive community of microbes enhances one or more than one characteristic of the plurality of active microbes when administered to an animal selected from the group consisting of: a) gastrointestinal engraftment, b) biomass, c) first metabolic substrate metabolism, and d) longitudinal stability as compared to administration of the plurality of active microbes in the absence of the supportive community of microbes, and
4) the supportive community of microbes catalyzes one or more than one reaction selected from the group consisting of: fermentation of polysaccharides to one or more than one of the group consisting of acetate, acetoin, 2-oxoglutarate, propionate, 1,3-propanediol, succinate, ethanol, lactate, butyrate, 2,3-butanediol, acetone, butanol, formate, H₂, and CO₂, fermentation of amino acids to one or more than one of the group consisting of acetate, propionate, butanoate, butyrate, isobutyrate, 2-methylbutyrate, isovalerate, isocaproate, 3-phenylpropanoate, phloretate, 3-(1H-indol-3-yl)propanoate, 5-aminopentanoate, H₂, H₂S, and CO₂, synthesis of one or more than one of the group consisting of methane from H₂ and CO₂, methane from formate and H₂, acetate from H₂ and CO₂, acetate from formate and H₂, acetate and sulfide from H₂, CO₂, and sulfate, propionate and CO₂ from succinate, succinate from H₂ and fumarate; synthesis of succinate from formate and fumarate, and butyrate, acetate, H₂, and CO₂ from lactate, deconjugation of conjugated bile acids to produce primary bile acids, conversion of cholic acid (CA) to 7-oxocholic acid, conversion of 7-oxocholic acid to 7-beta-cholic acid (7betaCA), conversion of chenodeoxycholic acid (CDCA) to 7-oxochenodeoxycholic acid, and conversion of 7-oxochenodeoxycholic acid to ursodeoxycholic acid (UDCA).

In some embodiments, the first metabolic substrate metabolizing activity of at least one of the plurality of active microbes is significantly different when measured in a standardized substrate metabolization assay at two pH values within a range of 4 to 8, and wherein the difference between the two pH values is at least one pH unit.

In some embodiments, the first metabolic substrate metabolizing activity of at least one of the plurality of active microbes is significantly different when measured in a standardized substrate metabolization assay at two first metabolic substrate concentrations within a 100 fold range, and wherein the difference between the two first metabolic substrate concentrations is at least 1.2-fold.

In some embodiments, the supportive community of microbes comprises at least three, at least four, at least five, or six phyla selected from Bacteroidetes, Firmicutes, Actinobacteria, Proteobacteria, Verrucomicrobia, and Euryarchaeota.

In some embodiments, the supportive community of microbes comprises one or more of the subclades Bacteroidales, Clostridiales, Erysipelotrichales. Negativicutes, Coriobacteriia, Bifidobacteriales, or Methanobacteriales.

In some embodiments, the first metabolic substrate is oxalate. In some embodiments, the supportive community of microbes catalyzes synthesis of methane from formate and H₂.

In some embodiments, the plurality of active microbes comprises Oxalobacter formigenes. In some embodiments the supportive community of microbes comprises a Bacteroidetes and a Euryarchaeota. In some embodiments, the supportive community of microbes comprises a Bateroides and Methanobrevibacter. In further embodiments, the supportive community of microbes comprises Bacteroides thetaiotaomicron and/or Bacteroides vulgatus, and Methanobrevibacter smithii.

In some embodiments, the supportive community of microbes metabolizes one or more than one metabolite produced by the plurality of active microbes, wherein the one or more than one metabolite inhibits metabolism of the plurality of active microbes.

In some embodiments, the supportive community of microbes enhances one or more than one characteristic of the plurality of active microbes when administered to an animal selected from the group consisting of gastrointestinal engraftment, biomass, first metabolic substrate metabolism, and longitudinal stability as compared to administration of the plurality of active microbes in the absence of the supportive community of microbes.

In some embodiments, the supportive community catalyzes one or more than one reaction selected from the group consisting of:
fermentation of polysaccharides to one or more than one of the group consisting of acetate, acetoin, 2-oxoglutarate, propionate, 1,3-propanediol, succinate, ethanol, lactate, butyrate, 2,3-butanediol, acetone, butanol, formate, H₂, and CO₂,
fermentation of amino acids to one or more than one of the group consisting of acetate, propionate, butanoate, butyrate, isobutyrate, 2-methylbutyrate, isovalerate, isocaproate, 3-phenylpropanoate, phloretate, 3-(1H-indol-3-yl)propanoate, 5-aminopentanoate, H₂, H₂S, and CO₂, synthesis of one or more than one of the group consisting of methane from H₂ and CO₂, methane from formate and H₂, acetate from H₂ and CO₂, acetate from formate and H₂, acetate and sulfide from H₂, CO₂, and sulfate, propionate and CO₂ from succinate, succinate from H₂ and fumarate; synthesis of succinate from formate and fumarate, and butyrate, acetate, H₂, and CO₂ from lactate, and
deconjugation of conjugated bile acids to produce primary bile acids, conversion of cholic acid (CA) to 7-oxocholic acid, conversion of 7-oxocholic acid to 7-beta-cholic acid (7betaCA), conversion of chenodeoxycholic acid (CDCA) to 7-oxochenodeoxycholic acid, and conversion of 7-oxochenodeoxycholic acid to ursodeoxycholic acid (UDCA).

In some embodiments, the supportive community of microbes comprises between 20 and 200 microbial strains. In some embodiments, the supportive community comprises at least 4 phyla selected from the group consisting of Bacteroidetes, Firmicutes, Actinobacteria, and Proteobacteria. In some embodiments, the supportive community comprises a Ruminococcus, Clostridium, Bacteroides, Neglecta, Bifidobacterium, Egerthella, Clostridiaceae, Parabacteroides, Bilophila, Dorea, Collinsella, and Faecalibacterium.

In some embodiments, the supportive community comprises Ruminococcus bromii, Clostridium citroniae, Bacteroides salyersiae, Neglecta timonensis, Bifidobacterium longum, Bifidobacterium pseudocatenulatum, Bacteroides thetaiotaomicron, Eggerthella lenta, Clostridiaceae sp., Bifidobacterium dentium, Parabacteroides merdae, Bilophila wadsworthia, Bacteroides caccae, Dorea longicatena, Collinsella aerofaciens, Clostridium scindens, Faecalibacterium prausnitzii, Clostridium symbiosum, and Bacteroides vulgatus.

In some embodiments, the supportive community comprises an Acidaminococcus, an Akkermansia, an Alistipes, an Anaerofustis, an Anaerostipes, an Anaerotruncus, a Bacteroides, a Barnesiella, a Bifidobacterium, a Bilophila, a Blautia, a Butyricimonas, a Catabacter hongkongensis, a Clostridiaceae, a Clostridiales, a Clostridium, a Collinsella, a Coprococcus, a Dialister, a Dielma, a Dorea, an Eggerthella, an Eisenbergiella, a Eubacterium, a Faecalibacterium, a Fusicatenibacter saccharivorans, a Gordonibacter pamelaeae, a Holdemanella, a Hungatella, a Lachnoclostridium, Lachnospiraceae, a Lactobacillus, a Longicatena, a Megasphaera, a Methanobrevibacter, a Monoglobus, a Neglecta, a Parabacteroides, a Paraprevotella, a Parasutterella, a Phascolarctobacterium, a Porphyromonas, a Roseburia hominis, a Ruminococcaceae, a Ruminococcus, a Ruthenibacterium, a Senegalimassilia, a Sutterella, and a Turicibacter.

In some embodiments, the supportive community comprises or consists of Acidaminococcus intestine, Akkermansia mucimphila, Alistipes onderdonkii, Alistipes putredinis, Alistipes senegalensis, Alistipes shahii, Alistipes sp., Alistipes timonensis, Anaerofustis stercorihominis, Anaerostipes hadrus, Anaerotruncus massiliensis, Bacteroides caccae, Bacteroides coprocola, Bacteroides faecis, Bacteroides finegoldii, Bacteroides fragilis, Bacteroides kribbi, Bacteroides massiliensis, Bacteroides nordii, Bacteroides ovatus, Bacteroides salyersiae, Bacteroides stercorirosoris, Bacteroides stercoris, Bacteroides thetaiotaomicron, Bacteroides uniformis, Bacteroides vulgatus, Bacteroides xylanisolvens, Barnesiella intestinihominis, Bifidobacterium adolescentis, Bifidobacterium bifidum, Bifidobacterium catenulatum, Bifidobacterium dentium, Bifidobacterium longum, Bifidobacterium pseudocatenulatum, Bilophila wadsworthia, Blautia faecis, Blautia hydrogenotrophica, Blautia massiliensis, Blautia obeum, Blautia wexlerae, Butyricimonas faecihominis, Catabacter hongkongensis, Clostridiaceae sp., Clostridiales sp., Clostridium aldenense, Clostridium bolteae, Clostridium citroniae, Clostridium clostridioforme, Clostridium fessum, Clostridium scindens, Collinsella aerofaciens, Coprococcus comes, Coprococcus eutactus, Dialister invisus, Dialister succinatiphilus, Dielma fastidiosa, Dorea formicigenerans, Dorea longicatena, Eggerthella lenta, Eisenbergiella tayi, Eubacterium eligens, Eubacterium hallii, Eubacterium rectale, Eubacterium siraeum, Eubacterium ventriosum, Eubacterium xylanophilum, Faecalibacterium prausnitzii, Fusicatenibacter saccharivorans, Gordonibacter pamelaeae, Holdemanella biformis, Hungatella effluvia, Lachnoclostridium pacaense, Lachnospiraceae sp., Lactobacillus rogosae, Longicatena caecimuris, Megasphaera massiliensis, Methanobrevibacter smithii, Monoglobus pectinilyticus, Neglecta timonensis, Parabacteroides distasonis, Parabacteroides merdae, Paraprevotella clara, Parasutterella excrementihominis, Phascolarctobacterium faecium, Porphyromonas asaccharolytica, Roseburia hominis, Ruminococcaceae sp., Ruminococcus bromii, Ruminococcus faecis, Ruthenibacterium lactatiformans, Senegalimassilia anaerobia, Sutterella massiliensis, Sutterella wadsworthensis, and Turicibacter sanguinis.

In some embodiments, the supportive community of microbes comprises an Akkermansia, an Alistipes, an Anaerostipes, a Bacteroides, a Bifidobacterium, a Bilophila, a Blautia, a Clostridium, a Collinsella aerofaciens, a Coprococcus, Dialister, a Dorea, an Eggerthella, an Eisenbergiella, a Eubacterium, a Faecalibacterium, a Fusicatenibacter, a Gordonibacter, a Holdemanella, a Hungatella, a Lachnoclostridium, a Lachnospiraceae, a Lactobacillus, aMonoglobus, a Neglecta, a Parabacteroides, a Paraprevotella, a Parasutterella, a Phascolarctobacterium, a Porphyromonas, a Roseburia, a Ruminococcaceae, a Ruminococcus, a Ruthenibacterium, and a Sutterella.

In some embodiments, the supportive community of microbes comprises or consists of Akkermansia mucimphila, Alistipes onderdonkii, Alistipes putredinis, Alistipes shahii, Alistipes timonensis, Anaerostipes hadrus, Bacteroides caccae, Bacteroides fragilis, Bacteroides kribbi, Bacteroides koreensis, Bacteroides massiliensis, Bacteroides nordii, Bacteroides salyersiae, Bacteroides stercorirosoris, Bacteroides stercoris, Bacteroides thetaiotaomicron, Bacteroides uniformis, Bacteroides vulgatus, Bacteroides xylanisolvens, Bifidobacterium adolescentis, Bifidobacterium catenulatum, Bifidobacterium dentium, Bifidobacterium longum, Bifidobacterium pseudocatenulatum, Bilophila wadsworthia, Bilophila wadsworthia, Blautia faecis, Blautia obeum, Blautia wexlerae, Clostridium aldenense, Clostridium bolteae, Clostridium citroniae, Clostridium clostridioforme, Clostridium fessum, Clostridium scindens, Collinsella aerofaciens, Coprococcus comes, Coprococcus eutactus, Dialister invisus, Dialister succinatiphilus, Dorea formicigenerans, Dorea longicatena, Eggerthella lenta, Eisenbergiella tayi, Eubacterium eligens, Eubacterium rectale, Faecalibacterium prausnitzii, Fusicatenibacter saccharivorans, Gordonibacter pamelaeae, Holdemanella biformis, Hungatella effluvia, Lachnoclostridium pacaense, Lachnospiraceae sp., Lactobacillus rogosae, Monoglobus pectinilyticus, Neglecta timonensis, Parabacteroides distasonis, Parabacteroides merdae, Paraprevotella clara, Parasutterella excrementihominis, Phascolarctobacterium faecium, Porphyromonas asaccharolytica, Roseburia hominis, Ruminococcaceae sp., Ruminococcus bromii, Ruminococcus faecis, Ruthenibacterium lactatiformans, Sutterella massiliensis, and Sutterella wadsworthensis.

In some embodiments, the microbial consortium or the supportive community of microbes comprises 20 to 200, 70 to 80, 80 to 90, 100 to 110, or 150 to 160 microbial strains.

In some embodiments, the supportive community of microbes comprises between 100 and 150 microbial strains.

In some embodiments, the plurality of active microbes and the supportive community of microbes are selected from a group of microbes each comprising a 16S sequence at least 80% identical, at least 90% identical, or at least 97% identical to any one of the microbes listed in Table 4, 22, 23, 20, 16, 17, 18 or 19.

In some embodiments, the plurality of active microbes and the supportive community of microbes consist of a group of microbes each comprising a 16S sequence at least 80% identical, at least 90% identical, or at least 97% identical to any one of the microbes listed in Table 22, 23, 20, 16, 17, 18 or 19.

In some embodiments, the first metabolic substrate metabolizing activity of one of the plurality of active microbes is significantly different compared to the first metabolic substrate activity of at least one other of the plurality of active microbes when measured in a standardized substrate metabolization assay under the same conditions.

In some embodiments, one of the plurality of active microbes has a higher first metabolic substrate metabolizing activity at a lower pH compared to at least one other of the plurality of active microbes at the same lower pH. In some embodiments, one of the plurality of active microbes has a higher first metabolic substrate metabolizing activity at a lower pH compared to a first metabolic substrate metabolizing activity of the same active microbe at a higher pH. In some embodiments the lower pH is at 4.5±0.5.

In some embodiments, one of the plurality of active microbes has a higher first metabolic substrate metabolizing activity at a higher pH compared to at least one other of the plurality of active microbes at the same higher pH. In some embodiments, one of the plurality of active microbes has a higher first metabolic substrate metabolizing activity at a higher pH compared to a first metabolic substrate activity of the same active microbe at a lower pH. In some embodiments, the higher pH is at 7.5±0.5.

In some embodiments, one of the plurality of active microbes has a higher first metabolic substrate metabolizing activity at a lower pH and one of the plurality of active microbes has a higher first metabolic substrate metabolizing activity at a higher pH. In some embodiments, the difference between the two pH values is at least 1.5, 2.0, 2.5, 3.0, 3.5, or 4.0 pH units.

In some embodiments, one of the plurality of active microbes has a higher first metabolic substrate metabolizing activity at a lower concentration of first metabolic substrate compared to the first metabolic substrate activity of at least one other of the plurality of active microbes when measured in a standardized substrate metabolization assay under the same conditions. In some embodiments, one of the plurality of active microbes has a higher first metabolic substrate metabolizing activity at a lower concentration of first metabolic substrate compared to a first metabolic substrate metabolizing activity of the same active microbe at a higher concentration of first metabolic substrate. In some embodiments, one of the plurality of active microbes has a higher first metabolic substrate metabolizing activity at a higher concentration of first metabolic substrate compared to the first metabolic substrate activity of at least one other of the plurality of active microbes when measured in a standardized substrate metabolization assay under the same conditions. In some embodiments, one of the plurality of active microbes has a higher first metabolic substrate metabolizing activity at a lower concentration of first metabolic substrate compared to a first metabolic substrate metabolizing activity of the same active microbe at a higher concentration of first metabolic substrate.

In some embodiments one of the plurality of active microbes has a higher first metabolic substrate metabolizing activity at a lower first metabolic substrate concentration and one of the plurality of active microbes has a higher first metabolic substrate metabolizing activity at a higher first metabolic substrate concentration. In some embodiments, the difference between the two first metabolic substrate concentrations is at least 1.2 fold, 2.0 fold, 3.0 fold, 4.0 fold, 5.0 fold, 6.0 fold, 7.0 fold, 8.0 fold, 9.0 fold, 10 fold, 20 fold, 30 fold, 40 fold, 50 fold, 60 fold, 70 fold, 80 fold, 90 fold, 100 fold, or greater than 100 fold.

In some embodiments, the microbial consortium of the present invention comprises a plurality of active microbes comprising 2 to 200 microbial strains. In certain embodiments, the plurality of active microbes comprises 2 to 20 microbial strains.

In some embodiments of the present invention, the first metabolic substrate is oxalate. In some embodiments, the one or more than one metabolite is selected from the group consisting of formate and carbon dioxide (CO₂). In some embodiments, at least one of the plurality of active microbes has a higher oxalate metabolizing activity at 0.75 mM of oxalate compared to the oxalate metabolizing activity of at least one other of the plurality of active microbes when measured in a standardized oxalate metabolization assay under the same conditions. In some embodiments, one of the plurality of active microbes has a higher oxalate metabolizing activity at 0.75 mM of oxalate compared to an oxalate metabolizing activity of the same active microbe at a higher concentration of oxalate. In some embodiments, at least one of the plurality of active microbes has a higher oxalate metabolizing activity at 40 mM of oxalate compared to the oxalate metabolizing activity of at least one other of the plurality of active microbes when measured in a standardized oxalate metabolization assay under the same conditions. In some embodiments, one of the plurality of active microbes has a higher oxalate metabolizing activity at 40 mM of oxalate compared to an oxalate metabolizing activity of the same active microbe at a lower concentration of oxalate. In some embodiments, one of the plurality of active microbes has a higher first metabolic substrate metabolizing activity at 0.75 mM of oxalate and another one of the plurality of active microbes has a higher first metabolic substrate metabolizing activity at 40 mM of oxalate.

In some embodiments, the standardized substrate metabolization assay comprises analysis of sample microbial cultures using a colorimetric enzyme assay that measures the activity of oxalate oxidase in a culture sample comprising the microbial consortium, wherein the culture sample comprises three or more microbial strains in an appropriate culture medium incubated for 1 hour to 120 hours in the presence of oxalate at a concentration of 0.5 mM to 50 mM, at a pH of 3.5 to 8.0, and at a temperature of 35° C. to 40° C.

In some embodiments, the standardized substrate metabolization assay comprises liquid chromatography-mass spectrometry, wherein the culture sample comprises three or more microbial strains in an appropriate culture medium incubated for 1 hour to 120 hours in the presence of oxalate at a concentration of 0.5 mM to 50 mM, at a pH of 3.5 to 8.0, and at a temperature of 35° C. to 40° C.

In some embodiments, the microbial consortium of the present invention further comprises: a fermenting microbe that metabolizes a fermentation substrate to one or more than one fermentation product; and a synthesizing microbe that catalyzes a synthesis reaction that combines the one or more than one metabolite and the one or more than one fermentation product to generate one or more than one synthesis product.

In some embodiments the one or more than one fermentation product is a second metabolic substrate for the plurality of active microbes or a third metabolic substrate for the synthesizing microbe. In some embodiments the one or more than one synthesis product is a second metabolic substrate for the plurality of active microbes or a fourth metabolic substrate for the fermenting microbe. In some embodiments the fermentation substrate is a polysaccharide and the one or more than one fermentation product is selected from the group consisting of acetate, acetoin, 2-oxoglutarate, propionate, 1,3-propanediol, succinate, ethanol, lactate, butyrate, 2,3-butanediol, acetone, butanol, formate, H₂, and CO₂. In some embodiments, the fermentation substrate is an amino acid and the one or more than one fermentation product is selected from the group consisting of acetate, propionate, butanoate, butyrate, isobutyrate, 2-methylbutyrate, isovalerate, isocaproate, 3-phenylpropanoate, phloretate, 3-(1H-indol-3-yl)propanoate, 5-aminopentanoate, H₂, H₂S, and CO₂.

In some embodiments the reaction catalyzed by the synthesizing microbe is selected from the group consisting of: synthesis of methane from H₂ and CO₂, methane from formate and H₂, acetate from H₂ and CO₂, acetate from formate and H₂, acetate and sulfide from H₂, CO₂, and sulfate, propionate and CO₂ from succinate, succinate from H₂ and fumarate; synthesis of succinate from formate and fumarate, and butyrate, acetate, H₂, and CO₂ from lactate.

In some embodiments, the microbial consortium, when administered to an animal on a high oxalate diet, significantly reduces oxalate concentration in a sample selected from the group consisting of blood, serum, stool, or urine, as compared to a sample collected from a corresponding control animal on a high oxalate diet that has not been administered with the microbial consortium.

In some embodiments, the plurality of active microbes comprises 3 microbial strains. In some embodiments, the plurality of active microbes comprises 3 Proteobacteria strains. In some embodiments, the plurality of active microbes comprises 3 Oxalobacter formigenes strains.

In some embodiments, the first metabolic substrate is a bile acid. For example, in some embodiments, the bile acid is lithocholic acid (LCA) or deoxycholic acid (DCA). In In some embodiments, the one or more than one metabolite produced by the plurality of active microbes is a secondary bile acid. For example, in some embodiments, the secondary bile acid is selected from the group consisting of iso-lithocholic acid (iso-LCA), or iso-deoxycholic acid (iso-DCA). In some embodiments, the the supportive community of microbes enhances the conversion of one or more conjugated bile acids selected from the group consisting of taurochenodeoxycholic acid (TCDCA), glycochenodeoxycholic acid (GCDCA), taurocholic acid (TCA), and glycocholic acid (GCA), to cholic acid (CA) or chenodeoxycholic acid (CDCA). In some embodiments, the supportive community of microbes enhances the conversion of CA to 7-beta-cholic acid (7betaCA). In other embodiments, the supportive community of microbes enhances the conversion of CDCA to ursodeoxycholic acid (UDCA).

In some embodiments, at least one of the plurality of active microbes has a higher bile acid metabolization activity at a bile acid concentration of 0.1 mM compared to the bile acid metabolization activity of at least one other of the plurality of active microbes when measured in a standardized bile acid metabolization assay under the same conditions. In some embodiments, at least one of the plurality of active microbes has a higher bile acid metabolizing activity at a bile acid concentration of 0.1 mM compared to a bile acid metabolizing activity of the same active microbe at a higher bile acid concentration. In some embodiments, at least one of the plurality of active microbes has a higher bile acid metabolization activity at a bile acid concentration of 10 mM compared to the bile acid metabolization activity of at least one other of the plurality of active microbes when measured in a standardized bile acid metabolization assay under the same conditions. In some embodiments, at least one of the plurality of active microbes has a higher bile acid metabolizing activity at a bile acid concentration of 10 mM compared to a bile acid metabolizing activity of the same active microbe at a lower bile acid concentration. In some embodiments, one of the plurality of active microbes has a higher bile acid metabolization activity at 0.1 mM of bile acid and another one of the plurality of active microbes has a higher bile acid metabolization activity at 10 mM of bile acid.

In some embodiments, the standardized substrate metabolization assay comprises using liquid chromatography-mass spectrometry to determine the bile acid profile in a culture sample comprising the microbial consortium, wherein the culture sample comprises three or more microbial strains in an appropriate culture media incubated for 1 hour to 96 hours in the presence of bile acids at a concentration of 0.1 mM to 10 mM, at a pH of 3.5 to 8.0, and at a temperature of 35° C. to 40° C.

In some embodiments, the plurality of active microbes comprises one or more microbial phyla selected from Firmicutes and Actinobacteria. In some embodiments, the plurality of active microbes comprises one or more microbial strain selected from Eggerthella lenta and Clostridium scindens.

In some embodiments, the microbial consortium of the present invention is administered as a pre-determined dose ranging from 1×10⁶ to 1×10¹³ total colony forming units (CFU)/kg.

In some embodiments, the microbial consortium, when administered to the animal, decreases a concentration of the first metabolic substrate in the animal.

In some embodiments the animal provides an experimental model of the disease.

The present disclosure also provides a pharmaceutical composition comprising a microbial consortium and a pharmaceutically acceptable carrier or excipient.

Also provided in the present disclosure is a method of treating a subject diagnosed with or at risk for a metabolic disease or condition selected from the group consisting of primary hyperoxaluria, secondary hyperoxaluria, cholestatic diseases (e.g. primary sclerosing cholangitis, primary biliary cholangitis, progressive familial intrahepatic cholestasis, or nonalcoholic steatohepatitis), and multiple sclerosis with a microbial consortium of the present invention.

In some embodiments, administration of the pharmaceutical composition disclosed herein reduces levels of the first metabolic substrate in a subject by at least 20%, at least 40%, at least 60%, or at least 80% as compared to an untreated control subject or as compared to pre-administration levels of the first metabolic substrate in the subject. In some embodiments, the first metabolic substrate is oxalate. In other embodiments, the first metabolic substrate is DCA or LCA. In some embodiments the level of first metabolic substrate is determined from a blood, serum, stool, or urine sample.

### BRIEF DESCRIPTION OF THE DRAWINGS

FIG. 1 shows a bar graph of % in vitro growth inhibition of supportive community strains in the presence of 0.5% oxalate (closed bars) or 0.125% oxalate (open bars) in culture media,

FIG. 2A shows a bar graph of in vitro oxalate-metabolizing activities of active microbial strains cultured for 72 hours in Mega Media, pH 7.5, containing 7.5 mM oxalate (closed bars) or 750 μM oxalate (open bars). FIG. 2B shows a bar graph of in vitro oxalate-metabolizing activities of active microbial strains cultured for 72 hours in Chopped Meat Media, pH 7.5, containing 7.5 mM oxalate (closed bars) or 750 μM oxalate (open bars).

FIG. 3A shows a bar graph of in vitro oxalate-metabolizing activities of active microbial strains cultured for 72 hours in Mega Media, at pH 4.5 (closed bars) or 7.2 (open bars), containing 7.5 mM oxalate. FIG. 3B shows a bar graph of in vitro oxalate-metabolizing activities of active microbial strains cultured for 72 hours in Chopped Meat Media, at pH 4.5 (closed bars) or 7.2 (open bars), containing 7.5 mM oxalate.

FIG. 4A shows a bar graph of in vitro oxalate levels (as measured by Absorbance₅₉₅) in microbial cultures comprising Oxalobacter formigenes only, active strains only, supportive strains only, or both active and supportive strains in Mega Medium. FIG. 4B shows a bar graph of in vitro oxalate levels (as measured by Absorbance₅₉₅) in microbial cultures comprising Oxalobacter formigenes only, active strains only, supportive strains only, or both active and supportive strains in Chopped Meat Medium at pH 7.2. Absorbance₅₉₅ was measured at the start of microbial culture incubation with 7.5 mM oxalate (t=0 hours, closed bars) and after 72 hours incubation with 7.5 mM oxalate (t=72 hours, open bars).

FIG. 5 shows the percent body weight gain (FIG. 5A), and food consumption (FIG. 5B) of gnotobiotic Balb/c mice on a normal or high oxalate diet, uncolonized or treated by gavage with Oxalobacter formigenes only, active strains only (actives), supportive strains only (supportives), or both active and supportive strains (full community).

FIG. 6 shows urinary oxalate concentrations of gnotobiotic Balb/c mice on a normal (no-oxalate) (FIG. 6A) or high oxalate (oxalate-supplemented) (FIG. 6B) diet, uncolonized (control) or treated by gavage with Oxalobacter formigenes only (formigenes), active strains only (Active), supportive strains only (Support), or both active and supportive strains (Active+Support).

FIG. 7 shows serum liver enzyme/function levels in gnotobiotic Balb/c mice on a normal (non-bold) or high oxalate diet (bold), treated by gavage with Oxalobacter formigenes only (O. formigenes), active strains only (Active), supportive strains only (Supportive), both active and supportive strains (Active+Supportive), or saline vehicle control (Saline). ALT=Alanine transaminase (FIG. 7A), AST=Aspartate transaminase (FIG. 7B), ALB=Albumin (FIG. 7C), ALP=Alanine phosphatase (FIG. 7D), A/G Ratio=Albumin/Globulin Ratio (FIG. 7E), TBIL=Total Bilirubin (FIG. 7F), GGT=Gamma-glutamyl transferase (FIG. 7G), TP=Prothrombin Time (FIG. 7H).

FIG. 8 shows serum kidney enzyme/function levels in gnotobiotic Balb/c mice on a normal (non-bold) or high oxalate diet (bold), treated by gavage with Oxalobacter formigenes only (O. formigenes), active strains only (Active), supportive strains only (Supportive), both active and supportive strains (Active+Supportive), or saline vehicle control (Saline). UREA=Urea (FIG. 8A), CREA=Creatinine (FIG. 8B), PHOS=Phosphorus (FIG. 8C), CA=Calcium (FIG. 8D), CL=Chloride FIG. 8E), NA=Sodium (FIG. 8F), K=Potassium (FIG. 8G), GLOB=Globulin (FIG. 8H).

FIG. 9 shows serum triglyceride (TRIG, FIG. 9A), cholesterol (CHOL, FIG. 9B), glucose (GLUC, FIG. 9C), and creatine kinase (CK, FIG. 9D) levels in gnotobiotic Balb/c mice on a normal (non-bold) or high oxalate diet (bold), treated by gavage with Oxalobacter formigenes only (O. formigenes), active strains only (Active), supportive strains only (Supportive), both active and supportive strains (Active+Supportive), or saline vehicle control.

FIG. 10 shows microbial species in fecal samples collected at the time of gavage or 2 weeks post-gavage from gnotobiotic Balb/c mice on a normal (Control; FIG. 10A, FIG. 10B, and FIG. 10C) or high oxalate (High-Ox; FIG. 10D, FIG. 10E, and FIG. 10F) diet, treated with active strains only (Actives; FIG. 10A and FIG. 10D), supportive strains only (Supportives; FIG. 10B and FIG. 10E), or active and supportive strains (Actives+Supportives; FIG. 10C and FIG. 10F).

FIG. 11 shows a bar graph of in vitro oxalate levels (as measured by LC-MS) in microbial cultures comprising a donor-derived strain grown in YCFAC base medium for 120 h at either pH 7.0 (white bars), pH 6.0 (grey bars), or pH 5.0 (black bars). % oxalate remaining is calculated relative to the amount of oxalate present at the start of the assay (2 mM). Oxalate levels in the pH 6.0 and pH 7.0 O. formigenes cultures (FBI00067) were below the limit of detection at the conclusion of the assay (<1.9% and <1.7% oxalate remaining, respectively).

FIG. 12 shows growth of cultures of donor-derived O. formigenes strains grown in YCFAC base medium supplemented with the indicated concentration of oxalate (0 mM, 2 mM, 40 mM, 80 mM, 120 mM, 160 mM) and grown for 144 hours (x-axis). Cultures are monitored by turbidity (OD600; y-axis). FIG. 12A-C show culture growth at pH 7.0 for the indicated strains, FIG. 12D-F show culture growth at pH 6.0 for the indicated strains, and FIG. 12G-I show culture growth at pH 5.0 for the indicated strains.

FIG. 13 shows urinary oxalate levels in germ-free C57Bl/6NTac mice (n=4 per condition) fed a low-complexity high-oxalate diet, uncolonized (−) or treated by gavage with one of 5 candidate microbial consortia (I to V) or a proof-of-concept consortium (+).

FIG. 14 shows urinary oxalate levels in germ-free C57Bl/6NTac mice (n=4 per condition) fed a high-complexity diet and given oxalate-supplemented drinking water, uncolonized (−) or treated by gavage with one of 5 candidate microbial consortia (I to V) or a positive-control consortium (+).

FIG. 15 shows urinary oxalate levels in germ-free C57Bl/6NTac mice (n=4 per condition) which were colonized with a non-oxalate-controlling human microbiome prior to the study. Mice were fed a high-complexity diet and given oxalate-supplemented drinking water, cleared of the human microbiome by antibiotic treatment, and were either left uncolonized (−) or were recolonized by gavage with one of 5 candidate microbial consortia (I to V), a positive-control consortium containing commercial strains (+), or a collection of donor-derived strains (“Putative Oxalate Degraders Only”) comprising 3 O. formigenes strains and a set of additional strains which had been preliminarily classified as oxalate-degrading.

FIG. 16 shows the diversity of microbial strains in fecal samples from the mice of FIG. 15 (measured by metagenomic sequencing).

FIG. 17 shows the relative abundance (FIG. 17A) and absolute abundance (FIG. 17B) of O. formigenes in feces of germ-free mice treated with a candidate microbial consortium (I to V) or a supportive community alone that lacks O. formigenes.

FIG. 18 shows the concentration of various bile acid compounds (including TCA, CA, and DCA) in cultures of commercial strains that were spiked with 100 μM TCA and incubated for 24 h at 37° C.

### DETAILED DESCRIPTION

Disclosed herein are microbial consortia for administration to an animal comprising a plurality of active microbes which metabolize a first metabolic substrate which causes or contributes to disease in the animal. The microbial consortia disclosed herein further comprise an effective amount of a supportive community of microbes that metabolize one or more than one metabolite produced by the plurality of active microbes, and wherein the one or more than one metabolite inhibits metabolism of the plurality of active microbes. These microbial consortia are advantageous in having enhanced characteristics when administered to an animal as compared to administration of the plurality of active microbes alone Enhanced characteristics of the microbial consortia include one or more of improved gastrointestinal engraftment, increased biomass, increased metabolism of the first metabolic substrate, and improved longitudinal stability.

To facilitate an understanding of the present invention, a number of terms and phrases are defined below.

The term “a” and “an” as used herein mean “one or more” and include the plural unless the context is appropriate

As used herein, the term “active microbes” refers to microbes that express sufficient amounts of one or more than one metabolic enzyme to metabolize a substrate that causes or contributes to disease in an animal.

As used herein, the term “biomass,” refers to the total mass of one or more than one microbe, or consortium in a given area or volume.

As used herein, the term “microbial consortium,” refers to a mixture of two or more microbial strains wherein one microbial strain in the mixture has a beneficial or desired effect on another microbial strain in the mixture.

As used herein, the term “gastrointestinal engraftment” refers to the establishment of one or more than one microbe, or microbial consortium, in one or more than one niche of the gastrointestinal tract that, prior to administration of the one or more than one microbe, or microbial consortium, is absent in the one or more than one microbe, or microbial consortium. Gastrointestinal engraftment may be transient, or may be persistent.

As used herein, the term “effective amount” refers to an amount sufficient to achieve a beneficial or desired result. In some embodiments, an effective amount can be improved gastrointestinal engraftment of one or more than one of the plurality of active microbes, increased biomass of one or more than one of the plurality of active microbes, increased metabolism of the first metabolic substrate, or improved longitudinal stability).

As used herein, the term “fermenting microbe” refers to a microbe that expresses sufficient amounts of one or more than one enzyme to catalyze a fermentation reaction in a gastrointestinal niche.

As used herein, the term “longitudinal stability” refers to the ability of one or more than one microbe, or microbial consortium to remain engrafted and metabolically active in one of more than one niche of the gastrointestinal tract despite transient or long-term environmental changes to the gastrointestinal niche.

As used herein, the term “metabolism,” “metabolize,” “metabolization,” or variants thereof refers to the biochemical conversion of a metabolic substrate to a metabolic product. In some embodiments, metabolization includes isomerization.

As used herein, the term “microbe” refers to a microbial organism including, but not limited to, bacteria, archaea, protozoa, and unicellular fungi.

As used herein, the term “microbial consortium” refers to a preparation of two or more microbes wherein the metabolic product of one of the two or more microbes is the metabolic substrate for one other microbe comprising the consortium.

As used herein, the term “pharmaceutical composition” refers to the combination of an active agent with a carrier, inert or active, making the composition especially suitable for therapeutic use in vivo or ex vivo.

As used herein, the term “pharmaceutically acceptable carrier” refers to any of the standard pharmaceutical carriers, such as phosphate buffered saline solution, water, emulsions (e.g., such as oil/water or water/oil emulsions), and various types of wetting agents. The compositions also can include stabilizers and preservatives. For examples of carriers, stabilizers, and adjuvants, see e.g., Martin, Remington's Pharmaceutical Sciences, 15^{th} Ed. Mack Publ. Co., Easton, Pa. [1975].

As used herein, “significantly” or “significant” refers to a change or alteration in a measurable parameter to a statistically significant degree as determined in accordance with an appropriate statistically relevant test. For example, in some embodiments, a change or alteration is significant if it is statistically significant in accordance with, e.g., a Student's t-test, chi-square, or Mann Whitney test.

As used herein, the term “standardized substrate metabolization assay” refers to an experimental assay known to persons of ordinary skill in the art used to quantify the amount of substrate converted to a metabolic product.

As used herein, the term “subject” refers to an organism to be treated by the microbial consortium and compositions described herein. Such organisms preferably include, but are not limited to, mammals (e.g., murines, simians, equines, bovines, porcines, canines, felines, and the like), and more preferably include humans.

As used herein, the term “supportive community” refers to one or more than one microbial strain that, when administered with an active microbe, enhances one or more than one characteristic of the active microbe selected from the group consisting of gastrointestinal engraftment, biomass, metabolic substrate metabolism, and longitudinal stability.

As used herein, the term “synthesizing microbe” refers to a microbe that expresses sufficient amounts of one or more than one enzyme to catalyze the combination of one or more than one metabolite produced by an active microbe, and one or more than one fermentation product produced by a fermenting microbe in a gastrointestinal niche.

The term percent “identity” or “sequence identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

When used in reference to 16S rRNA sequences, a “sequence identity” of at least 97% indicates that two microbial strains are likely to belong to the same species, whereas 16S rRNA sequences having less than 97% sequence identity indicate that two microbial strains likely belong to different species, and 16S rRNA sequences having less than 95% sequence identity indicates that two microbial strains likely belong to distinct genera (Stackebrandt E., and Goebel, B. M., Int J Syst Bact, 44 (1994) 846-849.).

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### Biological Niches

The present invention provides microbial consortia capable of engrafting into one or more than one niche of a gastrointestinal tract where it is capable of metabolizing a substrate that causes or contributes to disease in an animal. These niches comprise specific microbial communities whose composition varies according to a number of environmental factors including, but not limited to, the particular physical compartment of the gastrointestinal tract inhabited by a microbial community, the chemical and physicochemical properties of the environment inhabited, the metabolic substrate composition of the environment inhabited, and other co-inhabiting microbial species.

### Physical Compartments

A gastrointestinal tract comprises a number of physical compartments. For example, the human gastrointestinal tract includes the oral cavity, pharynx, esophagus, stomach, small intestine (duodenum, jejunum, ileum), cecum, large intestine (ascending colon, transverse colon, descending colon), and rectum. The pancreas, liver, gallbladder, and associated ducts, additionally comprise compartments of the human gastrointestinal tract. Each of these compartments has, for example, variable anatomical shape and dimension, aeration, water content, levels of mucus secretion, luminal presence of antimicrobial peptides, and presence or absence of peristaltic motility. Furthermore, the different gastrointestinal compartments vary in their pH. In humans, the pH of the oral cavity, upper stomach, lower stomach, duodenum, jejunum, ileum, and colon range from 6.5-7.5, 4.0-6.5, 1.5-4.0, 7.0-8.5, 4.0-7.0, and 4.0-7.0, respectively. Compartments of the gastrointestinal tract also differ in their levels of oxygenation which are subject to large degrees of fluctuation. For example, the luminal partial pressure of oxygen in the stomach of mice has been measured to be approximately 58 mm Hg, while the luminal partial pressure of oxygen in the distal sigmoid colon has been measured to be approximately 3 mm Hg (He et al., 1999). Oxygen levels of the gastrointestinal tract are highly determinative of the biochemical pathways utilized by commensal microbes. For example, commensal bacteria utilize aerobic respiration at oxygen concentrations above 5 mbar of 02, anaerobic respiration between 1-5 mbar of 02, and fermentation at 02 concentrations below 1 mbar. The sensitivity of microbes to 02 levels and their ability to carry out metabolic reactions under aerobic and/or anaerobic conditions influences which microbial species engraft in a particular gastrointestinal compartment.

### Metabolic Compartments

In addition to the various physical and chemical environments contributing to a gastrointestinal niche, different niches comprise different metabolic substrates.

Metabolic substrates that may be present in a gastrointestinal niche may include, but are not limited to, oxalate, fructan, inulin, glucuronoxylan, arabinoxylan, glucomannan, β-mannan, dextran, starch, arabinan, xyloglucan, galacturonan, β-glucan, galactomannan, rhamnogalacturonan I, rhamnogalacturonan II, arabinogalactan, mucin O-linked glycans, yeast α-mannan, yeast β-glucan, chitin, alginate, porphyrin, laminarin, carrageenan, agarose, alternan, levan, xanthan gum, galactooligosaccharides, hyaluronan, chondrointin sulfate, dermatan sulfate, heparin sulfate, keratan sulfate, phenylalanine, tyrosine, tryptophan, leucine, valine, isoleucine, glycine, proline, asparagine, glutamine, aspartate, glutamate, cysteine, lysine, arginine, serine, methionine, alanine, arginine, histidine, ornithine, citrulline, carnitine, hydroxyproline, cholic acid, chenodeoxycholic acid, taurochenodeoxycholic acid, glycochenodeoxycholic acid, cholesterol, cinnamic acid, coumaric acid, sinapinic acid, ferulic acid, caffeic acid, quinic acid, chlorogenic acid, catechin, epicatechin, gallic acid, pyrogallol, catechol, quercetin, myricetin, campherol, luteolin, apigenin, naringenin, and hesperidin.

### Microbial Consortia

The present invention provides microbial consortia comprising a plurality of active microbes and an effective amount of a supportive community of microbes. In some embodiments, a microbial consortium comprises 3 to 500 microbial strains. For example, in some embodiments, a microbial consortium comprises 3 to 500, 4 to 500, 5 to 500, 6 to 500, 7 to 500, 8 to 500, 9 to 500, 10 to 500, 11 to 500, 12 to 500, 13 to 500, 14 to 500, 15 to 500, 16 to 500, 17 to 500, 18 to 500, 19 to 500, 20 to 500, 21 to 500, 22 to 500, 23 to 500, 24 to 500, 25 to 500, 30 to 500, 35 to 500, 40 to 500, 45 to 500, 50 to 500, 60 to 500, 70 to 500, 80 to 500, 90 to 500, 100 to 500, 110 to 500, 120 to 500, 130 to 500, 140 to 500, 150 to 500, 160 to 500, 170 to 500, 180 to 500, 190 to 500, 200 to 500, 210 to 500, 220 to 500, 230 to 500, 240 to 500, 250 to 500, 260 to 500, 270 to 500, 280 to 500, 290 to 500, 300 to 500, 400 to 500, 3 to 300, 4 to 300, 5 to 300, 6 to 300, 7 to 300, 8 to 300, 9 to 300, 10 to 300, 11 to 300, 12 to 300, 13 to 300, 14 to 300, 15 to 300, 16 to 300, 17 to 300, 18 to 300, 19 to 300, 20 to 300, 21 to 300, 22 to 300, 23 to 300, 24 to 300, 25 to 300, 30 to 300, 35 to 300, 40 to 300, 45 to 300, 50 to 300, 60 to 300, 70 to 300, 80 to 300, 90 to 300, 100 to 300, 110 to 300, 120 to 300, 130 to 300, 140 to 300, 150 to 300, 160 to 300, 170 to 300, 180 to 300, 190 to 300, 200 to 300, 210 to 300, 220 to 300, 230 to 300, 240 to 300, 250 to 300, 260 to 300, 270 to 300, 280 to 300, 290 to 300, 3 to 250, 4 to 250, 5 to 250, 6 to 250, 7 to 250, 8 to 250, 9 to 250, 10 to 250, 11 to 250, 12 to 250, 13 to 250, 14 to 250, 15 to 250, 16 to 250, 17 to 250, 18 to 250, 19 to 250, 20 to 250, 21 to 250, 22 to 250, 23 to 250, 24 to 250, 25 to 250, 30 to 250, 35 to 250, 40 to 250, 45 to 250, 50 to 250, 60 to 250, 70 to 250, 80 to 250, 90 to 250, 100 to 250, 110 to 250, 120 to 250, 130 to 250, 140 to 250, 150 to 250, 160 to 250, 170 to 250, 180 to 250, 190 to 250, 200 to 250, 210 to 250, 220 to 250, 230 to 250, 240 to 250, 3 to 200, 4 to 200, 5 to 200, 6 to 200, 7 to 200, 8 to 200, 9 to 200, 10 to 200, 11 to 200, 12 to 200, 13 to 200, 14 to 200, 15 to 200, 16 to 200, 17 to 200, 18 to 200, 19 to 200, 20 to 200, 21 to 200, 22 to 200, 23 to 200, 24 to 200, 25 to 200, 30 to 200, 35 to 200, 40 to 200, 45 to 200, 50 to 200, 60 to 200, 70 to 200, 80 to 200, 90 to 200, 100 to 200, 110 to 200, 120 to 200, 130 to 200, 140 to 200, 150 to 200, 160 to 200, 170 to 200, 180 to 200, 190 to 200, 3 to 150, 4 to 150, 5 to 150, 6 to 150, 7 to 150, 8 to 150, 9 to 150, 10 to 150, 11 to 150, 12 to 150, 13 to 150, 14 to 150, 15 to 150, 16 to 150, 17 to 150, 18 to 150, 19 to 150, 20 to 150, 21 to 150, 22 to 150, 23 to 150, 24 to 150, 25 to 150, 30 to 150, 35 to 150, 40 to 150, 45 to 150, 50 to 150, 60 to 150, 70 to 150, 80 to 150, 90 to 150, 100 to 150, 110 to 150, 120 to 150, 130 to 150, 140 to 150, 3 to 100, 4 to 100, 5 to 100, 6 to 100, 7 to 100, 8 to 100, 9 to 100, 10 to 100, 11 to 100, 12 to 100, 13 to 100, 14 to 100, 15 to 100, 16 to 100, 17 to 100, 18 to 100, 19 to 100, 20 to 100, 21 to 100, 22 to 100, 23 to 100, 24 to 100, 25 to 100, 30 to 100, 35 to 100, 40 to 100, 45 to 100, 50 to 100, 60 to 100, 70 to 100, 80 to 100, 90 to 100, 3 to 75, 4 to 75, 5 to 75, 6 to 75, 7 to 75, 8 to 75, 9 to 75, 10 to 75, 11 to 75, 12 to 75, 13 to 75, 14 to 75, 15 to 75, 16 to 75, 17 to 75, 18 to 75, 19 to 75, 20 to 75, 21 to 75, 22 to 75, 23 to 75, 24 to 75, 25 to 75, 30 to 75, 35 to 75, 40 to 75, 45 to 75, 50 to 75, 60 to 75, 70 to 75, 3 to 50, 4 to 50, 5 to 50, 6 to 50, 7 to 50, 8 to 50, 9 to 50, 10 to 50, 11 to 50, 12 to 50, 13 to 50, 14 to 50, 15 to 50, 16 to 50, 17 to 50, 18 to 50, 19 to 50, 20 to 50, 21 to 50, 22 to 50, 23 to 50, 24 to 50, 25 to 50, 30 to 50, 35 to 50, 40 to 50, 45 to 50, 3 to 25, 4 to 25, 5 to 25, 6 to 25, 7 to 25, 8 to 25, 9 to 25, 10 to 25, 11 to 25, 12 to 25, 13 to 25, 14 to 25, 15 to 25, 16 to 25, 17 to 25, 18 to 25, 19 to 25, 20 to 25, 21 to 25, 22 to 25, 23 to 25, or 24 to 25 microbial strains. For example, in some embodiments, a microbial consortium comprises about 20 to about 200, about 70 to about 80, about 80 to about 90, about 100 to about 110, or about 150 to about 160 microbial strains.

In some embodiments, a microbial consortium described herein comprises a microbial strain having a relative abundance of approximately 90%, 80%, 70%, 60%, 50%, 40%, 30%, 20%, 10%, 5%, 1%, 0.1%, 0.01%, 0.001%, 0.0001%, 0.00001%, or 0.000001% of the total microbial consortium. In some embodiments, the relative abundance of a microbial strain is determined by metagenomic sequencing and calculated as the percentage of reads that are classified as an identified microbial strain, divided by the genome size. For example, in some embodiments, the relative abundance of a microbial strain of the invention is determined by metagenomic shotgun sequencing.

### Active Microbes

The microbial consortia of the present invention comprise a plurality of active microbes capable of metabolizing a first metabolic substrate that causes or contributes to disease in an animal. In some embodiments, the current invention provides a microbial consortium capable of metabolizing the first metabolic substrate at a pH within a range of 4 to 8. For example, in some embodiments, one or more than one of the plurality of active microbes is capable of metabolizing a first metabolic substrate at a pH within a range of 4 to 8, 4.2 to 8, 4.4 to 8, 4.6 to 8, 4.8 to 8, 5 to 8, 5.2 to 8, 5.4 to 8, 5.6 to 8, 5.8 to 8, 6 to 8, 6.2 to 8, 6.4 to 8, 6.6 to 8, 6.8 to 8, 7 to 8, 7.2 to 8, 7.4 to 8, 7.6 to 8, 7.8 to 8, 4 to 7, 4.2 to 7, 4.4 to 7, 4.6 to 7, 4.8 to 7, 5 to 7, 5.2 to 7, 5.4 to 7, 5.6 to 7, 5.8 to 7, 6 to 7, 6.2 to 7, 6.4 to 7, 6.6 to 7, 6.8 to 7, 4 to 6, 4.2 to 6, 4.4 to 6, 4.6 to 6, 4.8 to 6, 5 to 6, 5.2 to 6, 5.4 to 6, 5.6 to 6, 5.8 to 6, 4 to 6, 4.2 to 6, 4.4 to 6, 4.6 to 6, 4.8 to 6, 5 to 6, 5.2 to 6, 5.4 to 6, 5.6 to 6, or 5.8 to 6.

In some embodiments, the plurality of active microbes comprises one microbial strain having a significantly different first metabolic substrate-metabolizing activity in a standard substrate-metabolizing assay conducted at two pH values differing by 1 pH unit and within a pH range of 4 to 8. In some embodiments, the difference between the two pH values is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.2, 3.2, 3.3, 3.4, 3.5, 3.6, 3.7, 3.8, 3.9, or 4.0 pH units. For example, in some embodiments, one microbial strain has significantly different first metabolic substrate-metabolizing activities in a standard substrate metabolizing assay at pH 4 and pH 8, pH 5 and pH 8, pH 6 and pH 8, pH 7 and pH 8, pH 4 and pH 7, pH 5 and pH 7, pH 6 and pH 7, pH 4 and pH 6, pH 5 and pH 6, or pH 4 and pH 5.

As used herein, “lower pH” refers to a pH in a standardized substrate metabolization assay that is lower in value as compared to another pH value. For example, a standardized substrate metabolization assay performed at pH 4.5 has a lower pH as compared to a standardized substrate metabolization assay preformed at a pH of 7.5. “Higher pH,” as used herein, refers to a pH in a standardized substrate metabolization assay that is higher in value as compared to another pH value. For example a standardized substrate metabolization assay preformed at pH 7.5 has a higher pH as compared to a standardized substrate metabolization assay performed at a pH of 4.5.

As used herein, “higher first metabolic substrate-metabolizing activity” means either a first metabolic substrate-metabolizing activity of a microbial strain that is higher as compared to a first metabolic substrate-metabolizing activity of the same microbial strain under different conditions, and/or a first metabolic substrate-metabolizing activity of a microbial strain that is higher as compared to a first metabolic substrate-metabolizing activity of a different microbial strain under the same conditions.

In some embodiments, the plurality of active microbes comprises two microbial strains having significantly different first metabolic substrate-metabolizing activities. For example, in some embodiments, one of the plurality of active microbes has a significantly higher first metabolic substrate-metabolizing activity at a lower pH as compared to the first metabolic substrate-metabolizing activity of another microbial strain in the plurality of active microbes at the same lower pH. In some embodiments, one of the plurality of active microbes has a significantly higher first metabolic substrate-metabolizing activity at pH 4.0, 4.5, 5.0, 5.5, 6.0, or 6.5 as compared to the first metabolic substrate-metabolizing activity of another microbial strain in the plurality of active microbes at pH 4.0, 4.5, 5.0, 5.5, 6.0, or 6.5, respectively. In some embodiments, one of the plurality of active microbes has a significantly higher first metabolic substrate-metabolizing activity at a higher pH as compared to the first metabolic substrate-metabolizing activity of another microbial strain in the plurality of active microbes at the same higher pH. In some embodiments, one of the plurality of active microbes has a significantly higher first metabolic substrate-metabolizing activity at pH 7.5, 7.6, 7.7, 7.8, 7.9, or 8.0 as compared to the first metabolic substrate-metabolizing activity of another microbial strain in the plurality of active microbes at pH 7.5, 7.6, 7.7, 7.8, 7.9, or 8.0, respectively.

In some embodiments, one of the plurality of active microbes has a significantly higher first metabolic substrate-metabolizing activity at a lower pH as compared to its first metabolic substrate-metabolizing activity at a higher pH. For example, in some embodiments one of the plurality of active microbes has a significantly higher first metabolic substrate-metabolizing activity at pH 4.0, 4.5, 5.0, 5.5, 6.0, or 6.5 than it does at pH 7.5, 7.6, 7.7, 7.8, 7.9, or 8.0. In some embodiments, one of the plurality of active microbes has a significantly higher first metabolic substrate-metabolizing activity at a higher pH as compared to its first metabolic substrate-metabolizing activity at a lower pH. For example, in some embodiments one of the plurality of active microbes has a significantly higher first metabolic substrate-metabolizing activity at pH 7.5, 7.6, 7.7, 7.8, 7.9, or 8.0 than it does at pH 4.0, 4.5, 5.0, 5.5, 6.0, or 6.5.

In some embodiments, the plurality of active microbes comprises an active microbe having a higher first metabolic substrate-metabolizing activity at a lower pH and another microbe having a higher first metabolic substrate-metabolizing activity at a higher pH. For example, in some embodiments, the plurality of active microbes comprises an active microbe having a higher first metabolic substrate-metabolizing activity at pH 4.0 and another microbe having a higher first metabolic substrate-metabolizing activity at pH 7.5. In some embodiments, the plurality of active microbes comprises an active microbe having a higher first metabolic substrate-metabolizing activity at pH 4.0 and another microbe having a higher first metabolic substrate-metabolizing activity at pH 7.6. In some embodiments, the plurality of active microbes comprises an active microbe having a higher first metabolic substrate-metabolizing activity at pH 4.0 and another microbe having a higher first metabolic substrate-metabolizing activity at pH 7.7. In some embodiments, the plurality of active microbes comprises an active microbe having a higher first metabolic substrate-metabolizing activity at pH 4.0 and another microbe having a higher first metabolic substrate-metabolizing activity at pH 7.8. In some embodiments, the plurality of active microbes comprises an active microbe having a higher first metabolic substrate-metabolizing activity at pH 4.0 and another microbe having a higher first metabolic substrate-metabolizing activity at pH 7.9. In some embodiments, the plurality of active microbes comprises an active microbe having a higher first metabolic substrate-metabolizing activity at pH 4.0 and another microbe having a higher first metabolic substrate-metabolizing activity at pH 8.0. In some embodiments, the plurality of active microbes comprises an active microbe having a higher first metabolic substrate-metabolizing activity at pH 4.5 and another microbe having a higher first metabolic substrate-metabolizing activity at pH 7.5. In some embodiments, the plurality of active microbes comprises an active microbe having a higher first metabolic substrate-metabolizing activity at pH 4.5 and another microbe having a higher first metabolic substrate-metabolizing activity at pH 7.6. In some embodiments, the plurality of active microbes comprises an active microbe having a higher first metabolic substrate-metabolizing activity at pH 4.5 and another microbe having a higher first metabolic substrate-metabolizing activity at pH 7.7. In some embodiments, the plurality of active microbes comprises an active microbe having a higher first metabolic substrate-metabolizing activity at pH 4.5 and another microbe having a higher first metabolic substrate-metabolizing activity at pH 7.8. In some embodiments, the plurality of active microbes comprises an active microbe having a higher first metabolic substrate-metabolizing activity at pH 4.5 and another microbe having a higher first metabolic substrate-metabolizing activity at pH 7.9. In some embodiments, the plurality of active microbes comprises an active microbe having a higher first metabolic substrate-metabolizing activity at pH 4.5 and another microbe having a higher first metabolic substrate-metabolizing activity at pH 8.0. In some embodiments, the plurality of active microbes comprises an active microbe having a higher first metabolic substrate-metabolizing activity at pH 5.0 and another microbe having a higher first metabolic substrate-metabolizing activity at pH 7.5. In some embodiments, the plurality of active microbes comprises an active microbe having a higher first metabolic substrate-metabolizing activity at pH 5.0 and another microbe having a higher first metabolic substrate-metabolizing activity at pH 7.6. In some embodiments, the plurality of active microbes comprises an active microbe having a higher first metabolic substrate-metabolizing activity at pH 5.0 and another microbe having a higher first metabolic substrate-metabolizing activity at pH 7.7. In some embodiments, the plurality of active microbes comprises an active microbe having a higher first metabolic substrate-metabolizing activity at pH 5.0 and another microbe having a higher first metabolic substrate-metabolizing activity at pH 7.8. In some embodiments, the plurality of active microbes comprises an active microbe having a higher first metabolic substrate-metabolizing activity at pH 5.0 and another microbe having a higher first metabolic substrate-metabolizing activity at pH 7.9. In some embodiments, the plurality of active microbes comprises an active microbe having a higher first metabolic substrate-metabolizing activity at pH 5.0 and another microbe having a higher first metabolic substrate-metabolizing activity at pH 8.0. In some embodiments, the plurality of active microbes comprises an active microbe having a higher first metabolic substrate-metabolizing activity at pH 5.5 and another microbe having a higher first metabolic substrate-metabolizing activity at pH 7.5. In some embodiments, the plurality of active microbes comprises an active microbe having a higher first metabolic substrate-metabolizing activity at pH 5.5 and another microbe having a higher first metabolic substrate-metabolizing activity at pH 7.6. In some embodiments, the plurality of active microbes comprises an active microbe having a higher first metabolic substrate-metabolizing activity at pH 5.5 and another microbe having a higher first metabolic substrate-metabolizing activity at pH 7.7. In some embodiments, the plurality of active microbes comprises an active microbe having a higher first metabolic substrate-metabolizing activity at pH 5.5 and another microbe having a higher first metabolic substrate-metabolizing activity at pH 7.8. In some embodiments, the plurality of active microbes comprises an active microbe having a higher first metabolic substrate-metabolizing activity at pH 5.5 and another microbe having a higher first metabolic substrate-metabolizing activity at pH 7.9. In some embodiments, the plurality of active microbes comprises an active microbe having a higher first metabolic substrate-metabolizing activity at pH 5.5 and another microbe having a higher first metabolic substrate-metabolizing activity at pH 8.0. In some embodiments, the plurality of active microbes comprises an active microbe having a higher first metabolic substrate-metabolizing activity at pH 6.0 and another microbe having a higher first metabolic substrate-metabolizing activity at pH 7.5. In some embodiments, the plurality of active microbes comprises an active microbe having a higher first metabolic substrate-metabolizing activity at pH 6.0 and another microbe having a higher first metabolic substrate-metabolizing activity at pH 7.6. In some embodiments, the plurality of active microbes comprises an active microbe having a higher first metabolic substrate-metabolizing activity at pH 6.0 and another microbe having a higher first metabolic substrate-metabolizing activity at pH 7.7. In some embodiments, the plurality of active microbes comprises an active microbe having a higher first metabolic substrate-metabolizing activity at pH 6.0 and another microbe having a higher first metabolic substrate-metabolizing activity at pH 7.8. In some embodiments, the plurality of active microbes comprises an active microbe having a higher first metabolic substrate-metabolizing activity at pH 6.0 and another microbe having a higher first metabolic substrate-metabolizing activity at pH 7.9. In some embodiments, the plurality of active microbes comprises an active microbe having a higher first metabolic substrate-metabolizing activity at pH 6.0 and another microbe having a higher first metabolic substrate-metabolizing activity at pH 8.0. In some embodiments, the plurality of active microbes comprises an active microbe having a higher first metabolic substrate-metabolizing activity at pH 6.5 and another microbe having a higher first metabolic substrate-metabolizing activity at pH 7.5. In some embodiments, the plurality of active microbes comprises an active microbe having a higher first metabolic substrate-metabolizing activity at pH 6.5 and another microbe having a higher first metabolic substrate-metabolizing activity at pH 7.6. In some embodiments, the plurality of active microbes comprises an active microbe having a higher first metabolic substrate-metabolizing activity at pH 6.5 and another microbe having a higher first metabolic substrate-metabolizing activity at pH 7.7. In some embodiments, the plurality of active microbes comprises an active microbe having a higher first metabolic substrate-metabolizing activity at pH 6.5 and another microbe having a higher first metabolic substrate-metabolizing activity at pH 7.8. In some embodiments, the plurality of active microbes comprises an active microbe having a higher first metabolic substrate-metabolizing activity at pH 6.5 and another microbe having a higher first metabolic substrate-metabolizing activity at pH 7.9. In some embodiments, the plurality of active microbes comprises an active microbe having a higher first metabolic substrate-metabolizing activity at pH 6.5 and another microbe having a higher first metabolic substrate-metabolizing activity at pH 8.0.

In some embodiments, the plurality of active microbes comprises one microbial strain having a significantly different first metabolic substrate-metabolizing activity in a standard substrate-metabolizing assay conducted at a first metabolic substrate concentration as compared to its first metabolic substrate-metabolizing activity in a standard substrate-metabolizing assay conducted at a different first metabolic substrate concentration, wherein the difference between the two first metabolic substrate concentrations is within a 100 fold range. In some embodiments, the difference between the two first metabolic concentrations is 1.2 fold. For example, in some embodiments, the difference between the two first metabolic substrate concentrations is at least 1.2 fold, 1.4 fold, 1.6 fold, 1.8 fold, 2.0 fold, 4 fold, 6 fold, 8 fold, 10 fold, 20 fold, 30 fold, 40 fold, 50 fold, 60 fold, 70 fold, 80 fold, 90 fold, or 100 fold or greater.

As used herein, “lower concentration of first metabolic substrate” refers to a substrate concentration in a standardized substrate metabolization assay that is lower in value as compared to another substrate concentration. “Higher concentration of first metabolic substrate,” as used herein, refers to a substrate concentration in a standardized substrate metabolization assay that is higher in value as compared to another substrate concentration.

In some embodiments, the plurality of active microbes comprises two microbial strains having significantly different first metabolic substrate-metabolizing activities. For example, in some embodiments, one of the plurality of active microbes has a significantly higher first metabolic substrate-metabolizing activity at a lower concentration of first metabolic substrate as compared to the first metabolic substrate-metabolizing activity of another microbial strain in the plurality of active microbes at the same lower concentration of first metabolic substrate. In some embodiments, one of the plurality of active microbes has a significantly higher first metabolic substrate-metabolizing activity at a higher concentration of first metabolic substrate as compared to the first metabolic substrate-metabolizing activity of another microbial strain in the plurality of active microbes at the same higher concentration of first metabolic substrate.

In some embodiments, one of the plurality of active microbes has a significantly higher first metabolic substrate-metabolizing activity at a lower concentration of first metabolic substrate as compared to its first metabolic substrate-metabolizing activity at a higher concentration of first metabolic substrate. In some embodiments, one of the plurality of active microbes has a significantly higher first metabolic substrate-metabolizing activity at a higher concentration of first metabolic substrate as compared to its first metabolic substrate-metabolizing activity at a lower concentration of first metabolic substrate.

In some embodiments, the plurality of active microbes comprises an active microbe having a higher first metabolic substrate-metabolizing activity at a lower concentration of first metabolic substrate and another microbe having a higher first metabolic substrate-metabolizing activity at a higher concentration of first metabolic substrate. For example, in some embodiments, the difference between the lower concentration of first metabolic substrate and the higher concentration of first metabolic substrate is at least 1.2 fold, 1.4 fold, 1.6 fold, 1.8 fold, 2.0 fold, 4 fold, 6 fold, 8 fold, 10 fold, 20 fold, 30 fold, 40 fold, 50 fold, 60 fold, 70 fold, 80 fold, 90 fold, or 100 fold or greater.

In some embodiments, the plurality of active microbes comprises two microbial strains having significantly different growth rates. For example, in some embodiments, one of the plurality of active microbes has a significantly higher growth rate at a lower pH as compared to the growth rate of another microbial strain in the plurality of active microbes at the same lower pH. In some embodiments, one of the plurality of active microbes has a significantly higher growth rate at pH 4.0, 4.5, 5.0, 5.5, 6.0, or 6.5 as compared to the growth rate of another microbial strain in the plurality of active microbes at pH 4.0, 4.5, 5.0, 5.5, 6.0, or 6.5, respectively. In some embodiments, one of the plurality of active microbes has a significantly higher growth rate at a higher pH as compared to the growth rate of another microbial strain in the plurality of active microbes at the same higher pH. In some embodiments, one of the plurality of active microbes has a significantly higher growth rate at pH 7.5, 7.6, 7.7, 7.8, 7.9, or 8.0 as compared to the growth rate of another microbial strain in the plurality of active microbes at pH 7.5, 7.6, 7.7, 7.8, 7.9, or 8.0, respectively.

In some embodiments, one of the plurality of active microbes has a significantly higher growth rate at a lower pH as compared to its growth rate at a higher pH. For example, in some embodiments one of the plurality of active microbes has a significantly higher growth rate at pH 4.0, 4.5, 5.0, 5.5, 6.0, or 6.5 than it does at pH 7.5, 7.6, 7.7, 7.8, 7.9, or 8.0. In some embodiments, one of the plurality of active microbes has a significantly higher growth rate at a higher pH as compared to its growth rate at a lower pH. For example, in some embodiments one of the plurality of active microbes has a significantly higher growth rate at pH 7.5, 7.6, 7.7, 7.8, 7.9, or 8.0 than it does at pH 4.0, 4.5, 5.0, 5.5, 6.0, or 6.5.

In some embodiments, the plurality of active microbes comprises one microbial strain having a significantly higher growth rate when cultured in media containing a certain concentration of first metabolic substrate concentration as compared to the growth rate of another microbial strain in the plurality of active microbes cultured in the same media containing the same concentration of the first metabolic substrate. In some embodiments, the difference between the two growth rates is at least 0.2 fold, at least 0.4 fold, at least 0.6 fold, at least 0.8 fold, at least 1.0 fold, at least 1.2 fold, at least 1.4 fold, at least 1.6 fold, at least 1.8 fold, or at least 2.0 fold.

In some embodiments, the first metabolic substrate may be selected from, but not limited to, oxalate and a bile acid (e.g., lithocholic acid (LCA), deoxycholic acid (DCA)).

In some embodiments, the current disclosure provides a microbial consortium comprising a plurality of active microbes capable of metabolizing a first metabolic substrate to one or more than one metabolite. For example, in some embodiments, the one or more than one metabolite may be selected from, but not limited to, formate, CO₂, and a secondary bile acid (e.g., 3-oxo-deoxycholic acid (3 oxoDCA), 3-oxo-lithocholic acid (3oxoLCA), iso-lithocholic acid (iso-LCA), or iso-deoxycholic acid (iso-DCA)). In some embodiments, the plurality of active microbes can comprise 2 to 200 microbial strains. For example, in some embodiments, a microbial consortium comprises 2 to 10, 2 to 15, 2 to 20, 2 to 25, 2 to 30, 2 to 35, 2 to 40, 2 to 45, 2 to 50, 2 to 75, 2 to 100, 2 to 125, 2 to 150, 2 to 175, or 2 to 200 active microbial strains. In certain embodiments, the plurality of active microbes can comprise 2 to 20 microbial strains.

### Oxalate Metabolizing Active Microbes

In one aspect, the current disclosure provides a microbial consortium comprising a plurality of active microbes that metabolize oxalate. In some embodiments, each of the plurality of active microbes that metabolize oxalate express sufficient amounts of one or more than one enzyme involved in oxalate metabolism. For example, in some embodiments, one or more than one active microbe expresses formyl-CoA transferase (Frc), an oxalate-formate antiporter (e.g., OxIT), and oxalyl-CoA decarboxylase (e.g., OxC), and/or oxalate decarboxylase (e.g., OxD).

In some embodiments, the plurality of active microbes that metabolize oxalate comprise 2 to 20 oxalate-metabolizing microbial strains. For example, in some embodiments, a microbial consortium comprises 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, 19 to 20, 2 to 18, 3 to 18, 4 to 18, 5 to 18, 6 to 18, 7 to 18, 8 to 18, 9 to 18, 10 to 18, 11 to 18, 12 to 18, 13 to 18, 14 to 18, 15 to 18, 16 to 18, 17 to 18, 2 to 16, 3 to 16, 4 to 16, 5 to 16, 6 to 16, 7 to 16, 8 to 16, 9 to 16, 10 to 16, 11 to 16, 12 to 16, 13 to 16, 14 to 16, 15 to 16, 2 to 14, 3 to 14, 4 to 14, 5 to 14, 6 to 14, 7 to 14, 8 to 14, 9 to 14, 10 to 14, 11 to 14, 12 to 14, 13 to 14, 2 to 13, 3 to 13, 4 to 13, 5 to 13, 6 to 13, 7 to 13, 8 to 13, 9 to 13, 10 to 13, 11 to 13, 12 to 13, 2 to 12, 3 to 12, 4 to 12, 5 to 12, 6 to 12, 7 to 12, 8 to 12, 9 to 12, 10 to 12, 11 to 12, 2 to 12, 3 to 12, 4 to 12, 5 to 12, 6 to 12, 7 to 12, 8 to 12, 9 to 12, 10 to 12, 11 to 12, 2 to 10, 3 to 10, 4 to 10, 5 to 10, 6 to 10, 7 to 10, 8 to 10, 9 to 10, 2 to 10, 3 to 10, 4 to 10, 5 to 10, 6 to 10, 7 to 10, 8 to 10, 9 to 10, 2 to 8, 3 to 8, 4 to 8, 5 to 8, 6 to 8, 7 to 8, 2 to 6, 3 to 6, 4 to 6, 5 to 6, 2 to 4, or 3 to 4 oxalate-metabolizing strains of microbes. In some embodiments, the plurality of active microbes comprises 3 strains of oxalate-metabolizing microbes. In some embodiments the plurality of active microbes consists of 3 strains of oxalate-metabolizing microbes.

In some embodiments, the plurality of active microbes that metabolize oxalate may comprise one or more microbial species selected from, but not limited to Oxalobacter formigenes, Bifidobacterium sp., Bifidobacterium dentium, Dialister invisus, Lactobacillus acidophilus, Lactobacillus gasseri, Lactobacillus helveticus, Lactobacillus reuteri, Eggerthella lenta, Lactobacillus rhamnosus, Enterococcus faecalis, Enterococcus gallinarum, Enterococcus faecium, Providencia rettgeri, Streptococcus thermophilus, Lactobacillus plantarum, Lactobacillus casei, Lactobacillus salivarius, Lactobacillus johnsii, Bifidobacterium infantis, Bifidobacterium animalis, Clostridium sporogenes, Leuconostoc lactis, Leuconostoc mesenteroides.

In some embodiments the plurality of active microbes that metabolize oxalate may comprise two or more microbial species selected from, but not limited to, Bifidobacterium dentium ATCC 27678, Enterococcus faecalis HM-432, Lactobacillus helveticus DSM 20075, Bifidobacterium dentium ATCC 27680, Lactobacillus acidophilus ATCC 4357, Lactobacillus reuteri HM-102, Bifidobacterium dentium DSM 20221, Lactobacillus acidophilus DSM 20079, Lactobacillus rhamnosus ATCC 53103, Bifidobacterium dentium DSM 20436, Lactobacillus acidophilus DSM 20242, Lactobacillus rhamnosus DSM 20245, Bifidobacterium sp. HM-868, Lactobacillus gasseri ATCC 33323, Lactobacillus rhamnosus DSM 8746, Dialister invisus DSM 15470, Lactobacillus gasseri DSMZ 107525, Lactobacillus rhamnosus HM-106, Eggerthella lenta ATCC 43055, Lactobacillus gasseri DSMZ 20077, Oxalobacter formigenes ATCC 35274, Eggerthella lenta DSM 2243, Lactobacillus gasseri HM-104, Oxalobacter formigenes DSM 4420, Enterococcus faecalis HM-202, Lactobacillus gasseri HM-644, and Oxalobacter formigenes HM-1.

In some embodiments, the plurality of active microbes comprises an Oxalobacter formigenes strain having a 16S sequence at least 80% identical to SEQ ID NO: 67, SEQ ID NO: 133, or SEQ ID NO:289. In some embodiments, the plurality of active microbes comprises an Oxalobacter formigenes strain having a 16S sequence at least 90%, at least 91%, at least 92%, at least 93%, at least 94%, at least 95%, at least 96%, at least 97%, at least 98%, or at least 99% identical to SEQ ID NO: 67, SEQ ID NO: 133, or SEQ ID NO:289.

In some embodiments the plurality of active microbes comprises an Oxalobacter formigenes strain having a 16S sequence at least 80% identical to SEQ ID NO: 67 and an Oxalobacter formigenes strain having a 16S sequence at least 80% identical to SEQ ID NO: 133. In some embodiments, the plurality of active microbes comprises an Oxalobacter formigenes strain having a 16S sequence at least 90%, at least 91%, at least 92%, at least 93%, at least 94%, at least 95%, at least 96%, at least 97%, at least 98%, or at least 99%, identical to SEQ ID NO: 67 and an Oxalobacter formigenes strain having a 16S sequence at least 90%, at least 91%, at least 92%, at least 93%, at least 94%, at least 95%, at least 96%, at least 97%, at least 98%, or at least 99% identical to SEQ ID NO: 133.

In some embodiments the plurality of active microbes comprises an Oxalobacter formigenes strain having a 16S sequence at least 80% identical to SEQ ID NO: 133 and an Oxalobacter formigenes strain having a 16S sequence at least 80% identical to SEQ ID NO: 289. In some embodiments, the plurality of active microbes comprises an Oxalobacter formigenes strain having a 16S sequence at least 90%, at least 91%, at least 92%, at least 93%, at least 94%, at least 95%, at least 96%, at least 97%, at least 98%, or at least 99% identical to SEQ ID NO: 133 and an Oxalobacter formigenes strain having a 16S sequence at least 90%, at least 91%, at least 92%, at least 93%, at least 94%, at least 95%, at least 96%, at least 97%, at least 98%, or at least 99% identical to SEQ ID NO: 289.

In some embodiments the plurality of active microbes comprises an Oxalobacter formigenes strain having a 16S sequence at least 80% identical to SEQ ID NO: 67 and an Oxalobacter formigenes strain having a 16S sequence at least 80% identical to SEQ ID NO: 289. In some embodiments, the plurality of active microbes comprises an Oxalobacter formigenes strain having a 16S sequence at least 90%, at least 91%, at least 92%, at least 93%, at least 94%, at least 95%, at least 96%, at least 97%, at least 98%, or at least 99% identical to SEQ ID NO: 67 and an Oxalobacter formigenes strain having a 16S sequence at least 90%, at least 91%, at least 92%, at least 93%, at least 94%, at least 95%, at least 96%, at least 97%, at least 98%, or at least 99% identical to SEQ ID NO: 289.

In some embodiments the plurality of active microbes comprises an Oxalobacter formigenes strain having a 16S sequence at least 80% identical to SEQ ID NO: 67, an Oxalobacter formigenes strain having a 16S sequence at least 80% identical to SEQ ID NO: 133, and an Oxalobacter formigenes strain having a 16S sequence at least 80% identical to SEQ ID NO: 289. In some embodiments the plurality of active microbes comprises an Oxalobacter formigenes strain having a 16S sequence at least 90%, at least 91%, at least 92%, at least 93%, at least 94%, at least 95%, at least 96%, at least 97%, at least 98%, or at least 99% identical to SEQ ID NO: 67, an Oxalobacter formigenes strain having a 16S sequence at least 90%, at least 91%, at least 92%, at least 93%, at least 94%, at least 95%, at least 96%, at least 97%, at least 98%, or at least 99% identical to SEQ ID NO: 133, and an Oxalobacter formigenes strain having a 16S sequence at least 90%, at least 91%, at least 92%, at least 93%, at least 94%, at least 95%, at least 96%, at least 97%, at least 98%, or at least 99% identical to SEQ ID NO: 289.

In some embodiments the plurality of active microbes consists of an Oxalobacter formigenes strain having a 16S sequence at least 80% identical to SEQ ID NO: 67, an Oxalobacter formigenes strain having a 16S sequence at least 80% identical to SEQ ID NO: 133, and an Oxalobacter formigenes strain having a 16S sequence at least 80% identical to SEQ ID NO: 289. In some embodiments the plurality of active microbes consists of an Oxalobacter formigenes strain having a 16S sequence at least 90%, at least 91%, at least 92%, at least 93%, at least 94%, at least 95%, at least 96%, at least 97%, at least 98%, or at least 99% identical to SEQ ID NO: 67, an Oxalobacter formigenes strain having a 16S sequence at least 90%, at least 91%, at least 92%, at least 93%, at least 94%, at least 95%, at least 96%, at least 97%, at least 98%, or at least 99% identical to SEQ ID NO: 133, and an Oxalobacter formigenes strain having a 16S sequence at least 90%, at least 91%, at least 92%, at least 93%, at least 94%, at least 95%, at least 96%, at least 97%, at least 98%, or at least 99% identical to SEQ ID NO: 289.

As used herein, “substantially metabolizing oxalate,” “substantial metabolization of oxalate,” and variants thereof, refer to a statistically significant reduction in the amount of oxalate in an in vitro assay (for example, as described in Example 3). In some embodiments, one or more than one of the plurality of active microbes is capable of substantially metabolizing oxalate at a pH within a range of 4 to 8. For example, in some embodiments, one or more than one of the plurality of active microbes is capable of metabolizing oxalate at a pH within a range of 4 to 8, 4.2 to 8, 4.4 to 8, 4.6 to 8, 4.8 to 8, 5 to 8, 5.2 to 8, 5.4 to 8, 5.6 to 8, 5.8 to 8, 6 to 8, 6.2 to 8, 6.4 to 8, 6.6 to 8, 6.8 to 8, 7 to 8, 7.2 to 8, 7.4 to 8, 7.6 to 8, 7.8 to 8, 4 to 7, 4.2 to 7, 4.4 to 7, 4.6 to 7, 4.8 to 7, 5 to 7, 5.2 to 7, 5.4 to 7, 5.6 to 7, 5.8 to 7, 6 to 7, 6.2 to 7, 6.4 to 7, 6.6 to 7, 6.8 to 7, 4 to 6, 4.2 to 6, 4.4 to 6, 4.6 to 6, 4.8 to 6, 5 to 6, 5.2 to 6, 5.4 to 6, 5.6 to 6, 5.8 to 6, 4 to 6, 4.2 to 6, 4.4 to 6, 4.6 to 6, 4.8 to 6, 5 to 6, 5.2 to 6, 5.4 to 6, 5.6 to 6, or 5.8 to 6.

In some embodiments, the plurality of active microbes comprises one microbial strain having a significantly different oxalate-metabolizing activity in a standard oxalate metabolizing assay conducted at two pH values differing by at least 1 pH unit and within a pH range of 4 to 8. For example, in some embodiments, one microbial strain has significantly different oxalate-metabolizing activities in a standard oxalate metabolizing assay at pH 4 and pH 8, pH 5 and pH 8, pH 6 and pH 8, pH 7 and pH 8, pH 4 and pH 7, pH 5 and pH 7, pH 6 and pH 7, pH 4 and pH 6, pH 5 and pH 6, or pH 4 and pH 5.

In some embodiments, oxalate-metabolizing activity is detected using a standard oxalate metabolization assay. For example, in some embodiments, oxalate-metabolizing activity is detected using a colorimetric enzyme assay that measures the activity of oxalate oxidase. In certain embodiments, relative changes in oxalate abundance in culture media inoculated with microbial strains are measured using a commercial oxalate assay kit (e.g., Sigma-Aldrich, Catalog #MAK315). In some embodiments, oxalate-metabolizing activity is detected using liquid chromatography-mass spectrometry (LC-MS/MS). In some embodiments, relative changes in oxalate abundance is compared between the abundance of oxalate at the beginning of incubation (i.e. t=0), and after 2 hours, 4 hours, 6 hours, 8, hours, 10 hours, 12 hours, 14 hours, 16 hours, 18 hours, 24 hours, 30 hours, 36 hours, 48 hours, 60, hours, 72 hours, 84 hours, 96 hours, 120 hours, or 144 hours incubation.

As used herein, “higher oxalate metabolizing activity” means either an oxalate metabolizing activity of a microbial strain that is higher as compared to an oxalate metabolizing activity of the same microbial strain under different conditions, and/or an oxalate metabolizing activity of a microbial strain that is higher as compared to an oxalate metabolizing activity of a different microbial strain under the same conditions.

In some embodiments, the plurality of active microbes comprises two microbial strains having significantly different oxalate metabolizing activities. For example, in some embodiments, one of the plurality of active microbes has a significantly higher oxalate metabolizing activity at a lower pH as compared to the oxalate metabolizing activity of another microbial strain in the plurality of active microbes at the same lower pH. In some embodiments, one of the plurality of active microbes has a significantly higher oxalate metabolizing activity at pH 4.0, 4.5, 5.0, 5.5, 6.0, or 6.5 as compared to the oxalate metabolizing activity of another microbial strain in the plurality of active microbes at pH 4.0, 4.5, 5.0, 5.5, 6.0, or 6.5, respectively. In some embodiments, one of the plurality of active microbes has a significantly higher oxalate metabolizing activity at a higher pH as compared to the oxalate metabolizing activity of another microbial strain in the plurality of active microbes at the same higher pH. In some embodiments, one of the plurality of active microbes has a significantly higher oxalate metabolizing activity at pH 7.5, 7.6, 7.7, 7.8, 7.9, or 8.0 as compared to the oxalate metabolizing activity of another microbial strain in the plurality of active microbes at pH 7.5, 7.6, 7.7, 7.8, 7.9, or 8.0, respectively.

In some embodiments, one of the plurality of active microbes has a significantly higher oxalate metabolizing activity at a lower pH as compared to its oxalate metabolizing activity at a higher pH. For example, in some embodiments one of the plurality of active microbes has a significantly higher oxalate metabolizing activity at pH 4.0, 4.5, 5.0, 5.5, 6.0, or 6.5 than it does at pH 7.5, 7.6, 7.7, 7.8, 7.9, or 8.0. In some embodiments, one of the plurality of active microbes has a significantly higher oxalate metabolizing activity at a higher pH as compared to its oxalate metabolizing activity at a lower pH. For example, in some embodiments one of the plurality of active microbes has a significantly higher oxalate metabolizing activity at pH 7.5, 7.6, 7.7, 7.8, 7.9, or 8.0 than it does at pH 4.0, 4.5, 5.0, 5.5, 6.0, or 6.5.

In some embodiments, the plurality of active microbes comprises an active microbe having a higher oxalate metabolizing activity at a lower pH and another microbe having a higher oxalate metabolizing activity at a higher pH. For example, in some embodiments, the plurality of active microbes comprises an active microbe having a higher oxalate metabolizing activity at pH 4.0 and another microbe having a higher oxalate metabolizing activity at pH 7.5. In some embodiments, the plurality of active microbes comprises an active microbe having a higher oxalate metabolizing activity at pH 4.0 and another microbe having a higher oxalate metabolizing activity at pH 7.6. In some embodiments, the plurality of active microbes comprises an active microbe having a higher oxalate metabolizing activity at pH 4.0 and another microbe having a higher oxalate metabolizing activity at pH 7.7. In some embodiments, the plurality of active microbes comprises an active microbe having a higher oxalate metabolizing activity at pH 4.0 and another microbe having a higher oxalate metabolizing activity at pH 7.8. In some embodiments, the plurality of active microbes comprises an active microbe having a higher oxalate metabolizing activity at pH 4.0 and another microbe having a higher oxalate metabolizing activity at pH 7.9. In some embodiments, the plurality of active microbes comprises an active microbe having a higher oxalate metabolizing activity at pH 4.0 and another microbe having a higher oxalate metabolizing activity at pH 8.0. In some embodiments, the plurality of active microbes comprises an active microbe having a higher oxalate metabolizing activity at pH 4.5 and another microbe having a higher oxalate metabolizing activity at pH 7.5. In some embodiments, the plurality of active microbes comprises an active microbe having a higher oxalate metabolizing activity at pH 4.5 and another microbe having a higher oxalate metabolizing activity at pH 7.6. In some embodiments, the plurality of active microbes comprises an active microbe having a higher oxalate metabolizing activity at pH 4.5 and another microbe having a higher oxalate metabolizing activity at pH 7.7. In some embodiments, the plurality of active microbes comprises an active microbe having a higher oxalate metabolizing activity at pH 4.5 and another microbe having a higher oxalate metabolizing activity at pH 7.8. In some embodiments, the plurality of active microbes comprises an active microbe having a higher oxalate metabolizing activity at pH 4.5 and another microbe having a higher oxalate metabolizing activity at pH 7.9. In some embodiments, the plurality of active microbes comprises an active microbe having a higher oxalate metabolizing activity at pH 4.5 and another microbe having a higher oxalate metabolizing activity at pH 8.0. In some embodiments, the plurality of active microbes comprises an active microbe having a higher oxalate metabolizing activity at pH 5.0 and another microbe having a higher oxalate metabolizing activity at pH 7.5. In some embodiments, the plurality of active microbes comprises an active microbe having a higher oxalate metabolizing activity at pH 5.0 and another microbe having a higher oxalate metabolizing activity at pH 7.6. In some embodiments, the plurality of active microbes comprises an active microbe having a higher oxalate metabolizing activity at pH 5.0 and another microbe having a higher oxalate metabolizing activity at pH 7.7. In some embodiments, the plurality of active microbes comprises an active microbe having a higher oxalate metabolizing activity at pH 5.0 and another microbe having a higher oxalate metabolizing activity at pH 7.8. In some embodiments, the plurality of active microbes comprises an active microbe having a higher oxalate metabolizing activity at pH 5.0 and another microbe having a higher oxalate metabolizing activity at pH 7.9. In some embodiments, the plurality of active microbes comprises an active microbe having a higher oxalate metabolizing activity at pH 5.0 and another microbe having a higher oxalate metabolizing activity at pH 8.0. In some embodiments, the plurality of active microbes comprises an active microbe having a higher oxalate metabolizing activity at pH 5.5 and another microbe having a higher oxalate metabolizing activity at pH 7.5. In some embodiments, the plurality of active microbes comprises an active microbe having a higher oxalate metabolizing activity at pH 5.5 and another microbe having a higher oxalate metabolizing activity at pH 7.6. In some embodiments, the plurality of active microbes comprises an active microbe having a higher oxalate metabolizing activity at pH 5.5 and another microbe having a higher oxalate metabolizing activity at pH 7.7. In some embodiments, the plurality of active microbes comprises an active microbe having a higher oxalate metabolizing activity at pH 5.5 and another microbe having a higher oxalate metabolizing activity at pH 7.8. In some embodiments, the plurality of active microbes comprises an active microbe having a higher oxalate metabolizing activity at pH 5.5 and another microbe having a higher oxalate metabolizing activity at pH 7.9. In some embodiments, the plurality of active microbes comprises an active microbe having a higher oxalate metabolizing activity at pH 5.5 and another microbe having a higher oxalate metabolizing activity at pH 8.0. In some embodiments, the plurality of active microbes comprises an active microbe having a higher oxalate metabolizing activity at pH 6.0 and another microbe having a higher oxalate metabolizing activity at pH 7.5. In some embodiments, the plurality of active microbes comprises an active microbe having a higher oxalate metabolizing activity at pH 6.0 and another microbe having a higher oxalate metabolizing activity at pH 7.6. In some embodiments, the plurality of active microbes comprises an active microbe having a higher oxalate metabolizing activity at pH 6.0 and another microbe having a higher oxalate metabolizing activity at pH 7.7. In some embodiments, the plurality of active microbes comprises an active microbe having a higher oxalate metabolizing activity at pH 6.0 and another microbe having a higher oxalate metabolizing activity at pH 7.8. In some embodiments, the plurality of active microbes comprises an active microbe having a higher oxalate metabolizing activity at pH 6.0 and another microbe having a higher oxalate metabolizing activity at pH 7.9. In some embodiments, the plurality of active microbes comprises an active microbe having a higher oxalate metabolizing activity at pH 6.0 and another microbe having a higher oxalate metabolizing activity at pH 8.0. In some embodiments, the plurality of active microbes comprises an active microbe having a higher oxalate metabolizing activity at pH 6.5 and another microbe having a higher oxalate metabolizing activity at pH 7.5. In some embodiments, the plurality of active microbes comprises an active microbe having a higher oxalate metabolizing activity at pH 6.5 and another microbe having a higher oxalate metabolizing activity at pH 7.6. In some embodiments, the plurality of active microbes comprises an active microbe having a higher oxalate metabolizing activity at pH 6.5 and another microbe having a higher oxalate metabolizing activity at pH 7.7. In some embodiments, the plurality of active microbes comprises an active microbe having a higher oxalate metabolizing activity at pH 6.5 and another microbe having a higher oxalate metabolizing activity at pH 7.8. In some embodiments, the plurality of active microbes comprises an active microbe having a higher oxalate metabolizing activity at pH 6.5 and another microbe having a higher oxalate metabolizing activity at pH 7.9. In some embodiments, the plurality of active microbes comprises an active microbe having a higher oxalate metabolizing activity at pH 6.5 and another microbe having a higher oxalate metabolizing activity at pH 8.0.

In some embodiments, one or more than one of the plurality of active microbes is capable of substantially metabolizing oxalate at an oxalate concentration of about 0.75 mM to about 40 mM of oxalate. For example, in some embodiments, one or more than one of the plurality of active microbes is capable of substantially metabolizing oxalate at an oxalate concentration within a range of about 0.75 mM to about 40 mM, of about 1 mM to about 40 mM, of about 2.5 mM to about 40 mM, of about 5 mM to about 40 mM, of about 7.5 mM to about 40 mM, of about 10 mM to about 40 mM, of about 15 mM to about 40 mM, of about 20 mM to about 40 mM, of about 25 mM to about 40 mM, of about 30 mM to about 40 mM, of about 0.75 mM to about 30 mM, of about 1 mM to about 30 mM, of about 2.5 mM to about 30 mM, of about 5 mM to about 30 mM, of about 7.5 mM to about 30 mM, of about 10 mM to about 30 mM, of about 15 mM to about 30 mM, of about 20 mM to about 30 mM, of about 25 mM to about 30 mM, of about 0.75 mM to about 25 mM, of about 1 mM to about 25 mM, of about 2.5 mM to about 25 mM, of about 5 mM to about 25 mM, of about 7.5 mM to about 25 mM, of about 10 mM to about 25 mM, of about 15 mM to about 25 mM, of about 20 mM to about 25 mM, of about 0.75 mM to about 20 mM, of about 1 mM to about 20 mM, of about 2.5 mM to about 20 mM, of about 5 mM to about 20 mM, of about 7.5 mM to about 20 mM, of about 10 mM to about 20 mM, of about 15 mM to about 20 mM, of about 0.75 mM to about 15 mM, of about 1 mM to about 15 mM, of about 2.5 mM to about 15 mM, of about 5 mM to about 15 mM, of about 7.5 mM to about 15 mM, of about 10 mM to about 15 mM, of about 0.75 mM to about 10 mM, of about 1 mM to about 10 mM, of about 2.5 mM to about 10 mM, of about 5 mM to about 10 mM, of about 7.5 mM to about 10 mM, of about 0.75 mM to about 5 mM, of about 1 mM to about 5 mM, of about 2.5 mM to about 5 mM, or of about 0.75 mM to about 1 mM.

In some embodiments, the plurality of active microbes comprises one microbial strain having a significantly different oxalate-metabolizing activity in a standard in vitro oxalate metabolizing assay (for example, as described in Example 3) at an oxalate concentration as compared to its oxalate-metabolizing activity in a standard in vitro oxalate metabolizing assay conducted at a different oxalate concentration, wherein the difference between the two oxalate concentrations is within 100 fold. For example, in some embodiments, one microbial strain has significantly different oxalate-metabolizing activities in a standard oxalate metabolizing assay conducted at about 0.75 mM oxalate and about 40 mM oxalate, about 1 mM and about 40 mM, about 2.5 mM and about 40 mM, about 5 mM and about 40 mM, about 7.5 mM and about 40 mM, about 10 mM and about 40 mM, about 15 mM and about 40 mM, about 20 mM and about 40 mM, about 25 mM and about 40 mM, about 30 mM and about 40 mM, about 0.75 mM and about 30 mM, about 1 mM and about 30 mM, about 2.5 mM and about 30 mM, about 5 mM and about 30 mM, about 7.5 mM and about 30 mM, about 10 mM and about 30 mM, about 15 mM and about 30 mM, about 20 mM and about 30 mM, about 25 mM and about 30 mM, about 0.75 mM and about 25 mM, about 1 mM and about 25 mM, about 2.5 mM and about 25 mM, about 5 mM and about 25 mM, about 7.5 mM and about 25 mM, about 10 mM and about 25 mM, about 15 mM and about 25 mM, about 20 mM and about 25 mM, about 0.75 mM and about 20 mM, about 1 mM and about 20 mM, about 2.5 mM and about 20 mM, about 5 mM and about 20 mM, about 7.5 mM and about 20 mM, about 10 mM and about 20 mM, about 15 mM and about 20 mM, about 0.75 mM and about 15 mM, about 1 mM and about 15 mM, about 2.5 mM and about 15 mM, about 5 mM and about 15 mM, about 7.5 mM and about 15 mM, about 10 mM and about 15 mM, about 0.75 mM and about 10 mM, about 1 mM and about 10 mM, about 2.5 mM and about 10 mM, about 5 mM and about 10 mM, about 7.5 mM and about 10 mM, about 0.75 mM and about 5 mM, about 1 mM and about 5 mM, about 2.5 mM and about 5 mM, or about 0.75 mM and about 1 mM.

In some embodiments, the plurality of active microbes comprises two microbial strains having significantly different oxalate metabolizing activities. For example, in some embodiments, one of the plurality of active microbes has a significantly higher oxalate metabolizing activity at a lower concentration of oxalate as compared to the oxalate metabolizing activity of another microbial strain in the plurality of active microbes at the same lower concentration of oxalate. In some embodiments, one of the plurality of active microbes has a significantly higher oxalate metabolizing activity at an oxalate concentration of 0.75 mM, 1 mM, 2.5 mM, 5 mM, or 7.5 mM, as compared to the oxalate metabolizing activity of another microbial strain in the plurality of active microbes at an oxalate concentration of 0.75 mM, 1 mM, 2.5 mM, 5 mM, or 7.5 mM, respectively. In some embodiments, one of the plurality of active microbes has a significantly higher oxalate metabolizing activity at a higher concentration of oxalate as compared to the oxalate metabolizing activity of another microbial strain in the plurality of active microbes at the same higher concentration of oxalate. In some embodiments, one of the plurality of active microbes has a significantly higher oxalate metabolizing activity at an oxalate concentration of 15 mM, 20 mM, 25 mM 30 mM, or 40 mM as compared to the oxalate metabolizing activity of another microbial strain in the plurality of active microbes at an oxalate concentration of 15 mM, 20 mM, 25 mM 30 mM, or 40 mM, respectively.

In some embodiments, one of the plurality of active microbes has a significantly higher oxalate metabolizing activity at a lower oxalate concentration as compared to its oxalate metabolizing activity at a higher oxalate concentration. For example, in some embodiments one of the plurality of active microbes has a significantly higher oxalate metabolizing activity at 0.75 mM, 1 mM, 2.5 mM, 5 mM, or 7.5 mM of oxalate than it does at 15 mM, 20 mM, 25 mM 30 mM, or 40 mM of oxalate. In some embodiments, one of the plurality of active microbes has a significantly higher oxalate metabolizing activity at a higher oxalate concentration as compared to its oxalate metabolizing activity at a lower oxalate concentration. For example, in some embodiments one of the plurality of active microbes has a significantly higher oxalate metabolizing activity at 15 mM, 20 mM, 25 mM 30 mM, or 40 mM of oxalate than it does at 0.75 mM, 1 mM, 2.5 mM, 5 mM, or 7.5 mM of oxalate.

In some embodiments, the plurality of active microbes comprises an active microbe having a higher oxalate metabolizing activity at a lower concentration of oxalate and another microbe having a higher oxalate metabolizing activity at a higher concentration of oxalate. For example, in some embodiments, the plurality of active microbes comprises an active microbe having a higher oxalate metabolizing activity at about 0.75 mM oxalate and another active microbe having a higher oxalate metabolizing activity at about 40 mM oxalate. In some embodiments, the plurality of active microbes comprises an active microbe having a higher oxalate metabolizing activity at 1 mM oxalate and another active microbe having a higher oxalate metabolizing activity at about 40 mM oxalate. In some embodiments, the plurality of active microbes comprises an active microbe having a higher oxalate metabolizing activity at 2.5 mM oxalate and another active microbe having a higher oxalate metabolizing activity at about 40 mM oxalate. In some embodiments, the plurality of active microbes comprises an active microbe having a higher oxalate metabolizing activity at 5 mM oxalate and another active microbe having a higher oxalate metabolizing activity at about 40 mM oxalate. In some embodiments, the plurality of active microbes comprises an active microbe having a higher oxalate metabolizing activity at 7.5 mM oxalate and another active microbe having a higher oxalate metabolizing activity at about 40 mM oxalate. In some embodiments, the plurality of active microbes comprises an active microbe having a higher oxalate metabolizing activity at 0.75 mM oxalate and another active microbe having a higher oxalate metabolizing activity at about 30 mM oxalate. In some embodiments, the plurality of active microbes comprises an active microbe having a higher oxalate metabolizing activity at 1 mM oxalate and another active microbe having a higher oxalate metabolizing activity at about 30 mM oxalate. In some embodiments, the plurality of active microbes comprises an active microbe having a higher oxalate metabolizing activity at 2.5 mM oxalate and another active microbe having a higher oxalate metabolizing activity at about 30 mM oxalate. In some embodiments, the plurality of active microbes comprises an active microbe having a higher oxalate metabolizing activity at 5 mM oxalate and another active microbe having a higher oxalate metabolizing activity at about 30 mM oxalate. In some embodiments, the plurality of active microbes comprises an active microbe having a higher oxalate metabolizing activity at 7.5 mM oxalate and another active microbe having a higher oxalate metabolizing activity at about 30 mM oxalate. In some embodiments, the plurality of active microbes comprises an active microbe having a higher oxalate metabolizing activity at 0.75 mM oxalate and another active microbe having a higher oxalate metabolizing activity at about 25 mM oxalate. In some embodiments, the plurality of active microbes comprises an active microbe having a higher oxalate metabolizing activity at 1 mM oxalate and another active microbe having a higher oxalate metabolizing activity at about 25 mM oxalate. In some embodiments, the plurality of active microbes comprises an active microbe having a higher oxalate metabolizing activity at 2.5 mM oxalate and another active microbe having a higher oxalate metabolizing activity at about 25 mM oxalate. In some embodiments, the plurality of active microbes comprises an active microbe having a higher oxalate metabolizing activity at 5 mM oxalate and another active microbe having a higher oxalate metabolizing activity at about 25 mM oxalate. In some embodiments, the plurality of active microbes comprises an active microbe having a higher oxalate metabolizing activity at 7.5 mM oxalate and another active microbe having a higher oxalate metabolizing activity at about 25 mM oxalate. In some embodiments, the plurality of active microbes comprises an active microbe having a higher oxalate metabolizing activity at 0.75 mM oxalate and another active microbe having a higher oxalate metabolizing activity at about 20 mM oxalate. In some embodiments, the plurality of active microbes comprises an active microbe having a higher oxalate metabolizing activity at 1 mM oxalate and another active microbe having a higher oxalate metabolizing activity at about 20 mM oxalate. In some embodiments, the plurality of active microbes comprises an active microbe having a higher oxalate metabolizing activity at 2.5 mM oxalate and another active microbe having a higher oxalate metabolizing activity at about 20 mM oxalate. In some embodiments, the plurality of active microbes comprises an active microbe having a higher oxalate metabolizing activity at 5 mM oxalate and another active microbe having a higher oxalate metabolizing activity at about 20 mM oxalate. In some embodiments, the plurality of active microbes comprises an active microbe having a higher oxalate metabolizing activity at 7.5 mM oxalate and another active microbe having a higher oxalate metabolizing activity at about 20 mM oxalate. In some embodiments, the plurality of active microbes comprises an active microbe having a higher oxalate metabolizing activity at 0.75 mM oxalate and another active microbe having a higher oxalate metabolizing activity at about 15 mM oxalate. In some embodiments, the plurality of active microbes comprises an active microbe having a higher oxalate metabolizing activity at 1 mM oxalate and another active microbe having a higher oxalate metabolizing activity at about 15 mM oxalate. In some embodiments, the plurality of active microbes comprises an active microbe having a higher oxalate metabolizing activity at 2.5 mM oxalate and another active microbe having a higher oxalate metabolizing activity at about 15 mM oxalate. In some embodiments, the plurality of active microbes comprises an active microbe having a higher oxalate metabolizing activity at 5 mM oxalate and another active microbe having a higher oxalate metabolizing activity at about 15 mM oxalate. In some embodiments, the plurality of active microbes comprises an active microbe having a higher oxalate metabolizing activity at 7.5 mM oxalate and another active microbe having a higher oxalate metabolizing activity at about 15 mM oxalate.

In some embodiments, when tested in an in vitro oxalate metabolization assay (e.g., as described in Example 3 below), a plurality of active microbes of the present invention significantly reduces the concentration of oxalate present in a sample by at least 20%, by at least 30%, by at least 40%, by at least 50%, by at least 60%, by at least 70%, or by at least 80%.

In some embodiments, a plurality of active microbes of the present invention significantly reduces the concentration of oxalate present in a sample of blood, serum, bile, stool, or urine when administered to a subject by at least 20%, by at least 30%, by at least 40%, by at least 50%, by at least 60%, by at least 70%, or by at least 80% as compared to an untreated control subject or pre-administration levels. Concentrations of oxalate in a blood, serum, bile, stool or urine sample can be measured using a liquid chromatography-mass spectrometry (LC-MS), method as described in Example 4, below.

### Bile Salt Modifying Active Microbes

Unconjugated primary bile acids, cholic acid (CA) and chenodeoxycholic acid (CDCA), are substrates for 7α-dehydroxylation by select members of the gut microbiota. As shown below, 7α-dehydroxylation converts CA and CDCA to lithocholic acid (LCA) and deoxycholic acid (DCA), respectively. LCA and DCA are secondary bile acids that have been implicated in adverse health outcomes.

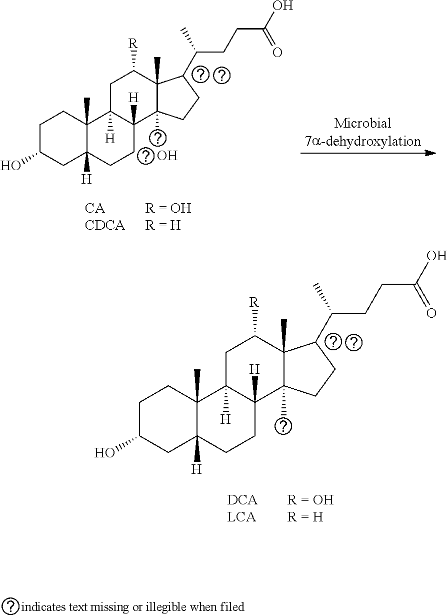

In some embodiments, a microbial consortium disclosed herein comprises microbial strains having robust 3α-hydroxysteroid dehydrogenase (3α-HSDH) and 3β-hydroxysteroid dehydrogenase (3β-HSDH) activity. As shown below, 3α-HSDH and 3β-HSDH convert DCA and LCA into alternative secondary bile acids isoDCA and isoLCA, respectively.

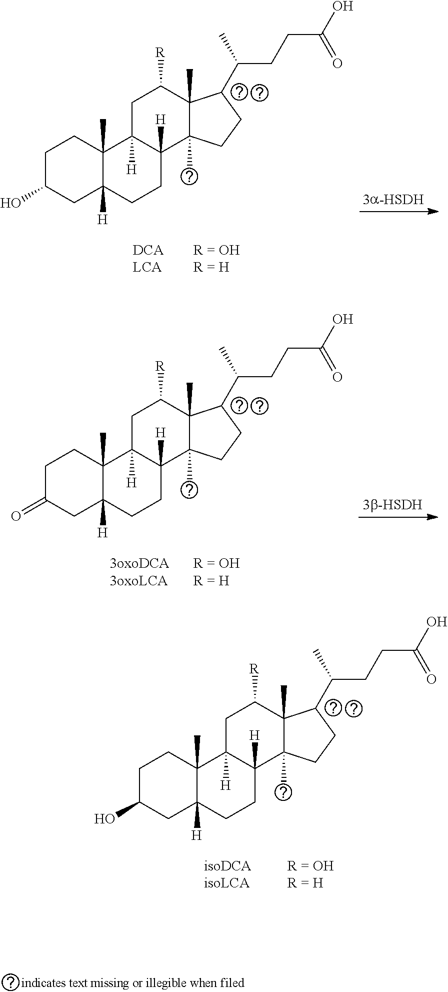

In some embodiments, microbial consortia provided herein comprise a plurality of active microbes expressing 3α-HSDH selected from one or more of Eggerthella lenta, Ruminococcus gnavus, Clostridium perfringens, Peptostreptococcus productus, and Clostridium scindens. In some embodiments, microbial consortia provided herein comprise a plurality of active microbes expressing 3β-HSDH selected from one or more of Peptostreptococcus productus, Clostridium innocuum, and Clostridium scindens.

In some embodiments, the plurality of active microbes comprises one or more than one microbial strain selected from: an Eggethella lenta strain having a 16S sequence at least 80% identical to SEQ ID NO: 30, an Eggethella lenta strain having a 16S sequence at least 80% identical to SEQ ID NO: 96, an Eggethella lenta strain having a 16S sequence at least 80% identical to SEQ ID NO: 170, an Eggethella lenta strain having a 16S sequence at least 80% identical to SEQ ID NO: 201, or a Clostridum scindens strain having a 16S sequence at least 80% identical to SEQ ID NO: 87.

In some embodiments, the plurality of active microbes comprises one or more than one microbial strain selected from: an Eggethella lenta strain having a 16S sequence at least 90%, at least 91%, at least 92%, at least 93%, at least 94%, at least 95%, at least 96%, at least 97%, at least 98%, or at least 99% identical to SEQ ID NO: 30, an Eggethella lenta strain having a 16S sequence at least 90%, at least 91%, at least 92%, at least 93%, at least 94%, at least 95%, at least 96%, at least 97%, at least 98%, or at least 99% identical to SEQ ID NO: 96, an Eggethella lenta strain having a 16S sequence at least 90%, at least 91%, at least 92%, at least 93%, at least 94%, at least 95%, at least 96%, at least 97%, at least 98%, or at least 99% identical to SEQ ID NO: 170, an Eggethella lenta strain having a 16S sequence at least 90%, at least 91%, at least 92%, at least 93%, at least 94%, at least 95%, at least 96%, at least 97%, at least 98%, or at least 99% identical to SEQ ID NO: 201, or a Clostridum scindens strain having a 16S sequence at least 90%, at least 91%, at least 92%, at least 93%, at least 94%, at least 95%, at least 96%, at least 97%, at least 98%, or at least 99% identical to SEQ ID NO: 87.

In some embodiments, the plurality of active microbes comprises two microbial strains having significantly different bile acid-metabolizing activities. For example, in some embodiments, one of the plurality of active microbes has a significantly higher bile acid-metabolizing activity at a lower concentration of bile acid as compared to the bile acid-metabolizing activity of another microbial strain in the plurality of active microbes at the same lower concentration of bile acid. In some embodiments, one of the plurality of active microbes has a significantly higher bile acid-metabolizing activity at a bile acid concentration of 0.1 mM, 0.2 mM, 0.3 mM, 0.4 mM, 0.5 mM, 0.6 mM, 0.7 mM, 0.8 mM, 0.9 mM, 1.0 mM, as compared to the bile acid-metabolizing activity of another microbial strain in the plurality of active microbes at an oxalate concentration of 0.1 mM, 0.2 mM, 0.3 mM, 0.4 mM, 0.5 mM, 0.6 mM, 0.7 mM, 0.8 mM, 0.9 mM, or 1.0 mM, respectively. In some embodiments, one of the plurality of active microbes has a significantly higher bile acid-metabolizing activity at a higher concentration of bile acid as compared to the bile acid-metabolizing activity of another microbial strain in the plurality of active microbes at the same higher concentration of bile acid. In some embodiments, one of the plurality of active microbes has a significantly higher bile acid metabolizing activity at a bile acid concentration of 5.0 mM, 5.5 mM, 6.0 mM, 6.5 mM, 7.0 mM, 7.5 mM, 8.0 mM, 8.5 mM, 9.0 mM, 9.5 mM, or 10.0 mM as compared to the oxalate metabolizing activity of another microbial strain in the plurality of active microbes at an oxalate concentration of 5.0 mM, 5.5 mM, 6.0 mM, 6.5 mM, 7.0 mM, 7.5 mM, 8.0 mM, 8.5 mM, 9.0 mM, 9.5 mM, or 10.0 mM, respectively.

In some embodiments, one of the plurality of active microbes has a significantly higher bile acid-metabolizing activity at a lower bile acid concentration as compared to its bile acid-metabolizing activity at a higher bile acid concentration. For example, in some embodiments one of the plurality of active microbes has a significantly higher bile acid-metabolizing activity at 0.1 mM, 0.2 mM, 0.3 mM, 0.4 mM, 0.5 mM, 0.6 mM, 0.7 mM, 0.8 mM, 0.9 mM, or 1.0 mM of bile acid than it does at. 5.0 mM, 5.5 mM, 6.0 mM, 6.5 mM, 7.0 mM, 7.5 mM, 8.0 mM, 8.5 mM, 9.0 mM, 9.5 mM, or 10.0 mM of bile acid. In some embodiments, one of the plurality of active microbes has a significantly higher bile acid-metabolizing activity at a higher bile acid concentration as compared to its bile acid metabolizing activity at a lower bile acid concentration. For example, in some embodiments one of the plurality of active microbes has a significantly higher bile acid-metabolizing activity at 5.0 mM, 5.5 mM, 6.0 mM, 6.5 mM, 7.0 mM, 7.5 mM, 8.0 mM, 8.5 mM, 9.0 mM, 9.5 mM, or 10.0 mM of bile acid than it does at 0.1 mM, 0.2 mM, 0.3 mM, 0.4 mM, 0.5 mM, 0.6 mM, 0.7 mM, 0.8 mM, 0.9 mM, or 1.0 mM of bile acid.

In some embodiments, the plurality of active microbes comprises an active microbe having a higher bile acid-metabolizing activity at a lower concentration of bile acid and another microbe having a higher bile acid-metabolizing activity at a higher concentration of bile acid. For example, in some embodiments, the plurality of active microbes comprises an active microbe having a higher bile acid metabolizing activity at about 0.1 mM bile acid and another active microbe having a higher bile acid-metabolizing activity at about 10 mM bile acid. In some embodiments, the plurality of active microbes comprises an active microbe having a higher bile acid metabolizing activity at about 0.2 mM bile acid and another active microbe having a higher bile acid-metabolizing activity at about 10 mM bile acid. In some embodiments, the plurality of active microbes comprises an active microbe having a higher bile acid metabolizing activity at about 0.3 mM bile acid and another active microbe having a higher bile acid-metabolizing activity at about 10 mM bile acid. In some embodiments, the plurality of active microbes comprises an active microbe having a higher bile acid metabolizing activity at about 0.4 mM bile acid and another active microbe having a higher bile acid-metabolizing activity at about 10 mM bile acid. In some embodiments, the plurality of active microbes comprises an active microbe having a higher bile acid metabolizing activity at about 0.5 mM bile acid and another active microbe having a higher bile acid-metabolizing activity at about 10 mM bile acid. In some embodiments, the plurality of active microbes comprises an active microbe having a higher bile acid metabolizing activity at about 0.1 mM bile acid and another active microbe having a higher bile acid-metabolizing activity at about 7.5 mM bile acid. In some embodiments, the plurality of active microbes comprises an active microbe having a higher bile acid metabolizing activity at about 0.2 mM bile acid and another active microbe having a higher bile acid-metabolizing activity at about 7.5 mM bile acid. In some embodiments, the plurality of active microbes comprises an active microbe having a higher bile acid metabolizing activity at about 0.3 mM bile acid and another active microbe having a higher bile acid-metabolizing activity at about 5.0 mM bile acid. In some embodiments, the plurality of active microbes comprises an active microbe having a higher bile acid metabolizing activity at about 0.4 mM bile acid and another active microbe having a higher bile acid-metabolizing activity at about 7.5 mM bile acid. In some embodiments, the plurality of active microbes comprises an active microbe having a higher bile acid metabolizing activity at about 0.5 mM bile acid and another active microbe having a higher bile acid-metabolizing activity at about 7.5 mM bile acid. In some embodiments, the plurality of active microbes comprises an active microbe having a higher bile acid metabolizing activity at about 0.1 mM bile acid and another active microbe having a higher bile acid-metabolizing activity at about 5.0 mM bile acid. In some embodiments, the plurality of active microbes comprises an active microbe having a higher bile acid metabolizing activity at about 0.2 mM bile acid and another active microbe having a higher bile acid-metabolizing activity at about 5.0 mM bile acid. In some embodiments, the plurality of active microbes comprises an active microbe having a higher bile acid metabolizing activity at about 0.3 mM bile acid and another active microbe having a higher bile acid-metabolizing activity at about 5.0 mM bile acid. In some embodiments, the plurality of active microbes comprises an active microbe having a higher bile acid metabolizing activity at about 0.4 mM bile acid and another active microbe having a higher bile acid-metabolizing activity at about 5.0 mM bile acid. In some embodiments, the plurality of active microbes comprises an active microbe having a higher bile acid metabolizing activity at about 0.5 mM bile acid and another active microbe having a higher bile acid-metabolizing activity at about 5.0 mM bile acid.

In some embodiments, when tested in a standard in vitro bile acid metabolization assay, a plurality of active microbes of the present invention significantly reduces the concentration of lithoholic acid (LCA) and or deoxycholic acid (DCA) present in a sample by at least 20%, by at least 30%, by at least 40%, by at least 50%, by at least 60%, by at least 70%, or by at least 80%.

In some embodiments, a plurality of active microbes of the present invention significantly reduces the concentration of LCA and/or DCA present in a sample of blood, serum, bile, stool, or urine when administered to a subject by at least 20%, by at least 30%, by at least 40%, by at least 50%, by at least 60%, by at least 70%, or by at least 80% as comparted to an untreated control subject or pre-administration levels.

### Supportive Community of Microbes

The microbial consortia of the present invention further comprise a supportive community of microbes that enhances one or more than one characteristic of the plurality of active microbes. For example, in some embodiments, the supportive community of microbes enhances gastrointestinal engraftment of the plurality of active microbes. In other embodiments, the supportive community of microbes enhances biomass of the plurality of active microbes. In other embodiments, the supportive community of microbes enhances metabolism of the first metabolic substrate by the plurality of active microbes. In other embodiments, the supportive community of microbes enhances longitudinal stability of the plurality of active microbes.

The supportive community of microbes disclosed herein metabolize one or more than one metabolite produced by the plurality of active microbes, wherein the one or more than one metabolite inhibits metabolism of the plurality of active microbes. For example, in some embodiments, the supportive community of microbes metabolizes formate produced by the plurality of active microbes, wherein the presence of formate inhibits the metabolism of oxalate by the plurality of active microbes. In some embodiments, the supportive community of microbes of the current invention catalyzes the fermentation of polysaccharides to one or more than one of the group consisting of acetate, acetoin, 2-oxoglutarate, propionate, 1,3-propanediol, succinate, ethanol, lactate, butyrate, 2,3-butanediol, acetone, butanol, formate, H₂, and CO₂. In some embodiments, the supportive community of microbes catalyzes the fermentation of amino acids to one or more than one of the group consisting of acetate, propionate, butanoate, butyrate, isobutyrate, 2-methylbutyrate, isovalerate, isocaproate, 3-phenylpropanoate, phloretate, 3-(1H-indol-3-yl)propanoate, 5-aminopentanoate, H₂, H₂S, and CO₂, In some embodiments, the supportive community catalyzes the synthesis of one or more than one of the group consisting of methane from H₂ and CO₂, methane from formate and H₂, acetate from H₂ and CO₂, acetate from formate and H₂, acetate and sulfide from H₂, CO₂, and sulfate, propionate and CO₂ from succinate, succinate from H₂ and fumarate; synthesis of succinate from formate and fumarate, and butyrate, acetate, H₂, and CO₂ from lactate. In some embodiments, the supportive community of microbes of the current invention catalyzes the deconjugation of conjugated bile acids to produce primary bile acids, the conversion of cholic acid (CA) to 7-oxocholic acid, the conversion of 7-oxocholic acid to 7-beta-cholic acid (7betaCA), the conversion of chenodeoxycholic acid (CDCA) to 7-oxochenodeoxycholic acid, and/or the conversion of 7-oxochenodeoxycholic acid to ursodeoxycholic acid (UDCA).

The supportive community of microbes of the current invention comprises between one and 300 microbial strains. For example, in some embodiments, the supportive community of microbes comprises between 1 and 300, 5 and 300, 10 and 300, 15 and 300, 20 and 300, 30 and 300, 40 and 300, 50 and 300, 60 and 300, 70 and 300, 80 and 300, 90 and 300, 100 and 300, 110 and 300, 120 and 300, 130 and 300, 140 and 300, 150 and 300, 160 and 300, 170 and 300, 180 and 300, 190 and 300, 200 and 300, 210 and 300, 220 and 300, 230 and 300, 240 and 300, 250 and 300, 260 and 300, 270 and 300, 280 and 300, 290 and 300, 1 and 250, 5 and 250, 10 and 250, 15 and 250, 20 and 250, 30 and 250, 40 and 250, 50 and 250, 60 and 250, 70 and 250, 80 and 250, 90 and 250, 100 and 250, 110 and 250, 120 and 250, 130 and 250, 140 and 250, 150 and 250, 160 and 250, 170 and 250, 180 and 250, 190 and 250, 200 and 250, 210 and 250, 220 and 250, 230 and 250, 240 and 250, 1 and 200, 5 and 200, 10 and 200, 15 and 200, 20 and 200, 30 and 200, 40 and 200, 50 and 200, 60 and 200, 70 and 200, 80 and 200, 90 and 200, 100 and 200, 110 and 200, 120 and 200, 130 and 200, 140 and 200, 150 and 200, 160 and 200, 170 and 200, 180 and 200, 190 and 200, 1 and 150, 5 and 150, 10 and 150, 15 and 150, 20 and 150, 30 and 150, 40 and 150, 50 and 150, 60 and 150, 70 and 150, 80 and 150, 90 and 150, 100 and 150, 110 and 150, 120 and 150, 130 and 150, 140 and 150, 1 and 100, 5 and 100, 10 and 100, 15 and 100, 20 and 100, 30 and 100, 40 and 100, 50 and 100, 60 and 100, 70 and 100, 80 and 100, 90 and 100, 1 and 50, 5 and 50, 10 and 50, 15 and 50, 20 and 50, 30 and 50, or 40 and 50 microbial strains. For example, in some embodiments, the supportive community of microbes comprises about 20 to about 200, about 70 to about 80, about 80 to about 90, about 100 to about 110, or about 150 to about 160 microbial strains.

In some embodiments, the supportive community of microbes comprises species of at least one, at least two, at least three, at least four, or at least five of the following phyla: Bacteroidetes, Firmicutes, Actinobacteria, Proteobacteria, Verrucomicrobia, and Euryarchaeota. In some embodiments, the supportive community of microbes comprises species of at least one, at least two, at least three, at least four, or at least five of the following subclades: Bacteroidales, Clostridiales, Erysipelotrichales, Negativicutes, Coriobacteriia, Bifidobacteriales, and Methanobacteriales.

In some embodiments, the supportive community of microbes of the current invention consumes one or more metabolites derived from an animal diet. For example, in some embodiments, the supportive community of microbes of the current invention consumes one or more than one of the following metabolites: a-mannan, acetate, agarose, alanine, arabinan, arabinogalactan, arabinoxylan, arginine, asparagine, aspartate, b-glucans, benzoic acids, carrageenan, catechol, chlorogenic acids, chondroitin sulfate, cysteine, dextran, enterodiol, flavan-3-ols, flavanones, flavones, flavonols, folate, formate, galactomannan, galacturonan, galacturonate, glucomannan, glutamine, glycine, hyaluronan, hydrogen, hydroxyproline, inulin, isoflavones, lactate, laminarin, leucine, levan, methionine, mucin O-linked glycans, phenylalanine, proline, rhamnogalacturonan I, rhamnogalacturonan II, secoisolariciresinol diglucoside, serine, starch, tyrosine, valine, xyloglucan, and xylooligosaccharides. In some embodiments, the supportive community of microbes is designed to maximize the number of metabolites derived from the host diet that the supportive community can consume.

In some embodiments, the supportive community of microbes of the current invention consumes one or more of the following dietary, host-derived, or microbial metabolites: thiamine, methanol, indole-3-acetate, L-glutamate, L-ornithine, niacin, 2-oxobutyrate, betaine, D-fructuronate, D-gluconate, D-tagaturonate, D-turanose, inosine, glycine, histidine, L-idonate, isoleucine, serine, N-acetyl-D-mannosamine, nitrate, thymidine, uridine, butyrate, propanoate, indole, glutamine, inositol, arginine, aspartate, malate, oxalate, phenol, succinate, ethanol, hydrogen, formate, lactate, aminobenzoate, lyxose, isomaltose, phenylalanine, tyrosine, pyruvate, mannitol, sorbitol, D-tagatose, glycerol, leucine, N-acetylgalactosamine, isovalerate, biotin, isobutyrate, 2-methylbutyrate, D-galactosamine, glycolithocholate, valine, melibiose, taurolithocholate, menaquinone, chenodeoxycholic acid, cholic acid, glycochenodeoxycholate, glycocholate, glycodeoxycholate, thiosulfate, pyridoxal, bicarbonate, N-acetyl-D-glucosamine, sulfate, riboflavin, methionine, N-acetylneuraminic acid, ribose, D-galacturonate, taurochenodeoxycholate, taurocholate, arabinose, rhamnose, pantothenic acid, xylooligosaccharide, acetate, D-glucuronic acid, cysteine, adenosylcobalamin, sucrose, trehalose, urea, xylose, cellobiose, mannose, L-fucose, D-galactose, D-glucosamine, D-psicose, fructooligosaccharide, carbon dioxide, maltose, ammonia, raffinose, dextrin, lactose, glucose, and fructose.

In some embodiments, the supportive community of microbes of the current invention produces one or more of the following metabolites: dimethylamine, folic acid, butylamine, phenylethylamine, 1,2-propanediol, acetone, trimethylamine, putrescine, tyramine, 4-aminobutyrate, valerate, 1,2-ethanediol, methylamine, phenylacetate, spermidine, hydrogen sulfide, linoleic acid, formaldehyde, trimethylamine N-oxide, cadaverine, alanine, threonine, methane, and pentanol.

In some embodiments of the invention, an original dosage form of the disclosed microbial consortium comprises active microbes and supportive microbes in a colony forming unit (CFU) ratio of about 5:1, 4:1, 3:1, 2:1, 1:1, 1:2, 1:3, 1:4, 1:5. In some embodiments, an original dosage form of the disclosed microbial consortium comprises active microbes and supportive microbes in total CFU amounts within about one order of magnitude, about two orders of magnitude, about three orders of magnitude, about four orders of magnitude, about 5 orders of magnitude, about 6 orders of magnitude, about 7 orders of magnitude, about 8 orders of magnitude, about 9 orders of magnitude, or about 10 orders of magnitude of each other.

In some embodiments, the supportive community of microbes may comprise one or more than one microbial strains selected from, but not limited to, Absiella dolichum, Bacteroides uniformis, Eubacterium siraeum, Acidaminococcus fermentans, Bacteroides vulgatus, Eubacterium ventriosum, Acidaminococcus sp., Bacteroides xylanisolvens, Faecalibacterium prausnitzii, Adlercreutzia equolifaciens, Bifidobacterium breve, Granulicatella adiacens, Akkermansia mucimphila, Bifidobacterium catenulatum, Holdemanella biformis, Alistipes finegoldii, Bifidobacterium pseudocatenulatum, Holdemania filiformis, Alistipes indistinctus, Bilophila wadsworthia, Hungatella hathewayi, Alistipes onderdonkii, Blautia hansenii, Intestinibacter bartlettii, Alistipes putredinis, Blautia hydrogenotrophica, Intestinimonas butyriciproducens, Alistipes senegalensis, Blautia obeum, Lactobacillus ruminis, Alistipes shahii, Blautia sp., Marvinbryantia formatexigens, Anaerobutyricum hallii, Blautia wexlerae, Megasphaera, Anaerofustis stercorihominis, Butyricimonas virosa, Methanobrevibacter smithii, Anaerostipes caccae, Butyrivibrio crossotus, Anaerotruncus colihominis, Catenibacterium mitsuokai, Bacteroides caccae, Clostridium asparagiforme, Bacteroides cellulosilyticus, Clostridium bolteae, Mitsuokella multacida, Bacteroides coprocola, Clostridium hiranonis, Odoribacter splanchnicus, Bacteroides coprophilus, Clostridium hylemonae, Olsenella uli, Bacteroides dorei, Clostridium leptum, Oscillibacter sp., Bacteroides dorei, Clostridium methylpentosum, Parabacteroides distasonis, Bacteroides eggerthii, Clostridium orbiscindens, Parabacteroides johnsonii, Bacteroides finegoldii, Clostridium saccharolyticum, Parabacteroides merdae, Bacteroides fragilis, Clostridium scindens, Parabacteroides sp., Bacteroides intestinalis, Clostridium sp., Prevotella buccalis, Bacteroides ovatus, Prevotella copri, Bacteroides pectinophilus, Roseburia inulinivorans, Bacteroides plebeius, Clostridium spiroforme, Ruminococcus gauvreauii, Bacteroides rodentium, Collinsella aerofaciens, Ruminococcus gnavus, Collinsella stercoris, Ruminococcus lactaris, Coprococcus comes, Ruminococcus torques, Coprococcus eutactus, Slackia exigua, Desulfovibrio piger, Slackia heliotrinireducens, Dorea formicigenerans, Solobacterium moorei, Dorea longicatena, Streptococcus salivarius subsp. Thermophilus, Bacteroides stercoris, Ethanoligenens harbinense, Subdoligranulum variabile, Bacteroides thetaiotaomicron, Eubacterium rectale, Turicibacter sanguinis, and Tyzzerella nexilis.

In some embodiments the supportive community of microbes may comprise one or more than one microbial strains selected from, but not limited to, Absiella dolichum DSM 3991, Bilophila wadsworthia ATCC 49260, Intestinibacter bartlettii DSM 16795, Acidaminococcus fermentans DSM 20731, Bilophila wadsworthia DSM 11045, Intestinimonas butyriciproducens DSM 26588, Acidaminococcus sp. HM-81, Blautia hansenii DSM 20583, Lactobacillus amylovorus DSM 20552, Adlercreutzia equolifaciens DSM 19450, Blautia hydrogenotrophica DSM 10507, Lactobacillus casei subsp. casei ATCC 393, Akkermansia muciniphila ATCC BAA-835, Blautia obeum DSMZ 25238, Lactobacillus casei subsp. casei ATCC 39539, Alistipes finegoldii DSM 17242, Blautia sp. HM-1032, Lactobacillus crispatus HM-370, Alistipes indistinctus DSM 22520, Blautia wexlerae DSM 19850, Lactobacillus johnsonii HM-643, Alistipes onderdonkii DSM 19147, Butyricimonas virosa DSM 23226, Lactobacillus parafarraginis HM-478, Alistipes putredinis DSM 17216, Butyrivibrio crossotus DSM 2876, Lactobacillus plantarum ATCC 14917, Alistipes senegalensis DSM 25460, Catenibacterium mitsuokai DSM 15897, Lactobacillus plantarum ATCC 202195, Alistipes shahii DSM 19121, Cetobacterium somerae DSM 23941, Lactobacillus ruminis ATCC 25644, Anaerobutyricum hallii DSM 3353, Clostridium asparagiforme DSM 15981, Lactobacillus ruminis DSM 20404, Anaerococcus lactolyticus DSM 7456, Clostridium bolteae DSM 15670, Lactobacillus ultunensis DSM 16048, Anaerofustis stercorihominis DSM 17244, Clostridium bolteae HM-1038, Lactococcus lactis Berridge DSM 20729, Anaerostipes caccae DSM 14662, Clostridium bolteae HM-318, Marvinbryantia formatexigens DSM 14469, Anaerotruncus colihominis DSM 17241, Clostridium cadaveris HM-1040, Megasphaera indica DSM 25562, Bacteroides caccae ATCC 43185, Clostridium citroniae HM-315, Megasphaera sp. DSM 102144, Bacteroides caccae HM-728, Clostridium hiranonis DSM 13275, Methanobrevibacter smithii DSM 11975, Bacteroides cellulosilyticus DSM 14838, Clostridium hylemonae DSM 15053, Methanobrevibacter smithii DSM 2374, Bacteroides cellulosilyticus HM-726, Clostridium innocuum HM-173, Methanobrevibacter smithii DSM 2375, Bacteroides coprocola DSM 17136, Clostridium leptum DSM 753, Methanobrevibacter smithii DSM 861, Bacteroides coprophilus DSM 18228, Clostridium methylpentosum DSM 5476, Methanomassiliicoccus luminyensis DSM 25720, Bacteroides dorei DSM 17855, Clostridium saccharolyticum DSM 2544, Methanosphaera stadtmanae DSMZ 3091, Bacteroides dorei HM-29, Clostridium scindens DSM 5676, Mitsuokella multacida DSM 20544, Bacteroides dorei HM-718, Clostridium scindens VPI 12708, Odoribacter splanchnicus DSM 20712, Bacteroides eggerthii DSM 20697, Clostridium sp. ATCC 29733, Olsenella uli DSM 7084, Bacteroides eggerthii HM-210, Clostridium sp. DSM 4029, Oscillibacter sp. HM-1030, Bacteroides finegoldii DSM 17565, Clostridium sp. HM-634, Parabacteroides distasonis ATCC 8503, Bacteroides finegoldii HM-727, Clostridium sp. HM-635, Parabacteroides goldsteinii HM-1050, Bacteroides fragilis HM-20, Clostridium spiroforme DSM 1552, Parabacteroides johnsonii DSM 18315, Bacteroides fragilis HM-709, Clostridium sporogenes ATCC 15579, Parabacteroides johnsonii HM-731, Bacteroides fragilis HM-710, Clostridium sporogenes ATCC 17889, Parabacteroides merdae DSM 19495, Bacteroides intestinalis DSM 17393, Clostridium sporogenes DSM 767, Parabacteroides merdae HM-729, Bacteroides ovatus ATCC 8483, Clostridium symbiosum HM-309, Parabacteroides merdae HM-730, Bacteroides ovatus HM-222, Clostridium symbiosum HM-319, Parabacteroides sp. HM-77, Bacteroides pectinophilus ATCC 43243, Collinsella aerofaciens ATCC 25986, Peptostreptococcus anaerobius DSM 2949, Bacteroides plebeius DSM 17135, Collinsella stercoris DSM 13279, Prevotella buccae HM-45, Bacteroides rodentium DSM 26882, Coprococcus catus ATCC 27761, Prevotella buccalis DSM 20616, Bacteroides salyersiae HM-725, Coprococcus comes ATCC 27758, Prevotella copri DSM 18205, Bacteroides sp. HM-18, Coprococcus eutactus ATCC 27759, Proteocatella sphenisci DSM 23131, Bacteroides sp. HM-19, Coprococcus eutactus ATCC 51897, Providencia rettgeri ATCC BAA-2525, Bacteroides sp. HM-23, Coprococcus sp. DSM 21649, Roseburia intestinalis DSM 14610, Bacteroides sp. HM-27, Desulfovibrio piger ATCC 29098, Roseburia inulinivorans DSM 16841, Bacteroides sp. HM-28, Dialister pneumosintes ATCC 51894, Ruminococcaceae sp. HM-79, Bacteroides sp. HM-58, Dorea formicigenerans ATCC 27755, Ruminococcus albus ATCC 27210, Bacteroides stercoris DSM 19555, Dorea longicatena DSM 13814, Ruminococcus bromii ATCC 27255, Bacteroides stercoris HM-1036, Eggerthella sp. DSM 11767, Ruminococcus bromii ATCC 51896, Bacteroides thetaiotaomicron ATCC 29148, Eggerthella sp. DSM 11863, Ruminococcus gauvreauii DSM 19829, Bacteroides uniformis ATCC 8492, Eggerthella sp. HM-1099, Ruminococcus gnavus ATCC 29149, Bacteroides vulgatus ATCC 8482, Ethanoligenens harbinense DSM 18485, Ruminococcus gnavus DSM 108212, Bacteroides vulgatus HM-720, Eubacterium eligens ATCC 27750, Ruminococcus gnavus HM-1056, Bacteroides xylanisolvens DSM 18836, Eubacterium rectale ATCC 33656, Ruminococcus lactaris ATCC 29176, Bifidobacterium adolescentis HM-633, Eubacterium siraeum DSM 15702, Ruminococcus lactaris HM-1057, Bifidobacterium angulatum HM-1189, Eubacterium ventriosum ATCC 27560, Ruminococcus torques ATCC 27756, Bifidobacterium animalis DSM 20104, Faecalibacterium prausnitzii ATCC 27766, Slackia exigua DSM 15923, Bifidobacterium animalis subsp. Lactis DSMZ 10140, Faecalibacterium prausnitzii ATCC 27768, Slackia heliotrinireducens DSM 20476, Bifidobacterium bifidum ATCC 11863, Faecalibacterium prausnitzii DSM 17677, Solobacterium moorei DSM 22971, Bifidobacterium breve DSM 20213, Faecalibacterium prausnitzii HM-473, Streptococcus salivarius subsp. thermophilus ATCC BAA-491, Bifidobacterium catenulatum DSM 16992, Flavonifractor plautii HM-1044, Streptococcus thermophilus ATCC 14485, Bifidobacterium longum infantis ATCC 55813, Flavonifractor plautii HM-303, Subdoligranulum variabile DSM 15176, Bifidobacterium longum subsp. longum HM-845, Granulicatella adiacens ATCC 49175, Turicibacter sanguinis DSM 14220, Bifidobacterium longum subsp. longum HM-846, Holdemanella biformis DSM 3989, Tyzzerella nexilis DSM 1787, Bifidobacterium longum subsp. longum HM-847, Holdemania filiformis DSM 12042, Veillonella dispar ATCC 17748, Bifidobacterium longum subsp. longum HM-848, Hungatella (prev. Clostridium) hathewayi HM-308, Veillonella sp. HM-49, Bifidobacterium pseudocatenulatum DSM 20438, Hungatella hathewayi DSM 13479, and Veillonella sp. HM-64.

Conjugated primary bile acids are synthesized in the liver from cholesterol, concentrated and stored in the gallbladder, and secreted into the duodenum to facilitate the solubilization and absorption of dietary lipids. Most bile acids are reabsorbed and recycled back to the liver through enterohepatic recirculation, but a sizable fraction (5%) escapes recycling, enters the large intestine, and is heavily metabolized into secondary bile acids by resident colonic microbes. Through microbial metabolism, four conjugated primary bile acids produced in the liver: taurochenoxycholic acid (TCDCA), glycochenodeoxycholic acid (GCDCA), taurocholic acid (TCA), and glycocholic acid (GCA), can be converted into over 100 molecules that have profound effects on host physiology. The unique profile of molecules produced is dependent on the metabolic capabilities of the resident colonic microbial community. As shown below, the first metabolic step upstream of secondary bile acid production is the deconjugation of conjugated primary bile acids by microbial bile salt hydrolases (BSH).

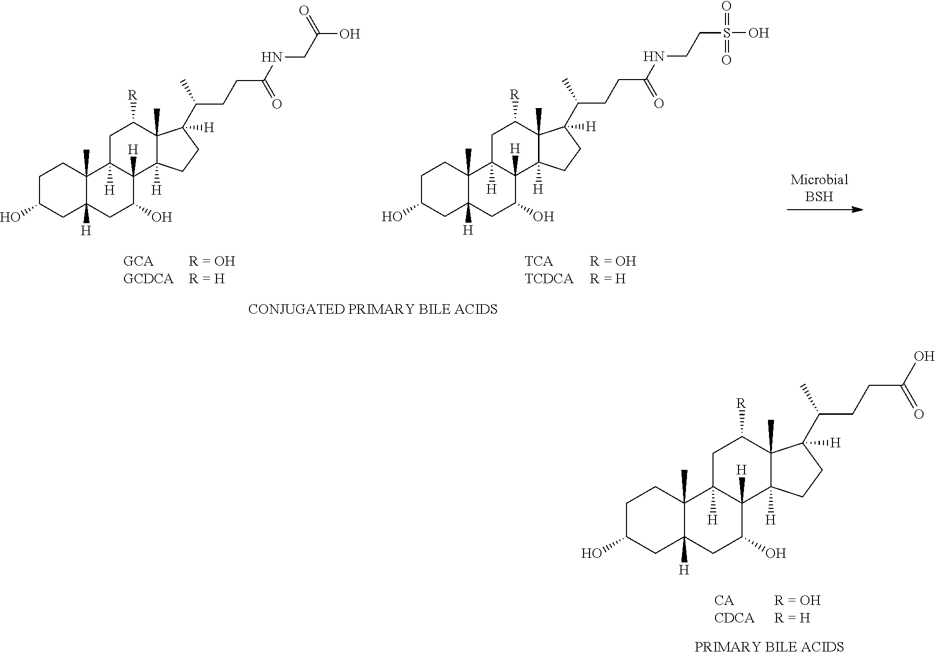

In some embodiments, the supportive community of microbes may comprise one or more microbial strains having robust and/or redundant BSH activity, so that deconjugation of primary bile acids can occur despite differences in host physiology, diet, plurality of active microbes present in the microbial consortium, or the pre-existing composition of the conjugated bile acid pool.

In some embodiments, the supportive community of microbes may comprise one or more than one microbial strains selected from, Alistipes indistinctus, Bacteroides ovatus, Bacteroides thetaiotaomicron, Bacteroides uniformis, Bacteroides vulgatus, Bacteroides xylanisolvens, Bifidobacterium angulatum, Bifidobacterium bifidum, Bifidobacterium breve, Bifidobacterium longum infantis, Bifidobacterium pseudocatenulatum, Blautia obeum, Clostridium hylemonae, Enterococcus faecalis, Hungatella hathewayi, Lactobacillus acidophilus, Methanobrevibacter smithii, Parabacteroides distasonis, Parabacteroides goldsteini, Providencia rettgeri, Roseburia inulinivorans, Ruminococcus bromii, Ruminococcus gnavus, and Turicibacter sanguinis.

In some embodiments, the current disclosure provides a microbial consortium comprising a plurality of active microbes that convert CA and CDCA into alternative secondary bile acids, thereby shifting the bile acid pool away from 7α-dehydroxylation products, LCA and DCA. For example, in some embodiments, a microbial consortium disclosed herein comprises microbial strains having robust 7α-hydroxysteroid dehydrogenase (7α-HSDH) and 7β-hydroxysteroid dehydrogenase (7β-HSDH) activity. As shown below, 7α-HSDH creates 7oxoCA and 7oxoCDCA intermediates, and 7β-HSDH converts CA and CDCA to 7βCA and ursodeoxycholic acid (UDCA).

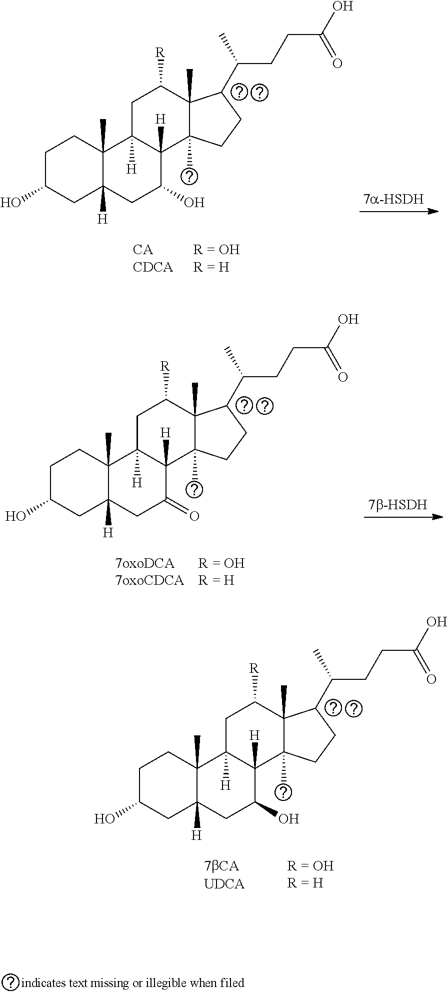

In some embodiments, microbial consortia provided herein comprise a plurality of active microbes expressing 7α-HSDH selected from one or more of Acinetobacter calcoaceticusi, Bacteroides thetaiotaomicron, Bacteroides intestinalis, Bacteroides fragilis, Eggerthella lenta, Ruminococcus sp. In some embodiments, microbial consortia provided herein comprises a plurality of active microbes expressing 7β-HSDH selected from one or both of Ruminococcus torques and Peptostreptococcus productus.

Fermenting and Synthesizing Microbes

In some embodiments, the microbial consortium of the current invention further comprises a fermenting microbe that metabolizes a fermentation substrate to generate one or more than one fermentation product. For example, in some embodiments, the fermentation product is a second metabolic substrate for one or more of the plurality of active microbes. In some embodiments, the fermentation product is a metabolic substrate for one or more of the supportive microbes. In some embodiments, the fermentation substrate is a polysaccharide and the generated fermentation product is one or more than one of acetate, acetoin, 2-oxoglutarate, propionate, 1,3-propanediol, succinate, ethanol, lactate, butyrate, 2,3-butanediol, acetone, butanol, formate, H₂, and CO₂. In some embodiments, the fermentation substrate is an amino acid and the generated fermentation product is one or more than one of acetate, propionate, butanoate, butyrate, isobutyrate, 2-methylbutyrate, isovalerate, isocaproate, 3-phenylpropanoate, phloretate, 3-(1H-indol-3-yl)propanoate, 5-aminopentanoate, H₂, H₂S, and CO₂.

In some embodiments, the microbial consortium of the current invention further comprises a synthesizing microbe that catalyzes a synthesis reaction that combines the one or more than one metabolite generated by the plurality of active microbes and the one or more than one fermentation product generated by the fermenting microbe to produce one or more than one synthesis product. In some embodiments the fermentation product generated by the fermenting microbe is a third metabolic substrate for the synthesizing microbe. In some embodiments, the one or more than one synthesis product is a second metabolic substrate for the plurality of active microbes. In some embodiments, the one or more than one synthesis product is a fourth metabolic substrate for the fermenting microbe.

In some embodiments, the synthesizing microbe catalyzes the synthesis of one or more than one of methane from H₂ and CO₂, methane from formate and H₂, acetate from H₂ and CO₂, acetate from formate and H₂, acetate and sulfide from H₂, CO₂, and sulfate, propionate and CO₂ from succinate, succinate from H₂ and fumarate; synthesis of succinate from formate and fumarate, and butyrate, acetate, H₂, and CO₂ from lactate.

In some embodiments, a fermenting microbe may be for example, but not limited to, Bacteroides thetaiotaomicron or Bactorides vulgatus. In some embodiments, a synthesizing microbe may be for example, but not limited to, Methanobrevibacter smithii or Methanomassiliicoccus luminyensis.

In some embodiments, the fermenting microbe is selected from a Bacteroides thetaiotaomicron strain having a 16S sequence at least 80% identical to SEQ ID NO: 20, SEQ ID NO: 76, SEQ ID NO: 139, or SEQ ID NO: 280. In some embodiments, the fermenting microbe is selected from a Bacteroides vulgatus strain having a 16S sequence at least 80% identical to SEQ ID NO: 39, SEQ ID NO: 111, SEQ ID NO: 121, SEQ ID NO: 173, SEQ ID NO: 211, SEQ ID NO: 308, SEQ ID NO: 321, or SEQ ID NO: 326. In some embodiments, the synthesizing microbe is selected from a Methanobrevibacter smithii strain having a 16S sequence at least 80% identical to SEQ ID NO: 292.

In some embodiments, the fermenting microbe is selected from a Bacteroides thetaiotaomicron strain having a 16S sequence at least 90%, at least 91%, at least 92%, at least 93%, at least 94%, at least 95%, at least 96%, at least 97%, at least 98%, or at least 99% identical to SEQ ID NO: 20, SEQ ID NO: 76, SEQ ID NO: 139, or SEQ ID NO: 280. In some embodiments, the fermenting microbe is selected from a Bacteroides vulgatus strain having a 16S sequence at least 90%, at least 91%, at least 92%, at least 93%, at least 94%, at least 95%, at least 96%, at least 97%, at least 98%, or at least 99% identical to SEQ ID NO: 39, SEQ ID NO: 111, SEQ ID NO: 121, SEQ ID NO: 173, SEQ ID NO: 211, SEQ ID NO: 308, SEQ ID NO: 321, or SEQ ID NO: 326. In some embodiments, the synthesizing microbe is selected from a Methanobrevibacter smithii strain having a 16S sequence at least 90%, at least 91%, at least 92%, at least 93%, at least 94%, at least 95%, at least 96%, at least 97%, at least 98%, or at least 99% identical to SEQ ID NO: 292.

In some embodiments, the microbial consortium disclosed herein comprises active microbes, fermenting microbes and synthesizing microbes in a colony forming unit (CFU) ratio selected from 1:1:1, 1:2:1, 1:1:2, 2:1:1, 2:1:2, 1:3:1, 1:1:3, 3:1:1, 3:1:3, 2:3:2, 2:2:3, 3:2:2, 3:2:3, 1:5:1, 1:1:5, 5:1:1, 5:1:5, 2:5:2, 2:2:5, 5:2:2, 5:2:5, 3:5:3, 3:3:5, 5:3:3, 5:3:5, 4:5:4, 4:4:5, 5:4:4, and 5:4:5. In some embodiments, an original dosage form of the disclosed microbial consortium comprises active microbes, fermenting microbes and synthesizing microbes in total CFU amounts within about one order of magnitude, about two orders of magnitude, about three orders of magnitude, about four orders of magnitude, about 5 orders of magnitude, about 6 orders of magnitude, about 7 orders of magnitude, about 8 orders of magnitude, about 9 orders of magnitude, or about 10 orders of magnitude of each other. In other embodiments, an original dosage form of the disclosed microbial consortium comprises active microbes, fermenting microbes and synthesizing microbes in CFU amounts within about two orders of magnitude of each other. In some embodiments, an original dosage form of the disclosed microbial consortium comprises active microbes and fermenting microbes in total CFU amounts within one order of magnitude, about two orders of magnitude, about three orders of magnitude, about four orders of magnitude, about 5 orders of magnitude, about 6 orders of magnitude, about 7 orders of magnitude, about 8 orders of magnitude, about 9 orders of magnitude, or about 10 orders of magnitude of each other. In some embodiments, an original dosage form of the disclosed microbial consortium comprises active microbes and synthesizing microbes in total CFU amounts within one order of magnitude, about two orders of magnitude, about three orders of magnitude, about four orders of magnitude, about 5 orders of magnitude, about 6 orders of magnitude, about 7 orders of magnitude, about 8 orders of magnitude, about 9 orders of magnitude, or about 10 orders of magnitude of each other. In some embodiments, an original dosage form of the disclosed microbial consortium comprises fermenting microbes and synthesizing microbes in total CFU amounts within one order of magnitude, about two orders of magnitude, about three orders of magnitude, about four orders of magnitude, about 5 orders of magnitude, about 6 orders of magnitude, about 7 orders of magnitude, about 8 orders of magnitude, about 9 orders of magnitude, or about 10 orders of magnitude of each other.

### Microbial Consortia Design

In some embodiments, microbial consortia disclosed herein are designed to meet one or more than one of the following criteria:
(i) an ability to eliminate or reduce levels of a first metabolic substrate causing or contributing to a disease in an animal;
(ii) an ability to metabolize or convert one or more than one metabolite produced by the metabolism of the first metabolic substrate;
(iii) an ability to metabolize one or more than one nutrient typically found in the human diet;
(iv) an ability to fulfill unique and potentially beneficial biological functions in the gastrointestinal (GI) tract (e.g., bile salt hydrolase activity or butyrate production);
(v) an ability to engraft in various biological niches and physical and metabolic compartments of the GI tract of an animal;
(vi) an ability to increase biomass upon engraftment in the GI tract;
(vii) an ability to have longitudinal stability in the GI tract of an animal;
(viii) an ability to increase the flux of a precursor of the first metabolic substrate into a biochemical pathway that converts said precursor into a metabolite that is not the first metabolic substrate;
(ix) diversity of component microbial species across one or more than one taxonomic phyla; and
(x) natural prevalence of component microbial species in the GI tract of healthy adults.

In some embodiments, the microbial consortia of the present invention are designed to comprise a plurality of active microbes capable of metabolizing a first metabolic substrate that causes or contributes to disease in an animal. For example, in some embodiments, the first metabolic substrate may be selected from, but not limited to, oxalate and a bile acid (e.g., lithocholic acid (LCA), deoxycholic acid (DCA)). In some embodiments, the microbial consortium is designed to be capable of metabolizing the first metabolic substrate across a variety of pH ranges found within the GI tract (e.g., pH 4 to 8). In some embodiments, the microbial consortium is designed to be capable of metabolizing the first metabolic substrate in the presence of various concentrations of first metabolic substrate as they exist in different regions of the GI tract.

For example, in designing which active microbes to include in a microbial consortium for the treatment of primary or secondary hyperoxaluria, an in vitro colorimetric assay (e.g., as described in Example 3 below) can be used to measure the capacity of a candidate microbe to metabolize oxalate in a sample. Microbes capable of reducing the concentration of oxalate present in a sample by at least 20%, by at least 30%, by at least 40%, by at least 50%, by at least 60%, by at least 70%, or by at least 80% can be included in a microbial consortium disclosed herein.

In other embodiments, an in vivo mouse assay can be used to measure the efficacy of a designed microbial consortium of the present invention in reducing the concentration of oxalate present in a sample of blood, serum, bile, stool, or urine when administered to a subject. Concentrations of oxalate in a blood, serum, bile, stool or urine sample can be measured using a liquid chromatography-mass spectrometry (LC-MS) method as described in Example 4, below. Microbial consortia capable of reducing blood, serum, bile, stool, or urine oxalate levels by at least 20%, by at least 30%, by at least 40%, by at least 50%, by at least 60%, by at least 70%, or by at least 80% as compared to levels in untreated controls or pre-administration levels can be candidates for further evaluation for the treatment of primary or secondary hyperoxaluria.

In some embodiments, a microbial consortium disclosed herein is designed to metabolize one or more than one metabolite produced by the plurality of active microbes, wherein the one or more than one metabolite inhibits metabolism of the plurality of active microbes. In some embodiments, the microbial consortia are designed to maximize consumption and/or production of a defined set of metabolites using a minimal number of strains. For example, in some embodiments, a microbial consortium is designed to include a microbe that metabolizes formate produced by the plurality of active microbes, wherein the presence of formate inhibits the metabolism of oxalate by the plurality of active microbes, e.g., in a negative feedback loop. In some embodiments, a microbial consortium is designed to include microbes that catalyze the fermentation of polysaccharides to one or more than one of acetate, acetoin, 2-oxoglutarate, propionate, 1,3-propanediol, succinate, ethanol, lactate, butyrate, 2,3-butanediol, acetone, butanol, formate, H₂, and CO₂. In some embodiments, a microbial consortium is designed to catalyze the fermentation of amino acids to one or more than one of acetate, propionate, butanoate, butyrate, isobutyrate, 2-methylbutyrate, isovalerate, isocaproate, 3-phenylpropanoate, phloretate, 3-(1H-indol-3-yl)propanoate, 5-aminopentanoate, H₂, H₂S, and CO₂. In some embodiments, the microbial consortium is designed to catalyze the synthesis of one or more than one of the group consisting of methane from H₂ and CO₂, methane from formate and H₂, acetate from H₂ and CO₂, acetate from formate and H₂, acetate and sulfide from H₂, CO₂, and sulfate, propionate and CO₂ from succinate, succinate from H₂ and fumarate; synthesis of succinate from formate and fumarate, and butyrate, acetate, H₂, and CO₂ from lactate. In some embodiments, the microbial consortium is designed to catalyze the deconjugation of conjugated bile acids to produce primary bile acids, the conversion of cholic acid (CA) to 7-oxocholic acid, the conversion of 7-oxocholic acid to 7-beta-cholic acid (7betaCA), the conversion of chenodeoxycholic acid (CDCA) to 7-oxochenodeoxycholic acid, and/or the conversion of 7-oxochenodeoxycholic acid to ursodeoxycholic acid (UDCA).

In some embodiments, a microbial consortium disclosed herein is designed to metabolize one or more than one metabolite produced by the plurality of active microbes, wherein the one or more than one metabolite inhibits metabolism of the plurality of active microbes. In some embodiments, the microbial consortia are designed to maximize consumption and/or production of a defined set of metabolites using a minimal number of strains. For example, in some embodiments, a microbial consortium is designed to include a microbe that metabolizes formate produced by the plurality of active microbes, wherein the presence of formate inhibits the metabolism of oxalate by the plurality of active microbes, e.g., in a negative feedback loop. In some embodiments, a microbial consortium is designed to include microbes that catalyze the fermentation of polysaccharides to one or more than one of acetate, propionate, succinate, lactate, butyrate, formate, H₂, and CO₂. In some embodiments, a microbial consortium is designed to catalyze the fermentation of amino acids to one or more than one of acetate, propionate, butyrate, isobutyrate, 2-methylbutyrate, isovalerate, isocaproate, H₂, H₂S, and CO₂. In other embodiments, a microbial consortium is designed to include microbes that catalyze the synthesis of one or more than one of methane from formate and H₂; acetate from H₂ and CO₂; acetate from formate and H₂; acetate and sulfide from H₂, CO₂, and sulfate; propionate and CO₂ from succinate; succinate from H₂ and fumarate; synthesis of succinate from formate and fumarate and butyrate, acetate, H₂, and CO₂ from lactate.

In some embodiments, microbial consortia are designed to include microbes capable of metabolizing one or more nutrient typically found in a broad spectrum of human diets. For example, in some embodiments, microbial consortia are designed include microbes capable of metabolizing one or more than one of oxalate, fructan, inulin, glucuronoxylan, arabinoxylan, glucomannan, β-mannan, dextran, starch, arabinan, xyloglucan, galacturonan, β-glucan, galactomannan, rhamnogalacturonan I, rhamnogalacturonan II, arabinogalactan, mucin O-linked glycans, yeast α-mannan, yeast β-glucan, chitin, alginate, porphyrin, laminarin, carrageenan, agarose, alternan, levan, xanthan gum, galactooligosaccharides, hyaluronan, chondrointin sulfate, dermatan sulfate, heparin sulfate, keratan sulfate, phenylalanine, tyrosine, tryptophan, leucine, valine, isoleucine, glycine, proline, asparagine, glutamine, aspartate, glutamate, cysteine, lysine, arginine, serine, methionine, alanine, arginine, histidine, ornithine, citrulline, carnitine, hydroxyproline, cholic acid, chenodeoxycholic acid, taurochenodeoxycholic acid, glycochenodeoxycholic acid, cholesterol, cinnamic acid, coumaric acid, sinapinic acid, ferulic acid, caffeic acid, quinic acid, chlorogenic acid, catechin, epicatechin, gallic acid, pyrogallol, catechol, quercetin, myricetin, campherol, luteolin, apigenin, naringenin, and hesperidin. In some embodiments, microbial consortia are designed to enrich for consumption of dietary carbon and energy sources. In other embodiments, microbial consortia are designed to enrich for the production or consumption of host metabolites, including bile acids, sugars, amino acids, vitamins, short-chain fatty acids, and gasses.

In some embodiments, microbial consortia are designed to include microbes having potentially beneficial biological functions in the GI tract. For example, microbial consortia are designed to include microbial strains having robust and/or redundant bile salt hydrolase (BSH) activity, so that deconjugation of primary bile acids can occur despite differences in host physiology, diet, plurality of active microbes present in the microbial consortium, or the pre-existing composition of the conjugated bile acid pool. In other embodiments, microbial consortia are designed to include microbial strains capable of producing butyrate from the fermentation of dietary fiber in the GI tract, which contributes to intestinal homeostasis, energy metabolism, anti-inflammatory processes, enhancement of intestinal barrier function, and mucosal immunity.

In some embodiments, microbial consortia described herein are designed to be able to engraft in various biological niches and physical and metabolic compartments of the GI tract of an animal (e.g., a human).

As used herein, “engraftment” (and grammatical variants thereof, e.g., “engraft”) refers to the ability of a microbial strain or microbial community to establish in one or more niches of the gut of an animal. Operationally, a microbial strain or microbial consortium is “engrafted” if evidence of its establishment, post-administration, can be obtained. In some embodiments, that evidence is obtained by molecular identification (e.g., Matrix-Assisted Laser Desorption/Ionization Time-Of-Flight Mass Spectrometry (MALDI-TOF MS), 16S rRNA sequencing, or genomic sequencing) of a sample obtained from the animal. In some embodiments, the sample is a stool sample. In some embodiments, the sample is a biopsy sample taken from the gut of the animal (e.g., from a location along the gastrointestinal tract of the animal). Engraftment may be transient or may be persistent. In some embodiments, transient engraftment means that the microbial strain or microbial community can no longer be detected in an animal to which it has been administered after the lapse of about 1 week, about 2 weeks, about three weeks, about 1 month, about 2 months, about 3 months, about 4 months, about 6 months, about 8 month, about 10 months, about 1 year, about 1.5 years, or about 2 years.

For example, microbial consortia are designed to be capable of engrafting into one or more than one niche of the gastrointestinal tract whose composition varies according to a number of environmental factors including, but not limited to, the particular physical compartment of the gastrointestinal tract, the chemical and physicochemical properties of the niche environment (e.g., gastrointestinal motility, pH), the metabolic substrate composition of the niche environment, and other co-inhabiting commensal microbial species. To analyze engraftment of a designed microbial consortium described herein, an in vivo assay can be used as described in Example 8, wherein stool samples from treated mice are analyzed for the presence of specific microbial strains comprising the microbial consortium by whole genome shotgun sequencing of microbial DNA extracted from fecal pellets and sequence reads mapped against a comprehensive database of complete, sequenced genomes of all the defined microbial strains comprising the microbial consortium.

In some embodiments, a microbial consortium described herein is designed to include microbes that support the growth and increase the biomass of one or more than one other microbe in the consortium when engrafted in the GI tract of an animal (e.g., a human). For example, in some embodiments, microbial consortia are designed to promote co-culturability and/or ecological stability of one or more than one microbial strain of the consortium.

In some embodiments, a microbial consortium described herein is designed to include one or more than one microbe having longitudinal stability in the GI tract of an animal (e.g., a human) despite transient or long-term changes to the gastrointestinal niche due to modifications in diet, the presence or absence of disease, or other physiological or environmental factors. In some embodiments, longitudinal stability of a community refers to the ability of a microbial consortium to persist (i.e. remain engrafted) in the GI tract of an animal following microbial challenge. In some embodiments, when given sufficient time to permit colonization of microbial challenge strains in the GI tract of an animal engrafted with a microbial consortium, longitudinal stability can be defined as one where at least 50%, at least 60%, at least 70%, at least 80%, at least 90%, at least 95%, at least 96%, at least 97%, at least 98%, or at least 99% of the defined microbial strains are detectable by metagenomic analysis. For example, in some embodiments, metagenomic analysis comprises whole genome shotgun sequencing analysis.

In other embodiments, longitudinal stability of a community refers to the characteristic of microbial strains comprising a consortium to maintain a metabolic phenotype over a period of time or following microbial challenge. For example, in some embodiments, defined microbial strains comprising a consortium can maintain a metabolic phenotype for at least 1 week, at least 2 weeks, at least 3 weeks, at least 4 weeks, at least 6 weeks, at least 8 weeks, at least 10 weeks, at least 12 weeks, at least 4 months, at least 6 months at least 8 months, at least 10 months, at least 1 year, at least 1.5 years, or at least 2 years.

In some embodiments, a longitudinal stability can be defined as one where the defined microbial strains comprising a consortium maintain the one or more metabolic phenotype of mucin degradation, polysaccharide fermentation, hydrogen utilization, succinate metabolism, butyrate production, amino acid metabolism, bile acid metabolism, CO₂ fixation, formate metabolism, methanogenesis, acetogenesis, hydrogen production, or propionate production over a period of time or following microbial challenge.

In some embodiments, a microbial consortium is designed to include one or more than one microbe capable of increasing the flux of a precursor of the first metabolic substrate into a biochemical pathway that converts said precursor into a metabolite that is not the first metabolic substrate. For example, in some embodiments, a microbial consortium can be designed to include microbial strains having robust 7α-HSDH and 7β-HSDH activity, which direct precursors of DCA and LCA first metabolic substrates (CA and CDCA, respectively) down biochemical pathways producing 7betaCA and UDCA.

In some embodiments, microbial consortia described herein are designed to include representative microbial strains isolated from a healthy donor fecal sample, with the exception of species known to be associated with pathogenesis, which represent microbial species belonging to a diverse array of taxonomic phyla including, Bacteroidetes, Firmicutes, Actinobacteria, Proteobacteria, Verrucomicrobia and Euryarchaeota. In some embodiments, microbial consortia having phylogenetic diversity are less sensitive to perturbations in the GI environment and are more stably engrafted For example, in some embodiments, microbial consortia can be designed to include one or more than one microbial species from Bacteroidetes, Firmicutes, Actinobacteria, Proteobacteria, Verrucomicrobia, or Euryarchaeota.

In some embodiments, microbial consortia can be designed to include one or more than one microbial species from Bacteroidetes and Firmicutes, Bacteroidetes and Actinobacteria, Bacteroidetes and Proteobacteria, Bacteroidetes and Verrucomicrobia, Bacteroidetes and Euryarchaeota, Firmicutes and Actinobacteria, Firmicutes and Proteobacteria, Firmicutes and Verrucomicrobia, Firmicutes and Euryarchaeota, Actinobacteria and Proteobacteria, Actinobacteria and Verrucomicrobia, Actinobacteria and Euryarchaeota, Proteobacteria and Verrucomicrobia, Proteobacteria and Euryarchaeota, or Verrucomicrobia and Euryarchaeota.

In some embodiments, microbial consortia can be designed to include one or more than one microbial species from: Bacteroidetes, Firmicutes, and Actinobacteria; Bacteroidetes, Firmicutes, and Proteobacteria; Bacteroidetes, Firmicutes, and Verrucomicrobia; Bacteroidetes, Firmicutes and Euryarchaeota; Bacteroidetes, Actinobacteria, and Proteobacteria; Bacteroidetes, Actinobacteria, and Verrucomicrobia; Bacteroidetes, Actinobacteria, and Euryarchaeota; Bacteroidetes, Proteobacteria, and Verrucomicrobia; Bacteroidetes, Proteobacteria, and Euryarchaeota; Bacteroidetes, Verrucomicrobia, and Euryarchaeota; Firmicutes, Actinobacteria, and Proteobacteria; Firmicuates, Actinobacteria, and Verrucomicrobia; Firmicuates, Actinobacteria, and Euryarchaeota; Firmicuates, Proteobacteria, and Verrucomicrobia; Firmicuates, Proteobacteria, and Euryarchaeota; Firmicutes, Verrucomicrobia, and Euryarchaeota; Actinobacteria, Proteobacteria, and Verrrucomicrobia; Actinobacteria, Proteobacteria, and Euryarchaeota; or Proteobacteria, Verrucomicrobia, and Euryarchaeota.

In some embodiments, microbial consortia can be designed to include one or more than one microbial species from: Bacteoidetes, Firmicutes, Actinobacteria, and Proteobacteria; Bacteoidetes, Firmicutes, Actinobacteria and Verrucomicrobia; Bacteoidetes, Firmicutes, Actinobacteria, and Euryarchaeota; Bacteroidetes, Actinobacteria, Proteobacteria, and Verrucomicrobia; Bacteroidetes, Actinobacteria, Proteobacteria, and Euryarchaeota; Bacteroidetes, Proteobacteria, Verrucomicrobia, and Euryarchaeota; Firmicutes, Actinobacteria, Proteobacteria, and Verrucomicrobia; Firmicutes, Actinobacteria, Proteobacteria, and Euryarchaeota; Firmicuates, Proteobacteria, Verrucomicrobia, and Euryarchaeota; or Actinobacteria, Proteobacteria, Verrucomicrobia, and Euryarchaeota.

In some embodiments, microbial consortia can be designed to include one or more than one microbial species from: Bacteoidetes, Firmicutes, Actinobacteria, Proteobacteria, and Verrucomicrobia; Bacteoidetes, Firmicutes, Actinobacteria, Proteobacteria, and Euryarchaeota; Bacteroidetes, Firmicutes, Actinobacteria, Verrucomicrobia, and Euryarchaeota; Bacteoidetes, Firmicutes, Proteobacteria, Verrucomicrobia, and Eurarchaeota; Bacteoidetes, Actinobacteria, Proteobacteria, Verrucomicrobia, and Eurarchaeota; or Firmicutes, Actinobacteria, Proteobacteria, Verrucomicrobia, and Eurarchaeota.

In some embodiments, microbial consortia can be designed to include one or more than one microbial species from: Bacteoidetes, Firmicutes, Actinobacteria, Proteobacteria, Verrucomicrobia, and Euryarchaeota.

For example, in some embodiments, a microbial consortium can be designed to include one or more than one Bacteroidetes strain listed in Table 4. In some embodiments, a microbial consortium can be designed to include a Bacteroidetes strain comprising a 16S sequence at least 90%, at least 91%, at least 92%, at least 93%, at least 94%, at least 95%, at least 96%, at least 97%, at least 98%, or at least 99% identical to any one of the Bacteroidetes microbes listed in Table 4. In some embodiments, a microbial consortium can be designed to include a Bacteroidetes strain comprising a 16S sequence at least 80% identical to any one of the Bacteroidetes microbes listed in Table 4.

In some embodiments, a microbial consortium can be designed to include one or more than one Firmicutes strain listed in Table 4. In some embodiments, a microbial consortium can be designed to include a Firmicutes strain comprising a 16S sequence at least 90%, at least 91%, at least 92%, at least 93%, at least 94%, at least 95%, at least 96%, at least 97%, at least 98%, or at least 99% identical to any one of the Firmicutes microbes listed in Table 4. In some embodiments, a microbial consortium can be designed to include a Firmicutes strain comprising a 16S sequence at least 80% identical to any one of the Firmicutes microbes listed in Table 4.

In some embodiments, a microbial consortium can be designed to include one or more than one Actinobacteria strain listed in Table 4. In some embodiments, a microbial consortium can be designed to include a Actinobacteria strain comprising a 16S sequence at least 90%, at least 91%, at least 92%, at least 93%, at least 94%, at least 95%, at least 96%, at least 97%, at least 98%, or at least 99% identical to any one of the Actinobacteria microbes listed in Table 4. In some embodiments, a microbial consortium can be designed to include a Actinobacteria strain comprising a 16S sequence at least 80% identical to any one of the Actinobacteria microbes listed in Table 4.

In some embodiments, a microbial consortium can be designed to include one or more than one Proteobacteria strain listed in Table 4. In some embodiments, a microbial consortium can be designed to include a Proteobacteria strain comprising a 16S sequence at least 90%, at least 91%, at least 92%, at least 93%, at least 94%, at least 95%, at least 96%, at least 97%, at least 98%, or at least 99% identical to any one of the Proteobacteria microbes listed in Table 4. In some embodiments, a microbial consortium can be designed to include a Proteobacteria strain comprising a 16S sequence at least 80% identical to any one of the Proteobacteria microbes listed in Table 4.

In some embodiments, a microbial consortium can be designed to include one or more than one Verrucomicrobia strain listed in Table 4. In some embodiments, a microbial consortium can be designed to include a Verrucomicrobia strain comprising a 16S sequence at least 90%, at least 91%, at least 92%, at least 93%, at least 94%, at least 95%, at least 96%, at least 97%, at least 98%, or at least 99% identical to any one of the Verrucomicrobia microbes listed in Table 4. In some embodiments, a microbial consortium can be designed to include a Verrucomicrobia strain comprising a 16S sequence at least 80% identical to any one of the Verrucomicrobia microbes listed in Table 4.

In some embodiments, a microbial consortium can be designed to include Methonobrevibacter smithii. In some embodiments, a microbial consortium can be designed to include a Methonobrevibacter smithii strain comprising a 16S sequence at least 90%, at least 91%, at least 92%, at least 93%, at least 94%, at least 95%, at least 96%, at least 97%, at least 98%, or at least 99% identical to SEQ ID NO: 292. In some embodiments, a microbial consortium can be designed to include a Methonobrevibacter smithii strain comprising a 16S sequence at least 80% identical to SEQ ID NO: 292.

In some embodiments, a microbial consortium is designed such that when administered to a subject the plurality of active microbes and the supportive community of microbes have one or more than one synergistic effect. For example, in some embodiments administration of a microbial consortium comprising the plurality of active microbes in combination with the supportive community of microbes results in an enhanced metabolization of a first metabolic substrate than achieved by administration of either the plurality of active microbes or supportive community of microbes alone. For example, in some embodiments administration of a microbial consortium results in enhanced oxalate metabolism (e.g., as measured by urinary oxalate levels) in a subject as compared to a subject administered with either a plurality of active microbes or a supportive community of microbes alone. In other embodiments, administration of a microbial consortium results in enhanced conversion of primary bile acids (e.g., DCA and/or LCA) in a subject as compared to a subject administered with either a plurality of active microbes or a supportive community of microbes alone. In some embodiments, a microbial composition comprising the plurality of active microbes in combination with the supportive community of microbes results in enhanced GI engraftment than the engraftment achieved by administration of either the plurality of active microbes or supportive community of microbes alone. In some embodiments, a microbial composition comprising the plurality of active microbes in combination with the supportive community of microbes results in greater biomass in the GI tract than the biomass achieved by administration of either the plurality of active microbes or supportive community of microbes alone. In some embodiments, a microbial composition comprising the plurality of active microbes in combination with the supportive community of microbes results in enhanced longitudinal stability than the stability achieved by administration of either the plurality of active microbes or supportive community of microbes alone. In some embodiments, a microbial composition comprising the plurality of active microbes in combination with the supportive community of microbes results in enhanced clinical efficacy in the treatment of a disease than the efficacy achieved by administration of either the plurality of active microbes or supportive community of microbes alone.

In some embodiments, a microbial consortium is designed to comprise 20 to 300, 20 to 250, 20 to 200, 20 to 190, 20 to 180, 20 to 170, 20 to 160, 20 to 150, 20 to 140, 20 to 130, 20 to 120, 20 to 110, 20 to 100, 20 to 90, 20 to 80, 20 to 70, 20 to 60, 20 to 50, 50 to 300, 50 to 250, 50 to 200, 50 to 190, 50 to 180, 50 to 170, 50 to 160, 50 to 150, 50 to 140, 50 to 130, 50 to 120, 50 to 110, 50 to 100, 50 to 90, 50 to 80, 50 to 70, 50 to 60, 100 to 300, 100 to 250, 100 to 200, 100 to 190, 100 to 180, 100 to 170, 100 to 160, 100 to 150, 100 to 140, 100 to 130, 100 to 120, 100 to 110, 70 to 80, 80 to 90, or 150 to 160 microbial strains, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 4.

In some embodiments, a microbial consortium is designed to comprise 20 to 160, 30 to 160, 40 to 160, 50 to 160, 60 to 160, 70 to 160, 80 to 160, 90 to 160, 100 to 160, 110 to 160, 120 to 160, 130 to 160, 140 to 160, 150 to 160, 20 to 140, 30 to 140, 40 to 140, 50 to 140, 60 to 140, 70 to 140, 80 to 140, 90 to 140, 100 to 140, 110 to 140, 120 to 140, 130 to 140, 20 to 120, 30 to 120, 40 to 120, 50 to 120, 60 to 120, 70 to 120, 80 to 120, 90 to 120, 100 to 120, 110 to 120, 20 to 100, 30 to 100, 40 to 100, 50 to 100, 60 to 100, 70 to 100, 80 to 100, 90 to 100, 20 to 80, 30 to 80, 40 to 80, 50 to 80, 60 to 80, or 70 to 80 microbial strains, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 22.

In some embodiments, a microbial consortium is designed to comprise 20 to 104, 40 to 104, 60 to 104, 80 to 104, 100 to 104, 20 to 80, 40 to 80, 60 to 80, 20 to 60, or 40 to 60 microbial strains, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 23.

In some embodiments, a microbial consortium is designed to comprise 20 to 104, 40 to 104, 60 to 104, 80 to 104, 100 to 104, 20 to 80, 40 to 80, 60 to 80, 20 to 60, or 40 to 60 microbial strains, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 24.

In some embodiments, a microbial consortium is designed to comprise 20 to 158, 30 to 158, 40 to 158, 50 to 158, 60 to 158, 70 to 158, 80 to 158, 90 to 158, 100 to 158, 110 to 158, 120 to 158, 130 to 158, 140 to 158, 150 to 158, 20 to 140, 30 to 140, 40 to 140, 50 to 140, 60 to 140, 70 to 140, 80 to 140, 90 to 140, 100 to 140, 110 to 140, 120 to 140, 130 to 140, 20 to 120, 30 to 120, 40 to 120, 50 to 120, 60 to 120, 70 to 120, 80 to 120, 90 to 120, 100 to 120, 110 to 120, 20 to 100, 30 to 100, 40 to 100, 50 to 100, 60 to 100, 70 to 100, 80 to 100, 90 to 100, 20 to 80, 30 to 80, 40 to 80, 50 to 80, 60 to 80, or 70 to 80 microbial strains, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 20.

In some embodiments, a microbial consortium is designed to comprise 20 to 152, 30 to 152, 40 to 152, 50 to 152, 60 to 152, 70 to 152, 80 to 152, 90 to 152, 100 to 152, 110 to 152, 120 to 152, 130 to 152, 140 to 152, 150 to 152, 20 to 140, 30 to 140, 40 to 140, 50 to 140, 60 to 140, 70 to 140, 80 to 140, 90 to 140, 100 to 140, 110 to 140, 120 to 140, 130 to 140, 20 to 120, 30 to 120, 40 to 120, 50 to 120, 60 to 120, 70 to 120, 80 to 120, 90 to 120, 100 to 120, 110 to 120, 20 to 100, 30 to 100, 40 to 100, 50 to 100, 60 to 100, 70 to 100, 80 to 100, 90 to 100, 20 to 80, 30 to 80, 40 to 80, 50 to 80, 60 to 80, or 70 to 80 microbial strains, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 16.

In some embodiments, a microbial consortium is designed to comprise 20 to 88, 40 to 88, 60 to 88, 80 to 88, 20 to 80, 40 to 80, 60 to 80, 20 to 60, or 40 to 60 microbial strains, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 17.

In some embodiments a microbial consortium is designed to comprise 20 to 89, 40 to 89, 60 to 89, 80 to 89, 20 to 80, 40 to 80, 60 to 80, 20 to 60, or 40 to 60 microbial strains, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 18.

In some embodiments, a microbial consortium is designed to comprise 20 to 75, 40 to 75, 60 to 75, 80 to 75, 20 to 60, or 40 to 60 microbial strains, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 19.

In some embodiments, a microbial consortium is designed to comprise 2 to 51, 5 to 51, 10 to 51, 20 to 51, 30 to 51, or 40 to 51 Actinobacteria; 10 to 102, 20 to 102, 30 to 102, 40 to 102, 50 to 102, 60 to 102, 70 to 102, 80 to 102, 90 to 102, 10 to 50, 20 to 50, 30 to 50, or 40 to 50 Bacteroidetes; 1 or 2 Euryacrchaeota; 20 to 197, 40 to 197, 60 to 197, 80 to 197, 100 to 197, 120 to 197, 140 to 197, 160 to 197, 180 to 197, 20 to 150, 40 to 150, 60 to 150, 80 to 150, 100 to 150, 120 to 150, 140 to 150, 20 to 100, 40 to 100, 60 to 100, or 80 to 100 Firmicutes; 2 to 24, 8 to 24, 12 to 24, 18 to 24, or 20 to 24 Proteobacteria; and 1 Verrucomicrobia, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 4.

In some embodiments, a microbial consortium is designed to comprise 2 to 20, 5 to 20, 10 to 20, or 15 to 20 Actinobacteria; 2 to 48, 10 to 48, 20 to 48, 30 to 48, 40 to 48 Bacteroidetes; 2 to 76, 10 to 76, 20 to 76, 30 to 76, 40 to 76, 50 to 76, 60 to 76, 70 to 76, 2 to 50, 10 to 50, 20 to 50, 30 to 50, 40 to 50 Firmicutes; 2 to 7 Proteobacteria; and 1 Verrucomicrobia, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 16.

In some embodiments, a microbial consortium is designed to comprise 2 to 22, 10 to 22, or 20 to 22 Actinobacteria; 2 to 27, 10 to 27, or 20 to 27 Bacteroidetes; 2 to 29, 10 to 29, or 20 to 29 Firmicutes; 1 to 9 Proteobacteria; and 1 Verrucomicrobia, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 17.

In some embodiments, a microbial consortium is designed to comprise 2 to 18 or 10 to 18 Actinobacteria; 2 to 27, 10 to 27, or 20 to 27 Bacteroidetes; 2 to 38, 10 to 38, 20 to 38, 30 to 38 Firmicutes; and 2 to 6 Proteobacteria, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 18.

In some embodiments, a microbial consortium is designed to comprise 2 to 7 Actinobacteria; 2 to 20 or 10 to 20 Bacteroidetes; 2 to 38, 10 to 38, 20 to 38, or 30 to 38 Firmicutes; 2 to 8 Proteobacteria; and 1 Verrucomicrobia, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 19.

In some embodiments, a microbial consortium is designed to comprise 2 to 20 or 10 to 20 Actinobacteria; 2 to 42, 10 to 42, 20 to 42, 30 to 42, or 40 to 42 Bacteroidetes; 2 to 84, 10 to 84, 20 to 84, 30 to 84, 40 to 84, 50 to 84, 60 to 84, 70 to 84, 80 to 84, 2 to 50, 10 to 50, 20 to 50, 30 to 50, or 40 to 50 Firmicutes; 2 to 11 Proteobacteria; and 1 Verrucomicrobia, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 20.

In some embodiments, a microbial consortium is designed to comprise 2 to 20 or 10 to 20 Actinobacteria; 2 to 44, 10 to 44, 20 to 44, 30 to 44, or 40 to 44 Bacteroidetes; 1 or 2 Euryarcheota; 2 to 83, 10 to 83, 20 to 83, 30 to 83, 40 to 83, 50 to 83, 60 to 83, 70 to 83, 80 to 83, 2 to 50, 10 to 50, 20 to 50, 30 to 50, or 40 to 50 Firmicutes; 2 to 10 Proteobacteria; and 1 Verrucomicrobia, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 22.

In some embodiments, a microbial consortium is designed to comprise 2 to 15 or 10 to 15 Actinobacteria; 2 to 25, 10 to 25, or 20 to 25 Bacteroidetes; 2 to 55, 10 to 55, 20 to 55, 30 to 55, 40 to 55, 50 to 55, 2 to 25, 10 to 25, or 20 to 25 Firmicutes; 2 to 8 Proteobacteria; and 1 Verrucomicrobia, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 23.

In some embodiments, a microbial consortium is designed to comprise 2 to 11 Actinobacteria; 2 to 28, 10 to 28, or 20 to 28 Bacteroidetes; 1 Euryarchaeota; 2 to 56, 10 to 56, 20 to 56, 30 to 56, 40 to 56, 50 to 56, 2 to 25, 10 to 25, or 20 to 25 Firmicutes; 2 to 7 Proteobacteria; and 1 Verrucomicrobia, each comprising a 16S sequence at least 80%, at least 90%, at least 91%, at least 92%, at least 93%, at least 94%, at least 95%, at least 96%, at least 97%, at least 98%, or at least 99% identical to any one of the microbes listed in Table 24.

### Isolation and Propagation of Microbial Strains

Active and supportive microbial strains can be derived from human donor fecal samples, or purchased from the American Type Culture Collection (ATCC; www.atcc.org), the Leibniz institute DSMZ (www.dsmz.de), or BEI Resources (www.beiresources.org). Microbial strains purchased from a depository can be cultured according to depository instructions and microbial strains derived from human donors can be cultured according to the media conditions described in Table 3, below.

Fecal donors can be selected based on multiple criteria, including a health and medical history questionnaire, physical exam, and blood and stool tests for assessing pathogen-free status. Upon collection of a stool sample from a donor, stool samples can cultured in an anaerobic chamber (5% CO₂, 5% H₂, 90% N₂) and microbial strains isolated by making serial dilution aliquots of the stool samples and plating said aliquots on a variety of microbial cultivation media suitable for growth of anaerobes. Specific enrichment techniques can be performed for species having particular metabolic capabilities, such as consumption or tolerance of oxalate or bile acids. In order to enrich for strains having oxalate metabolism capabilities, aliquots of the serially-diluted stool samples can be plated on agar growth media supplemented with varying concentrations of potassium oxalate (20 mM, 40 mM, 80 mM, 160 mM, or 200 mM). In order to enrich for species capable of metabolizing bile acids, aliquots of serially diluted stool samples can be plated on growth media supplemented with 2% bile. Archaea can be isolated by diluting fecal samples and plating on culture media containing a mixture of antibiotics that is lethal to both gram-positive and gram-negative bacteria. Microbial strain identification can be performed either by 16S rRNA gene sequencing or proteomic fingerprinting using high-throughput Matrix-Assisted Laser Desorption/Ionization Time-Of-Flight Mass Spectrometry (MALDI-TOF MS).

In some embodiments, methods of producing a microbial consortium described herein comprise individually culturing each of a plurality of active microbes and supportive microbes prior to combining the microbes to form the consortium. In other embodiments, methods of producing a microbial consortium described herein comprise culturing all of a plurality of active microbes and supportive microbes together. In still other embodiments, methods of producing a microbial consortium comprise individually culturing one or more than one microbial strain and co-culturing two or more microbial strains having compatible culture growth conditions, then combining together the individually-cultured microbial strains and co-cultured defined microbial strains to form a microbial consortium. In other embodiments, methods of producing a microbial consortium comprise individually culturing one or more than one microbial strain and co-culturing two or more microbial strains having compatible culture growth conditions, then combining together the individually-cultured microbial strains and co-cultured defined microbial strains to form a microbial consortium.

### Pharmaceutical Compositions

The present disclosure also provides pharmaceutical compositions that contain an effective amount of a microbial consortium described herein. The composition can be formulated for use in a variety of delivery systems. One or more physiologically acceptable buffer(s) or carrier(s) can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In some embodiments, microbial cells of the present invention are harvested by microfiltration and centrifugation. In some embodiments, microfiltration is done with a membrane comprising a nonreactive polymer. For example, in some embodiments, said membrane comprises Polyvinylidene fluoride, Polysulfones, or nitrocellulose. In some embodiments, a membrane for microfiltration has a pore size of approximately 0.2 to 0.45 μm. In some embodiments, the cells are centrifuged at approximately 1000 to 30000, 5000 to 30000, 10000 to 30000, 15000 to 30000, 20000 to 30000, 25000 to 30000, 1000 to 25000, 5000 to 25000, 10000 to 25000, 15000 to 25000, 20000 to 25000, 1000 to 20000, 5000 to 20000, 10000 to 20000, 15000 to 20000, 1000 to 15000, 5000 to 15000, 10000 to 15000, 1000 to 10000, 5000 to 10000, 1000 to 5000 g force. In some embodiments, the cells are concentrated to approximately 1×10⁶ to 1×10¹², 1×10⁷ to 1×10¹², 1×10⁸ to 1×10¹², 1×10⁹ to 1×10¹², 1×10¹⁰ to 1×10¹², 1×10¹¹ to 1×10¹², 1×10⁶ to 1×10¹¹, 1×10⁷ to 1×10¹¹, 1×10⁸ to 1×10¹¹, 1×10⁹ to 1×10¹¹, 1×10¹⁰ to 1×10¹¹, 1×10⁶ to 1×10¹⁰, 1×10⁷ to 1×10¹⁰, 1×10⁸ to 1×10¹⁰, 1×10⁹ to 1×10¹⁰, 1×10⁶ to 1×10⁹, 1×10⁷ to 1×10⁹, 1×10⁸ to 1×10⁹, 1×10⁶ to 1×10⁸, 1×10⁷ to 1×10⁸ 1×10⁶ to 1×10⁷ CFUs per milliliter.

In some embodiments, microbial cells of the present invention are frozen. In some embodiments, the microbial cells of the present invention are mixed with one or more cryoprotective agents (CPAs) before freezing. In some embodiments, the ratio of cells to CPA is approximately 25:1, 10:1, 5:1, 4:1, 3:1, 2:1, 1:1, 1:2, 1:3, 1:4, 1:5, 1:10, or 1:25. In some embodiments, a CPA comprises one or more of glycerol, maltodextrin, sucrose, inulin, trehalose, and alginate. In some embodiments, a CPA further comprises one or more antioxidants. In some embodiments, an antioxidant is selected from the list of cysteine, ascorbic acid, and riboflavin.

In some embodiments, the microbial cells of the present invention are lyophilized. In some embodiments, the lyophilized cells are used to make an orally-administered dose of the invention. In some embodiments, primary drying is conducted below approximately −20° C. In some embodiments, primary drying is followed by a secondary drying at a higher temperature, e.g. greater than 0° C., greater than 5° C., or greater than 10° C.

In some embodiments a pharmaceutical composition disclosed herein may comprise a microbial consortium of the present invention and one or more than one agent selected from, but not limited to: carbohydrates (e.g., glucose, sucrose, galactose, mannose, ribose, arabinose, xylose, fructose, maltose, cellobiose, lactose, deoxyribose, hexose); lipids (e.g. lauric acid (12:0) myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0)); minerals (e.g., chloride, sodium, calcium, iron, chromium, copper, iodine, zinc, magnesium, manganese, molybdenum, phosphorus, potassium, and selenium); vitamins (e.g., vitamin C, vitamin A, vitamin E, vitamin B 12, vitamin K, riboflavin, niacin, vitamin D, vitamin B6, folic acid, pyridoxine, thiamine, pantothenic acid, and biotin); buffering agents (e.g. sodium citrate, magnesium carbonate, magnesium bicarbonate, calcium carbonate, and calcium bicarbonate); preservatives (e.g., alpha-tocopherol, ascorbate, parabens, chlorobutanol, and phenol); binders (e.g., starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides); lubricants (e.g. magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil); dispersants (e.g., starch, alginic acid, polyvinylpyrrolidones, guar gum, kaolin, bentonite, purified wood cellulose, sodium starch glycolate, isoamorphous silicate, and microcrystalline cellulose); disintegrants (e.g., corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, tragacanth, sodium bicarbonate in combination with citric acid, and sodium bicarbonate in combination with tartaric acid); flavoring agents; sweeteners; and coloring agents. In some embodiments, additional nutrients such as oxalate or formate are added to support robust revival of specific strains from the capsule.

In certain embodiments, a microbial consortium of the present invention is administered orally as a lyophilized powder, capsule, tablet, troche, lozenge, granule, gel or liquid. In some embodiments, a microbial consortium of the present invention is administered as a tablet or pill and can be compressed, multiply compressed, multiply layered, and/or coated. For example, in some embodiments, a lyophilized powder is filled in “0”, “00”, or “000” size capsules to accommodate various strengths. In some embodiments the tablet or pill comprises an enteric coating.

### Therapeutic Applications

The present invention provides microbial consortia capable of engrafting into one or more than one niche of a gastrointestinal tract where it is capable of metabolizing a first metabolic substrate that causes or contributes to disease in an animal. In some embodiments, the animal is a mouse. In some embodiments, the animal is a germ-free mouse. In some embodiments, the animal is a mouse engrafted with a human microbiome. In some embodiments, the animal is a human.

In some embodiments of the invention, when administered to an animal, the animal is pre-treated with one or more antibiotics prior to administration of the microbial consortium. In some embodiments, the one or more antibiotics is selected from ampicillin, enrofloxacin, clarithromycin, and metronidazole. In some embodiments, the animal is pre-treated with a polyethylene glycol bowel-preparation procedure.

In some embodiments, when administered to an animal, the microbial consortium of the present invention significantly reduces the concentration of a first metabolic substrate present in the blood, serum, bile, stool or urine as compared to samples collected pretreatment from the same animal or from corresponding control animal that have not been administered with the microbial consortium. For example, in some embodiments, when administered to an animal on a high oxalate diet, the microbial consortium of the present invention significantly reduces the concentration of oxalate present in a sample of blood, serum, bile, stool or urine as compared to samples collected pretreatment from the same animal or from a corresponding control animal that has not been administered with the microbial consortium. As used herein, a “high oxalate diet” refers to a diet that induces a hyperoxaluria phenotype in an animal. For example, in some embodiments, an animal may be maintained on a high oxalate diet for 7 days to 1 month. In some embodiments, an animal may be maintained on a high oxalate diet for 7 days, 14 days, 21 days, or 1 month. In some embodiments, a high oxalate diet can have a calcium to oxalate molar ratio of less than 2.0. For example, in some embodiments, a high oxalate diet can have a calcium to oxalate molar ratio of about 0.1 to about 0.8. In some embodiments, an animal may be maintained on a grain-based diet that is rich in complex polysaccharides and nutritionally complete and given ad libitum drinking water supplemented with about 0.5% to 1% oxalate. In some embodiments, a control animal may be maintained on a diet as shown in Table 1 or an animal may be maintained on a high oxalate diet as shown in Table 2.

TABLE 1
Control Diet

| | | |
|---|---|---|
| Casein | 200 | mg/g |
| DL-Methionine | 3.0 | mg/g |
| Sucrose | 302.8 | mg/g |
| Corn Starch | 280.0 | mg/g |
| Corn Oil | 50.0 | mg/g |
| Inulin | 35.0 | mg/g |
| Pectin | 35.0 | mg/g |
| Cellulose | 25.0 | mg/g |
| Mineral Mix, Ca—P Deficient (79055) | 13.37 | mg/g |
| Potassium phosphate, monobasic | 11.4 | mg/g |
| Calcium chloride | 14.94 | mg/g |
| Sodium chloride | 19.48 | mg/g |
| Vitamin Mix, Teklad (40060) | 10.0 | mg/g |
| Ethoxyquin, antioxidant | 0.01 | mg/g |

TABLE 2
Oxalate Diet

| | | |
|---|---|---|
| Casein | 200 | mg/g |
| DL-Methionine | 3.0 | mg/g |
| Sucrose | 316.2 | mg/g |
| Corn Starch | 280.0 | mg/g |
| Corn Oil | 50.0 | mg/g |
| Inulin | 35.0 | mg/g |
| Pectin | 35.0 | mg/g |
| Cellulose | 25.0 | mg/g |
| Mineral Mix, Ca—P Deficient (79055) | 13.37 | mg/g |
| Potassium phosphate, monobasic | 11.4 | mg/g |
| Calcium chloride | 1.05 | mg/g |
| Sodium chloride | 16.23 | mg/g |
| Vitamin Mix, Teklad (40060) | 10.0 | mg/g |
| Ethoxyquin, antioxidant | 0.01 | mg/g |
| Sodium oxalate | 3.72 | mg/g |

In some embodiments, a microbial consortium of the present invention is administered to an animal on a diet supplemented with one or more bile acids. In some embodiments, the diet is supplemented with one or more of TCDCA, GCDCA, TCA, GCA, CA, CDCA, LCA, or DCA. For example, in some embodiments, an animal may be maintained on a diet supplemented with one or more bile acids for 7 days to 1 month. In some embodiments, an animal may be maintained on a diet supplemented with bile acids for 7 days, 14 days, 21 days, or 1 month.

In some embodiments, a microbial consortium of the present invention is used to treat a subject having or at risk of developing a metabolic disease or condition. For example, in some embodiments, the metabolic disease is primary hyperoxaluria. In some embodiments, the metabolic disease is secondary hyperoxaluria. In some embodiments, the metabolic disease is secondary hyperoxaluria associated with bowel resection surgery or IBD. In some embodiments, a microbial consortium of the present invention significantly reduces the concentration of oxalate present in a sample of blood, serum, bile, stool, or urine when administered to a subject by at least 20%, by at least 30%, by at least 40%, by at least 50%, by at least 60%, by at least 70%, or by at least 80% as compared to untreated subjects or pre-administration concentrations.

In some embodiments, a microbial consortium of the present invention significantly alters the profile and/or concentration of bile acids present in an animal. For example, in some embodiments, a microbial consortium of the present invention significantly alters the profile and/or concentration of Tβ-MCA, Tα-MCA, TUDCA, THDCA, TCA, 7β-CA, 7-oxo-CA, TCDCA, Tω-MCA, TDCA, α-MCA, β-MCA, ω-MCA, Muro-CA, d4-CA, CA, TLCA, UDCA, HDCA, CDCA, DCA, and LCA in an animal.

In some embodiments, a high-complexity defined gut microbial community of the present invention can be used to treat an animal having a cholestatic disease, such as, for example, primary sclerosing cholangitis, primary biliary cholangitis, progressive familial intrahepatic cholestasis, or nonalcoholic steatohepatitis. For example in some embodiments, the animal may be a mammal, and more particularly a human.

In some embodiments, a microbial consortium of the present invention can be administered via an enteric route. For example, in some embodiments, a microbial consortium is administered orally, rectally (e.g., by enema, suppository, or colonoscope), or by oral or nasal tube.

In some embodiments, a microbial consortium of the present invention can be administered to a specific location along the gastrointestinal tract. For example, in some embodiments, a microbial consortium can be administered into one or more than one gastrointestinal location including the mouth, esophagus, stomach, small intestine (duodenum, jejunum, ileum), large intestine (cecum, ascending colon, transverse colon, descending colon), or rectum. In some embodiments, a microbial consortium can be administered in all regions of the gastrointestinal tract.

### Dosages

In some embodiments, a microbial consortium of the present invention is administered in a dosage form having a total amount of microbial consortium of at least 1×10⁶ colony forming units (CFU) or above, at least 2×10⁶ CFU or above, at least 3×10⁶ CFU or above, at least 4×10⁶ CFU or above, at least 5×10⁶ CFU or above, at least 6×10⁶ CFU or above, at least 7×10⁶ CFU or above, at least 8×10⁶ CFU or above, at least 9×10⁶ CFU or above, at least 1×10⁷ CFU or above, at least 2×10⁷ CFU or above, at least 3×10⁷ CFU or above, at least 4×10⁷ CFU or above, at least 5×10⁷ CFU or above, at least 6×10⁷ CFU or above, at least 7×10⁷ CFU or above, at least 8×10⁷ CFU or above, at least 9×10⁷ CFU or above, 1×10⁸ CFU or above, at least 2×10⁸ CFU or above, at least 3×10⁸ CFU or above, at least 4×10⁸ CFU or above, at least 5×10⁸ CFU or above, at least 6×10⁸ CFU or above, at least 7×10⁸ CFU or above, at least 8×10⁸ CFU or above, at least 9×10⁸ CFU or above, 1×10⁹ CFU or above, at least 2×10⁹ CFU or above, at least 3×10⁹ CFU or above, at least 4×10⁹ CFU or above, at least 5×10⁹ CFU or above, at least 6×10⁹ CFU or above, at least 7×10⁹ CFU or above, at least 8×10⁹ CFU or above, at least 9×10⁹ CFU or above, 1×10¹⁰ CFU or above, at least 2×10¹⁰ CFU or above, at least 3×10¹⁰ CFU or above, at least 4×10¹⁰ CFU or above, at least 5×10¹⁰ CFU or above, at least 6×10¹⁰ CFU or above, at least 7×10¹⁰ CFU or above, at least 8×10¹⁰ CFU or above, at least 9×10¹⁰ CFU or above, 1×10¹¹ CFU or above, at least 2×10¹¹ CFU or above, at least 3×10¹¹ CFU or above, at least 4×10¹¹ CFU or above, at least 5×10¹¹ CFU or above, at least 6×10¹¹ CFU or above, at least 7×10¹¹ CFU or above, at least 8×10¹¹ CFU or above, at least 9×10¹¹ CFU or above, 1×10¹² CFU or above, at least 2×10¹² CFU or above, at least 3×10¹² CFU or above, at least 4×10¹² CFU or above, at least 5×10¹² CFU or above, at least 6×10¹² CFU or above, at least 7×10¹² CFU or above, at least 8×10¹² CFU or above, or at least 9×10¹² CFU or above.

In some embodiments, a microbial consortium of the present invention is administered in a dosage form having a total amount of microbial consortium of 0.1 ng to 500 mg, 0.5 ng to 500 mg, 1 ng to 500 mg, 5 ng to 500 mg, 10 ng to 500 mg, 50 ng to 500 mg, 100 ng to 500 mg, 500 ng to 500 mg, 1 μg to 500 mg, 5 μg to 500 mg, 10 μg to 500 mg, 50 μg to 500 mg, 100 μg to 500 mg, 500 μg to 500 mg, 1 mg to 500 mg, 5 mg to 500 mg, 10 mg to 500 mg, 50 mg to 500 mg, 100 mg to 500 mg, 0.1 ng to 100 mg, 0.5 ng to 100 mg, 1 ng to 100 mg, 5 ng to 100 mg, 10 ng to 100 mg, 50 ng to 100 mg, 100 ng to 100 mg, 500 ng to 500 mg, 1 μg to 100 mg, 5 μg to 100 mg, 10 us to 100 mg, 50 us to 100 mg, 100 μg to 100 mg, 500 μg to 100 mg, 1 mg to 500 mg, 5 mg to 100 mg, 10 mg to 100 mg, 50 mg to 100 mg, 0.1 ng to 50 mg, 0.5 ng to 50 mg, 1 ng to 50 mg, 5 ng to 50 mg, 10 ng to 50 mg, 50 ng to 50 mg, 100 ng to 50 mg, 500 ng to 500 mg, 1 μg to 50 mg, 5 μg to 50 mg, 10 μg to 50 mg, 50 us to 50 mg, 100 μg to 50 mg, 500 μg to 50 mg, 1 mg to 500 mg, 5 mg to 50 mg, 10 mg to 50 mg, 0.1 ng to 10 mg, 0.5 ng to 10 mg, 1 ng to 10 mg, 5 ng to 10 mg, 10 ng to 10 mg, 50 ng to 10 mg, 100 ng to 10 mg, 500 ng to 500 mg, 1 μg to 10 mg, 5 μg to 10 mg, 10 us to 10 mg, 50 μg to 10 mg, 100 μg to 10 mg, 500 μg to 10 mg, 1 mg to 500 mg, 5 mg to 10 mg, 0.1 ng to 5 mg, 0.5 ng to 5 mg, 1 ng to 5 mg, 5 ng to 5 mg, 10 ng to 5 mg, 50 ng to 5 mg, 100 ng to 5 mg, 500 ng to 500 mg, 1 μg to 5 mg, 5 μg to 5 mg, 10 μg to 5 mg, 50 us to 5 mg, 100 us to 5 mg, 500 μg to 5 mg, 1 mg to 500 mg, 0.1 ng to 1 mg, 0.5 ng to 1 mg, 1 ng to 1 mg, 5 ng to 1 mg, 10 ng to 1 mg, 50 ng to 1 mg, 100 ng to 1 mg, 500 ng to 500 mg, 1 μg to 1 mg, 5 μg to 1 mg, 10 μg to 1 mg, 50 μg to 1 mg, 100 μg to 1 mg, 500 us to 1 mg, 0.1 ng to 500 μg, 0.5 ng to 500 μg, 1 ng to 500 μg, 5 ng to 500 μg, 10 ng to 500 μg, 50 ng to 500 μg, 100 ng to 500 μg, 500 ng to 500 μg, 1 μg to 500 μg, 5 us to 500 μg, 10 us to 500 μg, 50 us to 500 μg, 100 μg to 500 μg, 0.1 ng to 100 μg, 0.5 ng to 100 μg, 1 ng to 100 μg, 5 ng to 100 μg, 10 ng to 100 μg, 50 ng to 100 μg, 100 ng to 100 μg, 500 ng to 100 μg, 1 μg to 100 μg, 5 μg to 100 μg, 10 μg to 100 μg, 50 μg to 100 μg, 0.1 ng to 50 μg, 0.5 ng to 50 μg, 1 ng to 50 μg, 5 ng to 50 μg, 10 ng to 50 μg, 50 ng to 50 μg, 100 ng to 50 μg, 500 ng to 50 μg, 1 μg to 50 μg, 5 μg to 50 μg, 10 μg to 50 μg, 0.1 ng to 10 μg, 0.5 ng to 10 μg, 1 ng to 10 μg, 5 ng to 10 μg, 10 ng to 10 μg, 50 ng to 10 μg, 100 ng to 10 μg, 500 ng to 10 μg, 1 μg to 10 μg, 5 μg to 10 μg, 0.1 ng to 5 μg, 0.5 ng to 5 μg, 1 ng to 5 μg, 5 ng to 5 μg, 10 ng to 5 μg, 50 ng to 5 μg, 100 ng to 5 μg, 500 ng to 5 μg, 1 μg to 5 μg, 0.1 ng to 1 μg, 0.5 ng to 1 μg, 1 ng to 1 μg, 5 ng to 1 μg, 10 ng to 1 μg, 50 ng to 1 μg, 100 ng to 1 μg, 500 ng to 1 μg, 0.1 ng to 500 ng, 0.5 ng to 500 ng, 1 ng to 500 ng, 5 ng to 500 ng, 10 ng to 500 ng, 50 ng to 500 ng, 100 ng to 500 ng, 0.1 ng to 100 ng, 0.5 ng to 100 ng, 1 ng to 100 ng, 5 ng to 100 ng, 10 ng to 100 ng, 50 ng to 100 ng, 0.1 ng to 50 ng, 0.5 ng to 50 ng, 1 ng to 50 ng, 5 ng to 50 ng, 10 ng to 50 ng, 0.1 ng to 10 ng, 0.5 ng to 10 ng, 1 ng to 10 ng, 5 ng to 10 ng, 0.1 ng to 5 ng, 0.5 ng to 5 ng, 1 ng to 5 ng, 0.1 ng to 1 ng, 0.1 ng to 1 ng, or 0.1 ng to 0.5 ng total dry weight.

In other embodiments, a microbial consortium of the present invention is consumed at a rate of 0.1 ng to 500 mg a day, 0.5 ng to 500 mg a day, 1 ng to 500 mg a day, 5 ng to 500 mg a day, 10 ng to 500 mg a day, 50 ng to 500 mg a day, 100 ng to 500 mg a day, 500 ng to 500 mg a day, 1 μg to 500 mg a day, 5 μg to 500 mg a day, 10 μg to 500 mg a day, 50 μg to 500 mg a day, 100 μg to 500 mg a day, 500 μg to 500 mg a day, 1 mg to 500 mg a day, 5 mg to 500 mg a day, 10 mg to 500 mg a day, 50 mg to 500 mg a day, 100 mg to 500 mg a day, 0.1 ng to 100 mg a day, 0.5 ng to 100 mg a day, 1 ng to 100 mg a day, 5 ng to 100 mg a day, 10 ng to 100 mg a day, 50 ng to 100 mg a day, 100 ng to 100 mg a day, 500 ng to 500 mg a day, 1 μg to 100 mg a day, 5 μg to 100 mg a day, 10 μg to 100 mg a day, 50 μg to 100 mg a day, 100 μg to 100 mg a day, 500 μg to 100 mg a day, 1 mg to 500 mg a day, 5 mg to 100 mg a day, 10 mg to 100 mg a day, 50 mg to 100 mg a day, 0.1 ng to 50 mg a day, 0.5 ng to 50 mg a day, 1 ng to 50 mg a day, 5 ng to 50 mg a day, 10 ng to 50 mg a day, 50 ng to 50 mg a day, 100 ng to 50 mg a day, 500 ng to 500 mg a day, 1 μg to 50 mg a day, 5 μg to 50 mg a day, 10 μg to 50 mg a day, 50 μg to 50 mg a day, 100 μg to 50 mg a day, 500 μg to 50 mg a day, 1 mg to 500 mg a day, 5 mg to 50 mg a day, 10 mg to 50 mg a day, 0.1 ng to 10 mg a day, 0.5 ng to 10 mg a day, 1 ng to 10 mg a day, 5 ng to 10 mg a day, 10 ng to 10 mg a day, 50 ng to 10 mg a day, 100 ng to 10 mg a day, 500 ng to 500 mg a day, 1 μg to 10 mg a day, 5 μg to 10 mg a day, 10 μg to 10 mg a day, 50 μg to 10 mg a day, 100 μg to 10 mg a day, 500 μg to 10 mg a day, 1 mg to 500 mg a day, 5 mg to 10 mg a day, 0.1 ng to 5 mg a day, 0.5 ng to 5 mg a day, 1 ng to 5 mg a day, 5 ng to 5 mg a day, 10 ng to 5 mg a day, 50 ng to 5 mg a day, 100 ng to 5 mg a day, 500 ng to 500 mg a day, 1 μg to 5 mg a day, 5 μg to 5 mg a day, 10 μg to 5 mg a day, 50 μg to 5 mg a day, 100 μg to 5 mg a day, 500 μg to 5 mg a day, 1 mg to 500 mg a day, 0.1 ng to 1 mg a day, 0.5 ng to 1 mg a day, 1 ng to 1 mg a day, 5 ng to 1 mg a day, 10 ng to 1 mg a day, 50 ng to 1 mg a day, 100 ng to 1 mg a day, 500 ng to 500 mg a day, 1 μg to 1 mg a day, 5 μg to 1 mg a day, 10 μg to 1 mg a day, 50 μg to 1 mg a day, 100 μg to 1 mg a day, 500 μg to 1 mg a day, 0.1 ng to 500 μg a day, 0.5 ng to 500 μg a day, 1 ng to 500 μg a day, 5 ng to 500 μg a day, 10 ng to 500 μg a day, 50 ng to 500 μg a day, 100 ng to 500 μg a day, 500 ng to 500 μg a day, 1 μg to 500 μg a day, 5 μg to 500 μg a day, 10 μg to 500 μg a day, 50 μg to 500 μg a day, 100 μg to 500 μg a day, 0.1 ng to 100 μg a day, 0.5 ng to 100 μg a day, 1 ng to 100 μg a day, 5 ng to 100 μg a day, 10 ng to 100 μg a day, 50 ng to 100 μg a day, 100 ng to 100 μg a day, 500 ng to 100 μg a day, 1 μg to 100 μg a day, 5 μg to 100 μg a day, 10 μg to 100 μg a day, 50 μg to 100 μg a day, 0.1 ng to 50 μg a day, 0.5 ng to 50 μg a day, 1 ng to 50 μg a day, 5 ng to 50 μg a day, 10 ng to 50 μg a day, 50 ng to 50 μg a day, 100 ng to 50 μg a day, 500 ng to 50 μg a day, 1 μg to 50 μg a day, 5 μg to 50 μg a day, 10 μg to 50 μg a day, 0.1 ng to 10 μg a day, 0.5 ng to 10 μg a day, 1 ng to 10 μg a day, 5 ng to 10 μg a day, 10 ng to 10 μg a day, 50 ng to 10 μg a day, 100 ng to 10 μg a day, 500 ng to 10 μg a day, 1 μg to 10 μg a day, 5 μg to 10 μg a day, 0.1 ng to 5 μg a day, 0.5 ng to 5 μg a day, 1 ng to 5 μg a day, 5 ng to 5 μg a day, 10 ng to 5 μg a day, 50 ng to 5 μg a day, 100 ng to 5 μg a day, 500 ng to 5 μg a day, 1 μg to 5 μg a day, 0.1 ng to 1 μg a day, 0.5 ng to 1 μg a day, 1 ng to 1 μg a day, 5 ng to 1 μg a day, 10 ng to 1 μg a day, 50 ng to 1 μg a day, 100 ng to 1 μg a day, 500 ng to 1 μg a day, 0.1 ng to 500 ng a day, 0.5 ng to 500 ng a day, 1 ng to 500 ng a day, 5 ng to 500 ng a day, 10 ng to 500 ng a day, 50 ng to 500 ng a day, 100 ng to 500 ng a day, 0.1 ng to 100 ng a day, 0.5 ng to 100 ng a day, 1 ng to 100 ng a day, 5 ng to 100 ng a day, 10 ng to 100 ng a day, 50 ng to 100 ng a day, 0.1 ng to 50 ng a day, 0.5 ng to 50 ng a day, 1 ng to 50 ng a day, 5 ng to 50 ng a day, 10 ng to 50 ng a day, 0.1 ng to 10 ng a day, 0.5 ng to 10 ng a day, 1 ng to 10 ng a day, 5 ng to 10 ng a day, 0.1 ng to 5 ng a day, 0.5 ng to 5 ng a day, 1 ng to 5 ng a day, 0.1 ng to 1 ng a day, 0.1 ng to 1 ng a day, or 0.1 ng to 0.5 ng a day.

In some embodiments, the microbial composition of the present invention is administered for a period of at least 1 day to 1 week, 1 week to 1 month, 1 month to 3 months, 3 months to 6 months, 6 months to 1 year, or more than 1 year. For example, in some embodiments, the microbial composition of the present invention is administered for a period of at least 1 day, 2 days, 3 days, 4 days, 5 days, 6 days, 1 week, 2 weeks, 3 weeks, 1 month, 2 months, 3 months, 4 months, 5 months, 6 months, or 1 year.

In some embodiments, a microbial consortium of the present invention is administered as a single dose or as multiple doses. For example, in some embodiments, a microbial consortium of the present invention is administered once a day for 2 days, 3 days, 4 days, 5 days, 6 days, 1 week, 2 weeks 3 weeks, 1 month, 2 months, 3 months, 4 months, 5 months, 6 months, or 1 year. In some embodiments, a microbial consortium of the present invention is administered multiple times daily. For example, in some embodiments, a microbial consortium of the present invention is administered twice daily, three times daily, 4 times daily, or 5 times daily. In some embodiments, a microbial consortium of the present invention is administered intermittently. For example, in some embodiments, a microbial consortium of the present invention is administered once weekly, once monthly, or when a subject is in need thereof.

### Combination Therapy

In some embodiments, a microbial consortium of the present invention can be administered in combination with other agents. For example, in some embodiments, a microbial consortium of the present invention can be administered with an antimicrobial agent, an antifungal agent, an antiviral agent, an antiparasitic agent or a prebiotic. In some embodiments, a microbial consortium of the present invention can be administered subsequent to administration of an antimicrobial agent, an antifungal agent, an antiviral agent, an antiparasitic agent or a prebiotic. In some embodiments, administration may be sequential over a period of hours or days, or simultaneously.

For example, in some embodiments, a microbial consortium can be administered with, or pre-administered with, one or more than one antibacterial agent selected from fluoroquinolone antibiotics (ciprofloxacin, Levaquin, floxin, tequin, avelox, and norflox); cephalosporin antibiotics (cephalexin, cefuroxime, cefadroxil, cefazolin, cephalothin, cefaclor, cefamandole, cefoxitin, cefprozil, and ceftobiprole); penicillin antibiotics (amoxicillin, ampicillin, penicillin V, dicloxacillin, carbenicillin, vancomycin, and methicillin); tetracycline antibiotics (tetracycline, minocycline, oxytetracycline, and doxycycline); and carbapenem antibiotics (ertapenem, doripenem, imipenem/cilastatin, and meropenem).

For example, in some embodiments, a microbial consortium can be administered with one or more than one antiviral agent selected from Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuviltide, Etravirine, Famciclovir, Foscamet, Fomivirsen, Ganciclovir, Indinavir, Idoxuridine, Lamivudine, Lopinavir Maraviroc, MK-2048, Nelfinavir, Nevirapine, Penciclovir, Raltegravir, Rilpivirine, Ritonavir, Saquinavir, Stavudine, Tenofovir Trifluridine, Valaciclovir, Valganciclovir, Vidarabine, Ibacitabine, Amantadine, Oseltamivir, Rimantidine, Tipranavir, Zalcitabine, Zanamivir, and Zidovudine.

In some embodiments, a microbial consortium can be administered with one or more than one antifungal agent selected from miconazole, ketoconazole, clotrimazole, econazole, omoconazole, bifonazole, butoconazole, fenticonazole, isoconazole, oxiconazole, sertaconazole, sulconazole, and tioconazole; triazole antifungals such as fluconazole, itraconazole, isavuconazole, ravuconazole, posaconazole, voriconazok, terconazole, and albaconazole; thiazole antifungals such as abafungin; allylamine antifungals such as terbinafine, naftifine, and butenafine; and echinocandin antifungals such as anidulafungin, caspofungin, and micafungin; polygodial; benzoic acid; ciclopirox; tolnaftate; undecylenic acid; flucytosine or 5-fluorocytosine; griseofulvin; and haloprogin.

In some embodiments, a microbial consortium can be administered with one or more than one anti-inflammatory and/or immunosuppressive agent selected from cyclophosphamide, mycophenolate mofetil, corticosteroids, mesalazine, mesalamine, sulfasalazine, sulfasalazine derivatives, cyclosporin A, mercaptopurine, azathiopurine, prednisone, methotrexate, antihistamines, glucocorticoids, epinephrine, theophylline, cromolyn sodium, anti-leukotrienes, anticholinergics, monoclonal anti-IgE, immunomodulatory peptides, immunomodulatory small molecules, immunomodulatory cytokines, immunomodulatory antibodies, and vaccines.

In some embodiments, a microbial consortium of the present invention can be administered with one or more than one prebiotic selected from, but not limited to, amino acids, biotin, fructooligosaccharides, galactooligosaccharides, inulin, lactulose, mannan oligosaccharides, oligofructose-enriched inulin, oligofructose, oligodextrose, tagatose, trans-galactooligosaccharide, and xylooligosaccharides.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: Sourcing and Identification of Active and Supportive Microbial Strains

Active and supportive microbial strains were derived from human donor fecal samples, or were purchased from one of three depositories: the American Type Culture Collection (ATCC; www.atcc.org), the Leibniz institute DSMZ (www.dsmz.de), or BEI Resources (www.beiresources.org).

Microbial strains purchased from a depository were cultured according to depository instructions.

### Isolation of Donor-Derived Active and Supportive Microbial Strains

Fecal donors were selected based on multiple criteria, including a health and medical history questionnaire, physical exam, and blood and stool tests for assessing pathogen-free status. Stool samples from donors who did not meet the inclusion criteria based on any of the above-mentioned assessment were discarded from quarantine.

Donors provided a stool sample sealed in a plastic container. Upon collection, stool samples were immediately transferred to an anaerobic chamber (5% CO₂, 5% H₂, 90% N₂) within 15 minutes of collection.

Once transferred to the anaerobic chamber, the fresh stool sample was labeled, weighed, evaluated for anomalies (presence of urine, toilet paper, etc.), and scored according to the Bristol scale. A stool sample weighing less than 45 g, or that failed to conform to a Bristol Stool Scale type 2, 3, 4 or 5, was rejected. Stool samples that met the acceptance criteria were processed and aliquoted. 45 g of the stool sample was transferred into a sterile container for specific pathogen testing. The remainder of the sample was mixed with cryopresertative, homogenized, and aliquoted into cryovials (approximately 2 g of sample per vial; 6 vials per stool sample). These vials were transferred from the anaerobic chamber to a −80° C. freezer for storage until shipping on dry ice.

Microbial strain isolation was performed by making serial dilution aliquots of the stool samples and plating said aliquots on a variety of microbial cultivation media suitable for growth of anaerobes. All cultures were grown under anaerobic conditions for the duration of culturing. Approximately 20 different media/culture conditions were used to isolate a variety of gut microbial species. Specific enrichment techniques were performed for species having particular metabolic capabilities, such as consumption or tolerance of oxalate or bile acids. In order to enrich for strains having oxalate metabolism capabilities, aliquots of the serially-diluted stool samples were plated on agar growth media supplemented with varying concentrations of potassium oxalate (20 mM, 40 mM, 80 mM, 160 mM, or 200 mM). In order to enrich for species capable of metabolizing bile acids, aliquots of serially diluted stool samples were plated on growth media supplemented with 2% bile. In order to isolate archaea, diluted fecal samples were plated on culture media containing a mixture of antibiotics that is lethal to both gram-positive and gram-negative bacteria. This archaeal isolation plate was co-incubated in a small enclosed container together with a separate plate containing a heterogenous population of microbes derived from a fecal sample; the heterogenous population contained hydrogen-producing microbes, thereby providing hydrogen (through diffusion within the small container) to allow archaea on the archaeal isolation plate to grow.

Single colonies from isolation or enrichment plates were picked for further isolation on appropriate microbial cultivation agar media plates (passage 2). After incubation at 37° C., if the single colony plating resulted in uniformly isolated colony morphology, the culture was further investigated for strain identification. Preliminary strain identification was performed either by 16S rRNA gene sequencing or by creating and analyzing proteomic fingerprinting using high-throughput Matrix-Assisted Laser Desorption/Ionization Time-Of-Flight Mass Spectrometry (MALDI-TOF MS). If the single-colony plating resulted in multiple colony morphologies, each unique colony type was picked for further isolation on an appropriate cultivation agar plate until uniform colony morphology was achieved (passage 3 or more). Monoculture identity was confirmed by 16S rRNA gene sequencing.

Isolated colonies of strains purified to monocultures for species of interest, as well as novel strains of unknown species, were inoculated into culture tubes containing appropriate broth media and incubated under anaerobic conditions at 37° C. For most strains, sufficient growth was visualized during this first broth passaging as indicated by turbidity. However, some strains required more than one broth passaging to achieve sufficient growth. Sterile glycerol solution was added to the microbial culture to achieve a final glycerol concentration of 25% prior to mixing and aliquoting into cryovials. The cryovials were removed from the anaerobic gas chambers and were promptly transferred to −80° C.

After at least 10 hours of freezing, one vial of each purified frozen strain isolate was retrieved from the freezer and thawed under anaerobic conditions followed by plating on agar plates containing appropriate growth media. The plates were incubated under anaerobic conditions at 37° C. Growth on the plate was observed to confirm revival and uniform morphology for each purified isolate. Individual colonies of the isolates were subsequently analyzed by 16S rRNA gene sequencing to confirm the identity and colony purity of each frozen strain against the National Center for Biotechnology Information (NCBI) 16S rRNA gene databases.

A list of donor-derived isolates and a summary of their corresponding isolation media and growth/banking media is reported in Table 3. Additional identifying information for the isolates is reported in Table 4.

in vitro activity-based assays, bioinformatic screens to identify strains with the genetic capability to metabolize oxalate, and identification of target species with known oxalate metabolizing activity based on scientific literature, were utilized to identify candidate active strains. Active oxalate-metabolizing strains obtained from depositories (“commercial strains”) include those listed in Table 5. Supportive commercial strains include those listed in Table 6. Strains in Table 5 and Table 6 are identified by their genus/species and by the depository catalog number. “ATCC” strains were obtained from ATCC, “DSM” strains were obtained from the Liebniz Institute DSMZ, and “HM” strains were obtained from BEI Resources.

### MALDI-TOF MS

MALDI-TOF mass spectrometry was used for preliminary identification of bacterial strains (genus and/or species) using a BD Bruker MALDI Biotyper. Briefly, an α-cyano-4-hydroxycinnamic acid (HCCA) matrix was prepared in Bruker standard solvent (acetonitrile 50%, water 47.5% and trifluoroacetic acid 2.5%). A disposable MALDI Biotyper Biotarget plate was loaded with a smear of the sample bacterial colony, overlaid with HCCA matrix and allowed to dry. For strains that required extended extraction, 70% formic acid was added to the sample smear prior to adding HCCA matrix. Bruker Bacterial Testing Standards (BTS) were also loaded onto the Biotarget for quality control analysis. The Biotarget as then loaded into a Biotyper MALDI-TOF machine, and the sample was analyzed. The machine was configured to perform the quality control analysis of the BTS quality control samples first and aborted the run if the BTS quality control analysis failed. The generated spectrum of the test sample was then compared to a database of the reference proteomics spectra containing strains belonging to species which were previously characterized by their proteomic fingerprinting.

### DNA Extraction

DNA was extracted from fecal samples using a Qiagen DNeasy Power Soil Kit (Qiagen, Germantown, Md.) in accordance with the manufacturer's instructions. Alternative methods for extracting DNA from fecal samples are well-known and routinely practiced in the art (e.g., described by Sambrook and Russell, Molecular Cloning: A Laboratory Manual, 3d ed., 2001).

### Whole Genome Shotgun Sequencing

Sequencing of DNA samples was carried out using the TruSeq Nano DNA Library Preparation kit (Illumina, San Diego, Calif., US) and a NextSeq platform (Illumina, San Diego, Calif., US). In brief, sequencing libraries were prepared from DNA extracted from each sample. DNA was mechanically fragmented using an ultrasonicator. The fragmented DNA was subjected to end repair and size selection of fragments, adenylation of 3′ ends, linked with adaptors, and DNA fragments enriched according to the TruSeq Nano DNA Library Preparation kit manual (Illumina, San Diego, Calif., US). Samples were sequenced to generate more than 50 million paired-end reads of 150, 250, or 300 bp length.

16S rRNA Gene Sequencing and Species Identification

Microbial species identification was performed by full-length Sanger sequencing of the 16S rRNA gene using the 27F and 1492 primers (PMID 18296538). Species were identified by performing a bidirectional best-BLAST search against a database of curated 16S rRNA gene sequences of type species. To refine species identities, 16S rRNA gene sequences were inserted into a phylogenetic tree of curated 16S rRNA gene sequences of type species. If the sequence formed a monophyletic cluster with a known species, the strain was assigned to that species. Otherwise, the strain was assigned to a novel species. Optionally, isolates were additionally characterized by whole-genome sequencing. Genome assemblies were inserted into a phylogenetic tree of curated genomes of type species. If the sequence formed a monophyletic cluster with a known species, the strain was assigned to that species. Otherwise, the strain was assigned to a novel species.

TABLE 3
Summary of Isolation/Growth Media for Donor-Derived Isolates

| | | | | Isolated from | Isolated from | |
|---|---|---|---|---|---|---|
| | | | | media | media | |
| | | Stool plating agar media | Additives | containing | containing | Glycerol |
| Strain # | Species ID | type (Vendor, Cat #) | information | oxalate | 2% Ox bile | stock media |
| FBI00001 | Clostridium citroniae | Bifidobacterium Selective | 40 mM | 40 mM | NO | YCFAC + 40 mM |
| | | agar (Anaerobe Systems, | Potassium | | | oxalate |
| | | AS-6423) | oxalate | | | |
| FBI00002 | Bacteroides salyersiae | Bifidobacterium Selective | 40 mM | 40 mM | NO | YCFAC + 40 mM |
| | | agar (Anaerobe Systems, | Potassium | | | oxalate |
| | | AS-6423) | oxalate | | | |
| FBI00003 | Enterococcus faecalis | Bifidobacterium Selective | 40 mM | 40 mM | NO | YCFAC + 40 mM |
| | | agar (Anaerobe Systems, | Potassium | | | oxalate |
| | | AS-6423) | oxalate | | | |
| FBI00004 | Neglecta timonensis | YCFAC-B (Anaerobe | 80 mM | 80 mM | NO | YCFAC + 80 mM |
| | | Systems, AS-677) | Potassium | | | oxalate |
| | | | oxalate | | | |
| FBI00005 | Enterococcus | YCFAC-B (Anaerobe | 80 mM | 80 mM | NO | YCFAC + 80 mM |
| | casseliflavus | Systems, AS-677) | Potassium | | | oxalate |
| | | | oxalate | | | |
| FBI00006 | Enterobacter | YCFAC-B (Anaerobe | 80 mM | 80 mM | NO | YCFAC + 80 mM |
| | himalayensis | Systems, AS-677) | Potassium | | | oxalate |
| | | | oxalate | | | |
| FBI00007 | Enterococcus | YCFAC-B (Anaerobe | 80 mM | 80 mM | NO | YCFAC + 80 mM |
| | casseliflavus | Systems, AS-677) | Potassium | | | oxalate |
| | | | oxalate | | | |
| FBI00008 | Blautia luti | YCFAC-B (Anaerobe | 20 mM | 20 mM | NO | YCFAC + 20 mM |
| | | Systems, AS-677) | Potassium | | | oxalate |
| | | | oxalate | | | |
| FBI00009 | Bifidobacterium | YCFAC-B (Anaerobe | | NO | NO | YCFAC |
| | adolescentis | Systems, AS-677) | | | | |
| FBI00010 | Blautia obeum | YCFAC-B (Anaerobe | | NO | NO | YCFAC |
| | | Systems, AS-677) | | | | |
| FBI00011 | Bifidobacterium longum | Bifidobacterium Selective | | NO | NO | YCFAC |
| | | agar (Anaerobe Systems, | | | | |
| | | AS-6423) | | | | |
| FBI00012 | Alistipes onderdonkii | Bacteroides Bile Esculin | | NO | YES | YCFAC |
| | | (BBE) (Anaerobe Systems, | | | | |
| | | AS-144) | | | | |
| FBI00013 | Parabacteroides merdae | YCFAC-B (Anaerobe | | NO | NO | YCFAC |
| | | Systems, AS-677) | | | | |
| FBI00014 | Blautia luti | YCFAC-B (Anaerobe | 20 mM | 20 mM | NO | YCFAC + 20 mM |
| | | Systems, AS-677) | Potassium | | | oxalate |
| | | | oxalate | | | |
| FBI00015 | Bacteroides uniformis | YCFAC-B (Anaerobe | | NO | NO | YCFAC |
| | | Systems, AS-677) | | | | |
| FBI00016 | Bifidobacterium | YCFAC-B (Anaerobe | | NO | NO | YCFAC |
| | pseudocatenulatum | Systems, AS-677) | | | | |
| FBI00017 | Blautia obeum | YCFAC-B (Anaerobe | 20 mM | 20 mM | NO | YCFAC + 20 mM |
| | | Systems, AS-677) | Potassium | | | oxalate |
| | | | oxalate | | | |
| FBI00018 | Eubacterium rectale | Bifidobacterium Selective | | NO | NO | YCFAC |
| | | agar (Anaerobe Systems, | | | | |
| | | AS-6423) | | | | |
| FBI00019 | Alistipes timonensis | Bacteroides Bile Esculin | | NO | YES | YCFAC |
| | | (BBE) (Anaerobe Systems, | | | | |
| | | AS-144) | | | | |
| FBI00020 | Bacteroides | Bifidobacterium Selective | 40 mM | 40 mM | NO | YCFAC + 40 mM |
| | thetaiotaomicron | agar (Anaerobe Systems, | Potassium | | | oxalate |
| | | AS-6423) | oxalate | | | |
| FBI00021 | Bacteroides kribbi/ | YCFAC-B (Anaerobe | 80 mM | 80 mM | NO | YCFAC + 80 mM |
| | Bacteroides koreensis | Systems, AS-677) | Potassium | | | oxalate |
| | species cluster | | oxalate | | | |
| FBI00022 | Alistipes putredinrs | Bacteroides Bile Esculin | | NO | YES | YCFAC |
| | | (BBE) (Anaerobe Systems, | | | | |
| | | AS-144) | | | | |
| FBI00023 | Enterococcus | Strain Isolation Media 1 | | NO | NO | YCFAC |
| | casseliflavus | (SL1) | | | | |
| FBI00024 | Bacteroides kribbi/ | Chocolate Agar (Teknova, | | NO | NO | YCFAC |
| | Bacteroides koreensis | C4900) | | | | |
| | species cluster | | | | | |
| FBI00025 | Coprococcus comes | Chocolate Agar (Teknova, | | NO | NO | YCFAC |
| | | C4900) | | | | |
| FBI00026 | Enterobacter | YCFAC-BO 200 mM | | 200 mM | NO | YCFAC + 80 mM |
| | hormaechei | (Anaerobe Systems, AS- | | | | oxalate |
| | | 7529) | | | | |
| FBI00027 | Fusicatenibacter | Brain Heart Infusion | | NO | NO | YCFAC |
| | saccharivorans | (BHI), hemin, vitamin K | | | | |
| | | (Teknova, B1093) | | | | |
| FBI00028 | Oscillibacter sp. | Brain Heart Infusion | | NO | NO | YCFAC |
| | FBI00028 | (BHI), hemin, vitamin K | | | | |
| | | (Teknova, B1093) | | | | |
| FBI00029 | Parabacteroides | Brain Heart Infusion | | NO | NO | YCFAC |
| | distasonis | (BHI), hemin, vitamin K | | | | |
| | | (Teknova, B1093) | | | | |
| FBI00030 | Eggerthella lenta | YCFAC-B (Anaerobe | | NO | NO | YCFAC |
| | | Systems, AS-677) | | | | |
| FBI00031 | Enterobacter | YCFAC-BO 200 mM | | 200 mM | NO | YCFAC + 80 mM |
| | hormaechei | (Anaerobe Systems, AS- | | | | oxalate |
| | | 7529) | | | | |
| FBI00032 | Anaerostipes hadrus | Bifidobacterium Selective | | NO | NO | YCFAC |
| | | agar (Anaerobe Systems, | | | | |
| | | AS-6423) | | | | |
| FBI00033 | Lachnospiraceae sp. | YCFAC-B (Anaerobe | 20 mM | 20 mM | NO | YCFAC + 20 mM |
| | FBI00033 | Systems, AS-677) | Potassium | | | oxalate |
| | | | oxalate | | | |
| FBI00034 | Eubacterium eligens | Brain Heart Infusion | | NO | NO | YCFAC |
| | | (BHI), hemin, vitamin K | | | | |
| | | (Teknova, B1093) | | | | |
| FBI00035 | Enterococcus | YCFAC-BO 200 mM | | 200 mM | NO | YCFAC + 80 mM |
| | casseliflavus | (Anaerobe Systems, AS- | | | | oxalate |
| | | 7529) | | | | |
| FBI00036 | Blautia faecis | Chocolate Agar (Teknova, | | NO | NO | YCFAC |
| | | C4900) | | | | |
| FBI00037 | Enterococcus | YCFAC-BO 200 mM | | 200 mM | NO | YCFAC + 80 mM |
| | casseliflavus | (Anaerobe Systems, AS- | | | | oxalate |
| | | 7529) | | | | |
| FBI00038 | Coprococcus eutactus | Chocolate Agar (Teknova, | | NO | NO | YCFAC |
| | | C4900) | | | | |
| FBI00039 | Bacteroides vulgatus | Chocolate Agar (Teknova, | | NO | NO | YCFAC |
| | | C4900) | | | | |
| FBI00040 | Bilophila wadsworthia | Bacteroides Bile Esculin | | NO | YES | YCFAC |
| | | (BBE) (Anaerobe Systems, | | | | |
| | | AS-144) | | | | |
| FBI00041 | Phascolarctobacterium | YCFAC-B (Anaerobe | 80 mM | 80 mM | NO | YCFAC + 80 mM |
| | faecium | Systems, AS-677) | Potassium | | | oxalate |
| | | | oxalate | | | |
| FBI00042 | Bacteroides | Strain Isolation Media 1 | | NO | NO | YCFAC |
| | xylani solvens | (SL1) | | | | |
| FBI00043 | Bifidobacterium | Reinforced Clostridial | | NO | NO | YCFAC |
| | dentium | Agar (RCA) (Teknova, | | | | |
| | | C0205) | | | | |
| FBI00044 | Blautia wexlerae | Chocolate Agar (Teknova, | | NO | NO | YCFAC |
| | | C4900) | | | | |
| FBI00045 | Bifidobacterium | Reinforced Clostridial | | NO | NO | YCFAC |
| | adolescentis | Agar (RCA) (Teknova, | | | | |
| | | C0205) | | | | |
| FBI00046 | Bacteroides caccae | YCFAC-B (Anaerobe | | NO | NO | YCFAC |
| | | Systems, AS-677) | | | | |
| FBI00047 | Eubacterium eligens | YCFAC-B (Anaerobe | | NO | NO | YCFAC |
| | | Systems, AS-677) | | | | |
| FBI00048 | Fusicatenibacter | YCFAC-B (Anaerobe | | NO | NO | YCFAC |
| | saccharivorans | Systems, AS-677) | | | | |
| FBI00049 | Dialister succinatiphilus | YCFAC-B (Anaerobe | | NO | NO | YCFAC |
| | | Systems, AS-677) | | | | |
| FBI00050 | Bacteroides nordii | Bifidobacterium Selective | 40 mM | 40 mM | NO | YCFAC + 40 mM |
| | | agar (Anaerobe Systems, | Potassium | | | oxalate |
| | | AS-6423) | oxalate | | | |
| FBI00051 | Dorea formicigenerans | YCFAC-B (Anaerobe | | NO | NO | YCFAC |
| | | Systems, AS-677) | | | | |
| FBI00052 | Bacteroides | YCFAC-BO 40 mM | | 40 mM | NO | YCFAC + 40 mM |
| | xylanisolvens | (Anaerobe Systems, AS- | | | | oxalate |
| | | 7523) | | | | |
| FBI00053 | Lactobacillus rogosae | YCFAC-BO 40 mM | | 40 mM | NO | YCFAC + 40 mM |
| | | (Anaerobe Systems, AS- | | | | oxalate |
| | | 7523) | | | | |
| FBI00054 | Escherichia flexneri | YCFAC-BO 80 mM | | 80 mM | NO | YCFAC + 80 mM |
| | | (Anaerobe Systems, AS- | | | | oxalate |
| | | 7524) | | | | |
| FBI00055 | Bacteroides kribbi/ | YCFAC-BO 80 mM | | 80 mM | NO | YCFAC + 80 mM |
| | Bacteroides koreensis | (Anaerobe Systems, AS- | | | | oxalate |
| | species cluster | 7524) | | | | |
| FBI00056 | Clostridium citroniae | YCFAC-BO 80 mM | | 80 mM | NO | YCFAC + 80 mM |
| | | (Anaerobe Systems, AS- | | | | oxalate |
| | | 7524) | | | | |
| FBI00057 | Dorea longicatena | Reinforced Clostridial | | NO | NO | YCFAC |
| | | Agar (RCA) (Teknova, | | | | |
| | | C0205) | | | | |
| FBI00058 | Eubacterium rectale | Lactobacillus MRS | 40 mM | 40 mM | NO | YCFAC |
| | | (Anaerobe Systems, AS- | Potassium | | | |
| | | 6429) | oxalate | | | |
| FBI00059 | Bacteroides | Columbia agar, 5% sheep | | NO | NO | YCFAC |
| | stercorirosoris | blood (BD, 221165) | | | | |
| FBI00060 | Bifidobacterium longum | YCFAC-B (Anaerobe | | NO | NO | YCFAC |
| | | Systems, AS-677) | | | | |
| FBI00061 | Alistipes shahii | Bacteroides Bile Esculin | | NO | YES | YCFAC |
| | | (BBE) (Anaerobe Systems, | | | | |
| | | AS-144) | | | | |
| FBI00062 | Collinsella aerofaciens | YCFAC-B (Anaerobe | | NO | NO | YCFAC |
| | | Systems, AS-677) | | | | |
| FBI00063 | Lachnospira sp. | YCFAC-B (Anaerobe | | NO | NO | YCFAC |
| | FBI00063 FBI00285 | Systems, AS-677) | | | | |
| | FBI00364 | | | | | |
| FBI00064 | Dorea sp. FBI00064 | YCFAC-B (Anaerobe | | NO | NO | YCFAC |
| | | Systems, AS-677) | | | | |
| FBI00065 | Sutterellaceae sp. | Bacteroides Bile Esculin | | NO | YES | YCFAC |
| | FBI00065 | (BBE) (Anaerobe Systems, | | | | |
| | | AS-144) | | | | |
| FBI00066 | Parasutterella | Bacteroides Bile Esculin | | NO | YES | YCFAC |
| | excrementihominis | (BBE) (Anaerobe Systems, | | | | |
| | | AS-144) | | | | |
| FBI00067 | Oxalobacter formigenes | YCFAC-BO 40 mM | | 40 mM | NO | YCFAC + 100 |
| | | (Anaerobe Systems, AS- | | | | mM oxalate |
| | | 7523) | | | | |
| FBI00068 | Akkermansia | Strain Isolation Media 1 | | NO | NO | YCFAC |
| | muciniphila | (SL1) | | | | |
| FBI00069 | Ruminococcus bromii | Bifidobacterium Selective | 40 mM | 40 mM | NO | YCFAC |
| | | agar (Anaerobe Systems, | Potassium | | | |
| | | AS-6423) | oxalate | | | |
| FBI00070 | Bacteroides kribbi/ | YCFAC-BO 40 mM | | 40 mM | NO | YCFAC + 40 mM |
| | Bacteroides koreensis | (Anaerobe Systems, AS- | | | | oxalate |
| | species cluster | 7523) | | | | |
| FBI00071 | Lachnospiraceae sp. | YCFAC-B (Anaerobe | | NO | NO | YCFAC |
| | FBI00071 | Systems, AS-677) | | | | |
| FBI00072 | Coprococcus eutactus | Brain Heart Infusion | | NO | NO | YCFAC |
| | | (BHI), hemin, vitamin K | | | | |
| | | (Teknova, B1093) | | | | |
| FBI00073 | Parabacteroides | YCFAC-BO 40 mM | | 40 mM | NO | YCFAC |
| | distasonis | (Anaerobe Systems, AS- | | | | |
| | | 7523) | | | | |
| FBI00074 | Clostridium fessum | Brain Heart Infusion | | NO | NO | YCFAC |
| | | (BHI), hemin, vitamin K | | | | |
| | | (Teknova, B1093) | | | | |
| FBI00075 | Paraprevotella clara | YCFAC-B (Anaerobe | | NO | NO | YCFAC |
| | | Systems, AS-677) | | | | |
| FBI00076 | Bacteroides | YCFAC-BO 80 mM | | 80 mM | NO | YCFAC |
| | thetaiotaomicron | (Anaerobe Systems, AS- | | | | |
| | | 7524) | | | | |
| FBI00077 | Sutterella | Lactobacillus MRS | 20 mM | 20 mM | NO | YCFAC |
| | wadsworthensis | (Anaerobe Systems, AS- | Potassium | | | |
| | | 6429) | oxalate | | | |
| FBI00078 | Blautia obeum | YCFAC-B (Anaerobe | | NO | NO | YCFAC |
| | | Systems, AS-677) | | | | |
| FBI00079 | Clostridium | Chocolate Agar (Teknova, | | NO | NO | YCFAC |
| | clostridioforme | C4900) | | | | |
| FBI00080 | Sutterella massiliensis | Lactobacillus MRS | 40 mM | 40 mM | NO | YCFAC |
| | | (Anaerobe Systems, AS- | Potassium | | | |
| | | 6429) | oxalate | | | |
| FBI00081 | Porphyromonas | Columbia agar, 5% sheep | | NO | NO | YCFAC |
| | asaccharolytica | blood (BD, 221165) | | | | |
| FBI00082 | Ruminococcaceae sp. | Brain Heart Infusion | | NO | NO | YCFAC |
| | FBI00082 FBI00097 | (BHI), hemin, vitamin K | | | | |
| | | (Teknova, B1093) | | | | |
| FBI00083 | Alistipes shahii | Columbia agar, 5% sheep | Antibiotics | NO | NO | YCFAC |
| | | blood (BD, 221165) | | | | |
| FBI00084 | Bifidobacterium longum | Bifidobacterium Selective | | NO | NO | YCFAC |
| | | agar (Anaerobe Systems, | | | | |
| | | AS-6423) | | | | |
| FBI00085 | Ruminococcus bromii | Reinforced Clostridial | | NO | NO | YCFAC |
| | | Agar (RCA) (Teknova, | | | | |
| | | C0205) | | | | |
| FBI00086 | Ruminococcus bromii | Brain Heart Infusion | | NO | NO | YCFAC |
| | | (BHI), hemin, vitamin K | | | | |
| | | (Teknova, B1093) | | | | |
| FBI00087 | Clostridium scindens | Brain Heart Infusion | | NO | NO | YCFAC |
| | | (BHI), hemin, vitamin K | | | | |
| | | (Teknova, B1093) | | | | |
| FBI00088 | Lactobacillus rogosae | Reinforced Clostridial | | NO | NO | YCFAC |
| | | Agar (RCA) (Teknova, | | | | |
| | | C0205) | | | | |
| FBI00089 | Bifidobacterium longum | YCFAC-B (Anaerobe | | NO | NO | YCFAC |
| | | Systems, AS-677) | | | | |
| FBI00090 | Eubacterium eligens | Brain Heart Infusion | | NO | NO | YCFAC |
| | | (BHI), hemin, vitamin K | | | | |
| | | (Teknova, B1093) | | | | |
| FBI00091 | Eubacterium rectale | Brain Heart Infusion | | NO | NO | YCFAC |
| | | (BHI), hemin, vitamin K | | | | |
| | | (Teknova, B1093) | | | | |
| FBI00092 | Monoglobus | YCFAC-BO 40 mM | | 40 mM | NO | YCFAC |
| | pectinilyticus | (Anaerobe Systems, AS- | | | | |
| | | 7523) | | | | |
| FBI00093 | Roseburia hominis | YCFAC-BO 40 mM | | 40 mM | NO | YCFAC |
| | | (Anaerobe Systems, AS- | | | | |
| | | 7523) | | | | |
| FBI00094 | Enterococcus faecium | YCFAC-BO 40 mM | | 40 mM | NO | YCFAC |
| | | (Anaerobe Systems, AS- | | | | |
| | | 7523) | | | | |
| FBI00095 | Ruminococcus bromii | Reinforced Clostridial | | NO | NO | YCFAC |
| | | Agar (RCA) (Teknova, | | | | |
| | | C0205) | | | | |
| FBI00096 | Eggerthella lenta | Brain Heart Infusion | | NO | NO | YCFAC |
| | | (BHI), hemin, vitamin K | | | | |
| | | (Teknova, B1093) | | | | |
| FBI00097 | Ruminococcaceae sp. | Brain Heart Infusion | | NO | NO | YCFAC |
| | FBI00082 FBI00097 | (BHI), hemin, vitamin K | | | | |
| | | (Teknova, B1093) | | | | |
| FBI00098 | Bacteroides dorei | YCFAC-BO 40 mM | | 40 mM | NO | YCFAC |
| | | (Anaerobe Systems, AS- | | | | |
| | | 7523) | | | | |
| FBI00099 | Gordonibacter | Chocolate Agar (Teknova, | | NO | NO | YCFAC |
| | pamelaeae | C4900) | | | | |
| FBI00100 | Lachnospira sp. | Brain Heart Infusion | | NO | NO | YCFAC |
| | FBI00063 FBI00285 | (BHI), hemin, vitamin K | | | | |
| | FBI00364 | (Teknova, B1093) | | | | |
| FBI00101 | Faecalibacterium | Brain Heart Infusion | | NO | NO | YCFAC |
| | prausnitzii | (BHI), hemin, vitamin K | | | | |
| | | (Teknova, B1093) | | | | |
| FBI00102 | Clostridium fessum | YCFAC-BO 40 mM | | 40 mM | NO | YCFAC |
| | | (Anaerobe Systems, AS- | | | | |
| | | 7523) | | | | |
| FBI00103 | Bacteroides massiliensis | Bifidobacterium Selective | | NO | NO | YCFAC |
| | | agar (Anaerobe Systems, | | | | |
| | | AS-6423) | | | | |
| FBI00104 | Blautia wexlerae | Brain Heart Infusion | | NO | NO | YCFAC |
| | | (BHI), hemin, vitamin K | | | | |
| | | (Teknova, B1093) | | | | |
| FBI00105 | Ruminococcaceae sp. | Chocolate Agar (Teknova, | | NO | NO | YCFAC |
| | FBI00105 FBI00160 | C4900) | | | | |
| FBI00106 | Enterococcus durans | Brain Heart Infusion | | NO | NO | YCFAC |
| | | (BHI), hemin, vitamin K | | | | |
| | | (Teknova, B1093) | | | | |
| FBI00107 | Enterococcus durans | YCFAC-BO 80 mM | | 80 mM | NO | YCFAC |
| | | (Anaerobe Systems, AS- | | | | |
| | | 7524) | | | | |
| FBI00108 | Ruminococcaceae sp. | YCFAC-B (Anaerobe | | NO | NO | YCFAC |
| | FBI00108 | Systems, AS-677) | | | | |
| FBI00109 | Coprococcus comes | YCFAC-B (Anaerobe | | NO | NO | YCFAC |
| | | Systems, AS-677) | | | | |
| FBI00110 | Lachnoclostridium | YCFAC-BO 80 mM | | 80 mM | NO | YCFAC |
| | pacaense | (Anaerobe Systems, AS- | | | | |
| | | 7524) | | | | |
| FBI00111 | Bacteroides vulgatus | YCFAC-BO 80 mM | | 80 mM | NO | YCFAC |
| | | (Anaerobe Systems, AS- | | | | |
| | | 7524) | | | | |
| FBI00112 | Bacteroides uniformis | Brain Heart Infusion | | NO | NO | YCFAC |
| | | (BHI), hemin, vitamin K | | | | |
| | | (Teknova, B1093) | | | | |
| FBI00113 | Parabacteroides merdae | Brain Heart Infusion | | NO | NO | YCFAC |
| | | (BHI), hemin, vitamin K | | | | |
| | | (Teknova, B1093) | | | | |
| FBI00114 | Dorea formicigenerans | Brain Heart Infusion | | NO | NO | YCFAC |
| | | (BHI), hemin, vitamin K | | | | |
| | | (Teknova, B1093) | | | | |
| FBI00115 | Dorea formicigenerans | Brain Heart Infusion | | NO | NO | YCFAC |
| | | (BHI), hemin, vitamin K | | | | |
| | | (Teknova, B1093) | | | | |
| FBI00116 | Ruminococcus faecis | Bifidobacterium Selective | 40 mM | 40 mM | NO | YCFAC |
| | | agar (Anaerobe Systems, | Potassium | | | |
| | | AS-6423) | oxalate | | | |
| FBI00117 | Blautia faecis | YCFAC-BO 80 mM | | 80 mM | NO | YCFAC |
| | | (Anaerobe Systems, AS- | | | | |
| | | 7524) | | | | |
| FBI00118 | Blautia faecis | YCFAC-B (Anaerobe | | NO | NO | YCFAC |
| | | Systems, AS-677) | | | | |
| FBI00119 | Blautia obeum | YCFAC-BO 40 mM | | 40 mM | NO | YCFAC |
| | | (Anaerobe Systems, AS- | | | | |
| | | 7523) | | | | |
| FBI00120 | Hungatella effluvii | YCFAC-BO 80 mM | | 80 mM | NO | YCFAC |
| | | (Anaerobe Systems, AS- | | | | |
| | | 7524) | | | | |
| FBI00121 | Bacteroides vulgatus | YCFAC-BO 40 mM | | 40 mM | NO | YCFAC |
| | | (Anaerobe Systems, AS- | | | | |
| | | 7523) | | | | |
| FBI00122 | Bacteroides uniformis | YCFAC-BO 40 mM | | 40 mM | NO | YCFAC |
| | | (Anaerobe Systems, AS- | | | | |
| | | 7523) | | | | |
| FBI00123 | Roseburia hominis | YCFAC-BO 160 mM | | 160 mM | NO | YCFAC |
| | | (Anaerobe Systems, AS- | | | | |
| | | 7527) | | | | |
| FBI00124 | Anaerostipes hadrus | YCFAC-B (Anaerobe | | NO | NO | YCFAC |
| | | Systems, AS-677) | | | | |
| FBI00125 | Bacteroides stercoris | YCFAC-BO 80 mM | | 80 mM | NO | YCFAC |
| | | (Anaerobe Systems, AS- | | | | |
| | | 7524) | | | | |
| FBI00126 | Bifidobacterium | YCFAC-BO 40 mM | | 40 mM | NO | YCFAC |
| | adolescentis | (Anaerobe Systems, AS- | | | | |
| | | 7523) | | | | |
| FBI00127 | Collinsella aerofaciens | YCFAC-B (Anaerobe | | NO | NO | YCFAC |
| | | Systems, AS-677) | | | | |
| FBI00128 | Hungatella effluvii | YCFAC-BO 80 mM | | 80 mM | NO | YCFAC |
| | | (Anaerobe Systems, AS- | | | | |
| | | 7524) | | | | |
| FBI00129 | Escherichia flexneri | YCFAC-BO 200 mM | | 200 mM | NO | YCFAC |
| | | (Anaerobe Systems, AS- | | | | |
| | | 7529) | | | | |
| FBI00130 | Coprococcus comes | YCFAC-B (Anaerobe | | NO | NO | YCFAC |
| | | Systems, AS-677) | | | | |
| FBI00131 | Fusicatenibacter | YCFAC-B (Anaerobe | | NO | NO | YCFAC |
| | saccharivorans | Systems, AS-677) | | | | |
| FBI00132 | Gordonibacter | YCFAC-BO 80 mM | | 80 mM | NO | YCFAC |
| | pamelaeae | (Anaerobe Systems, AS- | | | | |
| | | 7524) | | | | |
| FBI00133 | Oxalobacter formigenes | YCFAC-BO 80 mM | | 80 mM | NO | YCFAC + 100 |
| | | (Anaerobe Systems, AS- | | | | mM oxalate |
| | | 7524) | | | | |
| FBI00134 | Bifidobacterium | YCFAC-B (Anaerobe | | NO | NO | YCFAC |
| | adolescentis | Systems, AS-677) | | | | |
| FBI00135 | Bifidobacterium | YCFAC-B (Anaerobe | | NO | NO | YCFAC |
| | pseudocatenulatum | Systems, AS-677) | | | | |
| FBI00136 | Eisenbergiella tayi | Brain Heart Infusion | | NO | NO | YCFAC |
| | | (BHI), hemin, vitamin K | | | | |
| | | (Teknova, B1093) | | | | |
| FBI00137 | Bacteroides fragilis | YCFAC-BO 40 mM | | 40 mM | NO | YCFAC |
| | | (Anaerobe Systems, AS- | | | | |
| | | 7523) | | | | |
| FBI00138 | Blautia massiliensis | YCFAC-BO 40 mM | | 40 mM | NO | YCFAC |
| | | (Anaerobe Systems, AS- | | | | |
| | | 7523) | | | | |
| FBI00139 | Bacteroides | YCFAC-BO 80 mM | | 80 mM | NO | YCFAC |
| | thetaiotaomicron | (Anaerobe Systems, AS- | | | | |
| | | 7524) | | | | |
| FBI00140 | Phascolarctobacterium | YCFAC-BO 160 mM | | 160 mM | NO | YCFAC + 80 mM |
| | faecium | (Anaerobe Systems, AS- | | | | oxalate |
| | | 7527) | | | | |
| FBI00141 | Phascolarctobacterium | YCFAC-BO 80 mM | | 80 mM | NO | YCFAC + 80 mM |
| | faecium | (Anaerobe Systems, AS- | | | | oxalate |
| | | 7524) | | | | |
| FBI00142 | Clostridium fessum | YCFAC-BO 40 mM | | 40 mM | NO | YCFAC |
| | | (Anaerobe Systems, AS- | | | | |
| | | 7523) | | | | |
| FBI00143 | Parabacteroides merdae | YCFAC-B (Anaerobe | | NO | NO | YCFAC |
| | | Systems, AS-677) | | | | |
| FBI00144 | Holdemanella biformis | Brain Heart Infusion | | NO | NO | YCFAC + |
| | | (BHI), hemin, vitamin K | | | | hemin/vitamin K |
| | | (Teknova, B1093) | | | | |
| FBI00145 | Bifidobacterium | YCFAC-B (Anaerobe | | NO | NO | YCFAC |
| | adolescentis | Systems, AS-677) | | | | |
| FBI00146 | Blautia faecis | YCFAC-BO 80 mM | | 80 mM | NO | YCFAC |
| | | (Anaerobe Systems, AS- | | | | |
| | | 7524) | | | | |
| FBI00147 | Clostridium bolteae | YCFAC-BO 80 mM | | 80 mM | NO | YCFAC |
| | | (Anaerobe Systems, AS- | | | | |
| | | 7524) | | | | |
| FBI00148 | Oscillibacter sp. | Brain Heart Infusion | | NO | NO | YCFAC |
| | FBI00028 | (BHI), hemin, vitamin K | | | | |
| | | (Teknova, B1093) | | | | |
| FBI00149 | Monoglobus | YCFAC-BO 80 mM | | 80 mM | NO | YCFAC |
| | pectinilyticus | (Anaerobe Systems, AS- | | | | |
| | | 7524) | | | | |
| FBI00150 | Lachnospiraceae sp. | YCFAC-B (Anaerobe | | NO | NO | YCFAC |
| | FBI00033 | Systems, AS-677) | | | | |
| FBI00151 | Clostridium aldenense | YCFAC-BO 80 mM | | 80 mM | NO | YCFAC |
| | | (Anaerobe Systems, AS- | | | | |
| | | 7524) | | | | |
| FBI00152 | Dialister invisus | YCFAC-B (Anaerobe | | NO | NO | YCFAC + |
| | | Systems, AS-677) | | | | hemin/vitamin K |
| FBI00153 | Dialister succinatiphilus | YCFAC-BO 80 mM | | 80 mM | NO | YCFAC + 3 mM |
| | | (Anaerobe Systems, AS- | | | | succinate + 7.3 |
| | | 7524) | | | | mM formate |
| FBI00154 | Bacteroides dorei | Brain Heart Infusion | | NO | NO | YCFAC |
| | | (BHI), hemin, vitamin K | | | | |
| | | (Teknova, B1093) | | | | |
| FBI00155 | Blautia obeum | YCFAC-BO 40 mM | | 40 mM | NO | YCFAC |
| | | (Anaerobe Systems, AS- | | | | |
| | | 7523) | | | | |
| FBI00156 | Enterococcus durans | Brain Heart Infusion | | NO | NO | YCFAC |
| | | (BHI), hemin, vitamin K | | | | |
| | | (Teknova, B1093) | | | | |
| FBI00157 | Lachnospiraceae sp. | Bifidobacterium Selective | | NO | NO | YCFAC |
| | FBI00157 | agar (Anaerobe Systems, | | | | |
| | | AS-6423) | | | | |
| FBI00158 | Butyricimonas sp. | Columbia agar, 5% sheep | Antibiotics | NO | NO | YCFAC |
| | FBI00158 | blood (BD, 221165) | | | | |
| FBI00159 | Eisenbergiella tayi | YCFAC-BO 160 mM | | 160 mM | NO | YCFAC |
| | | (Anaerobe Systems, AS- | | | | |
| | | 7527) | | | | |
| FBI00160 | Ruminococcaceae sp. | Brain Heart Infusion | | NO | NO | YCFAC |
| | FBI00105 FBI00160 | (BHI), hemin, vitamin K | | | | |
| | | (Teknova, B1093) | | | | |
| FBI00161 | Bacteroides | Brain Heart Infusion | | NO | NO | YCFAC |
| | cellulosilyticus | (BHI), hemin, vitamin K | | | | |
| | | (Teknova, B1093) | | | | |
| FBI00162 | Bifidobacterium | Reinforced Clostridial | | NO | NO | YCFAC |
| | catenulatum | Agar (RCA) (Teknova, | | | | |
| | | C0205) | | | | |
| FBI00163 | Acidaminococcus | Reinforced Clostridial | | NO | NO | YCFAC |
| | intestini | Agar (RCA) (Teknova, | | | | |
| | | C0205) | | | | |
| FBI00164 | Bacteroides stercoris | Brain Heart Infusion | | NO | NO | YCFAC |
| | | (BHI), hemin, vitamin K | | | | |
| | | (Teknova, B1093) | | | | |
| FBI00165 | Bacteroides massiliensis | Brain Heart Infusion | | NO | NO | YCFAC |
| | | (BHI), hemin, vitamin K | | | | |
| | | (Teknova, B1093) | | | | |
| FBI00166 | Blautia massiliensis | Brain Heart Infusion | | NO | NO | YCFAC |
| | | (BHI), hemin, vitamin K | | | | |
| | | (Teknova, B1093) | | | | |
| FBI00167 | Dorea longicatena | YCFAC-B (Anaerobe | | NO | NO | YCFAC |
| | | Systems, AS-677) | | | | |
| FBI00168 | Collinsella aerofaciens | Reinforced Clostridial | | NO | NO | YCFAC |
| | | Agar (RCA) (Teknova, | | | | |
| | | C0205) | | | | |
| FBI00169 | Parabacteroides | Brain Heart Infusion | | NO | NO | YCFAC |
| | distasonis | (BHI), hemin, vitamin K | | | | |
| | | (Teknova, B1093) | | | | |
| FBI00170 | Eggerthella lenta | YCFAC-BO 80 mM | | 80 mM | NO | YCFAC |
| | | (Anaerobe Systems, AS- | | | | |
| | | 7524) | | | | |
| FBI00171 | Bilophila wadsworthia | YCFAC-BO 80 mM | | 80 mM | NO | YCFAC |
| | | (Anaerobe Systems, AS- | | | | |
| | | 7524) | | | | |
| FBI00172 | Bifidobacterium longum | Reinforced Clostridial | | NO | NO | YCFAC |
| | | Agar (RCA) (Teknova, | | | | |
| | | C0205) | | | | |
| FBI00173 | Bacteroides vulgatus | Bacteroides Bile Esculin | | NO | YES | YCFAC |
| | | (BBE) (Anaerobe Systems, | | | | |
| | | AS-144) | | | | |
| FBI00174 | Lactobacillus rogosae | YCFAC-B (Anaerobe | | NO | NO | YCFAC |
| | | Systems, AS-677) | | | | |
| FBI00175 | Holdemanella biformis | YCFAC-B (Anaerobe | | NO | NO | YCFAC + |
| | | Systems, AS-677) | | | | hemin/vitamin K |
| FBI00176 | Ruthenibacterium | Brain Heart Infusion | | NO | NO | YCFAC |
| | lactatiformans | (BHI), hemin, vitamin K | | | | |
| | | (Teknova, B1093) | | | | |
| FBI00177 | Parasutterella | Bacteroides Bile Esculin | | NO | YES | YCFAC |
| | excrementihominis | (BBE) (Anaerobe Systems, | | | | |
| | | AS-144) | | | | |
| FBI00178 | Bifidobacterium | YCFAC-B (Anaerobe | | NO | NO | YCFAC |
| | adolescentis | Systems, AS-677) | | | | |
| FBI00179 | Bifidobacterium | Reinforced Clostridial | | NO | NO | YCFAC |
| | adolescentis | Agar (RCA) (Teknova, | | | | |
| | | C0205) | | | | |
| FBI00180 | Alistipes sp. FBI00180 | Bacteroides Bile Esculin | | NO | YES | YCFAC |
| | | (BBE) (Anaerobe Systems, | | | | |
| | | AS-144) | | | | |
| FBI00181 | Blautia wexlerae | Brain Heart Infusion | | NO | NO | YCFAC |
| | | (BHI), hemin, vitamin K | | | | |
| | | (Teknova, B1093) | | | | |
| FBI00182 | Bacteroides coprocola | YCFAC-B (Anaerobe | | NO | NO | YCFAC |
| | | Systems, AS-677) | | | | |
| FBI00183 | Bacteroides dorei | Brain Heart Infusion | | NO | NO | YCFAC |
| | | (BHI), hemin, vitamin K | | | | |
| | | (Teknova, B1093) | | | | |
| FBI00184 | Bacteroides faecis | YCFAC-B (Anaerobe | | NO | NO | YCFAC |
| | | Systems, AS-677) | | | | |
| FBI00185 | Eisenbergiella tayi | YCFAC-BO 160 mM | | 160 mM | NO | YCFAC |
| | | (Anaerobe Systems, AS- | | | | |
| | | 7527) | | | | |
| FBI00186 | Coprococcus comes | OxyPras Plus Brucella | | NO | NO | YCFAC |
| | | Blood Agar (Oxyrase, P- | | | | |
| | | BRU-BA) | | | | |
| FBI00187 | Eubacterium rectale | OxyPras Plus Brucella | | NO | NO | YCFAC |
| | | Blood Agar (Oxyrase, P- | | | | |
| | | BRU-BA) | | | | |
| FBI00188 | Blautia faecis | YCFAC-B (Anaerobe | | NO | NO | YCFAC |
| | | Systems, AS-677) | | | | |
| FBI00189 | Bacteroides ovatus | YCFAC-BO 40 mM | | 40 mM | NO | YCFAC |
| | | (Anaerobe Systems, AS- | | | | |
| | | 7523) | | | | |
| FBI00190 | Bacteroides finegoldii | Brain Heart Infusion | | NO | NO | YCFAC |
| | | (BHI), hemin, vitamin K | | | | |
| | | (Teknova, B1093) | | | | |
| FBI00191 | Clostridiaceae sp. | OxyPras Plus Brucella | | NO | NO | YCFAC + |
| | FBI00191 | Blood Agar (Oxyrase, P- | | | | hemin/vitamin K |
| | | BRU-BA) | | | | |
| FBI00192 | Sutterella | YCFAC-B (Anaerobe | | NO | NO | YCFAC |
| | wadsworthensis | Systems, AS-677) | | | | |
| FBI00193 | Alistipes onderdonkii | OxyPras Plus Brucella | | NO | NO | YCFAC |
| | | Blood Agar (Oxyrase, P- | | | | |
| | | BRU-BA) | | | | |
| FBI00194 | Ruminococcus faecis | Brain Heart Infusion | | NO | NO | YCFAC |
| | | (BHI), hemin, vitamin K | | | | |
| | | (Teknova, B1093) | | | | |
| FBI00195 | Parasutterella | YCFAC-B (Anaerobe | | NO | NO | YCFAC |
| | excrementihominis | Systems, AS-677) | | | | |
| FBI00196 | Blautia obeum | Columbia agar, 5% sheep | | NO | NO | YCFAC |
| | | blood (BD, 221165) | | | | |
| FBI00197 | Bifidobacterium | YCFAC-B (Anaerobe | | NO | NO | YCFAC |
| | bifidum | Systems, AS-677) | | | | |
| FBI00198 | Lachnoclostridium | YCFAC-BO 40 mM | | 40 mM | NO | YCFAC |
| | pacaense | (Anaerobe Systems, AS- | | | | |
| | | 7523) | | | | |
| FBI00199 | Clostridium bolteae | YCFAC-BO 40 mM | | 40 mM | NO | YCFAC |
| | | (Anaerobe Systems, AS- | | | | |
| | | 7523) | | | | |
| FBI00200 | Longicatena caecimuris | YCFAC-B (Anaerobe | | NO | NO | YCFAC |
| | | Systems, AS-677) | | | | |
| FBI00201 | Eggerthella lenta | Columbia agar, 5% sheep | | NO | NO | YCFAC |
| | | blood (BD, 221165) | | | | |
| FBI00202 | Erysipelotrichaceae sp. | YCFAC-B (Anaerobe | | NO | NO | YCFAC |
| | FBI00202 | Systems, AS-677) | | | | |
| FBI00203 | Bacteroides kribbi/ | YCFAC-BO 40 mM | | 40 mM | NO | YCFAC |
| | Bacteroides koreensis | (Anaerobe Systems, AS- | | | | |
| | species cluster | 7523) | | | | |
| FBI00204 | Escherichia flexneri | YCFAC-BO 40 mM | | 40 mM | NO | YCFAC |
| | | (Anaerobe Systems, AS- | | | | |
| | | 7523) | | | | |
| FBI00205 | Blautia massiliensis | YCFAC-BO 40 mM | | 40 mM | NO | YCFAC |
| | | (Anaerobe Systems, AS- | | | | |
| | | 7523) | | | | |
| FBI00206 | Bacteroides | YCFAC-BO 40 mM | | 40 mM | NO | YCFAC |
| | xylanisolvens | (Anaerobe Systems, AS- | | | | |
| | | 7523) | | | | |
| FBI00207 | Parabacteroides merdae | YCFAC-B (Anaerobe | | NO | NO | YCFAC |
| | | Systems, AS-677) | | | | |
| FBI00208 | Anaerotruncus | YCFAC-BO 40 mM | | 40 mM | NO | YCFAC |
| | massiliensis | (Anaerobe Systems, AS- | | | | |
| | | 7523) | | | | |
| FBI00209 | Bacteroides salyersiae | YCFAC-BO 40 mM | | 40 mM | NO | YCFAC |
| | | (Anaerobe Systems, AS- | | | | |
| | | 7523) | | | | |
| FBI00210 | Bifidobacterium | YCFAC-B (Anaerobe | | NO | NO | YCFAC |
| | bifidum | Systems, AS-677) | | | | |
| FBI00211 | Bacteroides vulgatus | YCFAC-B (Anaerobe | | NO | NO | YCFAC |
| | | Systems, AS-677) | | | | |
| FBI00212 | Clostridium aldenense | YCFAC-B (Anaerobe | | NO | NO | YCFAC |
| | | Systems, AS-677) | | | | |
| FBI00213 | Ruminococcus bromii | Brain Heart Infusion | | NO | NO | YCFAC |
| | | (BHI), hemin, vitamin K | | | | |
| | | (Teknova, B1093) | | | | |
| FBI00214 | Blautia obeum | YCFAC-BO 40 mM | | 40 mM | NO | YCFAC |
| | | (Anaerobe Systems, AS- | | | | |
| | | 7523) | | | | |
| FBI00215 | Eubacterium rectale | YCFAC-B (Anaerobe | | NO | NO | YCFAC |
| | | Systems, AS-677) | | | | |
| FBI00216 | Blautia faecis | Columbia agar, 5% sheep | | NO | NO | YCFAC |
| | | blood (BD, 221165) | | | | |
| FBI00217 | Alistipes shahii | Bacteroides Bile Esculin | | NO | YES | YCFAC |
| | | (BBE) (Anaerobe Systems, | | | | |
| | | AS-144) | | | | |
| FBI00218 | Bacteroides uniformis | YCFAC-BO 40 mM | | 40 mM | NO | YCFAC |
| | | (Anaerobe Systems, AS- | | | | |
| | | 7523) | | | | |
| FBI00219 | Roseburia hominis | YCFAC-BO 40 mM | | 40 mM | NO | YCFAC |
| | | (Anaerobe Systems, AS- | | | | |
| | | 7523) | | | | |
| FBI00220 | Megasphaera | Brain Heart Infusion | | NO | NO | YCFAC |
| | massiliensis | (BHI), hemin, vitamin K | | | | |
| | | (Teknova, B1093) | | | | |
| FBI00221 | Butyricimonas | Brain Heart Infusion | | NO | NO | YCFAC |
| | faecihominis | (BHI), hemin, vitamin K | | | | |
| | | (Teknova, B1093) | | | | |
| FBI00222 | Alistipes onderdonkii | Brain Heart Infusion | | NO | NO | YCFAC |
| | | (BHI), hemin, vitamin K | | | | |
| | | (Teknova, B1093) | | | | |
| FBI00223 | Alistipes onderdonkii | Brain Heart Infusion | | NO | NO | YCFAC |
| | | (BHI), hemin, vitamin K | | | | |
| | | (Teknova, B1093) | | | | |
| FBI00224 | Sutterella | YCFAC-BO 40 mM | | 40 mM | NO | YCFAC |
| | wadsworthensis | (Anaerobe Systems, AS- | | | | |
| | | 7523) | | | | |
| FBI00225 | Phascolarctobacterium | YCFAC-BO 40 mM | | 40 mM | NO | YCFAC + 80 mM |
| | faecium | (Anaerobe Systems, AS- | | | | oxalate |
| | | 7523) | | | | |
| FBI00226 | Catabacter | YCFAC-BO 40 mM | | 40 mM | NO | YCFAC + |
| | hongkongensis | (Anaerobe Systems, AS- | | | | hemin/vitamin K |
| | | 7523) | | | | |
| FBI00227 | Bacteroides | Brain Heart Infusion | | NO | NO | YCFAC |
| | cellulosilyticus | (BHI), hemin, vitamin K | | | | |
| | | (Teknova, B1093) | | | | |
| FBI00228 | Collinsella aerofaciens | YCFAC-B (Anaerobe | | NO | NO | YCFAC |
| | | Systems, AS-677) | | | | |
| FBI00229 | Alistipes senegalensis | Brain Heart Infusion | | NO | NO | Thioglycollate |
| | | (BHI), hemin, vitamin K | | | | with |
| | | (Teknova, B1093) | | | | hemin/vitamin K |
| FBI00230 | Eisenbergiella tayi | YCFAC-BO 40 mM | | 40 mM | NO | YCFAC |
| | | (Anaerobe Systems, AS- | | | | |
| | | 7523) | | | | |
| FBI00231 | Parabacteroides | Brain Heart Infusion | | NO | NO | YCFAC |
| | distasonis | (BHI), hemin, vitamin K | | | | |
| | | (Teknova, B1093) | | | | |
| FBI00232 | Bacteroides stercoris | Columbia agar, 5% sheep | | NO | NO | YCFAC |
| | | blood (BD, 221165) | | | | |
| FBI00233 | Ruminococcaceae sp. | Brain Heart Infusion | | NO | NO | YCFAC + |
| | FBI00233 | (BHI), hemin, vitamin K | | | | hemin/vitamin K |
| | | (Teknova, B1093) | | | | |
| FBI00234 | Faecalicatena contorta | Columbia agar, 5% sheep | | NO | NO | YCFAC |
| | | blood (BD, 221165) | | | | |
| FBI00235 | Alistipes shahii | Brain Heart Infusion | | NO | NO | YCFAC |
| | | (BHI), hemin, vitamin K | | | | |
| | | (Teknova, B1093) | | | | |
| FBI00236 | Eisenbergiella tayi | YCFAC-BO 40 mM | | 40 mM | NO | YCFAC |
| | | (Anaerobe Systems, AS- | | | | |
| | | 7523) | | | | |
| FBI00237 | Dielma fastidiosa | Brain Heart Infusion | | NO | NO | YCFAC |
| | | (BHI), hemin, vitamin K | | | | |
| | | (Teknova, B1093) | | | | |
| FBI00238 | Alistipes sp. FBI00238 | Columbia agar, 5% sheep | | NO | NO | YCFAC |
| | | blood (BD, 221165) | | | | |
| FBI00239 | Lactonifactor | Brain Heart Infusion | | NO | NO | YCFAC |
| | longoviformis | (BHI), hemin, vitamin K | | | | |
| | | (Teknova, B1093) | | | | |
| FBI00240 | Clostridium citroniae | Brain Heart Infusion | | NO | NO | YCFAC |
| | | (BHI), hemin, vitamin K | | | | |
| | | (Teknova, B1093) | | | | |
| FBI00241 | Collinsella aerofaciens | YCFAC-B (Anaerobe | | NO | NO | YCFAC + |
| | | Systems, AS-677) | | | | hemin/vitamin K |
| FBI00242 | Clostridium aldenense | Brain Heart Infusion | | NO | NO | YCFAC |
| | | (BHI), hemin, vitamin K | | | | |
| | | (Teknova, B1093) | | | | |
| FBI00243 | Eubacterium siraeum | Brain Heart Infusion | | NO | NO | YCFAC, pH 6 |
| | | (BHI), hemin, vitamin K | | | | |
| | | (Teknova, B1093) | | | | |
| FBI00244 | Faecalibacterium | YCFAC-BO 40 mM | | 40 mM | NO | YCFAC, pH 6 |
| | prausnitzii | (Anaerobe Systems, AS- | | | | |
| | | 7523) | | | | |
| FBI00245 | Acidaminococcus | Columbia agar, 5% sheep | | NO | NO | YCFAC + |
| | intestini | blood (BD, 221165) | | | | hemin/vitamin K |
| FBI00246 | Bifidobacterium longum | Brain Heart Infusion | | NO | NO | YCFAC |
| | | (BHI), hemin, vitamin K | | | | |
| | | (Teknova, B1093) | | | | |
| FBI00247 | Phascolarctobacterium | YCFAC-BO 80 mM | | 80 mM | NO | YCFAC + 80 mM |
| | faecium | (Anaerobe Systems, AS- | | | | oxalate |
| | | 7524) | | | | |
| FBI00248 | Neglecta timonensis | Brain Heart Infusion | | NO | NO | YCFAC |
| | | (BHI), hemin, vitamin K | | | | |
| | | (Teknova, B1093) | | | | |
| FBI00249 | Citrobacter | Strain Isolation Media 3 | | NO | NO | YCFAC |
| | portucalensis | (SL3) | | | | |
| FBI00250 | Bifidobacterium | Reinforced Clostridial | | NO | NO | YCFAC |
| | adolescentis | Agar (RCA) (Teknova, | | | | |
| | | C0205) | | | | |
| FBI00251 | Bifidobacterium | Reinforced Clostridial | | NO | NO | YCFAC |
| | pseudocatenulatum | Agar (RCA) (Teknova, | | | | |
| | | C0205) | | | | |
| FBI00252 | Oscillibacter sp. | Columbia agar, 5% sheep | | NO | NO | BHI + hemin/vitK |
| | FBI00028 | blood (BD, 221165) | | | | |
| FBI00253 | Roseburia hominis | Brain Heart Infusion | | NO | NO | YCFAC |
| | | (BHI), hemin, vitamin K | | | | |
| | | (Teknova, B1093) | | | | |
| FBI00254 | Eubacterium hallii | Brain Heart Infusion | | NO | NO | YCFAC |
| | | (BHI), hemin, vitamin K | | | | |
| | | (Teknova, B1093) | | | | |
| FBI00255 | Hungatella effluvii | YCFAC-BO 80 mM | | 80 mM | NO | YCFAC |
| | | (Anaerobe Systems, AS- | | | | |
| | | 7524) | | | | |
| FBI00256 | Blautia faecis | YCFAC-BO 80 mM | | 80 mM | NO | YCFAC |
| | | (Anaerobe Systems, AS- | | | | |
| | | 7524) | | | | |
| FBI00257 | Eubacterium eligens | Strain Isolation Media 3 | | NO | NO | YCFAC |
| | | (SL3) | | | | |
| FBI00258 | Turicibacter sanguinis | Strain Isolation Media 3 | | NO | NO | Thioglycollate |
| | | (SL3) | | | | with |
| | | | | | | hemin/vitamin K |
| FBI00259 | Dorea longicatena | Reinforced Clostridial | | NO | NO | YCFAC |
| | | Agar (RCA) (Teknova, | | | | |
| | | C0205) | | | | |
| FBI00260 | Eubacterium rectale | Brain Heart Infusion | | NO | NO | YCFAC |
| | | (BHI), hemin, vitamin K | | | | |
| | | (Teknova, B1093) | | | | |
| FBI00261 | Bacteroides uniformis | Brain Heart Infusion | | NO | NO | YCFAC |
| | | (BHI), hemin, vitamin K | | | | |
| | | (Teknova, B1093) | | | | |
| FBI00262 | Bacteroides massiliensis | Brain Heart Infusion | | NO | NO | YCFAC |
| | | (BHI), hemin, vitamin K | | | | |
| | | (Teknova, B1093) | | | | |
| FBI00263 | Bacteroides caccae | Strain Isolation Media 3 | | NO | NO | YCFAC |
| | | (SL3) | | | | |
| FBI00264 | Bifidobacterium | Reinforced Clostridial | | NO | NO | YCFAC |
| | adolescentis | Agar (RCA) (Teknova, | | | | |
| | | C0205) | | | | |
| FBI00265 | Bacteroides | YCFAC-BO 80 mM | | 80 mM | NO | YCFAC |
| | cellulosilyticus | (Anaerobe Systems, AS- | | | | |
| | | 7524) | | | | |
| FBI00266 | Coprococcus eutactus | Strain Isolation Media 3 | | NO | NO | YCFAC |
| | | (SL3) | | | | |
| FBI00267 | Anaerofustis | YCFAC-BO 80 mM | | 80 mM | NO | YCFAC |
| | stercorihominis | (Anaerobe Systems, AS- | | | | |
| | | 7524) | | | | |
| FBI00268 | Clostridiales sp. | Brain Heart Infusion | | NO | NO | Thioglycollate |
| | FBI00268 | (BHI), hemin, vitamin K | | | | with |
| | | (Teknova, B1093) | | | | hemin/vitamin K |
| FBI00269 | Alistipes putredinis | Brain Heart Infusion | | NO | NO | YCFAC |
| | | (BHI), hemin, vitamin K | | | | |
| | | (Teknova, B1093) | | | | |
| FBI00270 | Methanobrevibacter | Columbia agar, 5% sheep | Antibiotics | NO | NO | SAB |
| | smithii | blood (BD, 221165) | | | | |
| FBI00271 | Bacteroides | Brain Heart Infusion | | NO | NO | YCFAC |
| | xylanisolvens | (BHI), hemin, vitamin K | | | | |
| | | (Teknova, B1093) | | | | |
| FBI00272 | Clostridiales XIII sp. | Columbia agar, 5% sheep | | NO | NO | Thioglycollate |
| | FBI00272 | blood (BD, 221165) | | | | with |
| | | | | | | hemin/vitamin K |
| FBI00273 | Barnesiella | Brain Heart Infusion | | NO | NO | YCFAC |
| | intestinihominis | (BHI), hemin, vitamin K | | | | |
| | | (Teknova, B1093) | | | | |
| FBI00274 | Eubacterium | YCFAC-BO 80 mM | | 80 mM | NO | YCFAC, pH 6 |
| | xylanophilum | (Anaerobe Systems, AS- | | | | |
| | | 7524) | | | | |
| FBI00275 | Holdemanella biformis | Brain Heart Infusion | | NO | NO | YCFAC + |
| | | (BHI), hemin, vitamin K | | | | hemin/vitamin K |
| | | (Teknova, B1093) | | | | |
| FBI00276 | Dorea formicigenerans | Strain Isolation Media 3 | | NO | NO | YCFAC |
| | | (SL3) | | | | |
| FBI00277 | Alistipes onderdonkii | Brain Heart Infusion | | NO | NO | YCFAC |
| | | (BHI), hemin, vitamin K | | | | |
| | | (Teknova, B1093) | | | | |
| FBI00278 | Eubacterium ventriosum | Brain Heart Infusion | | NO | NO | YCFAC, pH 6 |
| | | (BHI), hemin, vitamin K | | | | |
| | | (Teknova, B1093) | | | | |
| FBI00279 | Coprococcus comes | YCFAC-BO 80 mM | | 80 mM | NO | YCFAC |
| | | (Anaerobe Systems, AS- | | | | |
| | | 7524) | | | | |
| FBI00280 | Bacteroides | YCFAC-BO 80 mM | | 80 mM | NO | YCFAC |
| | thetaiotaomicron | (Anaerobe Systems, AS- | | | | |
| | | 7524) | | | | |
| FBI00281 | Senegalimassilia | Reinforced Clostridial | | NO | NO | YCFAC |
| | anaerobia | Agar (RCA) (Teknova, | | | | |
| | | C0205) | | | | |
| FBI00282 | Porphyromonas | Columbia agar, 5% sheep | | NO | NO | YCFAC |
| | asaccharolytica | blood (BD, 221165) | | | | |
| FBI00283 | Ruminococcus bromii | Brain Heart Infusion | | NO | NO | YCFAC |
| | | (BHI), hemin, vitamin K | | | | |
| | | (Teknova, B1093) | | | | |
| FBI00284 | Blautia obeum | Strain Isolation Media 3 | | NO | NO | YCFAC |
| | | (SL3) | | | | |
| FBI00285 | Lachnospira sp. | Brain Heart Infusion | | NO | NO | YCFAC |
| | FBI00063 FBI00285 | (BHI), hemin, vitamin K | | | | |
| | FBI00364 | (Teknova, B1093) | | | | |
| FBI00286 | Fusicatenibacter | Brain Heart Infusion | | NO | NO | YCFAC |
| | saccharivorans | (BHI), hemin, vitamin K | | | | |
| | | (Teknova, B1093) | | | | |
| FBI00287 | Alistipes shahii | Strain Isolation Media 3 | | NO | NO | YCFAC |
| | | (SL3) | | | | |
| FBI00288 | Blautia | Brain Heart Infusion | | NO | NO | YCFAC |
| | hydrogenotrophica | (BHI), hemin, vitamin K | | | | |
| | | (Teknova, B1093) | | | | |
| FBI00289 | Oxalobacter formigenes | YCFAC-BO 80 mM | | 80 mM | NO | YCFAC + 80 mM |
| | | (Anaerobe Systems, AS- | | | | oxalate |
| | | 7524) | | | | |
| FBI00290 | Lachnospiraceae sp. | Brain Heart Infusion | | NO | NO | YCFAC |
| | FBI00290 | (BHI), hemin, vitamin K | | | | |
| | | (Teknova, B1093) | | | | |
| FBI00291 | Oribacterium sp. | Strain Isolation Media 3 | | NO | NO | YCFAC |
| | FBI00291 | (SL3) | | | | |
| FBI00292 | Methanobrevibacter | Columbia agar, 5% sheep | | NO | NO | SAB |
| | smithii | blood (BD, 221165) | | | | |
| FBI00293 | Bifidobacterium | Reinforced Clostridial | | NO | NO | YCFAC |
| | adolescentis | Agar (RCA) (Teknova, | | | | |
| | | C0205) | | | | |
| FBI00294 | Bacteroides stercoris | YCFAC-B (Anaerobe | | NO | NO | YCFAC |
| | | Systems, AS-677) | | | | |
| FBI00295 | Bifidobacterium | Reinforced Clostridial | | NO | NO | YCFAC |
| | adolescentis | Agar (RCA) (Teknova, | | | | |
| | | C0205) | | | | |
| FBI00296 | Dorea longicatena | Brain Heart Infusion | | NO | NO | YCFAC |
| | | (BHI), hemin, vitamin K | | | | |
| | | (Teknova, B1093) | | | | |
| FBI00297 | Alistipes obesi | Strain Isolation Media 3 | | NO | NO | YCFAC + |
| | | (SL3) | | | | hemin/vitamin K |
| FBI00298 | Faecalibacterium | Brain Heart Infusion | | NO | NO | YCFAC |
| | prausnitzii | (BHI), hemin, vitamin K | | | | |
| | | (Teknova, B1093) | | | | |
| FBI00299 | Streptococcus | Lactobacillus MRS | | NO | NO | YCFAC |
| | pasteurianus | (Anaerobe Systems, AS- | | | | |
| | | 6429) | | | | |
| FBI00300 | Collinsella aerofaciens | Columbia agar, 5% sheep | | NO | NO | YCFAC |
| | | blood (BD, 221165) | | | | |
| FBI00301 | Bifidobacterium | Brain Heart Infusion | | NO | NO | YCFAC |
| | adolescentis | (BHI), hemin, vitamin K | | | | |
| | | (Teknova, B1093) | | | | |
| FBI00302 | Blautia faecis | Columbia agar, 5% sheep | | NO | NO | YCFAC |
| | | blood (BD, 221165) | | | | |
| FBI00303 | Parabacteroides merdae | Brain Heart Infusion | | NO | NO | YCFAC |
| | | (BHI), hemin, vitamin K | | | | |
| | | (Teknova, B1093) | | | | |
| FBI00304 | Dorea longicatena | Strain Isolation Media 3 | | NO | NO | YCFAC |
| | | (SL3) | | | | |
| FBI00305 | Alistipes onderdonkii | Bacteroides Bile Esculin | | NO | YES | YCFAC |
| | | (BBE) (Anaerobe Systems, | | | | |
| | | AS-144) | | | | |
| FBI00306 | Parasutterella | Strain Isolation Media 3 | | NO | NO | BHI + hemin/vitK |
| | excrementihominis | (SL3) | | | | |
| FBI00307 | Parasutterella | Bacteroides Bile Esculin | | NO | YES | BHI + hemin/vitK |
| | excrementihominis | (BBE) (Anaerobe Systems, | | | | |
| | | AS-144) | | | | |
| FBI00308 | Bacteroides vulgatus | YCFAC-B (Anaerobe | | NO | NO | YCFAC |
| | | Systems, AS-677) | | | | |
| FBI00309 | Eubacterium rectale | Brain Heart Infusion | | NO | NO | YCFAC |
| | | (BHI), hemin, vitamin K | | | | |
| | | (Teknova, B1093) | | | | |
| FBI00310 | Butyricimonas | Brain Heart Infusion | | NO | NO | YCFAC |
| | faecihominis | (BHI), hemin, vitamin K | | | | |
| | | (Teknova, B1093) | | | | |
| FBI00311 | Anaerostipes hadrus | YCFAC-B (Anaerobe | | NO | NO | YCFAC |
| | | Systems, AS-677) | | | | |
| FBI00312 | Alistipes shahii | Bacteroides Bile Esculin | | NO | YES | YCFAC |
| | | (BBE) (Anaerobe Systems, | | | | |
| | | AS-144) | | | | |
| FBI00313 | Collinsella aerofaciens | Columbia agar, 5% sheep | | NO | NO | YCFAC |
| | | blood (BD, 221165) | | | | |
| FBI00314 | Anaerotignum | Brain Heart Infusion | | NO | NO | YCFAC |
| | lactatifermentans | (BHI), hemin, vitamin K | | | | |
| | | (Teknova, B1093) | | | | |
| FBI00315 | Blautia obeum | Brain Heart Infusion | | NO | NO | YCFAC |
| | | (BHI), hemin, vitamin K | | | | |
| | | (Teknova, B1093) | | | | |
| FBI00316 | Collinsella aerofaciens | Strain Isolation Media 3 | | NO | NO | YCFAC |
| | | (SL3) | | | | |
| FBI00317 | Bifidobacterium longum | YCFAC-B (Anaerobe | | NO | NO | YCFAC |
| | | Systems, AS-677) | | | | |
| FBI00318 | Collinsella aerofaciens | Brain Heart Infusion | | NO | NO | YCFAC |
| | | (BHI), hemin, vitamin K | | | | |
| | | (Teknova, B1093) | | | | |
| FBI00319 | Collinsella aerofaciens | Columbia agar, 5% sheep | | NO | NO | YCFAC |
| | | blood (BD, 221165) | | | | |
| FBI00320 | Dorea formicigenerans | Strain Isolation Media 3 | | NO | NO | YCFAC |
| | | (SL3) | | | | |
| FBI00321 | Bacteroides vulgatus | Strain Isolation Media 3 | | NO | NO | YCFAC |
| | | (SL3) | | | | |
| FBI00322 | Bifidobacterium | Brain Heart Infusion | | NO | NO | YCFAC |
| | adolescentis | (BHI), hemin, vitamin K | | | | |
| | | (Teknova, B1093) | | | | |
| FBI00323 | Blautia wexlerae | Brain Heart Infusion | | NO | NO | YCFAC |
| | | (BHI), hemin, vitamin K | | | | |
| | | (Teknova, B1093) | | | | |
| FBI00324 | Bilophila wadsworthia | Columbia agar, 5% sheep | | NO | NO | YCFAC |
| | | blood (BD, 221165) | | | | |
| FBI00325 | Alistipes indistinctus | Brain Heart Infusion | | NO | NO | YCFAC + |
| | | (BHI), hemin, vitamin K | | | | hemin/vitamin K |
| | | (Teknova, B1093) | | | | |
| FBI00326 | Bacteroides vulgatus | Chocolate Agar (Teknova, | | NO | NO | YCFAC |
| | | C4900) | | | | |
| FBI00327 | Coprococcus comes | Chocolate Agar (Teknova, | | NO | NO | YCFAC |
| | | C4900) | | | | |
| FBI00328 | Blautia luti | Strain Isolation Media 3 | | NO | NO | YCFAC |
| | | (SL3) | | | | |
| FBI00329 | Alistipes indistinctus | Brain Heart Infusion | | NO | NO | YCFAC |
| | | (BHI), hemin, vitamin K | | | | |
| | | (Teknova, B1093) | | | | |
| FBI00330 | Bifidobacterium longum | Lactobacillus MRS | | NO | NO | YCFAC |
| | | (Anaerobe Systems, AS- | | | | |
| | | 6429) | | | | |
| FBI003 31 | Bacillus circulans | YCFAC-B (Anaerobe | | NO | NO | YCFAC |
| | | Systems, AS-677) | | | | |
| FBI00332 | Clostridium intestinale | YCFAC-B (Anaerobe | | NO | NO | YCFAC |
| | | Systems, AS-677) | | | | |
| FBI00333 | Alistipes onderdonkii | Strain Isolation Media 3 | | NO | NO | YCFAC |
| | | (SL3) | | | | |
| FBI00334 | Bacteroides caccae | Brain Heart Infusion | | NO | NO | YCFAC |
| | | (BHI), hemin, vitamin K | | | | |
| | | (Teknova, B1093) | | | | |
| FBI00335 | Anaerostipes hadrus | Brain Heart Infusion | | NO | NO | YCFAC |
| | | (BHI), hemin, vitamin K | | | | |
| | | (Teknova, B1093) | | | | |
| FBI00336 | Staphylococcus | Brain Heart Infusion | | NO | NO | YCFAC |
| | epidermidis | (BHI), hemin, vitamin K | | | | |
| | | (Teknova, B1093) | | | | |
| FBI00337 | Coprococcus comes | Strain Isolation Media 3 | | NO | NO | YCFAC |
| | | (SL3) | | | | |
| FBI00338 | Blautia obeum | Chocolate Agar (Teknova, | | NO | NO | YCFAC |
| | | C4900) | | | | |
| FBI00339 | Eubacterium rectale | Brain Heart Infusion | | NO | NO | YCFAC |
| | | (BHI), hemin, vitamin K | | | | |
| | | (Teknova, B1093) | | | | |
| FBI00340 | Lachnospiraceae sp. | Brain Heart Infusion | | NO | NO | YCFAC |
| | FBI00033 | (BHI), hemin, vitamin K | | | | |
| | | (Teknova, B1093) | | | | |
| FBI00341 | Lachnospiraceae sp. | Columbia agar, 5% sheep | | NO | NO | YCFAC |
| | FBI00071 | blood (BD, 221165) | | | | |
| FBI00342 | Alistipes indistinctus | Bacteroides Bile Esculin | | NO | YES | YCFAC |
| | | (BBE) (Anaerobe Systems, | | | | |
| | | AS-144) | | | | |
| FBI00343 | Sutterella massiliensis | Brain Heart Infusion | | NO | NO | YCFAC |
| | | (BHI), hemin, vitamin K | | | | |
| | | (Teknova, B1093) | | | | |
| FBI00344 | Alistipes putredinis | Brain Heart Infusion | | NO | NO | YCFAC |
| | | (BHI), hemin, vitamin K | | | | |
| | | (Teknova, B1093) | | | | |
| FBI00345 | Roseburia inulinivorans | Strain Isolation Media 1 | | NO | NO | YCFAC |
| | | (SL1) | | | | |
| FBI00346 | Coriobacteriia sp. | Brain Heart Infusion | | NO | NO | YCFAC |
| | FBI00346 | (BHI), hemin, vitamin K | | | | |
| | | (Teknova, B1093) | | | | |
| FBI00347 | Bacteroides uniformis | Strain Isolation Media 1 | | NO | NO | YCFAC |
| | | (SL1) | | | | |
| FBI00348 | Parabacteroides merdae | Chocolate Agar (Teknova, | | NO | NO | YCFAC |
| | | C4900) | | | | |
| FBI00349 | Holdemanella biformis | Brain Heart Infusion | | NO | NO | BHI + hemin/vitK |
| | | (BHI), hemin, vitamin K | | | | |
| | | (Teknova, B1093) | | | | |
| FBI00350 | Alistipes putredinis | Bacteroides Bile Esculin | | NO | YES | YCFAC |
| | | (BBE) (Anaerobe Systems, | | | | |
| | | AS-144) | | | | |
| FBI00351 | Alistipes obesi | Columbia agar, 5% sheep | | NO | NO | YCFAC |
| | | blood (BD, 221165) | | | | |
| FBI00352 | Collinsella aerofaciens | Columbia agar, 5% sheep | | NO | NO | YCFAC |
| | | blood (BD, 221165) | | | | |
| FBI00353 | Bifidobacterium | Brain Heart Infusion | | NO | NO | YCFAC |
| | adolescentis | (BHI), hemin, vitamin K | | | | |
| | | (Teknova, B1093) | | | | |
| FBI00354 | Bifidobacterium | Strain Isolation Media 3 | | NO | NO | YCFAC |
| | adolescentis | (SL3) | | | | |
| FBI00355 | Blautia massiliensis | Strain Isolation Media 1 | | NO | NO | YCFAC |
| | | (SL1) | | | | |
| FBI00356 | Eubacterium eligens | Brain Heart Infusion | | NO | NO | YCFAC |
| | | (BHI), hemin, vitamin K | | | | |
| | | (Teknova, B1093) | | | | |
| FBI00357 | Bacteroides kribbi/ | Strain Isolation Media 1 | | NO | NO | YCFAC |
| | Bacteroides koreensis | (SL1) | | | | |
| | species cluster | | | | | |
| FBI00358 | Eubacterium hallii | Strain Isolation Media 1 | | NO | NO | YCFAC |
| | | (SL1) | | | | |
| FBI00359 | Eubacterium rectale | Brain Heart Infusion | | NO | NO | YCFAC |
| | | (BHI), hemin, vitamin K | | | | |
| | | (Teknova, B1093) | | | | |
| FBI00360 | Bacteroides massiliensis | Brain Heart Infusion | | NO | NO | YCFAC |
| | | (BHI), hemin, vitamin K | | | | |
| | | (Teknova, B1093) | | | | |
| FBI00361 | Bacteroides stercoris | Strain Isolation Media 3 | | NO | NO | YCFAC |
| | | (SL3) | | | | |
| FBI00362 | Prevotella copri | Chocolate Agar (Teknova, | | NO | NO | Thioglycollate |
| | | C4900) | | | | with |
| | | | | | | hemin/vitamin K |
| FBI00363 | Roseburia intestinalis | Strain Isolation Media 1 | | NO | NO | YCFAC |
| | | (SL1) | | | | |
| FBI00364 | Lachnospira sp. | Brain Heart Infusion | | NO | NO | YCFAC |
| | FBI00063 FBI00285 | (BHI), hemin, vitamin K | | | | |
| | FBI00364 | (Teknova, B1093) | | | | |
| FBI00365 | Paraprevotella clara | Chocolate Agar (Teknova, | | NO | NO | YCFAC |
| | | C4900) | | | | |
| FBI00366 | Eubacterium eligens | Brain Heart Infusion | | NO | NO | YCFAC |
| | | (BHI), hemin, vitamin K | | | | |
| | | (Teknova, B1093) | | | | |
| FBI00367 | Phascolarctobacterium | Strain Isolation Media 1 | | NO | NO | YCFAC |
| | faecium | (SL1) | | | | |
| FBI00368 | Alistipes putredinis | Brain Heart Infusion | | NO | NO | YCFAC |
| | | (BHI), hemin, vitamin K | | | | |
| | | (Teknova, B1093) | | | | |
| FBI00369 | Clostridiales sp. | Brain Heart Infusion | | NO | NO | BHI + hemin/vitK |
| | FBI00369 | (BHI), hemin, vitamin K | | | | |
| | | (Teknova, B1093) | | | | |
| FBI00370 | Bifidobacterium | Columbia agar, 5% sheep | | NO | NO | YCFAC |
| | adolescentis | blood (BD, 221165) | | | | |
| FBI00371 | Bacteroides stercoris | Brain Heart Infusion | | NO | NO | YCFAC |
| | | (BHI), hemin, vitamin K | | | | |
| | | (Teknova, B1093) | | | | |
| FBI00372 | Dorea longicatena | Chocolate Agar (Teknova, | | NO | NO | YCFAC |
| | | C4900) | | | | |
| FBI00373 | Coriobacteriaceae sp. | Columbia agar, 5% sheep | | NO | NO | Thioglycollate |
| | FBI00373 FBI00374 | blood (BD, 221165) | | | | with |
| | | | | | | hemin/vitamin K |
| FBI00374 | Coriobacteriaceae sp. | Columbia agar, 5% sheep | | NO | NO | Thioglycollate |
| | FBI00373 FBI00374 | blood (BD, 221165) | | | | with |
| | | | | | | hemin/vitamin K |
| FBI00375 | Dorea longicatena | Chocolate Agar (Teknova, | | NO | NO | YCFAC |
| | | C4900) | | | | |
| FBI00376 | Dialister succinatiphilus | Brain Heart Infusion | | NO | NO | BHI + hemin/vitK |
| | | (BHI), hemin, vitamin K | | | | |
| | | (Teknova, B1093) | | | | |
| FBI00377 | Clostridiales sp. | Brain Heart Infusion | | NO | NO | Thioglycollate |
| | FBI00377 | (BHI), hemin, vitamin K | | | | with |
| | | (Teknova, B1093) | | | | hemin/vitamin K |

TABLE 4

| | | | | NCBI | | | |
|---|---|---|---|---|---|---|---|
| | | | | Taxonomy | Closest 16S | % Match | SEQ ID |
| Strain # | Species ID | Kingdom | Phylum | ID | Species | (16S) | NO: X |
| FBI00001 | Clostridium citroniae | bacteria | firmicutes | 358743 | Clostridium citroniae | 99.64 | 1 |
| FBI00002 | Bacteroides salyersiae | bacteria | bacteroidetes | 291644 | Bacteroides salyersiae | 99.5 | 2 |
| FBI00003 | Enterococcus faecalis | bacteria | firmicutes | 1351 | Enterococcus faecalis | 99.93 | 3 |
| FBI00004 | Neglecta timonensis | bacteria | firmicutes | 1776382 | Neglecta timonensis | 99.14 | 4 |
| FBI00005 | Enterococcus casseliflavus | bacteria | firmicutes | 37734 | Enterococcus gallinarum | 99.65 | 5 |
| FBI00006 | Enterobacter himalayensis | bacteria | proteobacteria | 547 | Enterobacter hormaechei | 99 | 6 |
| FBI00007 | Enterococcus casseliflavus | bacteria | firmicutes | 37734 | Enterococcus | 99.05 | 7 |
| | | | | | casseliflavus | | |
| FBI00008 | Blautia luti | bacteria | firmicutes | 89014 | Blautia luti | 97.02 | 8 |
| FBI00009 | Bifidobacterium adolescentis | bacteria | actinobacteria | 1680 | Bifidobacterium faecale | 98.6 | 9 |
| FBI00010 | Blautia obeum | bacteria | firmicutes | 40520 | Blautia obeum | 98.12 | 10 |
| FBI00011 | Bifidobacterium longum | bacteria | actinobacteria | 216816 | Bifidobacterium longum | 99.28 | 11 |
| FBI00012 | Alistipes onderdonkii | bacteria | bacteroidetes | 328813 | Alistipes onderdonkii | 99.71 | 12 |
| FBI00013 | Parabacteroides merdae | bacteria | bacteroidetes | 46503 | Parabacteroides merdae | 99.5 | 13 |
| FBI00014 | Blautia luti | bacteria | firmicutes | 89014 | Blautia luti | 97.02 | 14 |
| FBI00015 | Bacteroides uniformis | bacteria | bacteroidetes | 820 | Bacteroides uniformis | 99.78 | 15 |
| FBI00016 | Bifidobacterium | bacteria | actinobacteria | 28026 | Bifidobacterium | 99.64 | 16 |
| | pseudocatenulatum | | | | pseudocatenulatum | | |
| FBI00017 | Blautia obeum | bacteria | firmicutes | 40520 | Blautia obeum | 98.34 | 17 |
| FBI00018 | Eubacterium rectale | bacteria | firmicutes | 39491 | Eubacterium rectale | 99.71 | 18 |
| FBI00019 | Alistipes timonensis | bacteria | bacteroidetes | 1465754 | Alistipes timonensis | 99.78 | 19 |
| FBI00020 | Bacteroides thetaiotaomicron | bacteria | bacteroidetes | 818 | Bacteroides | 99.57 | 20 |
| | | | | | thetaiotaomicron | | |
| FBI00021 | Bacteroides kribbi/ | bacteria | bacteroidetes | 816 | Bacteroides kribbi | 99.07 | 21 |
| | Bacteroides koreensis species | | | | | | |
| | cluster | | | | | | |
| FBI00022 | Alistipes putredinis | bacteria | bacteroidetes | 28117 | Alistipes putredinis | 99.93 | 22 |
| FBI00023 | Enterococcus casseliflavus | bacteria | firmicutes | 37734 | Enterococcus gallinarum | 99.65 | 23 |
| FBI00024 | Bacteroides kribbi/ | bacteria | bacteroidetes | 816 | Bacteroides koreensis | 99.07 | 24 |
| | Bacteroides koreensis species | | | | | | |
| | cluster | | | | | | |
| FBI00025 | Coprococcus comes | bacteria | firmicutes | 410072 | Coprococcus comes | 99.21 | 25 |
| FBI00026 | Enterobacter hormaechei | bacteria | proteobacteria | 158836 | Enterobacter hormaechei | 99.14 | 26 |
| FBI00027 | Fusicatenibacter | bacteria | firmicutes | 1150298 | Fusicatenibacter | 97.6 | 27 |
| | saccharivorans | | | | saccharivorans | | |
| FBI00028 | Oscillibacter sp. FBI00028 | bacteria | firmicutes | 459786 | Oscillibacter | 93.85 | 28 |
| | | | | | valericigenes | | |
| FBI00029 | Parabacteroides distasonis | bacteria | bacteroidetes | 823 | Parabacteroides | 99.26 | 29 |
| | | | | | distasonis | | |
| FBI00030 | Eggerthella lenta | bacteria | firmicutes | 84112 | Eggerthella lenta | 98.47 | 30 |
| FBI00031 | Enterobacter hormaechei | bacteria | proteobacteria | 158836 | Enterobacter hormaechei | 99.43 | 31 |
| FBI00032 | Anaerostipes hadrus | bacteria | firmicutes | 649756 | Anaerostipes hadrus | 99.64 | 32 |
| FBI00033 | Lachnospiraceae sp. | bacteria | firmicutes | 186803 | Clostridium | 93.56 | 33 |
| | FBI00033 | | | | amygdalinum | | |
| FBI00034 | Eubacterium eligens | bacteria | firmicutes | 39485 | Eubacterium eligens | 98.78 | 34 |
| FBI00035 | Enterococcus casseliflavus | bacteria | firmicutes | 37734 | Enterococcus | 98.24 | 35 |
| | | | | | casseliflavus | | |
| FBI00036 | Blautia faecis | bacteria | firmicutes | 871665 | Blautia faecis | 99.53 | 36 |
| FBI00037 | Enterococcus casseliflavus | bacteria | firmicutes | 37734 | Enterococcus gallinarum | 99.65 | 37 |
| FBI00038 | Coprococcus eutactus | bacteria | firmicutes | 33043 | Coprococcus eutactus | 95.96 | 38 |
| FBI00039 | Bacteroides vulgatus | bacteria | bacteroidetes | 821 | Bacteroides vulgatus | 99.71 | 39 |
| FBI00040 | Bilophila wadsworthia | bacteria | proteobacteria | 35833 | Desulfovibrio | 91.38 | 40 |
| | | | | | desulfuricans | | |
| FBI00041 | Phascolarctobacterium | bacteria | firmicutes | 33025 | Phascolarctobacterium | 99.23 | 41 |
| | faecium | | | | faecium | | |
| FBI00042 | Bacteroides xylanisolvens | bacteria | bacteroidetes | 371601 | Bacteroides | 99.71 | 42 |
| | | | | | xylanisolvens | | |
| FBI00043 | Bifidobacterium dentium | bacteria | actinobacteria | 1689 | Bifidobacterium | 99.35 | 43 |
| | | | | | dentium | | |
| FBI00044 | Blautia wexlerae | bacteria | firmicutes | 418240 | Blautia wexlerae | 98.69 | 44 |
| FBI00045 | Bifidobacterium adolescentis | bacteria | actinobacteria | 1680 | Bifidobacterium faecale | 99.56 | 45 |
| FBI00046 | Bacteroides caccae | bacteria | bacteroidetes | 47678 | Bacteroides caccae | 99.71 | 46 |
| FBI00047 | Eubacterium eligens | bacteria | firmicutes | 39485 | Eubacterium eligens | 98.79 | 47 |
| FBI00048 | Fusicatenibacter | bacteria | firmicutes | 1150298 | Fusicatenibacter | 97.95 | 48 |
| | saccharivorans | | | | saccharivorans | | |
| FBI00049 | Dialister succinatiphilus | bacteria | firmicutes | 487173 | Dialister succinatiphilus | 95.74 | 49 |
| FBI00050 | Bacteroides nordii | bacteria | bacteroidetes | 291645 | Bacteroides nordii | 98.63 | 50 |
| FBI00051 | Dorea formicigenerans | bacteria | firmicutes | 39486 | Dorea formicigenerans | 98.07 | 51 |
| FBI00052 | Bacteroides xylanisolvens | bacteria | bacteroidetes | 371601 | Bacteroides | 99.14 | 52 |
| | | | | | xylanisolvens | | |
| FBI00053 | Lactobacillus rogosae | bacteria | firmicutes | 706562 | Lachnospira | 97.36 | 53 |
| | | | | | pectinoschiza | | |
| FBI00054 | Escherichia flexneri | bacteria | proteobacteria | 623 | Escherichia fergusonii | 99.71 | 54 |
| FBI00055 | Bacteroides kribbi/ | bacteria | bacteroidetes | 816 | Bacteroides kribbi | 99.64 | 55 |
| | Bacteroides koreensis species | | | | | | |
| | cluster | | | | | | |
| FBI00056 | Clostridium citroniae | bacteria | firmicutes | 358743 | Clostridium citroniae | 99.2 | 56 |
| FBI00057 | Dorea longicatena | bacteria | firmicutes | 88431 | Dorea longicatena | 99.7 | 57 |
| FBI00058 | Eubacterium rectale | bacteria | firmicutes | 39491 | Eubacterium rectale | 99.71 | 58 |
| FBI00059 | Bacteroides stercorirosoris | bacteria | bacteroidetes | 871324 | Bacteroides oleiciplenus | 98.81 | 59 |
| FBI00060 | Bifidobacterium longum | bacteria | actinobacteria | 216816 | Bifidobacterium longum | 99.49 | 60 |
| FBI00061 | Alistipes shahii | bacteria | bacteroidetes | 328814 | Alistipes shahii | 99.19 | 61 |
| FBI00062 | Collinsella aerofaciens | bacteria | actinobacteria | 74426 | Collinsella aerofaciens | 99.48 | 62 |
| FBI00063 | Lachnospira sp. FBI00063 | bacteria | firmicutes | 28050 | Lactobacillus rogosae | 95.3 | 63 |
| | FBI00285 FBI00364 | | | | | | |
| FBI00064 | Dorea sp. FBI00064 | bacteria | firmicutes | 189330 | Ruminococcus gnavus | 95.58 | 64 |
| FBI00065 | Sutterellaceae sp. FBI00065 | bacteria | proteobacteria | 995019 | Turicimonas muris | 91.55 | 65 |
| FBI00066 | Parasutterella | bacteria | proteobacteria | 487175 | Parasutterella | 99.13 | 66 |
| | excrementihominis | | | | excrementihominis | | |
| FBI00067 | Oxalobacter formigenes | bacteria | proteobacteria | 847 | Oxalobacter formigenes | 98.84 | 67 |
| FBI00068 | Akkermansia muciniphila | bacteria | vemicomicrobia | 239935 | Akkermansia | 99.42 | 68 |
| | | | | | muciniphila | | |
| FBI00069 | Ruminococcus bromii | bacteria | firmicutes | 40518 | Ruminococcus bromii | 98.84 | 69 |
| FBI00070 | Bacteroides kribbi/ | bacteria | bacteroidetes | 816 | Bacteroides koreensis | 99.71 | 70 |
| | Bacteroides koreensis species | | | | | | |
| | cluster | | | | | | |
| FBI00071 | Lachnospiraceae sp. | bacteria | firmicutes | 186803 | Roseburia faecis | 94.92 | 71 |
| | FBI00071 | | | | | | |
| FBI00072 | Coprococcus eutactus | bacteria | firmicutes | 33043 | Coprococcus eutactus | 96.17 | 72 |
| FBI00073 | Parabacteroides distasonis | bacteria | bacteroidetes | 823 | Parabacteroides | 98.99 | 73 |
| | | | | | distasonis | | |
| FBI00074 | Clostridium fessum | bacteria | firmicutes | 2126740 | Clostridium symbiosum | 94.03 | 74 |
| FBI00075 | Paraprevotella clara | bacteria | bacteroidetes | 454154 | Paraprevotella clara | 98.85 | 75 |
| FBI00076 | Bacteroides thetaiotaomicron | bacteria | bacteroidetes | 818 | Bacteroides | 99.78 | 76 |
| | | | | | thetaiotaomicron | | |
| FBI00077 | Sutterella wadsworthensis | bacteria | proteobacteria | 40545 | Sutterella | 99.86 | 77 |
| | | | | | wadsworthensis | | |
| FBI00078 | Blautia obeum | bacteria | firmicutes | 40520 | Blautia obeum | 98.34 | 78 |
| FBI00079 | Clostridium clostridioforme | bacteria | firmicutes | 1531 | Clostridium | 99.14 | 79 |
| | | | | | clostridioforme | | |
| FBI00080 | Sutterella massiliensis | bacteria | proteobacteria | 1816689 | Sutterella massiliensis | 99.78 | 80 |
| FBI00081 | Porphyromonas | bacteria | bacteroidetes | 28123 | Porphyromonas | 99.35 | 81 |
| | asaccharolytica | | | | asaccharolytica | | |
| FBI00082 | Ruminococcaceae sp. | bacteria | firmicutes | 541000 | Phocea massiliensis | 93.08 | 82 |
| | FBI00082 FBI00097 | | | | | | |
| FBI00083 | Alistipes shahii | bacteria | bacteroidetes | 328814 | Alistipes shahii | 99.64 | 83 |
| FBI00084 | Bifidobacterium longum | bacteria | actinobacteria | 216816 | Bifidobacterium longum | 98.07 | 84 |
| FBI00085 | Ruminococcus bromii | bacteria | firmicutes | 40518 | Ruminococcus bromii | 98.62 | 85 |
| FBI00086 | Ruminococcus bromii | bacteria | firmicutes | 40518 | Ruminococcus bromii | 98.77 | 86 |
| FBI00087 | Clostridium scindens | bacteria | firmicutes | 29347 | Clostridium scindens | 98.28 | 87 |
| FBI00088 | Lactobacillus rogosae | bacteria | firmicutes | 706562 | Lactobacillus rogosae | 99.64 | 88 |
| FBI00089 | Bifidobacterium longum | bacteria | actinobacteria | 216816 | Bifidobacterium longum | 98.49 | 89 |
| FBI00090 | Eubacterium eligens | bacteria | firmicutes | 39485 | Eubacterium eligens | 98.71 | 90 |
| FBI00091 | Eubacterium rectale | bacteria | firmicutes | 39491 | Eubacterium rectale | 99.86 | 91 |
| FBI00092 | Monoglobus pectinilyticus | bacteria | firmicutes | 1981510 | Monoglobus | 99.5 | 92 |
| | | | | | pectinilyticus | | |
| FBI00093 | Roseburia hominis | bacteria | firmicutes | 301301 | Roseburia hominis | 99.71 | 93 |
| FBI00094 | Enterococcus faecium | bacteria | firmicutes | 1352 | Enterococcus faecium | 99.38 | 94 |
| FBI00095 | Ruminococcus bromii | bacteria | firmicutes | 40518 | Ruminococcus bromii | 98.7 | 95 |
| FBI00096 | Eggerthella lenta | bacteria | actinobacteria | 84112 | Eggerthella lenta | 98.76 | 96 |
| FBI00097 | Ruminococcaceae sp. | bacteria | firmicutes | 541000 | Phocea massiliensis | 93.07 | 97 |
| | FBI00082 FBI00097 | | | | | | |
| FBI00098 | Bacteroides dorei | bacteria | bacteroidetes | 357276 | Bacteroides dorei | 99.93 | 98 |
| FBI00099 | Gordonibacter pamelaeae | bacteria | actinobacteria | 471189 | Gordonibacter | 99.56 | 99 |
| | | | | | pamelaeae | | |
| FBI00100 | Lachnospira sp. FBI00063 | bacteria | firmicutes | 28050 | Lactobacillus rogosae | 95.35 | 100 |
| | FBI00285 FBI00364 | | | | | | |
| FBI00101 | Faecalibacterium prausnitzii | bacteria | firmicutes | 853 | Faecalibacterium | 97.97 | 101 |
| | | | | | prausnitzii | | |
| FBI00102 | Clostridium fessum | bacteria | firmicutes | 2126740 | Clostridium symbiosum | 94.31 | 102 |
| FBI00103 | Bacteroides massiliensis | bacteria | bacteroidetes | 204516 | Bacteroides massiliensis | 99.86 | 103 |
| FBI00104 | Blautia wexlerae | bacteria | firmicutes | 418240 | Blautia luti | 97.18 | 104 |
| FBI00105 | Ruminococcaceae sp. | bacteria | firmicutes | 541000 | Pseudoflavonifractor | 95.06 | 105 |
| | FBI00105 FBI00160 | | | | phocaeensis | | |
| FBI00106 | Enterococcus durans | bacteria | firmicutes | 53345 | Enterococcus lactis | 96.68 | 106 |
| FBI00107 | Enterococcus durans | bacteria | firmicutes | 53345 | Enterococcus faecium | 98.83 | 107 |
| FBI00108 | Ruminococcaceae sp. | bacteria | firmicutes | 541000 | Gemmiger formicilis | 96.96 | 108 |
| | FBI00108 | | | | | | |
| FBI00109 | Coprococcus comes | bacteria | firmicutes | 410072 | Coprococcus comes | 98.39 | 109 |
| FBI00110 | Lachnoclostridium pacaense | bacteria | firmicutes | 1917870 | Lachnoclostridium | 98.92 | 110 |
| | | | | | pacaense | | |
| FBI00111 | Bacteroides vulgatus | bacteria | bacteroidetes | 821 | Bacteroides vulgatus | 99.43 | 111 |
| FBI00112 | Bacteroides uniformis | bacteria | bacteroidetes | 820 | Bacteroides uniformis | 99.78 | 112 |
| FBI00113 | Parabacteroides merdae | bacteria | bacteroidetes | 46503 | Parabacteroides merdae | 99.79 | 113 |
| FBI00114 | Dorea formicigenerans | bacteria | firmicutes | 39486 | Dorea formicigenerans | 97.83 | 114 |
| FBI00115 | Dorea formicigenerans | bacteria | firmicutes | 39486 | Dorea formicigenerans | 97.98 | 115 |
| FBI00116 | Ruminococcus faecis | bacteria | firmicutes | 592978 | Ruminococcus faecis | 99.57 | 116 |
| FBI00117 | Blautia faecis | bacteria | firmicutes | 871665 | Blautia faecis | 99.52 | 117 |
| FBI00118 | Blautia faecis | bacteria | firmicutes | 871665 | Blautia faecis | 99.84 | 118 |
| FBI00119 | Blautia obeum | bacteria | firmicutes | 40520 | Blautia obeum | 98.02 | 119 |
| FBI00120 | Hungatella effluvii | bacteria | firmicutes | 154046 | Hungatella hathewayi | 98.78 | 120 |
| FBI00121 | Bacteroides vulgatus | bacteria | bacteroidetes | 821 | Bacteroides vulgatus | 99.86 | 121 |
| FBI00122 | Bacteroides uniformis | bacteria | bacteroidetes | 820 | Bacteroides uniformis | 99.57 | 122 |
| FBI00123 | Roseburia hominis | bacteria | firmicutes | 301301 | Roseburia hominis | 100 | 123 |
| FBI00124 | Anaerostipes hadrus | bacteria | firmicutes | 649756 | Anaerostipes hadrus | 99.86 | 124 |
| FBI00125 | Bacteroides stercoris | bacteria | bacteroidetes | 46506 | Bacteroides stercoris | 99.64 | 125 |
| FBI00126 | Bifidobacterium adolescentis | bacteria | actinobacteria | 1680 | Bifidobacterium | 98.98 | 126 |
| | | | | | adolescentis | | |
| FBI00127 | Collinsella aerofaciens | bacteria | actinobacteria | 74426 | Collinsella aerofaciens | 98.81 | 127 |
| FBI00128 | Hungatella effluvii | bacteria | firmicutes | 1096246 | Hungatella effluvii | 98.71 | 128 |
| FBI00129 | Escherichia flexneri | bacteria | proteobacteria | 623 | Escherichia fergusonii | 99.43 | 129 |
| FBI00130 | Coprococcus comes | bacteria | firmicutes | 410072 | Coprococcus comes | 99.35 | 130 |
| FBI00131 | Fusicatenibacter | bacteria | firmicutes | 1150298 | Fusicatenibacter | 99.2 | 131 |
| | saccharivorans | | | | saccharivorans | | |
| FBI00132 | Gordonibacter pamelaeae | bacteria | actinobacteria | 471189 | Gordonibacter | 99.48 | 132 |
| | | | | | pamelaeae | | |
| FBI00133 | Oxalobacter formigenes | bacteria | proteobacteria | 847 | Oxalobacter formigenes | 99.21 | 133 |
| FBI00134 | Bifidobacterium adolescentis | bacteria | actinobacteria | 1680 | Bifidobacterium faecale | 98.92 | 134 |
| FBI00135 | Bifidobacterium | bacteria | actinobacteria | 28026 | Bifidobacterium | 99.57 | 135 |
| | pseudocatenulatum | | | | pseudocatenulatum | | |
| FBI00136 | Eisenbergiella tayi | bacteria | firmicutes | 1432052 | Eisenbergiella tayi | 99.77 | 136 |
| FBI00137 | Bacteroides fragilis | bacteria | bacteroidetes | 817 | Bacteroides fragilis | 99.71 | 137 |
| FBI00138 | Blautia massiliensis | bacteria | firmicutes | 1737424 | Blautia luti | 97.94 | 138 |
| FBI00139 | Bacteroides thetaiotaomicron | bacteria | bacteroidetes | 818 | Bacteroides | 99.5 | 139 |
| | | | | | thetaiotaomicron | | |
| FBI00140 | Phascolarctobacterium | bacteria | firmicutes | 33025 | Phascolarctobacterium | 99.58 | 140 |
| | faecium | | | | faecium | | |
| FBI00141 | Phascolarctobacterium | bacteria | firmicutes | 33025 | Phascolarctobacterium | 99.15 | 141 |
| | faecium | | | | faecium | | |
| FBI00142 | Clostridium fessum | bacteria | firmicutes | 2126740 | Clostridium symbiosum | 94.07 | 142 |
| FBI00143 | Parabacteroides merdae | bacteria | bacteroidetes | 46503 | Parabacteroides merdae | 99.07 | 143 |
| FBI00144 | Holdemanella biformis | bacteria | firmicutes | 1735 | Holdemanella biformis | 97.73 | 144 |
| FBI00145 | Bifidobacterium adolescentis | bacteria | actinobacteria | 1680 | Bifidobacterium | 99.14 | 145 |
| | | | | | adolescentis | | |
| FBI00146 | Blautia faecis | bacteria | firmicutes | 871665 | Blautia faecis | 99.68 | 146 |
| FBI00147 | Clostridium bolteae | bacteria | firmicutes | 208479 | Clostridium bolteae | 99.28 | 147 |
| FBI00148 | Oscillibacter sp. FBI00028 | bacteria | firmicutes | 459786 | Oscillibacter | 94.28 | 148 |
| | | | | | ruminantium | | |
| FBI00149 | Monoglobus pectinilyticus | bacteria | firmicutes | 1981510 | Monoglobus | 99.5 | 149 |
| | | | | | pectinilyticus | | |
| FBI00150 | Lachnospiraceae sp. | bacteria | firmicutes | 186803 | Clostridium | 93.55 | 150 |
| | FBI00033 | | | | amygdalinum | | |
| FBI00151 | Clostridium aldenense | bacteria | firmicutes | 358742 | Clostridium aldenense | 98.55 | 151 |
| FBI00152 | Dialister invisus | bacteria | firmicutes | 218538 | Dialister invisus | 99.58 | 152 |
| FBI00153 | Dialister succinatiphilus | bacteria | firmicutes | 487173 | Dialister succinatiphilus | 95.72 | 153 |
| FBI00154 | Bacteroides dorei | bacteria | bacteroidetes | 357276 | Bacteroides dorei | 100 | 154 |
| FBI00155 | Blautia obeum | bacteria | firmicutes | 40520 | Blautia obeum | 98.7 | 155 |
| FBI00156 | Enterococcus durans | bacteria | firmicutes | 53345 | Enterococcus sp. | 96.45 | 156 |
| FBI00157 | Lachnospiraceae sp. | bacteria | firmicutes | 186803 | Cuneatibacter | 91.24 | 157 |
| | FBI00157 | | | | caecimuris | | |
| FBI00158 | Butyricimonas sp. FBI00158 | bacteria | bacteroidetes | 574697 | Butyricimonas sp. | 97.54 | 158 |
| FBI00159 | Eisenbergiella tayi | bacteria | firmicutes | 1432052 | Eisenbergiella tayi | 99.03 | 159 |
| FBI00160 | Ruminococcaceae sp. | bacteria | firmicutes | 541000 | Pseudoflavonifractor | 97.17 | 160 |
| | FBI00105 FBI00160 | | | | capillosus | | |
| FBI00161 | Bacteroides cellulosilyticus | bacteria | bacteroidetes | 246787 | Bacteroides | 99.14 | 161 |
| | | | | | cellulosilyticus | | |
| FBI00162 | Bifidobacterium catenulatum | bacteria | actinobacteria | 1686 | Bifidobacterium | 99.14 | 162 |
| | | | | | catenulatum | | |
| FBI00163 | Acidaminococcus intestini | bacteria | firmicutes | 187327 | Acidaminococcus | 99.72 | 163 |
| | | | | | intestini | | |
| FBI00164 | Bacteroides stercoris | bacteria | bacteroidetes | 46506 | Bacteroides stercoris | 98.56 | 164 |
| FBI00165 | Bacteroides massiliensis | bacteria | bacteroidetes | 204516 | Bacteroides massiliensis | 99.71 | 165 |
| FBI00166 | Blautia massiliensis | bacteria | firmicutes | 1737424 | Blautia luti | 97.55 | 166 |
| FBI00167 | Dorea longicatena | bacteria | firmicutes | 88431 | Dorea longicatena | 99.39 | 167 |
| FBI00168 | Collinsella aerofaciens | bacteria | actinobacteria | 74426 | Collinsella aerofaciens | 99.26 | 168 |
| FBI00169 | Parabacteroides distasonis | bacteria | bacteroidetes | 823 | Parabacteroides | 98.7 | 169 |
| | | | | | distasonis | | |
| FBI00170 | Eggerthella lenta | bacteria | actinobacteria | 84112 | Eggerthella lenta | 98.61 | 170 |
| FBI00171 | Bilophila wadsworthia | bacteria | proteobacteria | 35833 | Desulfovibrio | 91.45 | 171 |
| | | | | | desulfuricans | | |
| FBI00172 | Bifidobacterium longum | bacteria | actinobacteria | 216816 | Bifidobacterium longum | 99.05 | 172 |
| FBI00173 | Bacteroides vulgatus | bacteria | bacteroidetes | 821 | Bacteroides vulgatus | 100 | 173 |
| FBI00174 | Lactobacillus rogosae | bacteria | firmicutes | 706562 | Lachnospira | 97.92 | 174 |
| | | | | | pectinoschiza | | |
| FBI00175 | Holdemanella biformis | bacteria | firmicutes | 1735 | Holdemanella biformis | 98.19 | 175 |
| FBI00176 | Ruthenibacterium | bacteria | firmicutes | 1550024 | Ruthenibacterium | 99.71 | 176 |
| | lactatiformans | | | | lactatiformans | | |
| FBI00177 | Parasutterella | bacteria | proteobacteria | 487175 | Parasutterella | 99.71 | 177 |
| | excrementihominis | | | | excrementihominis | | |
| FBI00178 | Bifidobacterium adolescentis | bacteria | actinobacteria | 1680 | Bifidobacterium faecale | 98.99 | 178 |
| FBI00179 | Bifidobacterium adolescentis | bacteria | actinobacteria | 1680 | Bifidobacterium faecale | 98.91 | 179 |
| FBI00180 | Alistipes sp. FBI00180 | bacteria | bacteroidetes | 239759 | Alistipes senegalensis | 97.56 | 180 |
| FBI00181 | Blautia wexlerae | bacteria | firmicutes | 418240 | Blautia wexlerae | 97.17 | 181 |
| FBI00182 | Bacteroides coprocola | bacteria | bacteroidetes | 310298 | Bacteroides coprocola | 99.64 | 182 |
| FBI00183 | Bacteroides dorei | bacteria | bacteroidetes | 357276 | Bacteroides dorei | 99.86 | 183 |
| FBI00184 | Bacteroides faecis | bacteria | bacteroidetes | 674529 | Bacteroides faecis | 99.78 | 184 |
| FBI00185 | Eisenbergiella tayi | bacteria | firmicutes | 1432052 | Eisenbergiella tayi | 98.96 | 185 |
| FBI00186 | Coprococcus comes | bacteria | firmicutes | 410072 | Coprococcus comes | 99.06 | 186 |
| FBI00187 | Eubacterium rectale | bacteria | firmicutes | 39491 | Eubacterium rectale | 99.57 | 187 |
| FBI00188 | Blautia faecis | bacteria | firmicutes | 871665 | Blautia faecis | 99.05 | 188 |
| FBI00189 | Bacteroides ovatus | bacteria | bacteroidetes | 28116 | Bacteroides koreensis | 99.93 | 189 |
| FBI00190 | Bacteroides finegoldii | bacteria | bacteroidetes | 338188 | Bacteroides finegoldii | 98.91 | 190 |
| FBI00191 | Clostridiaceae sp. FBI00191 | bacteria | firmicutes | 31979 | Clostridium | 96.24 | 191 |
| | | | | | swellfunianum | | |
| FBI00192 | Sutterella wadsworthensis | bacteria | proteobacteria | 40545 | Sutterella | 99.71 | 192 |
| | | | | | wadsworthensis | | |
| FBI00193 | Alistipes onderdonkii | bacteria | bacteroidetes | 328813 | Alistipes onderdonkii | 99.64 | 193 |
| FBI00194 | Ruminococcus faecis | bacteria | firmicutes | 592978 | Ruminococcus faecis | 98.41 | 194 |
| FBI00195 | Parasutterella | bacteria | proteobacteria | 487175 | Parasutterella | 99.06 | 195 |
| | excrementihominis | | | | excrementihominis | | |
| FBI00196 | Blautia obeum | bacteria | firmicutes | 40520 | Blautia obeum | 98.3 | 196 |
| FBI00197 | Bifidobacterium bifidum | bacteria | actinobacteria | 1681 | Bifidobacterium bifidum | 99.85 | 197 |
| FBI00198 | Lachnoclostridium pacaense | bacteria | firmicutes | 1917870 | Lachnoclostridium | 99.71 | 198 |
| | | | | | pacaense | | |
| FBI00199 | Clostridium bolteae | bacteria | firmicutes | 208479 | Clostridium bolteae | 99.28 | 199 |
| FBI00200 | Longicatena caecimuris | bacteria | firmicutes | 1796635 | Longicatena caecimuris | 99.71 | 200 |
| FBI00201 | Eggerthella lenta | bacteria | actinobacteria | 84112 | Eggerthella lenta | 98.83 | 201 |
| FBI00202 | Erysipelotrichaceae sp. | bacteria | firmicutes | 128827 | Longibaculum muris | 92.85 | 202 |
| | FBI00202 | | | | | | |
| FBI00203 | Bacteroides kribbi/ | bacteria | bacteroidetes | 816 | Bacteroides koreensis | 100 | 203 |
| | Bacteroides koreensis species | | | | | | |
| | cluster | | | | | | |
| FBI00204 | Escherichia flexneri | bacteria | proteobacteria | 623 | Escherichia fergusonii | 98.98 | 204 |
| FBI00205 | Blautia massiliensis | bacteria | firmicutes | 1737424 | Blautia luti | 97.55 | 205 |
| FBI00206 | Bacteroides xylanisolvens | bacteria | bacteroidetes | 371601 | Bacteroides | 99.56 | 206 |
| | | | | | xylanisolvens | | |
| FBI00207 | Parabacteroides merdae | bacteria | bacteroidetes | 46503 | Parabacteroides merdae | 99.49 | 207 |
| FBI00208 | Anaerotruncus massiliensis | bacteria | firmicutes | 1673720 | Anaerotruncus | 96.52 | 208 |
| | | | | | colihominis | | |
| FBI00209 | Bacteroides salyersiae | bacteria | bacteroidetes | 291644 | Bacteroides salyersiae | 99.63 | 209 |
| FBI00210 | Bifidobacterium bifidum | bacteria | actinobacteria | 1681 | Bifidobacterium bifidum | 99.93 | 210 |
| FBI00211 | Bacteroides vulgatus | bacteria | bacteroidetes | 821 | Bacteroides vulgatus | 99.78 | 211 |
| FBI00212 | Clostridium aldenense | bacteria | firmicutes | 358742 | Clostridium aldenense | 99.1 | 212 |
| FBI00213 | Ruminococcus bromii | bacteria | firmicutes | 40518 | Ruminococcus bromii | 98.99 | 213 |
| FBI00214 | Blautia obeum | bacteria | firmicutes | 40520 | Blautia obeum | 98.67 | 214 |
| FBI00215 | Eubacterium rectale | bacteria | firmicutes | 39491 | Eubacterium rectale | 99.78 | 215 |
| FBI00216 | Blautia faecis | bacteria | firmicutes | 871665 | Blautia faecis | 99.76 | 216 |
| FBI00217 | Alistipes shahii | bacteria | bacteroidetes | 328814 | Alistipes shahii | 98.77 | 217 |
| FBI00218 | Bacteroides uniformis | bacteria | bacteroidetes | 820 | Bacteroides uniformis | 99.42 | 218 |
| FBI00219 | Roseburia hominis | bacteria | firmicutes | 301301 | Roseburia hominis | 99.78 | 219 |
| FBI00220 | Megasphaera massiliensis | bacteria | firmicutes | 1232428 | Megasphaera | 98.8 | 220 |
| | | | | | massiliensis | | |
| FBI00221 | Butyricimonas faecihominis | bacteria | bacteroidetes | 1472416 | Butyricimonas | 98.61 | 221 |
| | | | | | faecihominis | | |
| FBI00222 | Alistipes onderdonkii | bacteria | bacteroidetes | 328813 | Alistipes onderdonkii | 99.78 | 222 |
| FBI00223 | Alistipes onderdonkii | bacteria | bacteroidetes | 328813 | Alistipes onderdonkii | 99.7 | 223 |
| FBI00224 | Sutterella wadsworthensis | bacteria | proteobacteria | 40545 | Sutterella | 99.71 | 224 |
| | | | | | wadsworthensis | | |
| FBI00225 | Phascolarctobacterium | bacteria | firmicutes | 33025 | Phascolarctobacterium | 99.37 | 225 |
| | faecium | | | | faecium | | |
| FBI00226 | Catabacter hongkongensis | bacteria | firmicutes | 270498 | Catabacter | 99.71 | 226 |
| | | | | | hongkongensis | | |
| FBI00227 | Bacteroides cellulosilyticus | bacteria | bacteroidetes | 246787 | Bacteroides | 99.2 | 227 |
| | | | | | cellulosilyticus | | |
| FBI00228 | Collinsella aerofaciens | bacteria | actinobacteria | 74426 | Collinsella aerofaciens | 99.27 | 228 |
| FBI00229 | Alistipes senegalensis | bacteria | bacteroidetes | 1288121 | Alistipes senegalensis | 99.19 | 229 |
| FBI00230 | Eisenbergiella tayi | bacteria | firmicutes | 1432052 | Eisenbergiella tayi | 99.48 | 230 |
| FBI00231 | Parabacteroides distasonis | bacteria | bacteroidetes | 823 | Parabacteroides | 99.11 | 231 |
| | | | | | distasonis | | |
| FBI00232 | Bacteroides stercoris | bacteria | bacteroidetes | 46506 | Bacteroides stercoris | 98.84 | 232 |
| FBI00233 | Ruminococcaceae sp. | bacteria | firmicutes | 474960 | Anaerotruncus | 91.63 | 233 |
| | FBI00233 | | | | colihominis | | |
| FBI00234 | Faecalicatena contorta | bacteria | firmicutes | 39482 | Faecalicatena contorta | 99.21 | 234 |
| FBI00235 | Alistipes shahii | bacteria | bacteroidetes | 328814 | Alistipes shahii | 99.86 | 235 |
| FBI00236 | Eisenbergiella tayi | bacteria | firmicutes | 1432052 | Eisenbergiella tayi | 99.41 | 236 |
| FBI00237 | Dielma fastidiosa | bacteria | firmicutes | 1034346 | Dielma fastidiosa | 99.78 | 237 |
| FBI00238 | Alistipes sp. FBI00238 | bacteria | bacteroidetes | 239759 | Alistipes finegoldii | 95.84 | 238 |
| FBI00239 | Lactonifactor longoviformis | bacteria | firmicutes | 341220 | Lactonifactor | 98.99 | 239 |
| | | | | | longoviformis | | |
| FBI00240 | Clostridium citroniae | bacteria | firmicutes | 358743 | Clostridium citroniae | 99.57 | 240 |
| FBI00241 | Collinsella aerofaciens | bacteria | actinobacteria | 74426 | Collinsella aerofaciens | 99.34 | 241 |
| FBI00242 | Clostridium aldenense | bacteria | firmicutes | 358742 | Clostridium aldenense | 99.05 | 242 |
| FBI00243 | Eubacterium siraeum | bacteria | firmicutes | 39492 | Eubacterium siraeum | 98.53 | 243 |
| FBI00244 | Faecalibacterium prausnitzii | bacteria | firmicutes | 853 | Faecalibacterium | 98.69 | 244 |
| | | | | | prausnitzii | | |
| FBI00245 | Acidaminococcus intestini | bacteria | firmicutes | 187327 | Acidaminococcus | 99.72 | 245 |
| | | | | | intestini | | |
| FBI00246 | Bifidobacterium longum | bacteria | actinobacteria | 216816 | Bifidobacterium longum | 99.7 | 246 |
| FBI00247 | Phascolarctobacterium | bacteria | firmicutes | 33025 | Phascolarctobacterium | 99.79 | 247 |
| | faecium | | | | faecium | | |
| FBI00248 | Neglecta timonensis | bacteria | firmicutes | 1776382 | Emergencia timonensis | 99.64 | 248 |
| FBI00249 | Citrobacter portucalensis | bacteria | proteobacteria | 1639133 | Citrobacter freundii | 99.79 | 249 |
| FBI00250 | Bifidobacterium adolescentis | bacteria | actinobacteria | 1680 | Bifidobacterium faecale | 99.24 | 250 |
| FBI00251 | Bifidobacterium | bacteria | actinobacteria | 28026 | Bifidobacterium | 99.85 | 251 |
| | pseudocatenulatum | | | | pseudocatenulatum | | |
| FBI00252 | Oscillibacter sp. FBI00028 | bacteria | firmicutes | 459786 | Oscillibacter | 95.79 | 252 |
| | | | | | ruminantium | | |
| FBI00253 | Roseburia hominis | bacteria | firmicutes | 301301 | Roseburia hominis | 99.71 | 253 |
| FBI00254 | Eubacterium hallii | bacteria | firmicutes | 39488 | Eubacterium hallii | 99.08 | 254 |
| FBI00255 | Hungatella effluvii | bacteria | firmicutes | 1096246 | Hungatella hathewayi | 98.56 | 255 |
| FBI00256 | Blautia faecis | bacteria | firmicutes | 871665 | Blautia faecis | 97.86 | 256 |
| FBI00257 | Eubacterium eligens | bacteria | firmicutes | 39485 | Eubacterium eligens | 99.28 | 257 |
| FBI00258 | Turicibacter sanguinis | bacteria | firmicutes | 154288 | Turicibacter sanguinis | 99.93 | 258 |
| FBI00259 | Dorea longicatena | bacteria | firmicutes | 88431 | Dorea longicatena | 99.7 | 259 |
| FBI00260 | Eubacterium rectale | bacteria | firmicutes | 39491 | Eubacterium rectale | 99.64 | 260 |
| FBI00261 | Bacteroides uniformis | bacteria | bacteroidetes | 820 | Bacteroides uniformis | 99.21 | 261 |
| FBI00262 | Bacteroides massiliensis | bacteria | bacteroidetes | 204516 | Bacteroides massiliensis | 99.71 | 262 |
| FBI00263 | Bacteroides caccae | bacteria | bacteroidetes | 47678 | Bacteroides caccae | 99.56 | 263 |
| FBI00264 | Bifidobacterium adolescentis | bacteria | actinobacteria | 1680 | Bifidobacterium faecale | 99.2 | 264 |
| FBI00265 | Bacteroides cellulosilyticus | bacteria | bacteroidetes | 246787 | Bacteroides | 99.21 | 265 |
| | | | | | cellulosilyticus | | |
| FBI00266 | Coprococcus eutactus | bacteria | firmicutes | 33043 | Coprococcus eutactus | 99.2 | 266 |
| FBI00267 | Anaerofustis stercorihominis | bacteria | firmicutes | 214853 | Anaerofustis | 97.29 | 267 |
| | | | | | stercorihominis | | |
| FBI00268 | Clostridiales sp. FBI00268 | bacteria | firmicutes | 186802 | Catabacter | 86.05 | 268 |
| | | | | | hongkongensis | | |
| FBI00269 | Alistipes putredinis | bacteria | bacteroidetes | 28117 | Alistipes putredinis | 100 | 269 |
| FBI00270 | Methanobrevibacter smithii | archaea | euryarchaeota | 2173 | Methanobrevibacter | 99.69 | 270 |
| | | | | | smithii | | |
| FBI00271 | Bacteroides xylanisolvens | bacteria | bacteroidetes | 371601 | Bacteroides | 98.42 | 271 |
| | | | | | xylanisolvens | | |
| FBI00272 | Clostridiales XIII sp. | bacteria | firmicutes | 543314 | Anaerovorax | 93.12 | 272 |
| | FBI00272 | | | | odorimutans | | |
| FBI00273 | Barnesiella intestinihominis | bacteria | bacteroidetes | 487174 | Barnesiella | 99.43 | 273 |
| | | | | | intestinihominis | | |
| FBI00274 | Eubacterium xylanophilum | bacteria | firmicutes | 39497 | Eubacterium | 93.5 | 274 |
| | | | | | xylanophilum | | |
| FBI00275 | Holdemanella biformis | bacteria | firmicutes | 1735 | Holdemanella biformis | 98.99 | 275 |
| FBI00276 | Dorea formicigenerans | bacteria | firmicutes | 39486 | Dorea formicigenerans | 98.19 | 276 |
| FBI00277 | Alistipes onderdonkii | bacteria | bacteroidetes | 328813 | Alistipes onderdonkii | 99.63 | 277 |
| FBI00278 | Eubacterium ventriosum | bacteria | firmicutes | 39496 | Eubacterium ventriosum | 94.14 | 278 |
| FBI00279 | Coprococcus comes | bacteria | firmicutes | 410072 | Coprococcus comes | 98.48 | 279 |
| FBI00280 | Bacteroides thetaiotaomicron | bacteria | bacteroidetes | 818 | Bacteroides | 100 | 280 |
| | | | | | thetaiotaomicron | | |
| FBI00281 | Senegalimassilia anaerobia | bacteria | actinobacteria | 1473216 | Senegalimassilia | 99.45 | 281 |
| | | | | | anaerobia | | |
| FBI00282 | Porphyromonas | bacteria | bacteroidetes | 28123 | Porphyromonas | 99.35 | 282 |
| | asaccharolytica | | | | asaccharolytica | | |
| FBI00283 | Ruminococcus bromii | bacteria | firmicutes | 40518 | Ruminococcus bromii | 96.02 | 283 |
| FBI00284 | Blautia obeum | bacteria | firmicutes | 40520 | Blautia obeum | 98.67 | 284 |
| FBI00285 | Lachnospira sp. FBI00063 | bacteria | firmicutes | 28050 | Lactobacillus rogosae | 95.3 | 285 |
| | FBI00285 FBI00364 | | | | | | |
| FBI00286 | Fusicatenibacter | bacteria | firmicutes | 1150298 | Fusicatenibacter | 96.32 | 286 |
| | saccharivorans | | | | saccharivorans | | |
| FBI00287 | Alistipes shahii | bacteria | bacteroidetes | 328814 | Alistipes shahii | 98.47 | 287 |
| FBI00288 | Blautia hydrogenotrophica | bacteria | firmicutes | 53443 | Blautia | 99.57 | 288 |
| | | | | | hydrogenotrophica | | |
| FBI00289 | Oxalobacter formigenes | bacteria | proteobacteria | 847 | Oxalobacter formigenes | 99.21 | 289 |
| FBI00290 | Lachnospiraceae sp. | bacteria | firmicutes | 186803 | Eubacterium | 94.81 | 290 |
| | FBI00290 | | | | ruminantium | | |
| FBI00291 | Oribacterium sp. FBI00291 | bacteria | firmicutes | 265975 | Lactobacillus rogosae | 91.63 | 291 |
| FBI00292 | Methanobrevibacter smithii | archaea | euryarchaeota | 2173 | Methanobrevibacter | 99.44 | 292 |
| | | | | | smithii | | |
| FBI00293 | Bifidobacterium adolescentis | bacteria | actinobacteria | 1680 | Bifidobacterium | 98.77 | 293 |
| | | | | | adolescentis | | |
| FBI00294 | Bacteroides stercoris | bacteria | bacteroidetes | 46506 | Bacteroides stercoris | 99.35 | 294 |
| FBI00295 | Bifidobacterium adolescentis | bacteria | actinobacteria | 1680 | Bifidobacterium | 98.48 | 295 |
| | | | | | adolescentis | | |
| FBI00296 | Dorea longicatena | bacteria | firmicutes | 88431 | Dorea longicatena | 99.21 | 296 |
| FBI00297 | Alistipes obesi | bacteria | bacteroidetes | 2585118 | Alistipes obesi | 99.49 | 297 |
| FBI00298 | Faecalibacterium prausnitzii | bacteria | firmicutes | 853 | Faecalibacterium | 97.24 | 298 |
| | | | | | prausnitzii | | |
| FBI00299 | Streptococcus pasteurianus | bacteria | firmicutes | 197614 | Streptococcus | 100 | 299 |
| | | | | | pasteurianus | | |
| FBI00300 | Collinsella aerofaciens | bacteria | actinobacteria | 74426 | Collinsella aerofaciens | 99.49 | 300 |
| FBI00301 | Bifidobacterium adolescentis | bacteria | actinobacteria | 1680 | Bifidobacterium | 98.64 | 301 |
| | | | | | adolescentis | | |
| FBI00302 | Blautia faecis | bacteria | firmicutes | 871665 | Blautia faecis | 99.6 | 302 |
| FBI00303 | Parabacteroides merdae | bacteria | bacteroidetes | 46503 | Parabacteroides merdae | 98.78 | 303 |
| FBI00304 | Dorea longicatena | bacteria | firmicutes | 88431 | Dorea longicatena | 99.7 | 304 |
| FBI00305 | Alistipes onderdonkii | bacteria | bacteroidetes | 328813 | Alistipes onderdonkii | 99.85 | 305 |
| FBI00306 | Parasutterella | bacteria | proteobacteria | 487175 | Parasutterella | 98.53 | 306 |
| | excrementihominis | | | | excrementihominis | | |
| FBI00307 | Parasutterella | bacteria | proteobacteria | 487175 | Parasutterella | 98.53 | 307 |
| | excrementihominis | | | | excrementihominis | | |
| FBI00308 | Bacteroides vulgatus | bacteria | bacteroidetes | 821 | Bacteroides vulgatus | 99.64 | 308 |
| FBI00309 | Eubacterium rectale | bacteria | firmicutes | 39491 | Eubacterium rectale | 99.71 | 309 |
| FBI00310 | Butyricimonas faecihominis | bacteria | bacteroidetes | 1472416 | Butyricimonas | 99.13 | 310 |
| | | | | | faecihominis | | |
| FBI00311 | Anaerostipes hadrus | bacteria | firmicutes | 649756 | Anaerostipes hadrus | 99.07 | 311 |
| FBI00312 | Alistipes shahii | bacteria | bacteroidetes | 328814 | Alistipes shahii | 99.35 | 312 |
| FBI00313 | Collinsella aerofaciens | bacteria | actinobacteria | 74426 | Collinsella aerofaciens | 99.56 | 313 |
| FBI00314 | Anaerotignum | bacteria | firmicutes | 160404 | Anaerotignum | 99.57 | 314 |
| | lactatifermentans | | | | lactatifermentans | | |
| FBI00315 | Blautia obeum | bacteria | firmicutes | 40520 | Blautia obeum | 98.82 | 315 |
| FBI00316 | Collinsella aerofaciens | bacteria | actinobacteria | 74426 | Collinsella aerofaciens | 99.26 | 316 |
| FBI00317 | Bifidobacterium longum | bacteria | actinobacteria | 216816 | Bifidobacterium longum | 99.49 | 317 |
| FBI00318 | Collinsella aerofaciens | bacteria | actinobacteria | 74426 | Collinsella aerofaciens | 99.41 | 318 |
| FBI00319 | Collinsella aerofaciens | bacteria | actinobacteria | 74426 | Collinsella aerofaciens | 99.56 | 319 |
| FBI00320 | Dorea formicigenerans | bacteria | firmicutes | 39486 | Dorea formicigenerans | 98.13 | 320 |
| FBI00321 | Bacteroides vulgatus | bacteria | bacteroidetes | 821 | Bacteroides vulgatus | 97.63 | 321 |
| FBI00322 | Bifidobacterium adolescentis | bacteria | actinobacteria | 1680 | Bifidobacterium | 98.7 | 322 |
| | | | | | adolescentis | | |
| FBI00323 | Blautia wexlerae | bacteria | firmicutes | 418240 | Blautia wexlerae | 99.28 | 323 |
| FBI00324 | Bilophila wadsworthia | bacteria | proteobacteria | 35833 | Desulfovibrio | 91.52 | 324 |
| | | | | | desulfuricans | | |
| FBI00325 | Alistipes indistinctus | bacteria | bacteroidetes | 626932 | Alistipes indistinctus | 99.93 | 325 |
| FBI00326 | Bacteroides vulgatus | bacteria | bacteroidetes | 821 | Bacteroides vulgatus | 98.63 | 326 |
| FBI00327 | Coprococcus comes | bacteria | firmicutes | 410072 | Coprococcus comes | 99.34 | 327 |
| FBI00328 | Blautia luti | bacteria | firmicutes | 89014 | Blautia luti | 97.78 | 328 |
| FBI00329 | Alistipes indistinctus | bacteria | bacteroidetes | 626932 | Alistipes indistinctus | 99.93 | 329 |
| FBI00330 | Bifidobacterium longum | bacteria | actinobacteria | 216816 | Bifidobacterium longum | 99.28 | 330 |
| FBI00331 | Bacillus circulans | bacteria | firmicutes | 1397 | Bacillus circulans | 96.84 | 331 |
| FBI00332 | Clostridium intestinale | bacteria | firmicutes | 36845 | Clostridium intestinale | 99.13 | 332 |
| FBI00333 | Alistipes onderdonkii | bacteria | bacteroidetes | 328813 | Alistipes onderdonkii | 99.93 | 333 |
| FBI00334 | Bacteroides caccae | bacteria | bacteroidetes | 47678 | Bacteroides caccae | 99.56 | 334 |
| FBI00335 | Anaerostipes hadrus | bacteria | firmicutes | 649756 | Anaerostipes hadrus | 99.64 | 335 |
| FBI00336 | Staphylococcus epidermidis | bacteria | firmicutes | 1282 | Staphylococcus | 99.93 | 336 |
| | | | | | epidermidis | | |
| FBI00337 | Coprococcus comes | bacteria | firmicutes | 410072 | Coprococcus comes | 99.21 | 337 |
| FBI00338 | Blautia obeum | bacteria | firmicutes | 40520 | Blautia obeum | 97.75 | 338 |
| FBI00339 | Eubacterium rectale | bacteria | firmicutes | 39491 | Eubacterium rectale | 99.86 | 339 |
| FBI00340 | Lachnospiraceae sp. | bacteria | firmicutes | 186803 | Clostridium | 93.57 | 340 |
| | FBI00033 | | | | amygdalinum | | |
| FBI00341 | Lachnospiraceae sp. | bacteria | firmicutes | 186803 | Roseburia faecis | 95.06 | 341 |
| | FBI00071 | | | | | | |
| FBI00342 | Alistipes indistinctus | bacteria | bacteroidetes | 626932 | Alistipes indistinctus | 100 | 342 |
| FBI00343 | Sutterella massiliensis | bacteria | proteobacteria | 1816689 | Sutterella massiliensis | 99.86 | 343 |
| FBI00344 | Alistipes putredinis | bacteria | bacteroidetes | 28117 | Alistipes putredinis | 99.93 | 344 |
| FBI00345 | Roseburia inulinivorans | bacteria | firmicutes | 360807 | Roseburia inulinivorans | 97.35 | 345 |
| FBI00346 | Coriobacteriia sp. FBI00346 | bacteria | actinobacteria | 84998 | Paraeggerthella | 93.84 | 346 |
| | | | | | hongkongensis | | |
| FBI00347 | Bacteroides uniformis | bacteria | bacteroidetes | 820 | Bacteroides uniformis | 99.85 | 347 |
| FBI00348 | Parabacteroides merdae | bacteria | bacteroidetes | 46503 | Parabacteroides merdae | 99.93 | 348 |
| FBI00349 | Holdemanella biformis | bacteria | firmicutes | 1735 | Holdemanella biformis | 98.14 | 349 |
| FBI00350 | Alistipes putredinis | bacteria | bacteroidetes | 28117 | Alistipes putredinis | 100 | 350 |
| FBI00351 | Alistipes obesi | bacteria | bacteroidetes | 2585118 | Alistipes obesi | 99.78 | 351 |
| FBI00352 | Collinsella aerofaciens | bacteria | actinobacteria | 74426 | Collinsella aerofaciens | 99.56 | 352 |
| FBI00353 | Bifidobacterium adolescentis | bacteria | actinobacteria | 1680 | Bifidobacterium | 98.85 | 353 |
| | | | | | adolescentis | | |
| FBI00354 | Bifidobacterium adolescentis | bacteria | actinobacteria | 1680 | Bifidobacterium | 98.7 | 354 |
| | | | | | stercoris | | |
| FBI00355 | Blautia massiliensis | bacteria | firmicutes | 1737424 | Blautia luti | 98.09 | 355 |
| FBI00356 | Eubacterium eligens | bacteria | firmicutes | 39485 | Eubacterium eligens | 98.5 | 356 |
| FBI00357 | Bacteroides kribbi/ | bacteria | bacteroidetes | 816 | Bacteroides koreensis | 99.71 | 357 |
| | Bacteroides koreensis species | | | | | | |
| | cluster | | | | | | |
| FBI00358 | Eubacterium hallii | bacteria | firmicutes | 39488 | Eubacterium hallii | 98.26 | 358 |
| FBI00359 | Eubacterium rectale | bacteria | firmicutes | 39491 | Eubacterium rectale | 99.79 | 359 |
| FBI00360 | Bacteroides massiliensis | bacteria | bacteroidetes | 204516 | Bacteroides massiliensis | 99.93 | 360 |
| FBI00361 | Bacteroides stercoris | bacteria | bacteroidetes | 46506 | Bacteroides stercoris | 99.36 | 361 |
| FBI00362 | Prevotella copri | bacteria | bacteroidetes | 165179 | Prevotella copri | 98.5 | 362 |
| FBI00363 | Roseburia intestinalis | bacteria | firmicutes | 166486 | Roseburia intestinalis | 99.78 | 363 |
| FBI00364 | Lachnospira sp. FBI00063 | bacteria | firmicutes | 28050 | Lactobacillus rogosae | 95.2 | 364 |
| | FBI00285 FBI00364 | | | | | | |
| FBI00365 | Paraprevotella clara | bacteria | bacteroidetes | 454154 | Paraprevotella clara | 98.78 | 365 |
| FBI00366 | Eubacterium eligens | bacteria | firmicutes | 39485 | Eubacterium eligens | 99 | 366 |
| FBI00367 | Phascolarctobacterium | bacteria | firmicutes | 33025 | Phascolarctobacterium | 99.58 | 367 |
| | faecium | | | | faecium | | |
| FBI00368 | Alistipes putredinis | bacteria | bacteroidetes | 28117 | Alistipes putredinis | 99.86 | 368 |
| FBI00369 | Clostridiales sp. FBI00369 | bacteria | firmicutes | 186802 | Catabacter | 86 | 369 |
| | | | | | hongkongensis | | |
| FBI00370 | Bifidobacterium adolescentis | bacteria | actinobacteria | 1680 | Bifidobacterium | 97.11 | 370 |
| | | | | | adolescentis | | |
| FBI00371 | Bacteroides stercoris | bacteria | bacteroidetes | 46506 | Bacteroides stercoris | 99.21 | 371 |
| FBI00372 | Dorea longicatena | bacteria | firmicutes | 88431 | Dorea longicatena | 99.7 | 372 |
| FBI00373 | Coriobacteriaceae sp. | bacteria | actinobacteria | 84107 | Parolsenella catena | 96.58 | 373 |
| | FBI00373 FBI00374 | | | | | | |
| FBI00374 | Coriobacteriaceae sp. | bacteria | actinobacteria | 84107 | Parolsenella catena | 97.11 | 374 |
| | FBI00373 FBI00374 | | | | | | |
| FBI00375 | Dorea longicatena | bacteria | firmicutes | 88431 | Dorea longicatena | 99.62 | 375 |
| FBI00376 | Dialister succinatiphilus | bacteria | firmicutes | 487173 | Dialister succinatiphilus | 95.82 | 376 |
| FBI00377 | Clostridiales sp. FBI00377 | bacteria | firmicutes | 186802 | Christensenella | 88.69 | 377 |
| | | | | | massiliensis | | |

TABLE 5
Commercial Oxalate-metabolizing strains

Bifidobacterium dentium ATCC 27678
Bifidobacterium dentium ATCC 27680
Bifidobacterium dentium DSM 20221
Bifidobacterium dentium DSM 20436
Bifidobacterium sp. HM-868
Dialister invisus DSM 15470
Eggerthella lenta ATCC 43055
Eggerthella lenta DSM 2243
Enterococcus faecalis HM-202
Enterococcus faecalis HM-432
Lactobacillus acidophilus ATCC 4357
Lactobacillus acidophilus DSM 20079
Lactobacillus acidophilus DSM 20242
Lactobacillus gasseri ATCC 33323
Lactobacillus gasseri DSMZ 107525
Lactobacillus gasseri DSMZ 20077
Lactobacillus gasseri HM-104
Lactobacillus gasseri HM-644
Lactobacillus helveticus DSM 20075
Lactobacillus reuteri HM-102
Lactobacillus rhamnosus ATCC 53103
Lactobacillus rhamnosus DSM 20245
Lactobacillus rhamnosus DSM 8746
Lactobacillus rhamnosus HM-106
Oxalobacter formigenes ATCC 35274
Oxalobacter formigenes DSM 4420
Oxalobacter formigenes HM-1

TABLE 6
Commercial Supportive strains

Absiella dolichum DSM 3991
Acidaminococcus fermentans DSM 20731
Acidaminococcus sp. HM-81
Adlercreutzia equolifaciens DSM 19450
Akkermansia muciniphila ATCC BAA-835
Alistipes finegoldii DSM 17242
Alistipes indistinctus DSM 22520
Alistipes onderdonkii DSM 19147
Alistipes putredinis DSM 17216
Alistipes senegalensis DSM 25460
Alistipes shahii DSM 19121
Anaerobutyricum hallii DSM 3353
Anaerococcus lactolyticus DSM 7456
Anaerofustis stercorihominis DSM 17244
Anaerostipes caccae DSM 14662
Anaerotruncus colihominis DSM 17241
Bacteroides caccae ATCC 43185
Bacteroides caccae HM-728
Bacteroides cellulosilyticus DSM 14838
Bacteroides cellulosilyticus HM-726
Bacteroides coprocola DSM 17136
Bacteroides coprophilus DSM 18228
Bacteroides dorei DSM 17855
Bacteroides dorei HM-29
Bacteroides dorei HM-718
Bacteroides eggerthii DSM 20697
Bacteroides eggerthii HM-210
Bacteroides finegoldii DSM 17565
Bacteroides finegoldii HM-727
Bacteroides fragilis HM-20
Bacteroides fragilis HM-709
Bacteroides fragilis HM-710
Bacteroides intestinalis DSM 17393
Bacteroides ovatus ATCC 8483
Bacteroides ovatus HM-222
Bacteroides pectinophilus ATCC 43243
Bacteroides plebeius DSM 17135
Bacteroides rodentium DSM 26882
Bacteroides salyersiae HM-725
Bacteroides sp. HM-18
Bacteroides sp. HM-19
Bacteroides sp. HM-23
Bacteroides sp. HM-27
Bacteroides sp. HM-28
Bacteroides sp. HM-58
Bacteroides stercoris DSM 19555
Bacteroides stercoris HM-1036
Bacteroides thetaiotaomicron ATCC 29148
Bacteroides uniformis ATCC 8492
Bacteroides vulgatus ATCC 8482
Bacteroides vulgatus HM-720
Bacteroides xylanisolvens DSM 18836
Bifidobacterium adolescentis HM-633
Bifidobacterium angulatum HM-1189
Bifidobacterium animalis DSM 20104
Bifidobacterium animalis subsp. Lactis DSMZ 10140
Bifidobacterium bifidum ATCC 11863
Bifidobacterium breve DSM 20213
Bifidobacterium catenulatum DSM 16992
Bifidobacterium longum infantis ATCC 55813
Bifidobacterium longum subsp. longum HM-845
Bifidobacterium longum subsp. longum HM-846
Bifidobacterium longum subsp. longum HM-847
Bifidobacterium longum subsp. longum HM-848
Bifidobacterium pseudocatenulatum DSM 20438
Bilophila wadsworthia ATCC 49260
Bilophila wadsworthia DSM 11045
Blautia hansenii DSM 20583
Blautia hydrogenotrophica DSM 10507
Blautia obeum DSMZ 25238
Blautia sp. HM-1032
Blautia wexlerae DSM 19850
Butyricimonas virosa DSM 23226
Butyrivibrio crossotus DSM 2876
Catenibacterium mitsuokai DSM 15897
Cetobacterium somerae DSM 23941
Clostridium asparagiforme DSM 15981
Clostridium bolteae DSM 15670
Clostridium bolteae HM-1038
Clostridium bolteae HM-318
Clostridium cadaveris HM-1040
Clostridium citroniae HM-315
Clostridium hiranonis DSM 13275
Clostridium hylemonae DSM 15053
Clostridium innocuum HM-173
Clostridium leptum DSM 753
Clostridium methylpentosum DSM 5476
Clostridium saccharolyticum DSM 2544
Clostridium scindens DSM 5676
Clostridium scindens VPI 12708
Clostridium sp. ATCC 29733
Clostridium sp. DSM 4029
Clostridium sp. HM-634
Clostridium sp. HM-635
Clostridium spiroforme DSM 1552
Clostridium sporogenes ATCC 15579
Clostridium sporogenes ATCC 17889
Clostridium sporogenes DSM 767
Clostridium symbiosum HM-309
Clostridium symbiosum HM-319
Collinsella aerofaciens ATCC 25986
Collinsella stercoris DSM 13279
Coprococcus catus ATCC 27761
Coprococcus comes ATCC 27758
Coprococcus eutactus ATCC 27759
Coprococcus eutactus ATCC 51897
Coprococcus sp. DSM 21649
Desulfovibrio piger ATCC 29098
Dialister pneumosintes ATCC 51894
Dorea formicigenerans ATCC 27755
Dorea longicatena DSM 13814
Eggerthella sp. DSM 11767
Eggerthella sp. DSM 11863
Eggerthella sp. HM-1099
Ethanoligenens harbinense DSM 18485
Eubacterium eligens ATCC 27750
Eubacterium rectale ATCC 33656
Eubacterium siraeum DSM 15702
Eubacterium ventriosum ATCC 27560
Faecalibacterium prausnitzii ATCC 27766
Faecalibacterium prausnitzii ATCC 27768
Faecalibacterium prausnitzii DSM 17677
Faecalibacterium prausnitzii HM-473
Flavonifractor plautii HM-1044
Flavonifractor plautii HM-303
Granulicatella adiacens ATCC 49175
Holdemanella biformis DSM 3989
Holdemania filiformis DSM 12042
Hungatella (prev. Clostridium) hathewayi HM-308
Hungatella hathewayi DSM 13479
Intestinibacter bartlettii DSM 16795
Intestinimonas butyriciproducens DSM 26588
Lactobacillus amylovorus DSM 20552
Lactobacillus casei subsp. casei ATCC 393
Lactobacillus casei subsp. casei ATCC 39539
Lactobacillus crispatus HM-370
Lactobacillus johnsonii HM-643
Lactobacillus parafarraginis HM-478
Lactobacillus plantarum ATCC 14917
Lactobacillus plantarum ATCC 202195
Lactobacillus ruminis ATCC 25644
Lactobacillus ruminis DSM 20404
Lactobacillus ultunensis DSM 16048
Lactococcus lactis Berridge DSM 20729
Marvinbryantia formatexigens DSM 14469
Megasphaera indica DSM 25562
Megasphaera sp. DSM 102144
Methanobrevibacter smithii DSM 11975
Methanobrevibacter smithii DSM 2374
Methanobrevibacter smithii DSM 2375
Methanobrevibacter smithii DSM 861
Methanomassiliicoccus luminyensis DSM 25720
Methanosphaera stadtmanae DSMZ 3091
Mitsuokella multacida DSM 20544
Odoribacter splanchnicus DSM 20712
Olsenella uli DSM 7084
Oscillibacter sp. HM-1030
Parabacteroides distasonis ATCC 8503
Parabacteroides goldsteinii HM-1050
Parabacteroides johnsonii DSM 18315
Parabacteroides johnsonii HM-731
Parabacteroides merdae DSM 19495
Parabacteroides merdae HM-729
Parabacteroides merdae HM-730
Parabacteroides sp. HM-77
Peptostreptococcus anaerobius DSM 2949
Prevotella buccae HM-45
Prevotella buccalis DSM 20616
Prevotella copri DSM 18205
Proteocatella sphenisci DSM 23131
Providencia rettgeri ATCC BAA-2525
Roseburia intestinalis DSM 14610
Roseburia inulinivorans DSM 16841
Ruminococcaceae sp. HM-79
Ruminococcus albus ATCC 27210
Ruminococcus bromii ATCC 27255
Ruminococcus bromii ATCC 51896
Ruminococcus gauvreauii DSM 19829
Ruminococcus gnavus ATCC 29149
Ruminococcus gnavus DSM 108212
Ruminococcus gnavus HM-1056
Ruminococcus lactaris ATCC 29176
Ruminococcus lactaris HM-1057
Ruminococcus torques ATCC 27756
Slackia exigua DSM 15923
Slackia heliotrinireducens DSM 20476
Solobacterium moorei DSM 22971
Streptococcus salivarius subsp. thermophilus ATCC BAA-491
Streptococcus thermophilus ATCC 14485
Subdoligranulum variabile DSM 15176
Turicibacter sanguinis DSM 14220
Tyzzerella nexilis DSM 1787
Veillonella dispar ATCC 17748
Veillonella sp. HM-49
Veillonella sp. HM-64

### Example 2: Commercial Microbial Strain Sensitivities to Oxalate Concentration

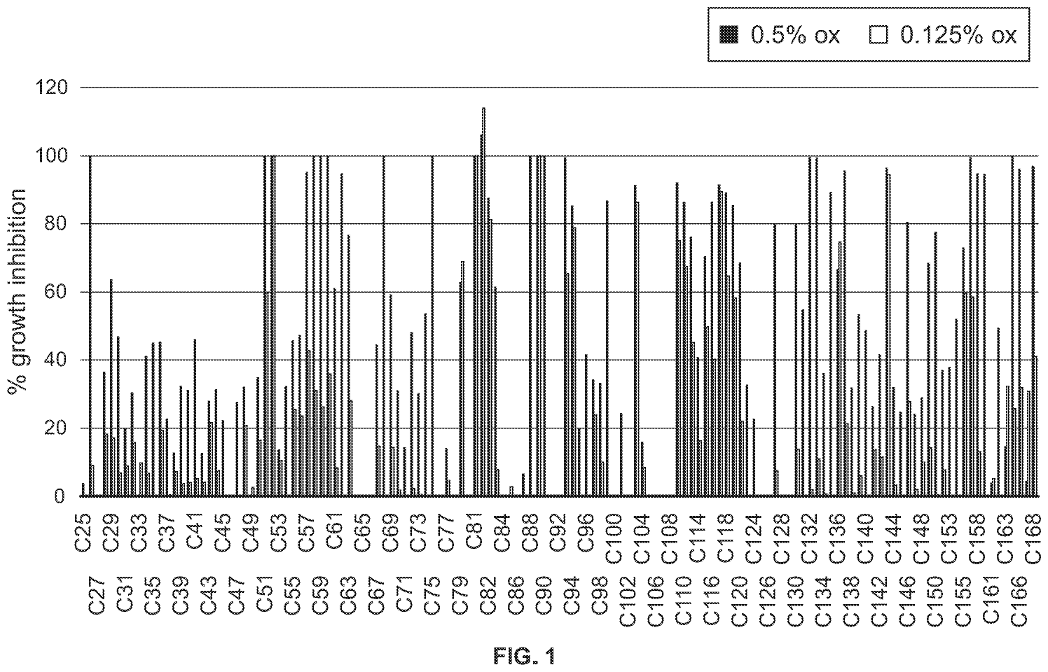

To determine the effect of the presence of oxalate on growth of commercial microbial strains, cultures were grown in their respective banking media (e.g., Mega Media, or Chopped Meat Media) to saturation and back-diluted into the same respective banking media containing no oxalate, 0.5% oxalate, or 0.125% oxalate. FIG. 1 shows % growth inhibition of microbial strains in the presence of 0.5% oxalate (closed bars) or 0.125% oxalate (open bars). % growth inhibition was calculated by determining the ratio of background-subtracted optical density (O.D.) of a microbial strain in the presence of oxalate to the O.D. of the same microbial strain grown in the absence of oxalate.

### Example 3: In Vitro Oxalate Metabolization by Commercial Microbial Strains

48-well deep well plates were filled with 2.5 mL of banking media per commercial microbial strain, per condition. Potassium oxalate was added to achieve final oxalate concentrations of 7.5 mM or 750 μM. 50 μl of each microbial strain in banking media was added to the appropriate well and mixed by trituration. 1 mL of each sample was transferred to an appropriate well of a 96-well collection plate containing 25 μl of 6N HCl and mixed by trituration. The collection plate was covered and incubated at 37° C. for 0, 24, or 72 hours under anaerobic conditions.

The oxalate metabolizing activity of the microbial strains was measured using a commercial colorimetric enzyme kit (Sigma Aldrich Oxalate Assay kit, Catalog No. MAK315) in accordance with the manufacturer's instructions.

In brief, acidified microbial suspensions were centrifuged for 1 minute at >10,000×g to pellet intact cells and cellular debris. 10 μl of sample supernatant was transferred into each of three separate wells of a multiwell plate designated as a “Sample Blank,” “Sample,” or “Internal Standard.” 10 μl of dH₂O was added to Sample Blank and Sample wells, and 10 μl of oxalate standard was added to the Internal Standard well. Blank reagent was prepared for all Sample Blank wells by mixing 155 μl of Reagent B and 1 μl of Horseradish peroxidase (“HRP”) enzyme per Sample Blank well. 157 μl of Working Reagent (155 μl of Reagent B, 1 μl of oxalate oxidase enzyme, and 1 μl of HRP) was prepared for each Sample and Internal Standard well. 150 μl of Blank Reagent was added to each Sample Blank well and 150 μl of Working Reagent was added to each Sample and Internal Standard Well. Solutions were mixed and incubated for 10 minutes at room temperature. Following incubation, optical density was measured for each sample well at 595 nm using a BioTek Epoch 2 plate reader. Sample and Internal Standard values were corrected by subtracting the measured OD₅₉₅ of the Sample Blank well from the measured OD₅₉₅ of the Sample and Internal Standard wells. The proportion of oxalate remaining in each sample after 24 or 72 hour incubation was determined by dividing the corrected OD₅₉₅ value from the Sample well for the initial timepoint (i.e., t=0 hours).

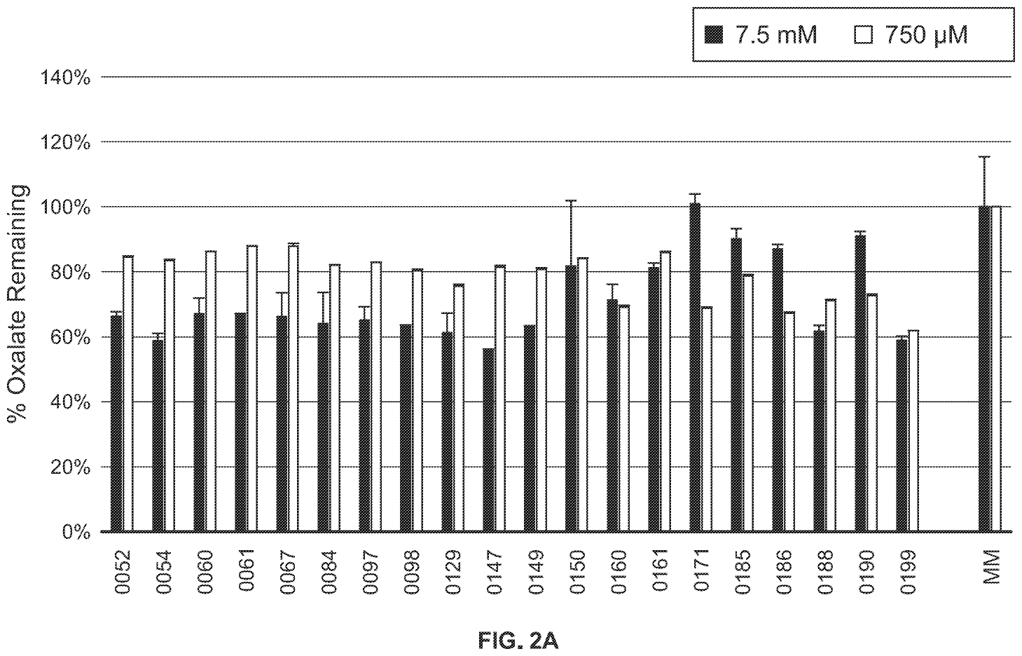

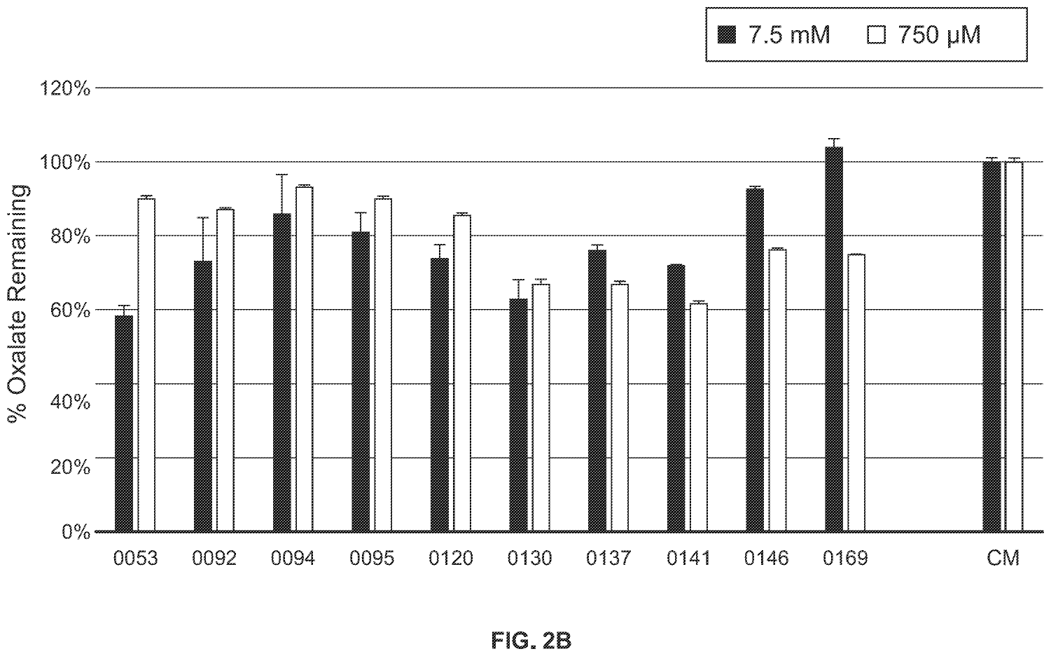

FIG. 2 shows % oxalate remaining in microbial strain cultures in Mega Media (FIG. 2A) or Chopped Meat Media (FIG. 2B) seeded with 7.5 mM oxalate (closed bars) or 750 μM oxalate (open bars) after 72 hours incubation at 37° C. under anaerobic conditions.

### In Vitro Oxalate Metabolizing Activities of Microbial Strains Cultured Under Different pH

To determine the effect of pH on oxalate metabolization, the in vitro oxalate metabolization assay as described above was performed at an oxalate concentration of 7.5 mM, in culture media at pH 7.2 or adjusted to pH 4.5 with NaOH.

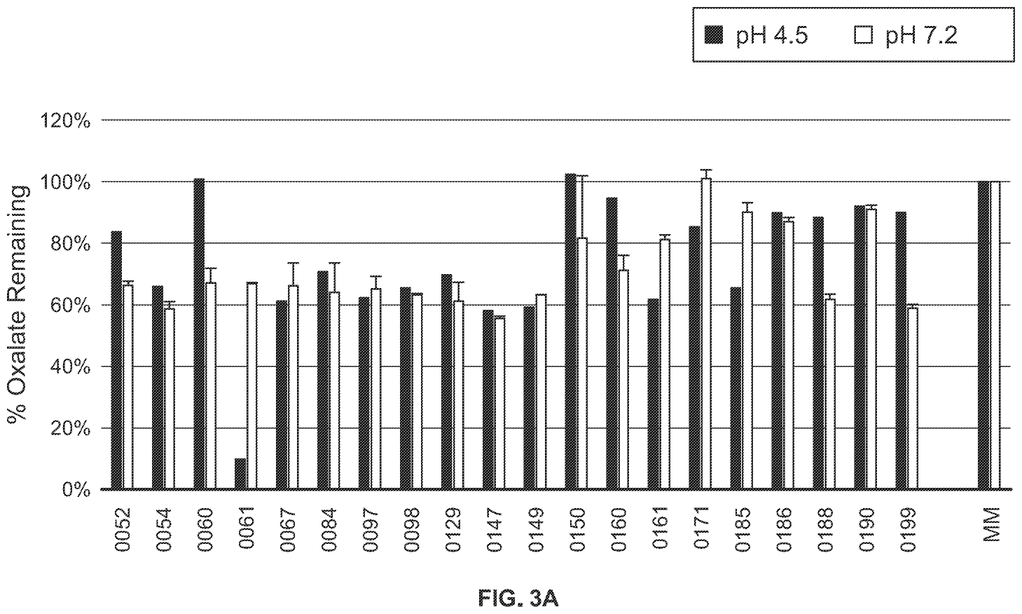

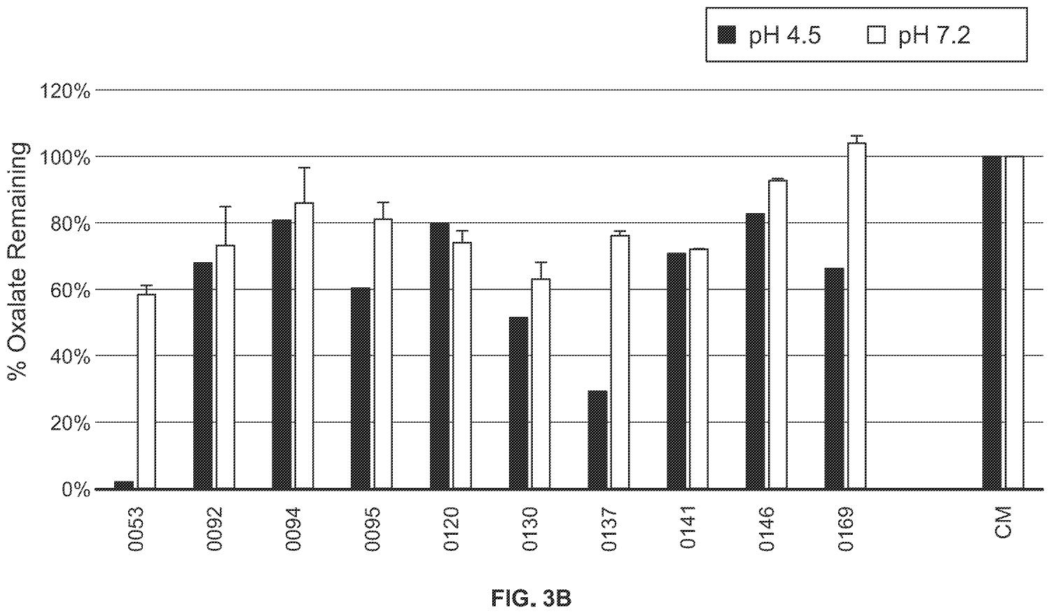

FIG. 3 shows % oxalate remaining in microbial strain cultures in Mega Media (FIG. 3A) or Chopped Meat Media (FIG. 3B) seeded with 7.5 mM oxalate at pH 4.5 (closed bars) or pH 7.2 (open bars) after 72 hours incubation at 37° C. under anaerobic conditions.

### In Vitro Oxalate-Metabolizing Activity of Microbial Consortia

To determine the oxalate-metabolizing activity of a microbial consortium, the in vitro oxalate metabolization assay was performed at an oxalate concentration of 7.5 mM.

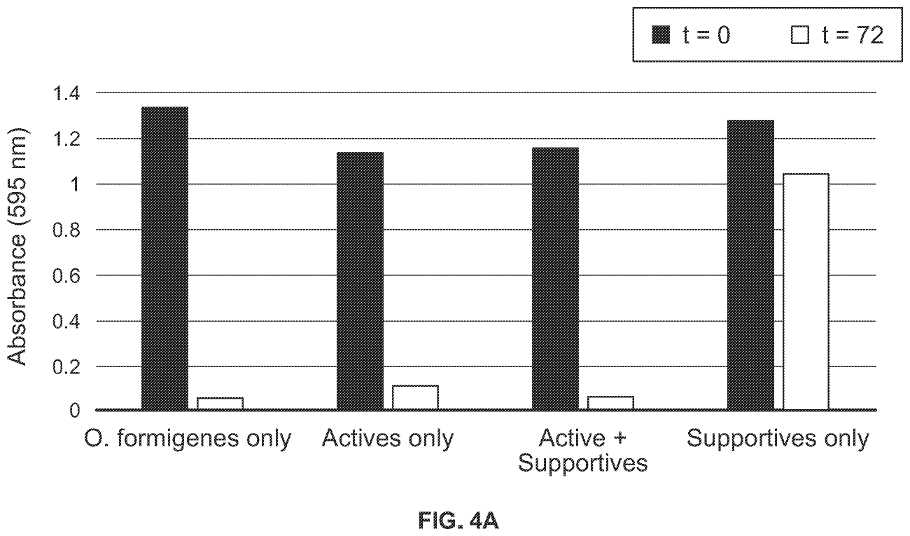

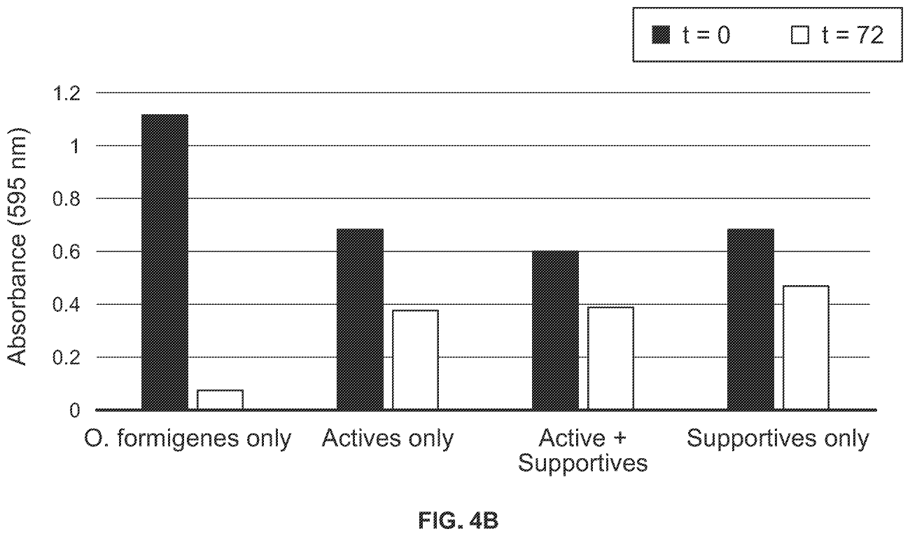

FIG. 4 shows the Absorbance (595 nm) of cultures comprising O. formigenes only, active microbial strains only, supportive microbial strains only, or a complete microbial consortium (i.e. both active and supportive microbial strains) in Mega Media (FIG. 4A) or Chopped Meat Media (FIG. 4B) at the time of oxalate addition (t=0, closed bars) or after 72 hours (open bars).

### Example 4: Oxalate Analysis by Liquid Chromatography Tandem Mass Spectrometry (LC-MS/MS)

In order to quantify oxalate levels in incubation medium, an aliquot of medium was transferred to a polypropylene tube containing 60 μL of 6N HCl/ml medium, vortex mixed, snap frozen, and then stored at −70° C. On the day of analysis, samples were thawed and vortexed to mix, and then 50 μL of media or media diluted with 0.1% formic acid were transferred with mixing to a polypropylene tube containing 20 μL of internal standard (1 mM ¹³C₂-oxalate in 0.1% formic acid) and vortex mixed. A 500 μL aliquot of 2% formic acid was added and vortex mixed. The entire sample was passed through a conditioned Strata-X-AW solid phase extraction plate (Phenomenex, 10 mg, 8E-S038-AGB), washed, and then eluted with 5% ammonium hydroxide in methanol. The eluent was then dried under nitrogen gas, re-constituted in 0.1% formic acid, and then placed on an API6500 autosampler and 5 μL were injected into a 2.1×50 mm Waters XBridge HILIC 3.5 μm particle size column. LC-MS/MS parameters were as indicated in Table 7.

In order to quantify oxalate levels in urine, urine samples were collected and immediately snap frozen and stored at −70° C. On the day of analysis, samples were thawed and vortexed, and then 50 μL of urine or urine diluted with 0.1% formic acid was transferred with mixing to a polypropylene tube containing 20 μL of internal standard (1 mM ¹³C₂-oxalate+5 mM ²H₃-creatinine in 0.1% formic acid) and vortex mixed. A 500 μL aliquot of 2% formic acid was added and vortex mixed. The entire sample was passed through a conditioned Strata-X-AW solid phase extraction plate (Phenomenex, 10 mg, 8E-5038-AGB), washed, and then eluted with 5% ammonium hydroxide in methanol. The eluent was then dried under nitrogen gas, re-constituted in 0.1% formic acid, and then placed on an API6500 autosampler and 5 μL were injected into a 2.1×50 mm Waters XBridge HILIC 3.5 μm particle size column. LC-MS/MS parameters were as indicated in Table 7.

TABLE 7

| Time (min) | % Eluent B |
|---|---|
| Initial | 89.5 |
| 1 | 89.5 |
| 2.5 | 0 |
| 4.20 | 0 |
| 4.21 | 89.5 |
| 6.69 | 89.5 |
| 6.70 | End |

Eluent A: 95:5 20 mM Ammonium Acetate pH 8:Acetonitrile
Eluent B: Acetonitrile
Autosampler Wash: 50% methanol
Flow rate: 500 μL/min
Column temperature: 50° C.
Divert output to waste from initial to 1.5 min and after 4.5 min

### Example 5: In Vivo Oxalate Metabolization in Balb/c Male Mice Treated with a Microbial Consortium Containing Commercial Strains of Microbes

This example describes a study testing the ability of a microbial consortium, containing commercial strains of microbes, to degrade oxalate in vivo in Balb/c male mice.

To determine the in vivo oxalate degrading activity of a microbial consortium described herein, 30 gnotobiotic (n=3 per condition) Balb/c male mice were weighed on Day 0 and colonized by oral gavage with either a plurality of active microbes alone, a supportive community alone, O. formigenes alone, or a complete microbial consortium (active and supportives). The plurality of active microbes and the supportive community of microbes contained the strains in Table 8 marked with an ‘X’ in the indicated column. Colonized mice were fed either a defined, low-complexity diet supplemented with excess oxalate in order to induce hyperoxaluria (see Table 2 above) or a nutritionally equivalent control diet lacking oxalate (see Table 1 above).

After a two-week period, mice were sacrificed and a variety of samples were collected including terminal urine, feces, serum, kidneys, liver, gall bladder, cecum and spleen.

TABLE 8

| | Catalog | Active or | Examples 5 and 8 | | Examples 6, 7, 12, 13, 14 | |
|---|---|---|---|---|---|---|
| Species | Number | Supportive | Active | Supportive | Active | Supportive |
| Acidaminococcus fermentans | DSM 20731 | Supportive | | | | X |
| Acidaminococcus intestini | HM-81 | Supportive | | X | | X |
| Adlercreutzia equolifaciens | DSM 19450 | Supportive | | X | | X |
| Akkermansia muciniphila | ATCC BAA-835 | Supportive | | X | | X |
| Alistipes finegoldii | DSM 17242 | Supportive | | X | | X |
| Alistipes indistinctus | DSM 22520 | Supportive | | X | | X |
| Alistipes onderdonkii | DSM 19147 | Supportive | | X | | X |
| Alistipes putredinis | DSM 17216 | Supportive | | | | X |
| Alistipes senegalensis | DSM 25460 | Supportive | | X | | X |
| Alistipes shahii | DSM 19121 | Supportive | | X | | X |
| Anaerobutyricum hallii | DSM 3353 | Supportive | | | | X |
| Anaerococcus lactolyticus | DSM 7456 | Supportive | | | | |
| Anaerofustis stercorihominis | DSM 17244 | Supportive | | | | X |
| Anaerostipes caccae | DSM 14662 | Supportive | | X | | X |
| Anaerotruncus colihominis | DSM 17241 | Supportive | | X | | X |
| Bacteroides caccae | ATCC 43185 | Supportive | | X | | X |
| Bacteroides caccae | HM-728 | Supportive | | | | |
| Bacteroides cellulosilyticus | DSM 14838 | Supportive | | X | | X |
| Bacteroides cellulosilyticus | HM-726 | Supportive | | | | |
| Bacteroides coprocola | DSM 17136 | Supportive | | X | | X |
| Bacteroides coprophilus | DSM 18228 | Supportive | | X | | X |
| Bacteroides dorei | DSM 17855 | Supportive | | X | | X |
| Bacteroides dorei | HM-27 | Supportive | | X | | X |
| Bacteroides dorei | HM-29 | Supportive | | X | | X |
| Bacteroides dorei | HM-718 | Supportive | | | | |
| Bacteroides eggerthii | DSM 20697 | Supportive | | X | | X |
| Bacteroides eggerthii | HM-210 | Supportive | | | | |
| Bacteroides finegoldii | DSM 17565 | Supportive | | X | | X |
| Bacteroides finegoldii | HM-727 | Supportive | | | | |
| Bacteroides fragilis | HM-20 | Supportive | | X | | X |
| Bacteroides fragilis | HM-58 | Supportive | | X | | X |
| Bacteroides fragilis | HM-709 | Supportive | | | | |
| Bacteroides fragilis | HM-710 | Supportive | | | | |
| Bacteroides intestinalis | DSM 17393 | Supportive | | X | | X |
| Bacteroides ovatus | ATCC 8483 | Supportive | | X | | X |
| Bacteroides ovatus | HM-222 | Supportive | | | | |
| Bacteroides pectinophilus | ATCC 43243 | Supportive | | | | X |
| Bacteroides plebeius | DSM 17135 | Supportive | | | | X |
| Bacteroides rodentium | DSM 26882 | Supportive | | X | | X |
| Bacteroides salyersiae | HM-725 | Supportive | | | | |
| Bacteroides sp. | HM-28 | Supportive | | X | | X |
| Bacteroides stercoris | DSM 19555 | Supportive | | X | | X |
| Bacteroides stercoris | HM-1036 | Supportive | | | | |
| Bacteroides thetaiotaomicron | ATCC 29148 | Supportive | | X | | X |
| Bacteroides thetaiotaomicron | HM-23 | Supportive | | | | X |
| Bacteroides uniformis | ATCC 8492 | Supportive | | X | | X |
| Bacteroides vulgatus | ATCC 8482 | Supportive | | X | | X |
| Bacteroides vulgatus | HM-720 | Supportive | | | | |
| Bacteroides xylanisolvens | DSM 18836 | Supportive | | X | | X |
| Bacteroides xylanisolvens | HM-18 | Supportive | | X | | X |
| Bifidobacterium adolescentis | HM-633 | Supportive | | | | |
| Bifidobacterium angulatum | HM-1189 | Supportive | | | | |
| Bifidobacterium animalis | DSM 20104 | Supportive | | | | |
| Bifidobacterium animalis | DSMZ 10140 | Supportive | | | | |
| Bifidobacterium bifidum | ATCC 11863 | Supportive | | | | |
| Bifidobacterium breve | DSM 20213 | Supportive | | X | | X |
| Bifidobacterium catenulatum | DSM 16992 | Supportive | | X | | X |
| Bifidobacterium dentium | ATCC 27678 | Active | X | | X | |
| Bifidobacterium dentium | ATCC 27680 | Active | X | | X | |
| Bifidobacterium dentium | DSM 20221 | Active | X | | X | |
| Bifidobacterium dentium | DSM 20436 | Active | X | | X | |
| Bifidobacterium dentium | HM-868 | Active | X | | X | |
| Bifidobacterium infantis | ATCC 55813 | Supportive | | | | |
| Bifidobacterium longum | HM-845 | Supportive | | | | |
| Bifidobacterium longum | HM-846 | Supportive | | | | |
| Bifidobacterium longum | HM-847 | Supportive | | | | |
| Bifidobacterium longum | HM-848 | Supportive | | | | |
| Bifidobacterium pseudocatenulatum | DSM 20438 | Supportive | | X | | X |
| Bilophila wadsworthia | ATCC 49260 | Supportive | | X | | X |
| Bilophila wadsworthia | DSM 11045 | Supportive | | | | |
| Bittarella massiliensis | ATCC 29733 | Supportive | | X | | X |
| Bittarella massiliensis | DSM 4029 | Supportive | | | | |
| Blautia hansenii | DSM 20583 | Supportive | | X | | X |
| Blautia hydrogenotrophica | DSM 10507 | Supportive | | | | X |
| Blautia massiliensis | HM-1032 | Supportive | | X | | X |
| Blautia obeum | DSMZ 25238 | Supportive | | X | | X |
| Blautia wexlerae | DSM 19850 | Supportive | | | | X |
| Butyricimonas virosa | DSM 23226 | Supportive | | X | | X |
| Butyrivibrio crossotus | DSM 2876 | Supportive | | | | X |
| Catenibacterium mitsuokai | DSM 15897 | Supportive | | X | | X |
| Cetobacterium somerae | DSM 23941 | Supportive | | | | |
| Clostridium asparagiforme | DSM 15981 | Supportive | | X | | X |
| Clostridium bolteae | DSM 15670 | Supportive | | | | X |
| Clostridium bolteae | HM-1038 | Supportive | | | | |
| Clostridium bolteae | HM-318 | Supportive | | | | |
| Clostridium cadaveris | HM-1040 | Supportive | | | | |
| Clostridium citroniae | HM-315 | Supportive | | | | |
| Clostridium hiranonis | DSM 13275 | Supportive | | X | | X |
| Clostridium hylemonae | DSM 15053 | Supportive | | X | | X |
| Clostridium innocuum | HM-173 | Supportive | | | | |
| Clostridium leptum | DSM 753 | Supportive | | X | | X |
| Clostridium methylpentosum | DSM 5476 | Supportive | | | | X |
| Clostridium nexile | DSM 1787 | Supportive | | X | | X |
| Clostridium orbiscindens | HM-303 | Supportive | | | | X |
| Clostridium orbiscindens | HM-1044 | Supportive | | | | |
| Clostridium saccharolyticum | DSM 2544 | Supportive | | X | | X |
| Clostridium saccharolyticum | HM-635 | Supportive | | | | X |
| Clostridium scindens | DSM 5676 | Supportive | | X | | X |
| Clostridium scindens | VPI 12708 | Supportive | | | | |
| Clostridium sp. | HM-634 | Supportive | | X | | X |
| Clostridium spiroforme | DSM 1552 | Supportive | | | | X |
| Clostridium sporogenes | ATCC 15579 | Supportive | | | | |
| Clostridium sporogenes | ATCC 17889 | Supportive | | | | |
| Clostridium sporogenes | DSM 767 | Supportive | | | | |
| Clostridium symbiosum | HM-309 | Supportive | | | | |
| Clostridium symbiosum | HM-319 | Supportive | | | | |
| Collinsella aerofaciens | ATCC 25986 | Supportive | | X | | X |
| Collinsella stercoris | DSM 13279 | Supportive | | X | | X |
| Coprococcus catus | ATCC 27761 | Supportive | | | | |
| Coprococcus comes | ATCC 27758 | Supportive | | X | | X |
| Coprococcus eutactus | ATCC 27759 | Supportive | | | | X |
| Coprococcus eutactus | ATCC 51897 | Supportive | | | | |
| Coprococcus sp. | DSM 21649 | Supportive | | | | |
| Desulfovibrio piger | ATCC 29098 | Supportive | | X | | X |
| Dialister invisus | DSM 15470 | Active | X | | X | |
| Dialister pneumosintes | ATCC 51894 | Supportive | | | | |
| Dorea formicigenerans | ATCC 27755 | Supportive | | X | | X |
| Dorea longicatena | DSM 13814 | Supportive | | X | | X |
| Eggerthella lenta | ATCC 43055 | Active | X | | X | |
| Eggerthella lenta | DSM 2243 | Active | X | | X | |
| Eggerthella lenta | DSM 11767 | Supportive | | | | |
| Eggerthella lenta | DSM 11863 | Supportive | | | | |
| Eggerthella lenta | EIM-1099 | Supportive | | | | |
| Enterococcus faecalis | HM-202 | Active | X | | X | |
| Enterococcus faecalis | HM-432 | Active | | | X | |
| Ethanoligenens harbinense | DSM 18485 | Supportive | | | | X |
| Eubacterium dolichum | DSM 3991 | Supportive | | X | | X |
| Eubacterium eligens | ATCC 27750 | Supportive | | | | |
| Eubacterium rectale | ATCC 33656 | Supportive | | X | | X |
| Eubacterium siraeum | DSM 15702 | Supportive | | X | | X |
| Eubacterium ventriosum | ATCC 27560 | Supportive | | X | | X |
| Faecalibacterium prausnitzii | DSM 17677 | Supportive | | X | | X |
| Faecalibacterium prausnitzii | ATCC 27766 | Supportive | | | | |
| Faecalibacterium prausnitzii | ATCC 27768 | Supportive | | | | |
| Faecalibacterium prausnitzii | HM-473 | Supportive | | | | |
| Granulicatella adiacens | ATCC 49175 | Supportive | | X | | X |
| Holdemanella biformis | DSM 3989 | Supportive | | X | | X |
| Holdemania filiformis | DSM 12042 | Supportive | | X | | X |
| Hungatella hathewayi | DSM 13479 | Supportive | | X | | X |
| Hungatella hathewayi | HM-308 | Supportive | | | | |
| Intestinibacter bartlettii | DSM 16795 | Supportive | | | | X |
| Intestinimonas butyriciproducens | DSM 26588 | Supportive | | | | X |
| Lactobacillus acidophilus | DSM 20079 | Active | X | | X | |
| Lactobacillus acidophilus | ATCC 4357 | Active | | | | |
| Lactobacillus acidophilus | DSM 20242 | Active | | | | |
| Lactobacillus amylovorus | DSM 20552 | Supportive | | | | |
| Lactobacillus casei | ATCC 393 | Supportive | | | | |
| Lactobacillus casei | ATCC 39539 | Supportive | | | | |
| Lactobacillus crispatus | HM-370 | Supportive | | | | |
| Lactobacillus gasseri | ATCC 33323 | Active | X | | X | |
| Lactobacillus gasseri | DSMZ 107525 | Active | | | | |
| Lactobacillus gasseri | DSMZ 20077 | Active | | | | |
| Lactobacillus gasseri | HM-104 | Active | | | | |
| Lactobacillus gasseri | HM-644 | Active | | | | |
| Lactobacillus helveticus | DSM 20075 | Active | X | | X | |
| Lactobacillus johnsonii | HM-643 | Supportive | | | | |
| Lactobacillus parafarraginis | HM-478 | Supportive | | | | |
| Lactobacillus plantarum | ATCC 14917 | Supportive | | | | |
| Lactobacillus plantarum | ATCC 202195 | Supportive | | | | |
| Lactobacillus reuteri | HM-102 | Active | X | | X | |
| Lactobacillus rhamnosus | DSM 20245 | Active | X | | X | |
| Lactobacillus rhamnosus | HM-106 | Active | X | | X | |
| Lactobacillus rhamnosus | ATCC 53103 | Active | | | | |
| Lactobacillus rhamnosus | DSM 8746 | Active | | | | |
| Lactobacillus ruminis | ATCC 25644 | Supportive | | X | | X |
| Lactobacillus ruminis | DSM 20404 | Supportive | | | | |
| Lactobacillus ultunensis | DSM 16048 | Supportive | | | | |
| Lactococcus lactis | DSM 20729 | Supportive | | | | |
| Marvinbryantia formatexigens | DSM 14469 | Supportive | | X | | X |
| Megasphaera indica | DSM 25562 | Supportive | | | | |
| Megasphaera stantonii | DSM 102144 | Supportive | | X | | X |
| Methanobrevibacter smithii | DSM 11975 | Supportive | | | | |
| Methanobrevibacter smithii | DSM 2374 | Supportive | | | | |
| Methanobrevibacter smithii | DSM 2375 | Supportive | | | | |
| Methanobrevibacter smithii | DSM 861 | Supportive | | | | |
| Methanomassiliicoccus luminyensis | DSM 25720 | Supportive | | | | |
| Methanosphaera stadtmanae | DSM 3091 | Supportive | | | | |
| Mitsuokella multacida | DSM 20544 | Supportive | | X | | X |
| Odoribacter splanchnicus | DSM 20712 | Supportive | | X | | X |
| Olsenella uli | DSM 7084 | Supportive | | X | | X |
| Oscillibacter welbionis | HM-1030 | Supportive | | | | X |
| Oxalobacter formigenes | ATCC 35274 | Active | X | | X | |
| Oxalobacter formigenes | HM-1 | Active | X | | X | |
| Oxalobacter formigenes | DSM 4420 | Active | | | | |
| Oxalobacter vibrioformis | DSM 5502 | Supportive | | | | |
| Oxalophagus oxalicus | ATCC 49686 | Supportive | | | | |
| Parabacteroides distasonis | HM-19 | Supportive | | X | | X |
| Parabacteroides distasonis | HM-77 | Supportive | | X | | X |
| Parabacteroides goldsteinii | HM-1050 | Supportive | | | | |
| Parabacteroides johnsonii | DSM 18315 | Supportive | | X | | X |
| Parabacteroides johnsonii | HM-731 | Supportive | | | | |
| Parabacteroides merdae | DSM 19495 | Supportive | | | | X |
| Parabacteroides merdae | HM-729 | Supportive | | | | |
| Parabacteroides merdae | HM-730 | Supportive | | | | |
| Peptostreptococcus anaerobius | DSM 2949 | Supportive | | | | |
| Prevotella buccae | HM-45 | Supportive | | | | |
| Prevotella buccalis | DSM 20616 | Supportive | | X | | X |
| Prevotella copri | DSM 18205 | Supportive | | | | X |
| Prevotella histicola | DSM 26979 | Supportive | | | | |
| Proteocatella sphenisci | DSM 23131 | Supportive | | | | |
| Providencia rettgeri | ATCC BAA-2525 | Supportive | | | | |
| Roseburia intestinalis | DSM 14610 | Supportive | | | | |
| Roseburia inulinivorans | DSM 16841 | Supportive | | X | | X |
| Ruminococcaceae sp. | HM-79 | Supportive | | | | |
| Ruminococcus albus | ATCC 27210 | Supportive | | | | |
| Ruminococcus bromii | ATCC 27255 | Supportive | | | | |
| Ruminococcus bromii | ATCC 51896 | Supportive | | | | |
| Ruminococcus gauvreauii | DSM 19829 | Supportive | | X | | X |
| Ruminococcus gnavus | ATCC 29149 | Supportive | | X | | X |
| Ruminococcus gnavus | DSM 108212 | Supportive | | | | |
| Ruminococcus gnavus | HM-1056 | Supportive | | | | |
| Ruminococcus lactaris | ATCC 29176 | Supportive | | | | X |
| Ruminococcus lactaris | HM-1057 | Supportive | | | | |
| Ruminococcus torques | ATCC 27756 | Supportive | | X | | X |
| s_Parabacteroides distasonis | ATCC 8503 | Supportive | | X | | X |
| Slackia exigua | DSM 15923 | Supportive | | | | X |
| Slackia heliotrinireducens | DSM 20476 | Supportive | | | | X |
| Solobacterium moorei | DSM 22971 | Supportive | | X | | X |
| Streptococcus thermophilus | ATCC BAA-491 | Supportive | | X | | X |
| Streptococcus thermophilus | ATCC 14485 | Supportive | | | | |
| Subdoligranulum variabile | DSM 15176 | Supportive | | | | X |
| Turicibacter sanguinis | DSM 14220 | Supportive | | | | X |
| Veillonella dispar | ATCC 17748 | Supportive | | | | |
| Veillonella parvula | HM-49 | Supportive | | | | |
| Veillonella parvula | HM-64 | Supportive | | | | |

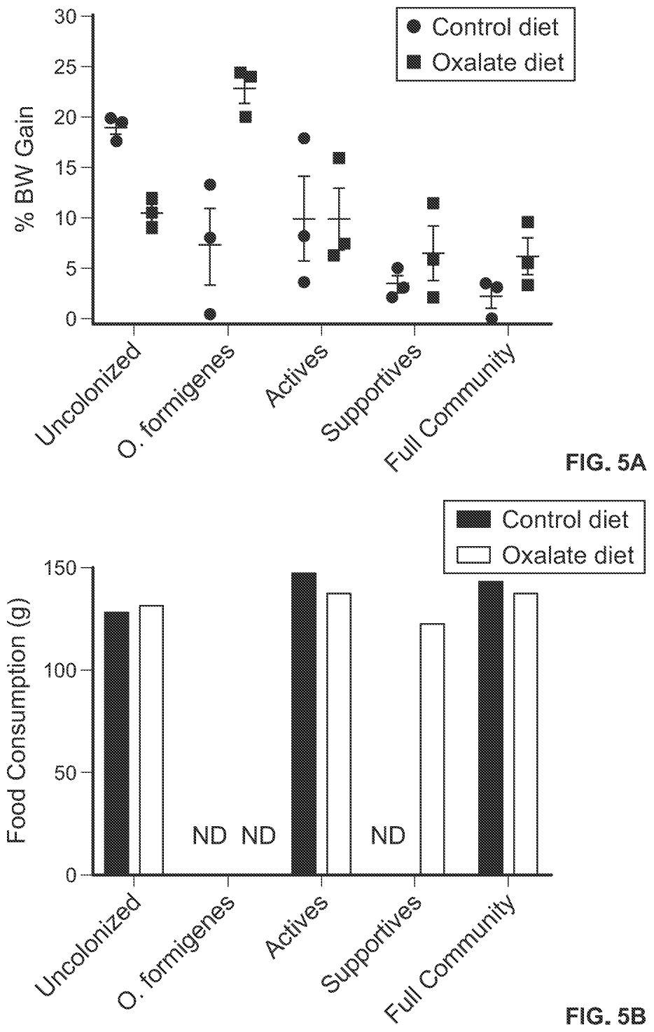

FIG. 5A and FIG. 5B show the % body weight gain and food consumption, respectively, of the uncolonized mice, mice gavaged with either O. formigenes alone, active microbes alone, supportive microbes alone, or a complete microbial consortium (active and supportives) as described above.

Table 9 shows the incidence of diarrhea in the uncolonized mice, mice gavaged with either O. formigenes alone, active microbes alone, supportive microbes alone, or a complete microbial consortium (active and supportives) as described above. Mice treated with a complete microbial consortium were observed to have normal stool pellets and a reduced incidence of diarrhea.

TABLE 9

| Uncolonized | | O. formigenes | | Actives | | Supportives | | Full Community | |
|---|---|---|---|---|---|---|---|---|---|
| Control | Oxalate | Control | Oxalate | Control | Oxalate | Control | Oxalate | Control | Oxalate |
| Diet | Diet | Diet | Diet | Diet | Diet | Diet | Diet | Diet | Diet |
| 3/3 | 3/3 | 3/3 | 3/3 | 3/3 | 3/3 | 3/3 | 3/3 | 0/3 | 0/3 |

| | | |
|---|---|---|
| Runny stool | Soft stool | Normal stool |

Table 10 shows the incidence of fatty liver in the uncolonized mice, mice gavaged with either O. formigenes alone, active microbes alone, supportive microbes alone, or a complete microbial consortium (active and supportives) as described above.

TABLE 10

| Uncolonized | | O. formigenes | | Actives | | Supportives | | Full Community | |
|---|---|---|---|---|---|---|---|---|---|
| Control | Oxalate | Control | Oxalate | Control | Oxalate | Control | Oxalate | Control | Oxalate |
| Diet | Diet | Diet | Diet | Diet | Diet | Diet | Diet | Diet | Diet |
| 0/3 | 0/3 | 1/3 | 2/3 | 0/3 | 1/3 | 0/3 | 2/3 | 0/3 | 0/3 |

| | | |
|---|---|---|
| No Fatty Liver | Fatty Liver observed in 6/18 mice | No Fatty Liver |

### Urinary Oxalate Concentrations

To assess the effect of a microbial consortium described herein on steady-state levels of oxalate in urine, which correlates well with human urolithiasis, urine was terminally collected from all test groups. Each mouse was transferred to the bottom of a standard petri dish, placed into a CO₂ chamber, and administered CO₂ for 90 seconds according to the approved IACUC protocol until the mouse ceased moving and was lying prone on the chamber floor. The CO₂ chamber lid was opened and the anaesthetized mouse was placed on its side on the petri dish. The CO₂ chamber lid was then replaced and terminal urination collected in the petri dish and transferred to a sterile microcentrifuge tube. Urine samples were processed and prepared for solid phase extraction followed by LC/MS-based analysis as described in Example 4 above.

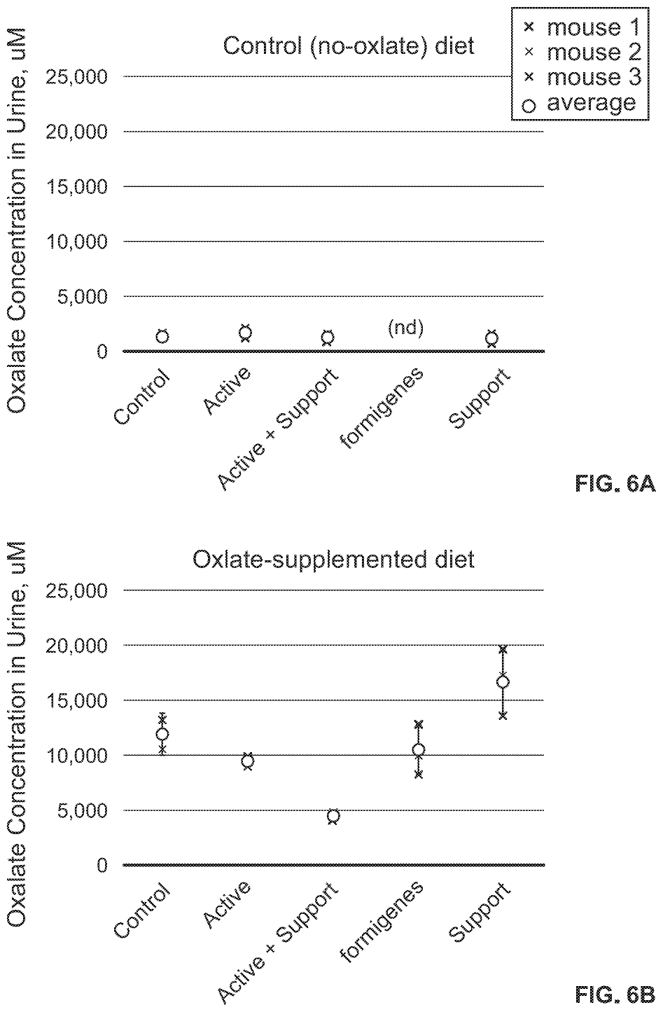

As shown in Table 11 and FIG. 6A-B, mice fed with control diet lacking supplemental oxalate predictably exhibited low levels of urinary oxalate (1.2 mM in uncolonized controls) compared with mice fed a diet containing excess oxalate (11.9 mM in uncolonized controls), showing that dietary supplementation with oxalate can induce hyperoxaluria in gnotobiotic mice.

Regardless of diet, the lowest levels of urinary oxalate were observed in mice colonized with the complete microbial consortium (active and supportives); average oxalate levels in consortium-colonized mice fed the oxalate-free (control) diet were approximately 50% lower than observed in uncolonized mice, and in animals fed the high oxalate diet, steady-state urinary oxalate levels were approximately 66% lower in consortium-colonized mice compared to uncolonized controls (4.5 mM vs. 11.9 mM).

Mice treated with a complete microbial consortium outperformed mice treated with the plurality of active microbes and supportive community of microbes alone, as well as mice treated with O. formigenes alone, with respect to urinary oxalate concentrations. Mice colonized with O. formigenes alone or the plurality of active microbes alone and fed the oxalate-supplemented diet exhibited urinary oxalate concentrations that were not significantly different from those observed in uncolonized mice. Furthermore, mice colonized with the supportive community of microbes alone exhibited significantly higher urinary oxalate levels than uncolonized controls (16.7 mM and 11.9 mM, respectively). Whereas colonization with either the plurality of active microbes alone or the supportive community alone did not reduce levels of urinary oxalate, colonization with the full consortium resulted in a synergistic drop in urinary oxalate concentration.

TABLE 11

Urinary Oxalate (mM)
n = 3 mice/group

| Communities | Oxalate Diet | Control Diet |
|---|---|---|
| Control (gnotobiotic) | 11.9 ± 1.8 | 1.2 ± 0.2 |
| Supportive Community only | 16.7 ± 3.0 | 0.6 ± 0.1 |
| O. formigenes only | 10.5 ± 2.2 | 1.2 ± 0.2 |
| Plurality of Active Microbes only | 9.5 ± 0.4 | 1.5 ± 0.2 |
| Full Microbial Consortium | 4.5 ± 0.1 | 0.6 ± 0.1 |
| (Active ± Supportive) | | |

### Serum Liver Enzyme Assay

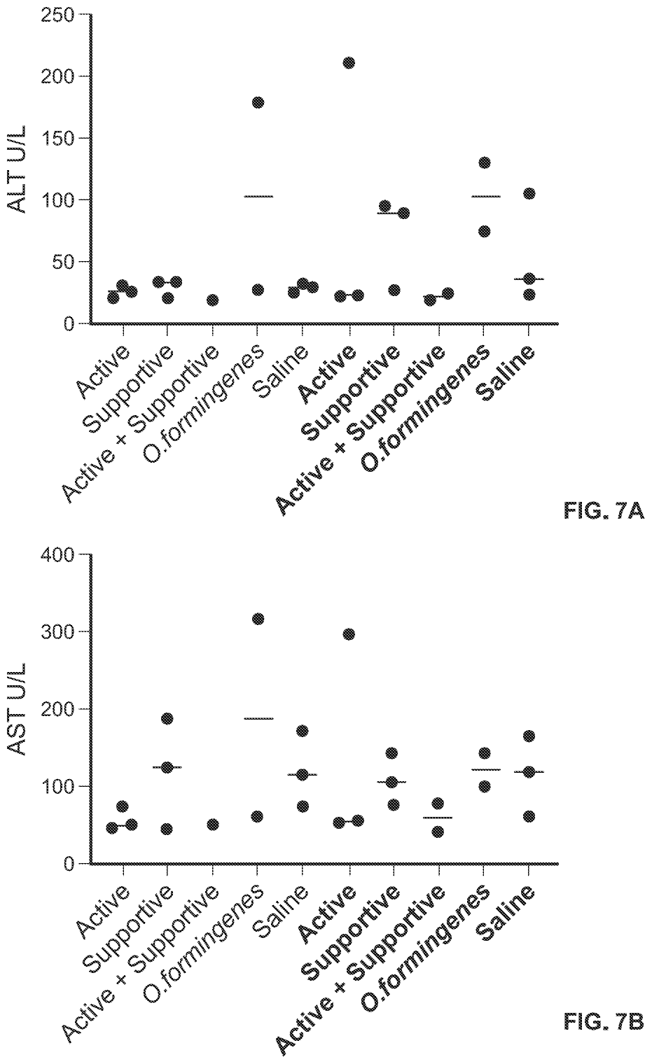

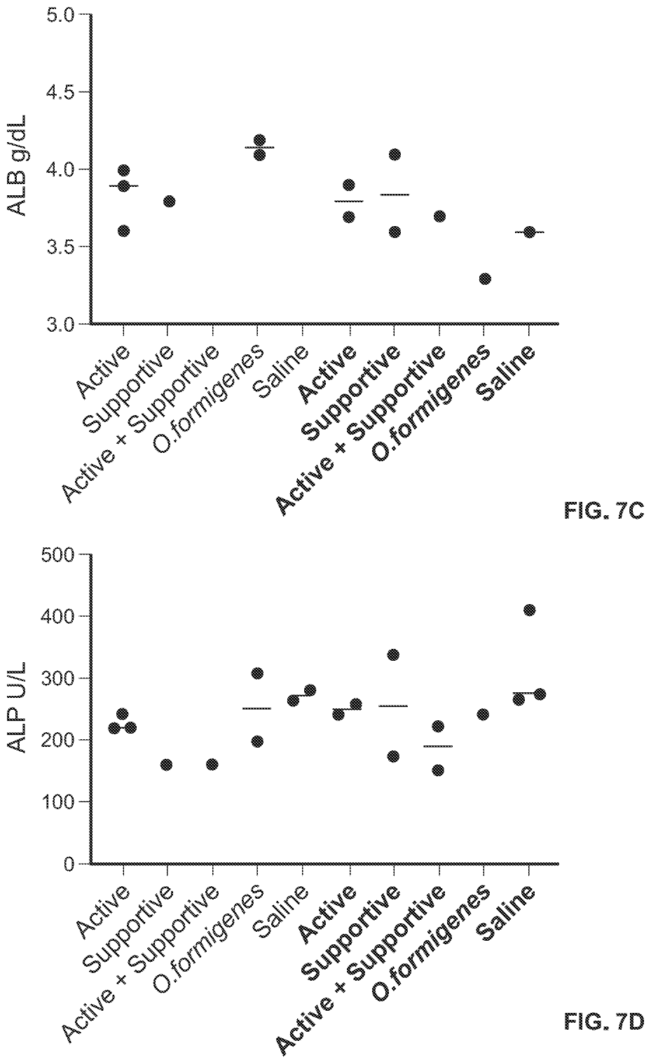

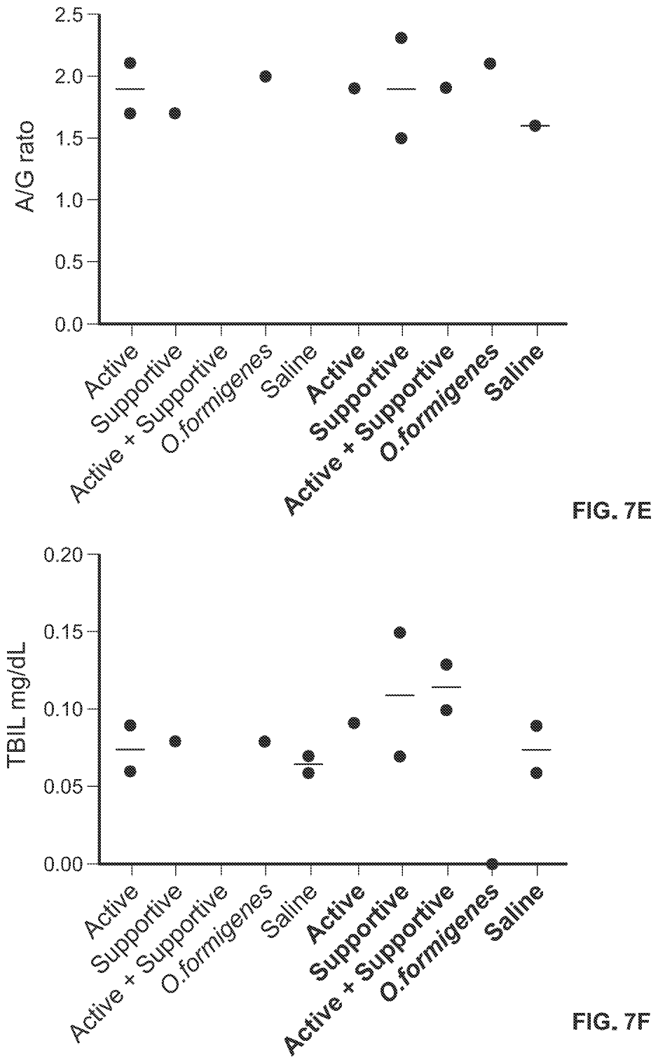

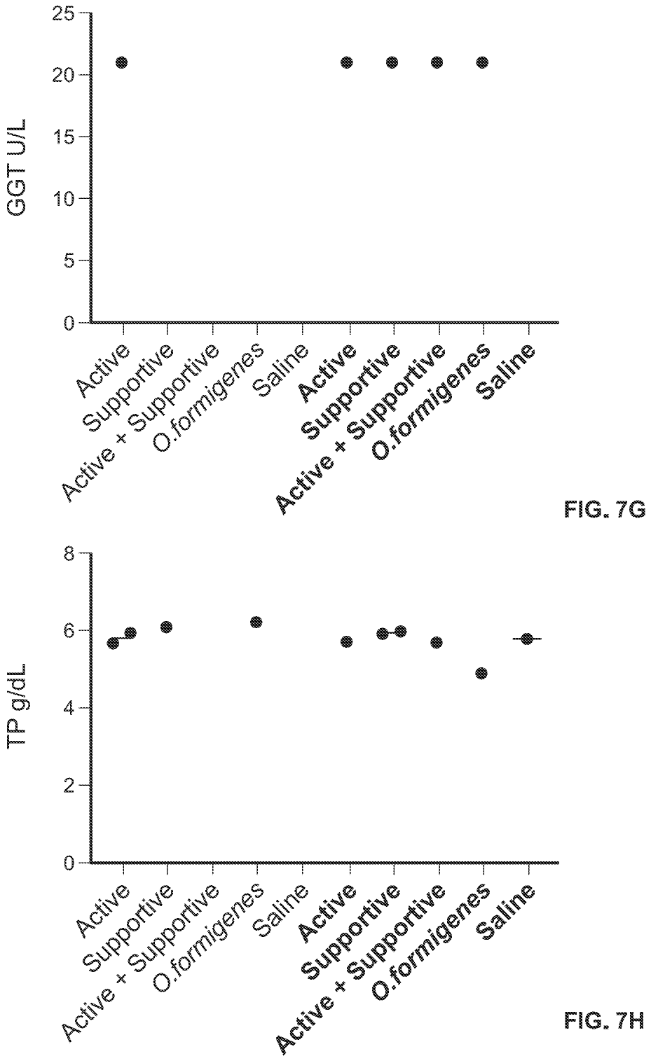

Mouse serum samples were analyzed for a standard panel of serum liver enzymes by the Charles River Laboratories. FIGS. 7A, 7B, 7C, 7D, 7E, 7F, 7G, and 7H show serum levels or function of alanine transaminase, aspartate transaminase, albumin, alanine phosphatase, albumin/globulin ratio, total bilirubin, gamma-glutamyl transferase, and prothrombin time, respectively, in gnotobiotic Balb/c mice on a normal (non-bold) or high oxalate diet (bold), treated by gavage with Oxalobacter formigenes only (O. formigenes), active strains only (Active), supportive strains only (Supportive), both active and supportive strains (Active and Supportive), or saline vehicle control (Saline) as described above.

### Kidney Function Assay

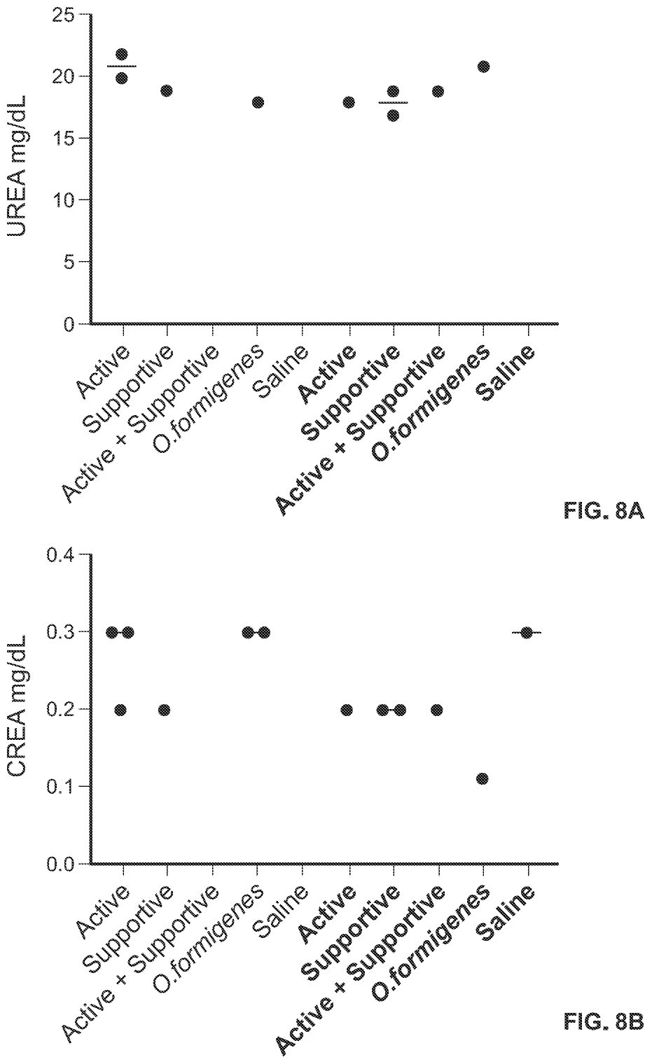

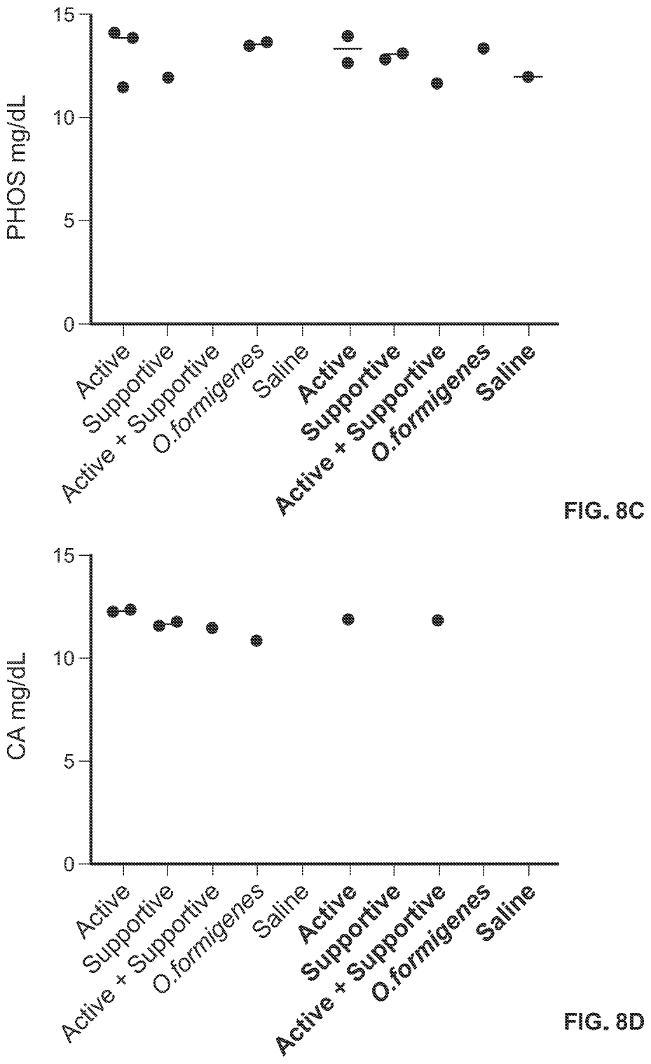

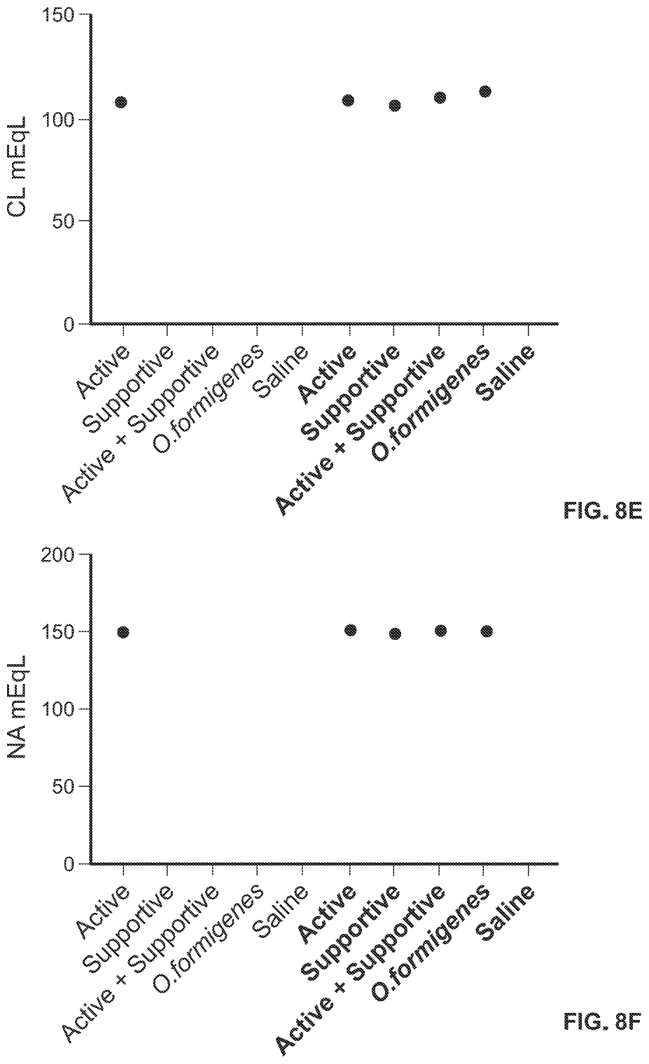

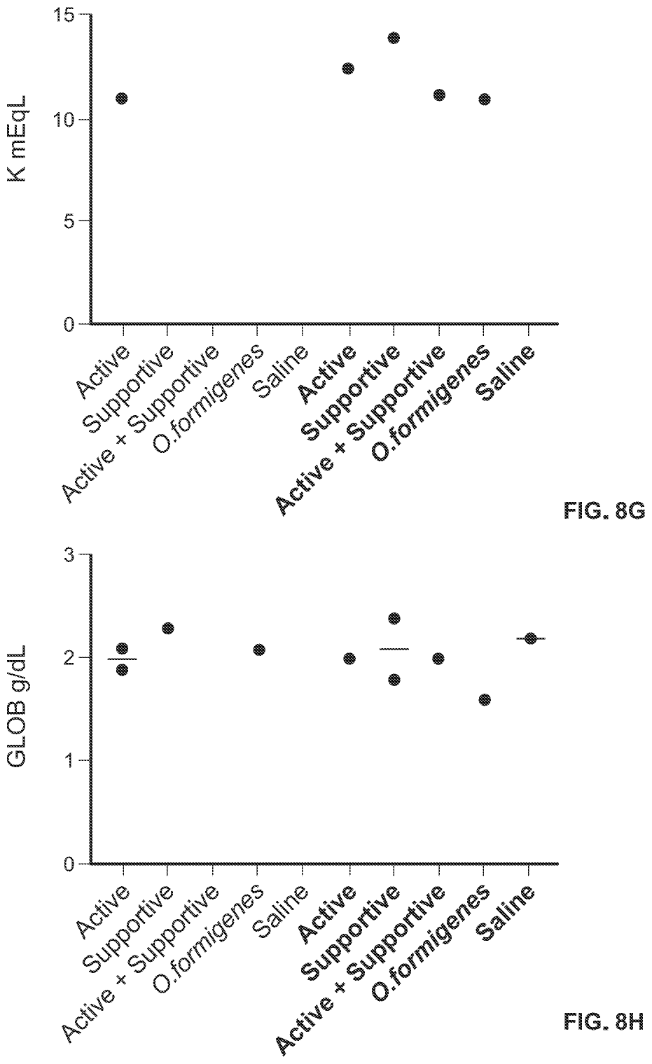

Mouse serum samples were analyzed for a standard panel of serum kidney metabolites/electrolytes by the Charles River Laboratories. FIGS. 8A, 8B, 8C, 8D, 8E, 8F, 8G, and 8H show serum levels of urea, creatinine, phosphorus, calcium, chloride, sodium, potassium, and globulin, respectively, in gnotobiotic Balb/c mice on a normal (non-bold) or high oxalate diet (bold), treated by gavage with Oxalobacter formigenes only (O. formigenes), active strains only (Active), supportive strains only (Supportive), both active and supportive strains (Active+Supportive), or saline vehicle control (Saline) as described above.

### Triglyceride, Cholesterol, Glucose and Creatine Kinase Assay

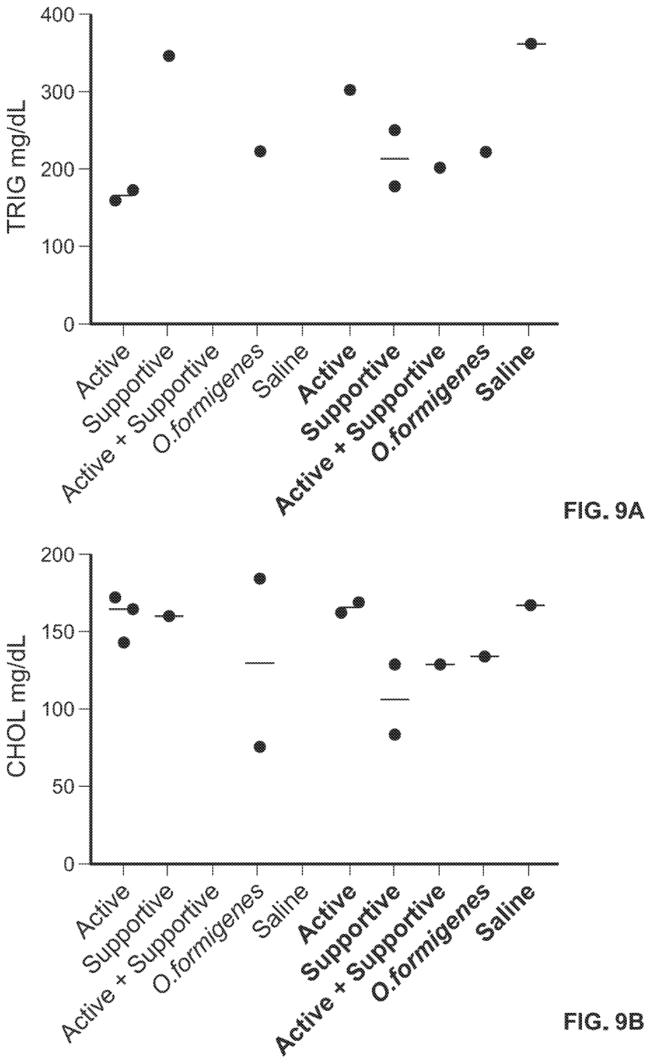

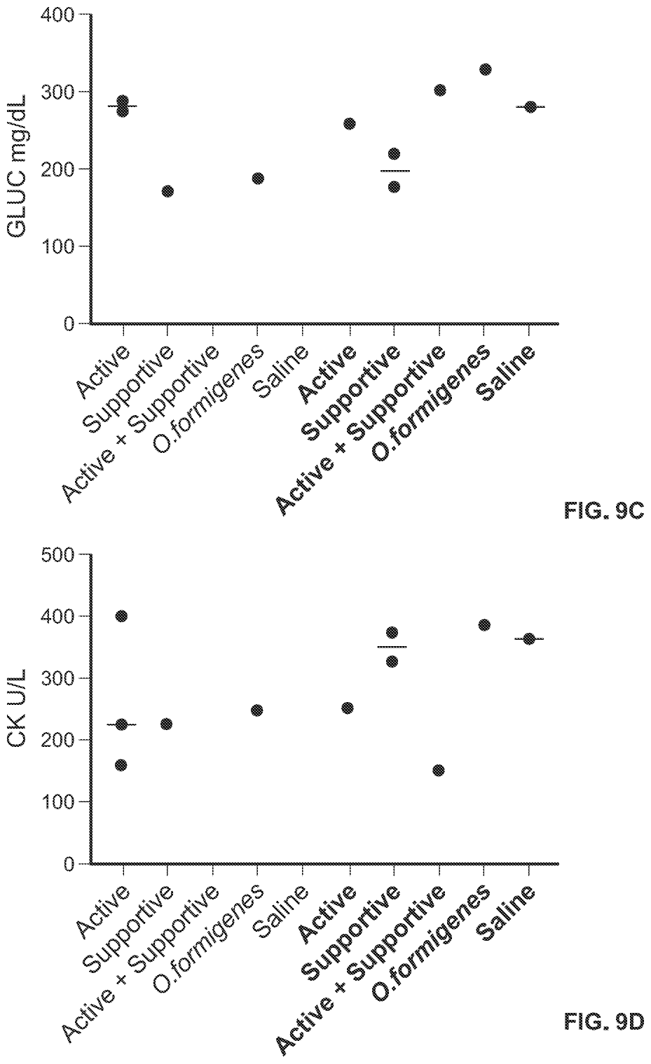

Mouse serum samples were analyzed for a standard triglyceride, cholesterol, glucose and creatine kinase panel by the Charles River Laboratories. FIGS. 9A, 9B, 9C, and 9D shows serum triglyceride, cholesterol, glucose, and creatine kinase levels, respectively, in gnotobiotic Balb/c mice on a normal (non-bold) or high oxalate diet (bold), treated by gavage with Oxalobacter formigenes only (O. formigenes), active strains only (Active), supportive strains only (Supportive), both active and supportive strains (Active+Supportive), or saline vehicle control as described above.

### Example 6: In Vivo Oxalate Metabolization in C57/B6 Female Mice Treated with a Microbial Consortium Containing Commercial Strains of Microbes

This example describes a study testing the ability of a microbial consortium, containing commercially-sourced strains of microbes, to degrade oxalate in vivo in C57/B6 female mice.

To test whether the in vivo activity of a microbial consortium as presently described was observed in a different sex and strain of study mouse, female C57/B6 mice (n=3 per condition) were colonized by oral gavage with either a plurality of active microbes alone, a supportive community alone, a supportive community plus O. formigenes alone, a supportive community plus a plurality of active microbes lacking O. formigenes, a complete microbial consortium (active and supportive s), or a fecal sample from a human donor found to be positive for O. formigenes DNA. The plurality of active microbes and the supportive community contained the strains in Table 8 marked with an ‘X’ in the indicated columns. Colonized mice were fed either a defined, low-complexity diet supplemented with excess oxalate in order to induce hyperoxaluria (see Table 2 above) or a nutritionally equivalent control diet lacking oxalate (see Table 1 above). After a two-week period, mice were sacrificed and urine, stool, serum and tissue samples were collected for analysis.

### Urinary Oxalate Concentrations

Urine was terminally collected from all groups and processed for solid phase extraction followed by LC-MS-based analysis of oxalate concentrations as described in Example 4. Absolute oxalate concentrations detected in individual urine samples were normalized based on the ratio of oxalate to creatinine.

As shown in Table 12, urinary oxalate levels were reduced in mice colonized with the complete microbial consortium. Partial reduction was also observed in mice colonized with the supportive community alone, the supportive community plus the plurality of active microbes lacking O. formigenes, and the plurality of active microbes alone.

TABLE 12

| | Normalized Urinary | |
|---|---|---|
| | Oxalate (mM) | |
| | n = 3 mice/group | |
| | | % of Urinary |
| | Oxalate:Cre- | Oxalate |
| | atinine | (normalized) in |
| Communities | (μM) | Supportive Only |
| Control (gnotobiotic) | 4.5 ± 0.73 | 120.4% ± 19.6 |
| Supportive Community only | 3.74 ± 1.27 | 100.0% ± 34.0 |
| Plurality of Active Microbes | 2.13 ± 1.81 | 57% ± 48.4 |
| only | | |
| Supportive Community + | 2.10 ± 0.90 | 56.1% ± 24.0 |
| O. formigenes | | |
| Supportive Community + | 2.82 ± 1.94 | 75.5% ± 52.0 |
| Plurality of Active | | |
| Microbes (minus O. formigenes) | | |
| Full Microbial Consortium | 0.91 ± 0.42 | 24.3% ± 11.3 |
| (Active + Supportive) | | |
| Human donor fecal sample | 1.64 ± 1.49 | 44.0% ± 40.0 |
| (O. formigenes positive) | | |

### Example 7: In Vivo Oxalate Metabolization in C57/B6 Female Mice Treated with Frozen Stocks of Microbial Consortium

To test whether a microbial consortium as presently described maintains in vivo activity after freezing, individual live microbial cultures of commercially-sourced strains were pooled in approximately equal proportions to form a supportive community alone, a supportive community plus a plurality of active microbes lacking O. formigenes, and an O. formigenes community comprising two commercial strains of O. formigenes, and frozen as aliquots in 30% glycerol in the vapor phase of a liquid nitrogen dewar for one month prior to administration to mice. The plurality of active microbes and the supportive community contained the strains in Table 8 marked with an ‘X’ in the indicated column.

Gnotobiotic, female, C57/B6 mice (n=3 per condition) were colonized by oral gavage with either a plurality of active microbes alone (including O. formigenes), a supportive community alone, or a complete microbial consortium (active and supportives). Hyperoxaluria was induced in colonized mice by providing ad libitum drinking water sweetened with sucralose and containing 0.875% oxalate. Control mice were provided with sucralose-sweetened drinking water without oxalate. All mice were maintained on a standard Autoclavable Mouse Breeder Diet (LabDiet®, St. Louis, Mo.). After a two week period, mice were sacrificed and a variety of samples were collected including urine, stool, serum, and kidneys.

### Urinary Oxalate Concentrations

As in Example 6, urine was terminally collected from all groups and processed for solid phase extraction followed by LC/MS-based analysis of oxalate concentrations. Absolute oxalate concentrations detected in individual urine samples were normalized based on the ratio of oxalate to creatinine.

As shown in Table 13, mice provided with drinking water containing 0.875% oxalate exhibited significantly elevated levels of urinary oxalate compared with mice given control water (e.g., an approximate 4-fold increase in both the mice administered with the plurality of active microbes alone and the mice administered with the supportive community alone). Consistent with Examples 5 and 6, mice colonized with the complete microbial consortium had significantly lower urinary oxalate levels compared with the mice administered with the plurality of active microbes alone or the mice administered with the supportive community alone. Furthermore, as compared to Examples 5 and 6, the complete microbial consortium still exhibited significant oxalate metabolizing activity in mice maintained on a markedly different standard dietary formulation

TABLE 13

| | | |
|---|---|---|
| | Oxalate:Creatinine | |
| Communities | (μM) | SD |

No Oxalate Treatment

| | | |
|---|---|---|
| Supportive Community + | 0.254 | 0.021 |
| Plurality of Active Microbes | | |
| Supportive Community only | 0.246 | 0.025 |
| Plurality of Active Microbes only | 0.279 | 0.019 |

0.875% Oxalate Treatment

| | | |
|---|---|---|
| Supportive Community + | 0.618 | 0.085 |
| Plurality of Active Microbes | | |
| Supportive Community only | 0.937 | 0.111 |
| Plurality of Active Microbes only | 2.427 | 2.284 |

### Example 8: In Vivo Engraftment of Oxalate-Metabolizing Microbial Strains

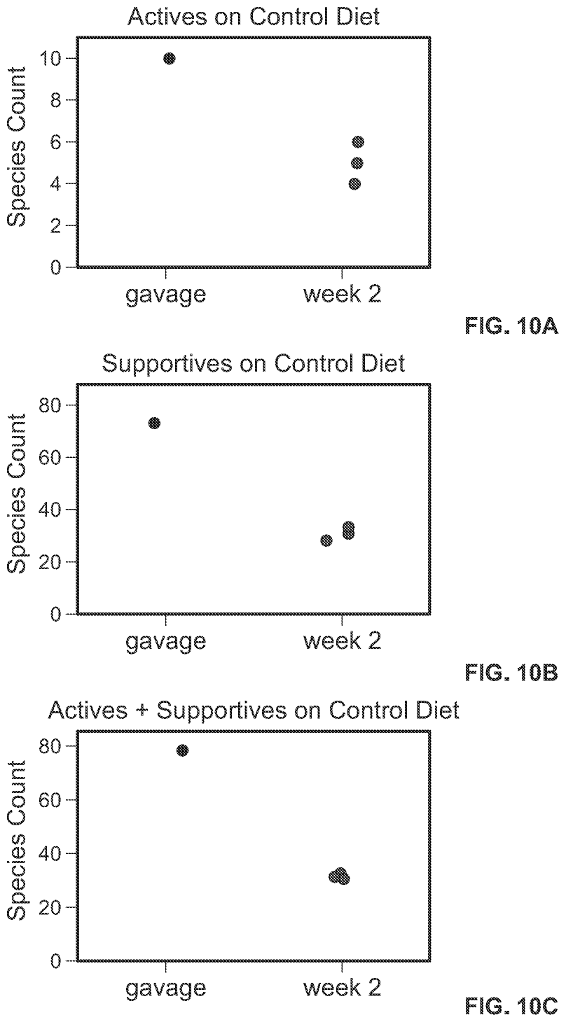

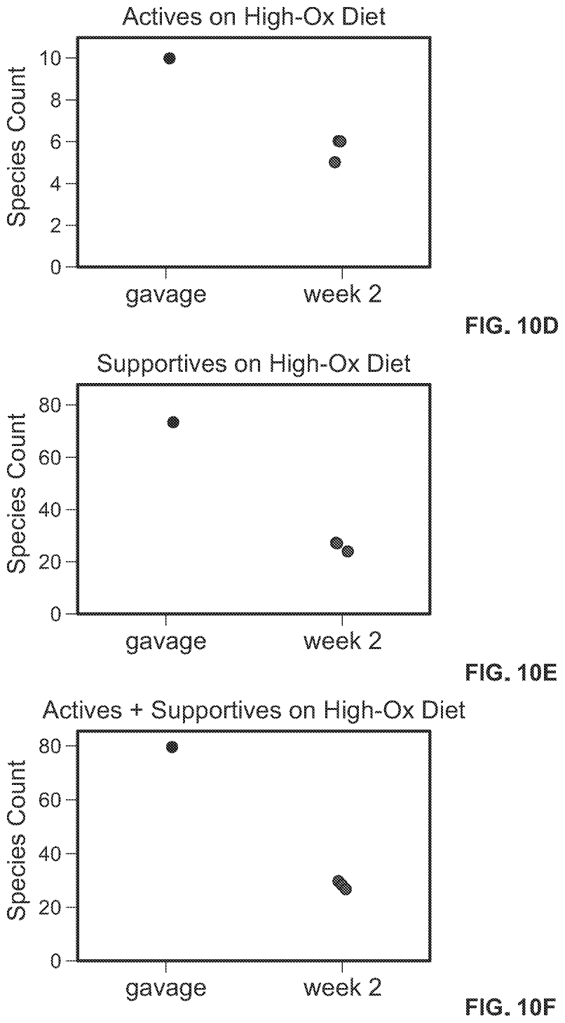

Stool samples from the treated mice described in Example 5 were analyzed for the presence of oxalate-metabolizing microbial strains by whole genome shotgun sequencing of microbial DNA extracted from fecal pellets. DNA extraction from fecal samples and whole genome shotgun sequencing were performed by methods as previously described in Example 1. Sequence reads were mapped against a comprehensive database of complete, sequenced genomes of all the defined microbial strains comprising the microbial consortium. The results of this experiment are summarized in FIGS. 10A-F.

Table 14 shows detection of engrafted oxalate-metabolizing active microbial strains in the treated mice described in Example 5. Microbial strains were counted as “detected” if their relative abundance was >0.1% of total sequence reads.

TABLE 14

| | | | Actives + | Actives + |
|---|---|---|---|---|
| | Actives- | Actives- | Sup- | Sup- |
| | only | only | portives | portives |
| Active community | (Control | (Oxalate | (Control | (Oxalate |
| component | Diet) | Diet) | Diet) | Diet) |
| Oxalobacter formigenes | 0% | 100% | 0% | 100% |
| Bifidobacterium dentium | 100% | 100% | 0% | 0% |
| Eggerthella lenta | 100% | 100% | 0% | 0% |
| Enterococcus faecalis | 100% | 100% | 0% | 0% |
| Lactobacillus rhamnosus | 100% | 100% | 0% | 0% |
| Dialister invisus | 33.3% | 66.6% | 0% | 0% |
| Lactobacillus helveticus | 33.3% | 0% | 0% | 0% |
| Lactobacillus acidophilus | 0% | 0% | 0% | 0% |
| Lactobacillus gasseri | 0% | 0% | 0% | 0% |
| Lactobacillus reuteri | 0% | 0% | 0% | 0% |

Table 15 shows detection of engrafted supportive microbial strains in the treated mice described in Example 5. Microbial strains were counted as “detected” if their relative abundance was >0.1% of total sequence reads.

TABLE 15

| | Sup- | Sup- | Actives + | Actives + |
|---|---|---|---|---|
| | portives- | portives- | Sup- | Sup- |
| | only | only | portives | portives |
| Supportive Community | (Control | (Oxalate | (Control | (Oxalate |
| Component | Diet) | Diet) | Diet) | Diet) |
| 13 species | 100% | 100% | 100% | 100% |
| Alistipes senegalensis | 100% | 0% | 66.6% | 100% |
| Catenibacterium | 100% | 0% | 66.6% | 100% |
| mitsuokai | | | | |
| Bacteroides coprocola | 0% | 0% | 0% | 100% |
| Clostridium scindens | 0% | 0% | 66.6% | 66.6% |
| Bacteroides | 100% | 0% | 66.6% | 0% |
| cellulosilyticus | | | | |
| Tyzzerella nexilis | 100% | 0% | 66.6% | 0% |
| 17 species | 0 to | 0 to | 0 to | 0 to |
| | 100% | 100% | 100% | 100% |
| 33 species | 0% | 0% | 0% | 0% |

### Example 9: In Vitro Oxalate Metabolization by Donor-Derived Strains

In order to determine the in vitro oxalate-metabolizing activity of three donor-derived O. formigenes strains, strains were grown in YFCAC base medium at either pH 7.0, 6.0, or 5.0 in the presence of 80 mM oxalate. Strains were incubated at 37° C. for 72, and at the conclusion of the protocol the amount of oxalate in the medium was quantified by LC-MS as described in Example 4. For all three strains, the amount of oxalate remaining in the culture medium after 72 hours was below the limit of detection when assayed at pH 7.0 or 6.0. No oxalate degradation was detected for cultures of any of the three strains when incubated at pH 5.0.

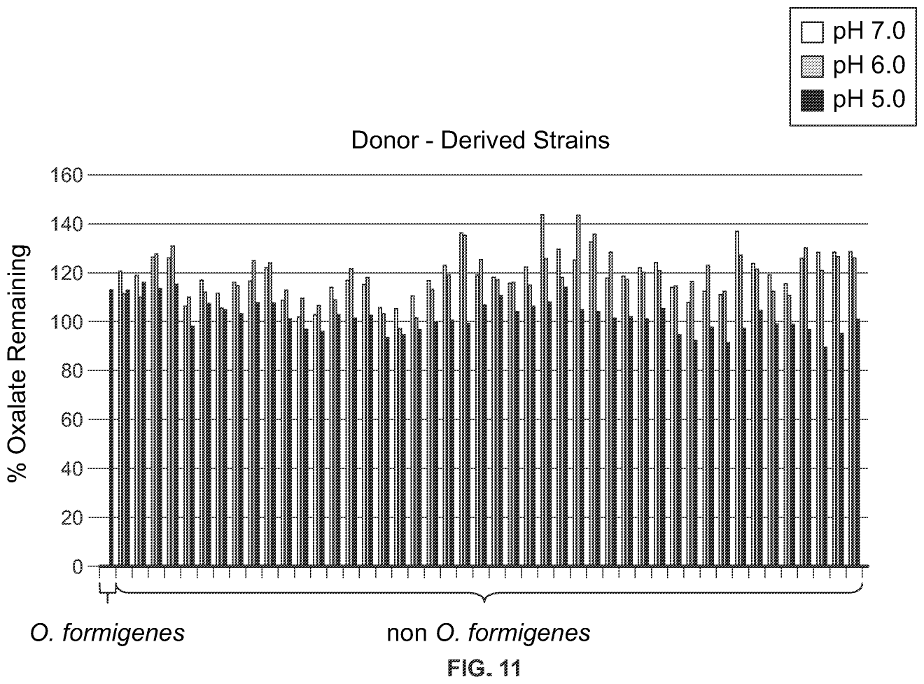

To determine the oxalate-metabolizing activity of additional donor-derived microbial strains, strains were grown in anaerobic conditions in YCFAC base medium at either pH 7.0, 6.0, or 5.0 in the presence of 2 mM oxalate. Strains were incubated at 37° C. for 120 hours, and at the conclusion of the protocol the amount of oxalate in the medium was quantified by LC/MS as described in Example 4. A donor-derived strain of O. formigenes was included as a positive control. Results are reported as the percentage of oxalate remaining in the media at the conclusion of the assay relative to the starting concentration (FIG. 11). As expected, the amount of oxalate remaining in a culture of donor-derived O. formigenes was below the limit of detection when assayed at pH 6 or pH 7, although no oxalate degradation was detected at pH 5. By contrast, none of the other tested donor-derived isolates were found to reduce oxalate by more than 11% at any pH tested.

### Example 10: Growth of Donor-Derived O. formigenes Strains at Different pHs and Oxalate Concentrations

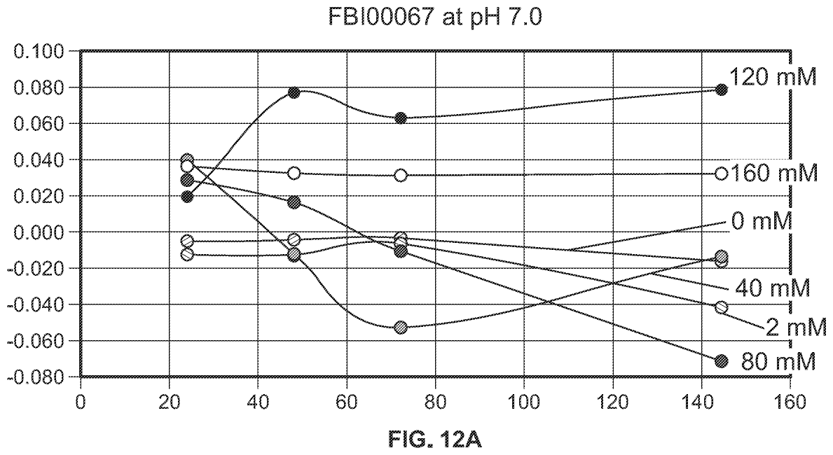

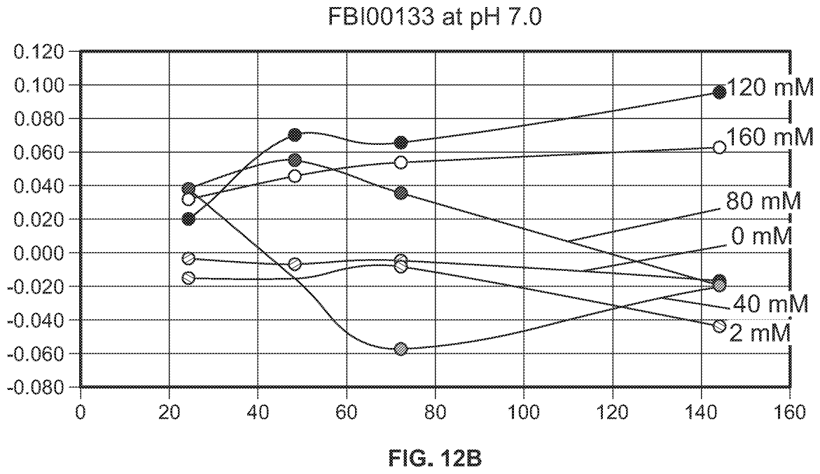

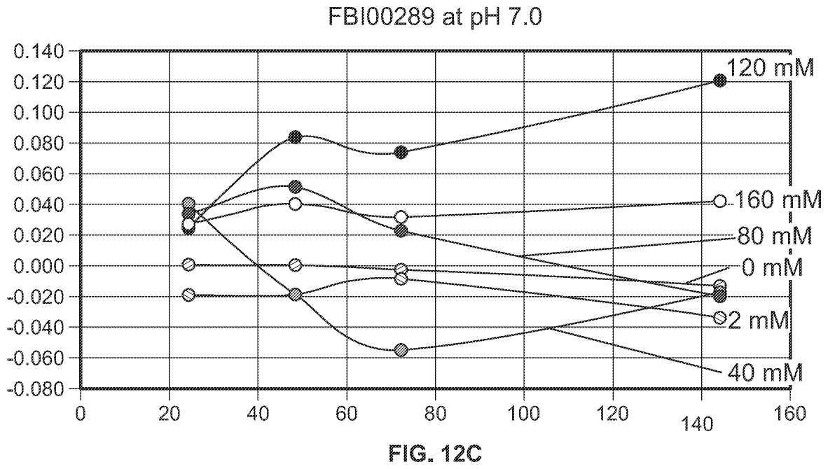

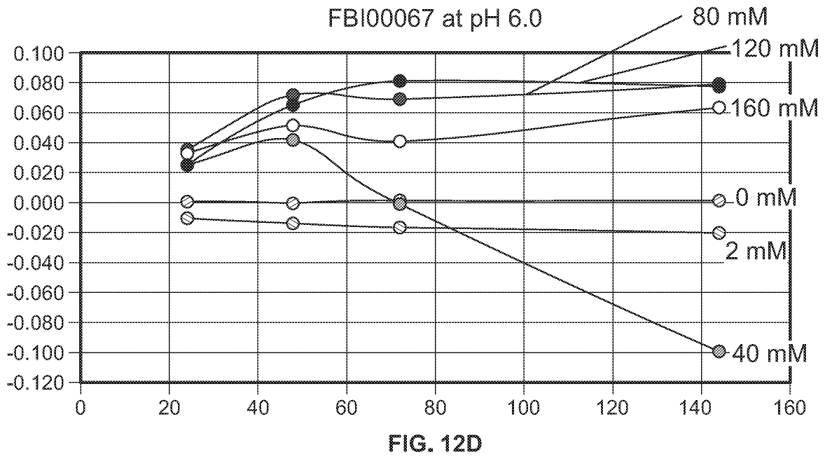

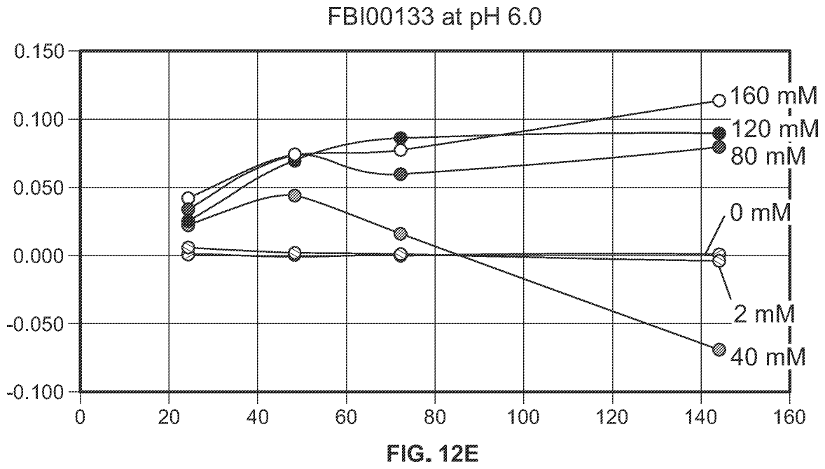

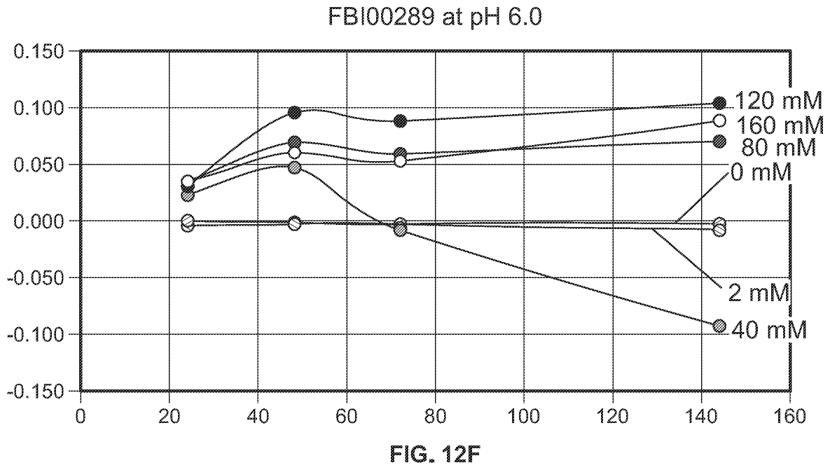

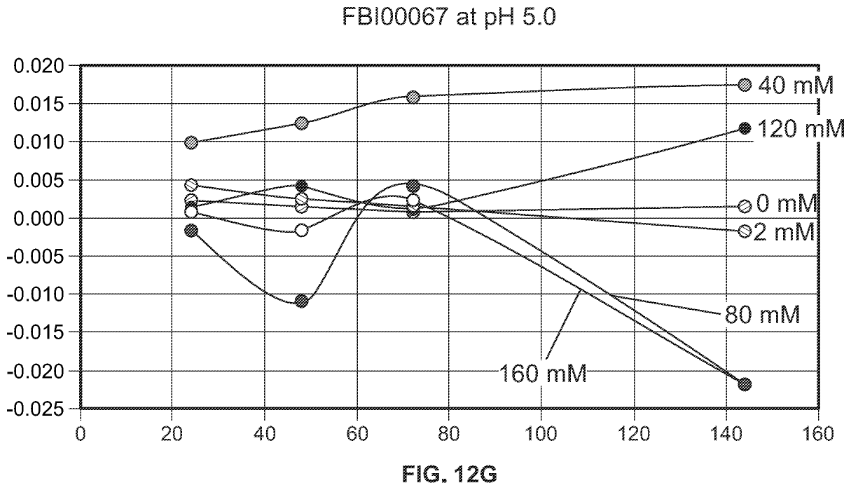

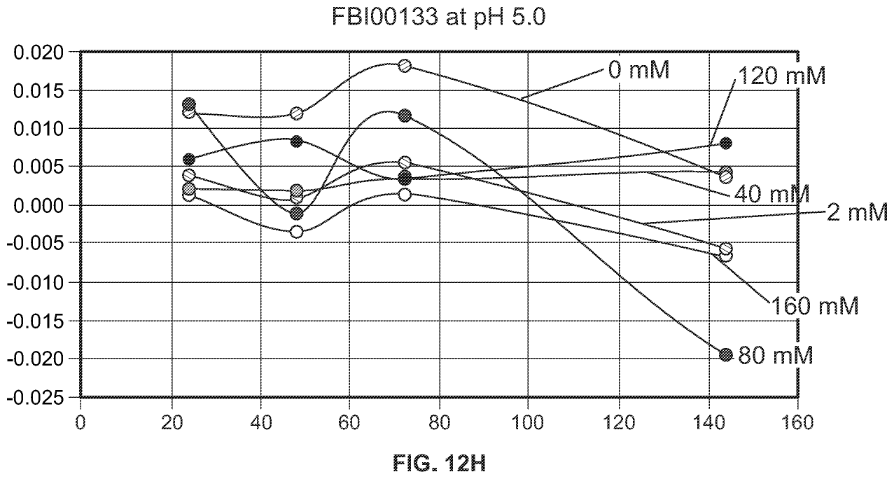

Three O. formigenes strains isolated from donor fecal samples were assayed for their ability to grow at different pHs (5.0, 6.0, or 7.0) and at different oxalate concentrations (0 mM, 2 mM, 40 mM, 80 mM, 120 mM, 160 mM). Strains were grown under anaerobic conditions in the appropriate banking medium, and culture turbidity was recorded after 24, 48, 72, and 144 hours. The results of this assay are reported in FIGS. 12A-12C. One O. formigenes strain (FBI00067) was observed to grow better at a lower pH; another strain (FBI00133) was observed to be more tolerant of higher oxalate concentrations.

### Example 11: Design of Supportive Communities Comprising Donor-Derived Strains

Supportive communities of microbes were designed using donor-derived strains. Five candidate communities were designed according to different design principles.

The supportive community of candidate consortium I was designed to incorporate all isolated species that were present in more than 50% of a set of healthy donor fecal samples. The community further included donor-derived strains whose identified species had been represented in the proof-of-concept consortium of commercial strains, or (if no matching species had been isolated) then a strain of the species that was the closest relative within the genus. The final consortium (actives and supportives) contained 152 strains and 70 species in total, listed in Table 16.

The supportive communities of candidate consortia II and III were designed to maximize consumption and/or production of a defined set of metabolites using a minimal number of strains. In both cases, metabolites of interest were identified by conducting a literature review, as well as by bioinformatic annotation of healthy microbiomes. Next, the genomes of donor-derived strains were bioinformatically analyzed to identify strains capable of producing or consuming said metabolites of interest. A literature review was also conducted to identify donor-derived strains belonging to species known to consume and/or produce each metabolite of interest. Donor-derived strains were scored for their ability to produce or consume said metabolite, and the community was designed to maximize the desired metabolic coverage with the fewest number of species. The supportive community of candidate consortium II was designed to enrich for consumption of 51 dietary carbon and energy sources. The supportive community of candidate consortia III was designed to enrich for the production or consumption of metabolites present in the host, including bile acids, sugars, amino acids, vitamins, SCFAs, and gasses. The strains included in candidate consortium II are listed in Table 17, and the strains included in candidate consortium III are listed in Table 18.

The supportive community of candidate consortium IV was constructed using strains isolated exclusively from fecal samples of two healthy donors. Sourcing many supportive strains from one or a small number of donors may have the benefit of enhancing co-culturability and/or ecological stability. The two specific donors selected both had stool that was found to be capable of reducing urinary oxalate in vivo, potentially enhancing the use of the community in embodiments of the invention designed to degrade oxalate. The strains included in candidate consortium IV are listed in Table 19.

The supportive community of candidate consortium V was designed to include all strains isolated from healthy donor fecal samples, with the exception of species known to be associated with pathogenesis. This diverse community incorporated species from all five major phyla that comprise normal gut commensals (Bacteroidetes, Firmicutes, Actinobacteria, Proteobacteria, and Verrucomicrobia). The final consortium contained 103 species and 158 strains in total, which are listed in Table 20.

TABLE 16

| | | | | NCBI | | | |
|---|---|---|---|---|---|---|---|
| Strain | | | | Taxonomy | | % Match | SEQ ID |
| # | Species ID | Kingdom | Phylum | ID | Closest 16S Species | (16S) | NO: X |
| FBI00001 | Clostridium citroniae | bacteria | firmicutes | 358743 | Clostridium citroniae | 99.64 | 1 |
| FBI00002 | Bacteroides salyersiae | bacteria | bacteroidetes | 291644 | Bacteroides salyersiae | 99.5 | 2 |
| FBI00004 | Neglecta timonensis | bacteria | firmicutes | 1776382 | Neglecta timonensis | 99.14 | 4 |
| FBI00008 | Blautia luti | bacteria | firmicutes | 89014 | Blautia luti | 97.02 | 8 |
| FBI00010 | Blautia obeum | bacteria | firmicutes | 40520 | Blautia obeum | 98.12 | 10 |
| FBI00011 | Bifidobacterium longum | bacteria | actinobacteria | 216816 | Bifidobacterium longum | 99.28 | 11 |
| FBI00012 | Alistipes onderdonkii | bacteria | bacteroidetes | 328813 | Alistipes onderdonkii | 99.71 | 12 |
| FBI00013 | Parabacteroides merdae | bacteria | bacteroidetes | 46503 | Parabacteroides merdae | 99.5 | 13 |
| FBI00015 | Bacteroides uniformis | bacteria | bacteroidetes | 820 | Bacteroides uniformis | 99.78 | 15 |
| FBI00016 | Bifidobacterium | bacteria | actinobacteria | 28026 | Bifidobacterium | 99.64 | 16 |
| | pseudocatenulatum | | | | pseudocatenulatum | | |
| FBI00017 | Blautia obeum | bacteria | firmicutes | 40520 | Blautia obeum | 98.34 | 17 |
| FBI00018 | Eubacterium rectale | bacteria | firmicutes | 39491 | Eubacterium rectale | 99.71 | 18 |
| FBI00019 | Alistipes timonensis | bacteria | bacteroidetes | 1465754 | Alistipes timonensis | 99.78 | 19 |
| FBI00020 | Bacteroides | bacteria | bacteroidetes | 818 | Bacteroides | 99.57 | 20 |
| | thetaiotaomicron | | | | thetaiotaomicron | | |
| FBI00022 | Alistipes putredinis | bacteria | bacteroidetes | 28117 | Alistipes putredinis | 99.93 | 22 |
| FBI00025 | Coprococcus comes | bacteria | firmicutes | 410072 | Coprococcus comes | 99.21 | 25 |
| FBI00029 | Parabacteroides distasonis | bacteria | bacteroidetes | 823 | Parabacteroides distasonis | 99.26 | 29 |
| FBI00030 | Eggerthella lenta | bacteria | firmicutes | 84112 | Eggerthella lenta | 98.47 | 30 |
| FBI00032 | Anaerostipes hadrus | bacteria | firmicutes | 649756 | Anaerostipes hadrus | 99.64 | 32 |
| FBI00033 | Lachnospiraceae sp. FBI00033 | bacteria | firmicutes | 186803 | Clostridium amygdalinum | 93.56 | 33 |
| FBI00034 | Eubacterium eligens | bacteria | firmicutes | 39485 | Eubacterium eligens | 98.78 | 34 |
| FBI00036 | Blautia faecis | bacteria | firmicutes | 871665 | Blautia faecis | 99.53 | 36 |
| FBI00038 | Coprococcus eutactus | bacteria | firmicutes | 33043 | Coprococcus eutactus | 95.96 | 38 |
| FBI00039 | Bacteroides vulgatus | bacteria | bacteroidetes | 821 | Bacteroides vulgatus | 99.71 | 39 |
| FBI00040 | Bilophila wadsworthia | bacteria | proteobacteria | 35833 | Desulfovibrio desulfuricans | 91.38 | 40 |
| FBI00042 | Bacteroides xylanisolvens | bacteria | bacteroidetes | 371601 | Bacteroides xylanisolvens | 99.71 | 42 |
| FBI00043 | Bifidobacterium dentium | bacteria | actinobacteria | 1689 | Bifidobacterium dentium | 99.35 | 43 |
| FBI00044 | Blautia wexlerae | bacteria | firmicutes | 418240 | Blautia wexlerae | 98.69 | 44 |
| FBI00046 | Bacteroides caccae | bacteria | bacteroidetes | 47678 | Bacteroides caccae | 99.71 | 46 |
| FBI00047 | Eubacterium eligens | bacteria | firmicutes | 39485 | Eubacterium eligens | 98.79 | 47 |
| FBI00050 | Bacteroides nordii | bacteria | bacteroidetes | 291645 | Bacteroides nordii | 98.63 | 50 |
| FBI00051 | Dorea formicigenerans | bacteria | firmicutes | 39486 | Dorea formicigenerans | 98.07 | 51 |
| FBI00052 | Bacteroides xylanisolvens | bacteria | bacteroidetes | 371601 | Bacteroides xylanisolvens | 99.14 | 52 |
| FBI00056 | Clostridium citroniae | bacteria | firmicutes | 358743 | Clostridium citroniae | 99.2 | 56 |
| FBI00057 | Dorea longicatena | bacteria | firmicutes | 88431 | Dorea longicatena | 99.7 | 57 |
| FBI00058 | Eubacterium rectale | bacteria | firmicutes | 39491 | Eubacterium rectale | 99.71 | 58 |
| FBI00059 | Bacteroides stercorirosoris | bacteria | bacteroidetes | 871324 | Bacteroides oleiciplenus | 98.81 | 59 |
| FBI00060 | Bifidobacterium longum | bacteria | actinobacteria | 216816 | Bifidobacterium longum | 99.49 | 60 |
| FBI00061 | Alistipes shahii | bacteria | bacteroidetes | 328814 | Alistipes shahii | 99.19 | 61 |
| FBI00062 | Collinsella aerofaciens | bacteria | actinobacteria | 74426 | Collinsella aerofaciens | 99.48 | 62 |
| FBI00066 | Parasutterella excrementihominis | bacteria | proteobacteria | 487175 | Parasutterella excrementihominis | 99.13 | 66 |
| FBI00067 | Oxalobacter formigenes | bacteria | proteobacteria | 847 | Oxalobacter formigenes | 98.84 | 67 |
| FBI00068 | Akkermansia muciniphila | bacteria | verrucomicrobia | 239935 | Akkermansia muciniphila | 99.42 | 68 |
| FBI00069 | Ruminococcus bromii | bacteria | firmicutes | 40518 | Ruminococcus bromii | 98.84 | 69 |
| FBI00072 | Coprococcus eutactus | bacteria | firmicutes | 33043 | Coprococcus eutactus | 96.17 | 72 |
| FBI00073 | Parabacteroides distasonis | bacteria | bacteroidetes | 823 | Parabacteroides distasonis | 98.99 | 73 |
| FBI00074 | Clostridium fessum | bacteria | firmicutes | 2126740 | Clostridium symbiosum | 94.03 | 74 |
| FBI00075 | Paraprevotella clara | bacteria | bacteroidetes | 454154 | Paraprevotella clara | 98.85 | 75 |
| FBI00076 | Bacteroides thetaiotaomicron | bacteria | bacteroidetes | 818 | Bacteroides thetaiotaomicron | 99.78 | 76 |
| FBI00078 | Blautia obeum | bacteria | firmicutes | 40520 | Blautia obeum | 98.34 | 78 |
| FBI00085 | Ruminococcus bromii | bacteria | firmicutes | 40518 | Ruminococcus bromii | 98.62 | 85 |
| FBI00086 | Ruminococcus bromii | bacteria | firmicutes | 40518 | Ruminococcus bromii | 98.77 | 86 |
| FBI00087 | Clostridium scindens | bacteria | firmicutes | 29347 | Clostridium scindens | 98.28 | 87 |
| FBI00090 | Eubacterium eligens | bacteria | firmicutes | 39485 | Eubacterium eligens | 98.71 | 90 |
| FBI00091 | Eubacterium rectale | bacteria | firmicutes | 39491 | Eubacterium rectale | 99.86 | 91 |
| FBI00093 | Roseburia hominis | bacteria | firmicutes | 301301 | Roseburia hominis | 99.71 | 93 |
| FBI00096 | Eggerthella lenta | bacteria | actinobacteria | 84112 | Eggerthella lenta | 98.76 | 96 |
| FBI00099 | Gordonibacter pamelaeae | bacteria | actinobacteria | 471189 | Gordonibacter pamelaeae | 99.56 | 99 |
| FBI00101 | Faecalibacterium prausnitzii | bacteria | firmicutes | 853 | Faecalibacterium prausnitzii | 97.97 | 101 |
| FBI00102 | Clostridium fessum | bacteria | firmicutes | 2126740 | Clostridium symbiosum | 94.31 | 102 |
| FBI00104 | Blautia wexlerae | bacteria | firmicutes | 418240 | Blautia luti | 97.18 | 104 |
| FBI00109 | Coprococcus comes | bacteria | firmicutes | 410072 | Coprococcus comes | 98.39 | 109 |
| FBI00111 | Bacteroides vulgatus | bacteria | bacteroidetes | 821 | Bacteroides vulgatus | 99.43 | 111 |
| FBI00112 | Bacteroides uniformis | bacteria | bacteroidetes | 820 | Bacteroides uniformis | 99.78 | 112 |
| FBI00113 | Parabacteroides merdae | bacteria | bacteroidetes | 46503 | Parabacteroides merdae | 99.79 | 113 |
| FBI00115 | Dorea formicigenerans | bacteria | firmicutes | 39486 | Dorea formicigenerans | 97.98 | 115 |
| FBI00116 | Ruminococcus faecis | bacteria | firmicutes | 592978 | Ruminococcus faecis | 99.57 | 116 |
| FBI00117 | Blautia faecis | bacteria | firmicutes | 871665 | Blautia faecis | 99.52 | 117 |
| FBI00118 | Blautia faecis | bacteria | firmicutes | 871665 | Blautia faecis | 99.84 | 118 |
| FBI00120 | Hungatella effluvii | bacteria | firmicutes | 154046 | Hungatella hathewayi | 98.78 | 120 |
| FBI00122 | Bacteroides uniformis | bacteria | bacteroidetes | 820 | Bacteroides uniformis | 99.57 | 122 |
| FBI00123 | Roseburia hominis | bacteria | firmicutes | 301301 | Roseburia hominis | 100 | 123 |
| FBI00124 | Anaerostipes hadrus | bacteria | firmicutes | 649756 | Anaerostipes hadrus | 99.86 | 124 |
| FBI00125 | Bacteroides stercoris | bacteria | bacteroidetes | 46506 | Bacteroides stercoris | 99.64 | 125 |
| FBI00126 | Bifidobacterium adolescentis | bacteria | actinobacteria | 1680 | Bifidobacterium adolescentis | 98.98 | 126 |
| FBI00127 | Collinsella aerofaciens | bacteria | actinobacteria | 74426 | Collinsella aerofaciens | 98.81 | 127 |
| FBI00130 | Coprococcus comes | bacteria | firmicutes | 410072 | Coprococcus comes | 99.35 | 130 |
| FBI00132 | Gordonibacter pamelaeae | bacteria | actinobacteria | 471189 | Gordonibacter pamelaeae | 99.48 | 132 |
| FBI00133 | Oxalobacter formigenes | bacteria | proteobacteria | 847 | Oxalobacter formigenes | 99.21 | 133 |
| FBI00135 | Bifidobacterium | bacteria | actinobacteria | 28026 | Bifidobacterium | 99.57 | 135 |
| | pseudocatenulatum | | | | pseudocatenulatum | | |
| FBI00137 | Bacteroides fragilis | bacteria | bacteroidetes | 817 | Bacteroides fragilis | 99.71 | 137 |
| FBI00138 | Blautia massiliensis | bacteria | firmicutes | 1737424 | Blautia luti | 97.94 | 138 |
| FBI00139 | Bacteroides thetaiotaomicron | bacteria | bacteroidetes | 818 | Bacteroides thetaiotaomicron | 99.5 | 139 |
| FBI00142 | Clostridium fessum | bacteria | firmicutes | 2126740 | Clostridium symbiosum | 94.07 | 142 |
| FBI00143 | Parabacteroides merdae | bacteria | bacteroidetes | 46503 | Parabacteroides merdae | 99.07 | 143 |
| FBI00145 | Bifidobacterium adolescentis | bacteria | actinobacteria | 1680 | Bifidobacterium adolescentis | 99.14 | 145 |
| FBI00147 | Clostridium bolteae | bacteria | firmicutes | 208479 | Clostridium bolteae | 99.28 | 147 |
| FBI00151 | Clostridium aldenense | bacteria | firmicutes | 358742 | Clostridium aldenense | 98.55 | 151 |
| FBI00152 | Dialister invisus | bacteria | firmicutes | 218538 | Dialister invisus | 99.58 | 152 |
| FBI00155 | Blautia obeum | bacteria | firmicutes | 40520 | Blautia obeum | 98.7 | 155 |
| FBI00162 | Bifidobacterium catenulatum | bacteria | actinobacteria | 1686 | Bifidobacterium catenulatum | 99.14 | 162 |
| FBI00164 | Bacteroides stercoris | bacteria | bacteroidetes | 46506 | Bacteroides stercoris | 98.56 | 164 |
| FBI00165 | Bacteroides massiliensis | bacteria | bacteroidetes | 204516 | Bacteroides massiliensis | 99.71 | 165 |
| FBI00167 | Dorea longicatena | bacteria | firmicutes | 88431 | Dorea longicatena | 99.39 | 167 |
| FBI00168 | Collinsella aerofaciens | bacteria | actinobacteria | 74426 | Collinsella aerofaciens | 99.26 | 168 |
| FBI00169 | Parabacteroides distasonis | bacteria | bacteroidetes | 823 | Parabacteroides distasonis | 98.7 | 169 |
| FBI00170 | Eggerthella lenta | bacteria | actinobacteria | 84112 | Eggerthella lenta | 98.61 | 170 |
| FBI00171 | Bilophila wadsworthia | bacteria | proteobacteria | 35833 | Desulfovibrio desulfuricans | 91.45 | 171 |
| FBI00172 | Bifidobacterium longum | bacteria | actinobacteria | 216816 | Bifidobacterium longum | 99.05 | 172 |
| FBI00173 | Bacteroides vulgatus | bacteria | bacteroidetes | 821 | Bacteroides vulgatus | 100 | 173 |
| FBI00175 | Holdemanella biformis | bacteria | firmicutes | 1735 | Holdemanella biformis | 98.19 | 175 |
| FBI00177 | Parasutterella excrementihominis | bacteria | proteobacteria | 487175 | Parasutterella excrementihominis | 99.71 | 177 |
| FBI00180 | Alistipes sp. FBI00180 | bacteria | bacteroidetes | 239759 | Alistipes senegalensis | 97.56 | 180 |
| FBI00181 | Blautia wexlerae | bacteria | firmicutes | 418240 | Blautia wexlerae | 97.17 | 181 |
| FBI00182 | Bacteroides coprocola | bacteria | bacteroidetes | 310298 | Bacteroides coprocola | 99.64 | 182 |
| FBI00186 | Coprococcus comes | bacteria | firmicutes | 410072 | Coprococcus comes | 99.06 | 186 |
| FBI00188 | Blautia faecis | bacteria | firmicutes | 871665 | Blautia faecis | 99.05 | 188 |
| FBI00190 | Bacteroides finegoldii | bacteria | bacteroidetes | 338188 | Bacteroides finegoldii | 98.91 | 190 |
| FBI00193 | Alistipes onderdonkii | bacteria | bacteroidetes | 328813 | Alistipes onderdonkii | 99.64 | 193 |
| FBI00194 | Ruminococcus faecis | bacteria | firmicutes | 592978 | Ruminococcus faecis | 98.41 | 194 |
| FBI00199 | Clostridium bolteae | bacteria | firmicutes | 208479 | Clostridium bolteae | 99.28 | 199 |
| FBI00201 | Eggerthella lenta | bacteria | actinobacteria | 84112 | Eggerthella lenta | 98.83 | 201 |
| FBI00205 | Blautia massiliensis | bacteria | firmicutes | 1737424 | Blautia luti | 97.55 | 205 |
| FBI00206 | Bacteroides xylanisolvens | bacteria | bacteroidetes | 371601 | Bacteroides xylanisolvens | 99.56 | 206 |
| FBI00208 | Anaerotruncus massiliensis | bacteria | firmicutes | 1673720 | Anaerotruncus colihominis | 96.52 | 208 |
| FBI00211 | Bacteroides vulgatus | bacteria | bacteroidetes | 821 | Bacteroides vulgatus | 99.78 | 211 |
| FBI00212 | Clostridium aldenense | bacteria | firmicutes | 358742 | Clostridium aldenense | 99.1 | 212 |
| FBI00217 | Alistipes shahii | bacteria | bacteroidetes | 328814 | Alistipes shahii | 98.77 | 217 |
| FBI00218 | Bacteroides uniformis | bacteria | bacteroidetes | 820 | Bacteroides uniformis | 99.42 | 218 |
| FBI00219 | Roseburia hominis | bacteria | firmicutes | 301301 | Roseburia hominis | 99.78 | 219 |
| FBI00229 | Alistipes senegalensis | bacteria | bacteroidetes | 1288121 | Alistipes senegalensis | 99.19 | 229 |
| FBI00232 | Bacteroides stercoris | bacteria | bacteroidetes | 46506 | Bacteroides stercoris | 98.84 | 232 |
| FBI00235 | Alistipes shahii | bacteria | bacteroidetes | 328814 | Alistipes shahii | 99.86 | 235 |
| FBI00238 | Alistipes sp. FBI00238 | bacteria | bacteroidetes | 239759 | Alistipes finegoldii | 95.84 | 238 |
| FBI00241 | Collinsella aerofaciens | bacteria | actinobacteria | 74426 | Collinsella aerofaciens | 99.34 | 241 |
| FBI00243 | Eubacterium siraeum | bacteria | firmicutes | 39492 | Eubacterium siraeum | 98.53 | 243 |
| FBI00244 | Faecalibacterium prausnitzii | bacteria | firmicutes | 853 | Faecalibacterium prausnitzii | 98.69 | 244 |
| FBI00245 | Acidaminococcus intestini | bacteria | firmicutes | 187327 | Acidaminococcus intestini | 99.72 | 245 |
| FBI00246 | Bifidobacterium longum | bacteria | actinobacteria | 216816 | Bifidobacterium longum | 99.7 | 246 |
| FBI00251 | Bifidobacterium | bacteria | actinobacteria | 28026 | Bifidobacterium | 99.85 | 251 |
| | pseudocatenulatum | | | | pseudocatenulatum | | |
| FBI00253 | Roseburia hominis | bacteria | firmicutes | 301301 | Roseburia hominis | 99.71 | 253 |
| FBI00254 | Eubacterium hallii | bacteria | firmicutes | 39488 | Eubacterium hallii | 99.08 | 254 |
| FBI00255 | Hungatella effluvii | bacteria | firmicutes | 1096246 | Hungatella hathewayi | 98.56 | 255 |
| FBI00257 | Eubacterium eligens | bacteria | firmicutes | 39485 | Eubacterium eligens | 99.28 | 257 |
| FBI00258 | Turicibacter sanguinis | bacteria | firmicutes | 154288 | Turicibacter sanguinis | 99.93 | 258 |
| FBI00259 | Dorea longicatena | bacteria | firmicutes | 88431 | Dorea longicatena | 99.7 | 259 |
| FBI00260 | Eubacterium rectale | bacteria | firmicutes | 39491 | Eubacterium rectale | 99.64 | 260 |
| FBI00262 | Bacteroides massiliensis | bacteria | bacteroidetes | 204516 | Bacteroides massiliensis | 99.71 | 262 |
| FBI00263 | Bacteroides caccae | bacteria | bacteroidetes | 47678 | Bacteroides caccae | 99.56 | 263 |
| FBI00265 | Bacteroides cellulosilyticus | bacteria | bacteroidetes | 246787 | Bacteroides cellulosilyticus | 99.21 | 265 |
| FBI00266 | Coprococcus eutactus | bacteria | firmicutes | 33043 | Coprococcus eutactus | 99.2 | 266 |
| FBI00267 | Anaerofustis stercorihominis | bacteria | firmicutes | 214853 | Anaerofustis stercorihominis | 97.29 | 267 |
| FBI00269 | Alistipes putredinis | bacteria | bacteroidetes | 28117 | Alistipes putredinis | 100 | 269 |
| FBI00271 | Bacteroides xylanisolvens | bacteria | bacteroidetes | 371601 | Bacteroides xylanisolvens | 98.42 | 271 |
| FBI00274 | Eubacterium xylanophilum | bacteria | firmicutes | 39497 | Eubacterium xylanophilum | 93.5 | 274 |
| FBI00275 | Holdemanella biformis | bacteria | firmicutes | 1735 | Holdemanella biformis | 98.99 | 275 |
| FBI00276 | Dorea formicigenerans | bacteria | firmicutes | 39486 | Dorea formicigenerans | 98.19 | 276 |
| FBI00277 | Alistipes onderdonkii | bacteria | bacteroidetes | 328813 | Alistipes onderdonkii | 99.63 | 277 |
| FBI00278 | Eubacterium ventriosum | bacteria | firmicutes | 39496 | Eubacterium ventriosum | 94.14 | 278 |
| FBI00283 | Ruminococcus bromii | bacteria | firmicutes | 40518 | Ruminococcus bromii | 96.02 | 283 |
| FBI00288 | Blautia hydrogenotrophica | bacteria | firmicutes | 53443 | Blautia hydrogenotrophica | 99.57 | 288 |
| FBI00289 | Oxalobacter formigenes | bacteria | proteobacteria | 847 | Oxalobacter formigenes | 99.21 | 289 |

TABLE 17

| | | | | | | % | |
|---|---|---|---|---|---|---|---|
| | | | | NCBI | | Match | SEQ ID |
| Strain # | Species ID | Kingdom | Phylum | Taxonomy ID | Closest 16S Species | (16S) | NO: X |
| FBI00001 | Clostridium citroniae | bacteria | firmicutes | 358743 | Clostridium citroniae | 99.64 | 1 |
| FBI00002 | Bacteroides salyersiae | bacteria | bacteroidetes | 291644 | Bacteroides salyersiae | 99.5 | 2 |
| FBI00004 | Neglecta timonensis | bacteria | firmicutes | 1776382 | Neglecta timonensis | 99.14 | 4 |
| FBI00011 | Bifidobacterium longum | bacteria | actinobacteria | 216816 | Bifidobacterium longum | 99.28 | 11 |
| FBI00013 | Parabacteroides merdae | bacteria | bacteroidetes | 46503 | Parabacteroides merdae | 99.5 | 13 |
| FBI00015 | Bacteroides uniformis | bacteria | bacteroidetes | 820 | Bacteroides uniformis | 99.78 | 15 |
| FBI00016 | Bifidobacterium | bacteria | actinobacteria | 28026 | Bifidobacterium | 99.64 | 16 |
| | pseudocatenulatum | | | | pseudocatenulatum | | |
| FBI00018 | Eubacterium rectale | bacteria | firmicutes | 39491 | Eubacterium rectale | 99.71 | 18 |
| FBI00020 | Bacteroides | bacteria | bacteroidetes | 818 | Bacteroides | 99.57 | 20 |
| | thetaiotaomicron | | | | thetaiotaomicron | | |
| FBI00029 | Parabacteroides | bacteria | bacteroidetes | 823 | Parabacteroides distasonis | 99.26 | 29 |
| | distasonis | | | | | | |
| FBI00030 | Eggerthella lenta | bacteria | firmicutes | 84112 | Eggerthella lenta | 98.47 | 30 |
| FBI00033 | Lachnospiraceae sp. | bacteria | firmicutes | 186803 | Clostridium amygdalinum | 93.56 | 33 |
| | FBI00033 | | | | | | |
| FBI00039 | Bacteroides vulgatus | bacteria | bacteroidetes | 821 | Bacteroides vulgatus | 99.71 | 39 |
| FBI00040 | Bilophila wadsworthia | bacteria | proteobacteria | 35833 | Desulfovibrio | 91.38 | 40 |
| | | | | | desulfuricans | | |
| FBI00043 | Bifidobacterium dentium | bacteria | actinobacteria | 1689 | Bifidobacterium dentium | 99.35 | 43 |
| FBI00046 | Bacteroides caccae | bacteria | bacteroidetes | 47678 | Bacteroides caccae | 99.71 | 46 |
| FBI00056 | Clostridium citroniae | bacteria | firmicutes | 358743 | Clostridium citroniae | 99.2 | 56 |
| FBI00057 | Dorea longicatena | bacteria | firmicutes | 88431 | Dorea longicatena | 99.7 | 57 |
| FBI00058 | Eubacterium rectale | bacteria | firmicutes | 39491 | Eubacterium rectale | 99.71 | 58 |
| FBI00060 | Bifidobacterium longum | bacteria | actinobacteria | 216816 | Bifidobacterium longum | 99.49 | 60 |
| FBI00062 | Collinsella aerofaciens | bacteria | actinobacteria | 74426 | Collinsella aerofaciens | 99.48 | 62 |
| FBI00064 | Dorea sp. FBI00064 | bacteria | firmicutes | 189330 | Ruminococcus gnavus | 95.58 | 64 |
| FBI00067 | Oxalobacter formigenes | bacteria | proteobacteria | 847 | Oxalobacter formigenes | 98.84 | 67 |
| FBI00068 | Akkermansia muciniphila | bacteria | verrucomicrobia | 239935 | Akkermansia muciniphila | 99.42 | 68 |
| FBI00069 | Ruminococcus bromii | bacteria | firmicutes | 40518 | Ruminococcus bromii | 98.84 | 69 |
| FBI00073 | Parabacteroides | bacteria | bacteroidetes | 823 | Parabacteroides distasonis | 98.99 | 73 |
| | distasonis | | | | | | |
| FBI00074 | Clostridium fessum | bacteria | firmicutes | 2126740 | Clostridium symbiosum | 94.03 | 74 |
| FBI00076 | Bacteroides | bacteria | bacteroidetes | 818 | Bacteroides | 99.78 | 76 |
| | thetaiotaomicron | | | | thetaiotaomicron | | |
| FBI00077 | Sutterella wadsworthensis | bacteria | proteobacteria | 40545 | Sutterella wadsworthensis | 99.86 | 77 |
| FBI00085 | Ruminococcus bromii | bacteria | firmicutes | 40518 | Ruminococcus bromii | 98.62 | 85 |
| FBI00086 | Ruminococcus bromii | bacteria | firmicutes | 40518 | Ruminococcus bromii | 98.77 | 86 |
| FBI00087 | Clostridium scindens | bacteria | firmicutes | 29347 | Clostridium scindens | 98.28 | 87 |
| FBI00091 | Eubacterium rectale | bacteria | firmicutes | 39491 | Eubacterium rectale | 99.86 | 91 |
| FBI00096 | Eggerthella lenta | bacteria | actinobacteria | 84112 | Eggerthella lenta | 98.76 | 96 |
| FBI00099 | Gordonibacter pamelaeae | bacteria | actinobacteria | 471189 | Gordonibacter pamelaeae | 99.56 | 99 |
| FBI00101 | Faecalibacterium | bacteria | firmicutes | 853 | Faecalibacterium | 97.97 | 101 |
| | prausnitzii | | | | prausnitzii | | |
| FBI00102 | Clostridium fessum | bacteria | firmicutes | 2126740 | Clostridium symbiosum | 94.31 | 102 |
| FBI00109 | Coprococcus comes | bacteria | firmicutes | 410072 | Coprococcus comes | 98.39 | 109 |
| FBI00111 | Bacteroides vulgatus | bacteria | bacteroidetes | 821 | Bacteroides vulgatus | 99.43 | 111 |
| FBI00112 | Bacteroides uniformis | bacteria | bacteroidetes | 820 | Bacteroides uniformis | 99.78 | 112 |
| FBI00113 | Parabacteroides merdae | bacteria | bacteroidetes | 46503 | Parabacteroides merdae | 99.79 | 113 |
| FBI00122 | Bacteroides uniformis | bacteria | bacteroidetes | 820 | Bacteroides uniformis | 99.57 | 122 |
| FBI00125 | Bacteroides stercoris | bacteria | bacteroidetes | 46506 | Bacteroides stercoris | 99.64 | 125 |
| FBI00126 | Bifidobacterium | bacteria | actinobacteria | 1680 | Bifidobacterium | 98.98 | 126 |
| | adolescentis | | | | adolescentis | | |
| FBI00127 | Collinsella aerofaciens | bacteria | actinobacteria | 74426 | Collinsella aerofaciens | 98.81 | 127 |
| FBI00130 | Coprococcus comes | bacteria | firmicutes | 410072 | Coprococcus comes | 99.35 | 130 |
| FBI00132 | Gordonibacter pamelaeae | bacteria | actinobacteria | 471189 | Gordonibacter pamelaeae | 99.48 | 132 |
| FBI00133 | Oxalobacter formigenes | bacteria | proteobacteria | 847 | Oxalobacter formigenes | 99.21 | 133 |
| FBI00135 | Bifidobacterium | bacteria | actinobacteria | 28026 | Bifidobacterium | 99.57 | 135 |
| | pseudocatenulatum | | | | pseudocatenulatum | | |
| FBI00137 | Bacteroides fragilis | bacteria | bacteroidetes | 817 | Bacteroides fragilis | 99.71 | 137 |
| FBI00139 | Bacteroides | bacteria | bacteroidetes | 818 | Bacteroides | 99.5 | 139 |
| | thetaiotaomicron | | | | thetaiotaomicron | | |
| FBI00142 | Clostridium fessum | bacteria | firmicutes | 2126740 | Clostridium symbiosum | 94.07 | 142 |
| FBI00143 | Parabacteroides merdae | bacteria | bacteroidetes | 46503 | Parabacteroides merdae | 99.07 | 143 |
| FBI00145 | Bifidobacterium | bacteria | actinobacteria | 1680 | Bifidobacterium | 99.14 | 145 |
| | adolescentis | | | | adolescentis | | |
| FBI00162 | Bifidobacterium | bacteria | actinobacteria | 1686 | Bifidobacterium | 99.14 | 162 |
| | catenulatum | | | | catenulatum | | |
| FBI00164 | Bacteroides stercoris | bacteria | bacteroidetes | 46506 | Bacteroides stercoris | 98.56 | 164 |
| FBI00165 | Bacteroides massiliensis | bacteria | bacteroidetes | 204516 | Bacteroides massiliensis | 99.71 | 165 |
| FBI00167 | Dorea longicatena | bacteria | firmicutes | 88431 | Dorea longicatena | 99.39 | 167 |
| FBI00168 | Collinsella aerofaciens | bacteria | actinobacteria | 74426 | Collinsella aerofaciens | 99.26 | 168 |
| FBI00169 | Parabacteroides | bacteria | bacteroidetes | 823 | Parabacteroides distasonis | 98.7 | 169 |
| | distasonis | | | | | | |
| FBI00170 | Eggerthella lenta | bacteria | actinobacteria | 84112 | Eggerthella lenta | 98.61 | 170 |
| FBI00171 | Bilophila wadsworthia | bacteria | proteobacteria | 35833 | Desulfovibrio | 91.45 | 171 |
| | | | | | desulfuricans | | |
| FBI00172 | Bifidobacterium longum | bacteria | actinobacteria | 216816 | Bifidobacterium longum | 99.05 | 172 |
| FBI00173 | Bacteroides vulgatus | bacteria | bacteroidetes | 821 | Bacteroides vulgatus | 100 | 173 |
| FBI00186 | Coprococcus comes | bacteria | firmicutes | 410072 | Coprococcus comes | 99.06 | 186 |
| FBI00192 | Sutterella wadsworthensis | bacteria | proteobacteria | 40545 | Sutterella wadsworthensis | 99.71 | 192 |
| FBI00197 | Bifidobacterium bifidum | bacteria | actinobacteria | 1681 | Bifidobacterium bifidum | 99.85 | 197 |
| FBI00201 | Eggerthella lenta | bacteria | actinobacteria | 84112 | Eggerthella lenta | 98.83 | 201 |
| FBI00208 | Anaerotruncus | bacteria | firmicutes | 1673720 | Anaerotruncus colihominis | 96.52 | 208 |
| | massiliensis | | | | | | |
| FBI00210 | Bifidobacterium bifidum | bacteria | actinobacteria | 1681 | Bifidobacterium bifidum | 99.93 | 210 |
| FBI00211 | Bacteroides vulgatus | bacteria | bacteroidetes | 821 | Bacteroides vulgatus | 99.78 | 211 |
| FBI00218 | Bacteroides uniforms | bacteria | bacteroidetes | 820 | Bacteroides uniformis | 99.42 | 218 |
| FBI00224 | Sutterella wadsworthensis | bacteria | proteobacteria | 40545 | Sutterella wadsworthensis | 99.71 | 224 |
| FBI00232 | Bacteroides stercoris | bacteria | bacteroidetes | 46506 | Bacteroides stercoris | 98.84 | 232 |
| FBI00233 | Ruminococcaceae sp. | bacteria | firmicutes | 474960 | Anaerotruncus colihominis | 91.63 | 233 |
| | FBI00233 | | | | | | |
| FBI00239 | Lactonifactor | bacteria | firmicutes | 341220 | Lactonifactor | 98.99 | 239 |
| | longoviformis | | | | longoviformis | | |
| FBI00241 | Collinsella aerofaciens | bacteria | actinobacteria | 74426 | Collinsella aerofaciens | 99.34 | 241 |
| FBI00243 | Eubacterium siraeum | bacteria | firmicutes | 39492 | Eubacterium siraeum | 98.53 | 243 |
| FBI00246 | Bifidobacterium longum | bacteria | actinobacteria | 216816 | Bifidobacterium longum | 99.7 | 246 |
| FBI00249 | Citrobacter portucalensis | bacteria | proteobacteria | 1639133 | Citrobacter freundii | 99.79 | 249 |
| FBI00251 | Bifidobacterium | bacteria | actinobacteria | 28026 | Bifidobacterium | 99.85 | 251 |
| | pseudocatenulatum | | | | pseudocatenulatum | | |
| FBI00259 | Dorea longicatena | bacteria | firmicutes | 88431 | Dorea longicatena | 99.7 | 259 |
| FBI00260 | Eubacterium rectale | bacteria | firmicutes | 39491 | Eubacterium rectale | 99.64 | 260 |
| FBI00262 | Bacteroides massiliensis | bacteria | bacteroidetes | 204516 | Bacteroides massiliensis | 99.71 | 262 |
| FBI00263 | Bacteroides caccae | bacteria | bacteroidetes | 47678 | Bacteroides caccae | 99.56 | 263 |
| FBI00265 | Bacteroides | bacteria | bacteroidetes | 246787 | Bacteroides | 99.21 | 265 |
| | cellulosilyticus | | | | cellulosilyticus | | |
| FBI00283 | Ruminococcus bromii | bacteria | firmicutes | 40518 | Ruminococcus bromii | 96.02 | 283 |
| FBI00289 | Oxalobacter formigenes | bacteria | proteobacteria | 847 | Oxalobacter formigenes | 99.21 | 289 |

TABLE 18

| | | | | | | % | |
|---|---|---|---|---|---|---|---|
| | | | | NCBI | | Match | SEQ ID |
| Strain # | Species ID | Kingdom | Phylum | Taxonomy ID | Closest 16S Species | (16S) | NO: X |
| FBI00001 | Clostridium citroniae | bacteria | firmicutes | 358743 | Clostridium citroniae | 99.64 | 1 |
| FBI00002 | Bacteroides salyersiae | bacteria | bacteroidetes | 291644 | Bacteroides salyersiae | 99.5 | 2 |
| FBI00004 | Neglecta timonensis | bacteria | firmicutes | 1776382 | Neglecta timonensis | 99.14 | 4 |
| FBI00010 | Blautia obeum | bacteria | firmicutes | 40520 | Blautia obeum | 98.12 | 10 |
| FBI00011 | Bifidobacterium longum | bacteria | actinobacteria | 216816 | Bifidobacterium longum | 99.28 | 11 |
| FBI00013 | Parabacteroides merdae | bacteria | bacteroidetes | 46503 | Parabacteroides merdae | 99.5 | 13 |
| FBI00016 | Bifidobacterium | bacteria | actinobacteria | 28026 | Bifidobacterium | 99.64 | 16 |
| | pseudocatenulatum | | | | pseudocatenulatum | | |
| FBI00017 | Blautia obeum | bacteria | firmicutes | 40520 | Blautia obeum | 98.34 | 17 |
| FBI00020 | Bacteroides thetaiotaomicron | bacteria | bacteroidetes | 818 | Bacteroides thetaiotaomicron | 99.57 | 20 |
| FBI00022 | Alistipes putredinis | bacteria | bacteroidetes | 28117 | Alistipes putredinis | 99.93 | 22 |
| FBI00030 | Eggerthella lenta | bacteria | firmicutes | 84112 | Eggerthella lenta | 98.47 | 30 |
| FBI00033 | Lachnospiraceae sp. | bacteria | firmicutes | 186803 | Clostridium amygdalinum | 93.56 | 33 |
| | FBI00033 | | | | | | |
| FBI00034 | Eubacterium eligens | bacteria | firmicutes | 39485 | Eubacterium eligens | 98.78 | 34 |
| FBI00038 | Coprococcus eutactus | bacteria | firmicutes | 33043 | Coprococcus eutactus | 95.96 | 38 |
| FBI00039 | Bacteroides vulgatus | bacteria | bacteroidetes | 821 | Bacteroides vulgatus | 99.71 | 39 |
| FBI00040 | Bilophila wadsworthia | bacteria | proteobacteria | 35833 | Desulfovibrio desulfuricans | 91.38 | 40 |
| FBI00042 | Bacteroides xylanisolvens | bacteria | bacteroidetes | 371601 | Bacteroides xylanisolvens | 99.71 | 42 |
| FBI00043 | Bifidobacterium dentium | bacteria | actinobacteria | 1689 | Bifidobacterium dentium | 99.35 | 43 |
| FBI00046 | Bacteroides caccae | bacteria | bacteroidetes | 47678 | Bacteroides caccae | 99.71 | 46 |
| FBI00047 | Eubacterium eligens | bacteria | firmicutes | 39485 | Eubacterium eligens | 98.79 | 47 |
| FBI00052 | Bacteroides xylanisolvens | bacteria | bacteroidetes | 371601 | Bacteroides xylanisolvens | 99.14 | 52 |
| FBI00056 | Clostridium citroniae | bacteria | firmicutes | 358743 | Clostridium citroniae | 99.2 | 56 |
| FBI00057 | Dorea longicatena | bacteria | firmicutes | 88431 | Dorea longicatena | 99.7 | 57 |
| FBI00060 | Bifidobacterium longum | bacteria | actinobacteria | 216816 | Bifidobacterium longum | 99.49 | 60 |
| FBI00062 | Collinsella aerofaciens | bacteria | actinobacteria | 74426 | Collinsella aerofaciens | 99.48 | 62 |
| FBI00067 | Oxalobacter formigenes | bacteria | proteobacteria | 847 | Oxalobacter formigenes | 98.84 | 67 |
| FBI00069 | Ruminococcus bromii | bacteria | firmicutes | 40518 | Ruminococcus bromii | 98.84 | 69 |
| FBI00072 | Coprococcus eutactus | bacteria | firmicutes | 33043 | Coprococcus eutactus | 96.17 | 72 |
| FBI00074 | Clostridium fessum | bacteria | firmicutes | 2126740 | Clostridium symbiosum | 94.03 | 74 |
| FBI00076 | Bacteroides thetaiotaomicron | bacteria | bacteroidetes | 818 | Bacteroides thetaiotaomicron | 99.78 | 76 |
| FBI00077 | Sutterella wadsworthensis | bacteria | proteobacteria | 40545 | Sutterella wadsworthensis | 99.86 | 77 |
| FBI00085 | Ruminococcus bromii | bacteria | firmicutes | 40518 | Ruminococcus bromii | 98.62 | 85 |
| FBI00086 | Ruminococcus bromii | bacteria | firmicutes | 40518 | Ruminococcus bromii | 98.77 | 86 |
| FBI00087 | Clostridium scindens | bacteria | firmicutes | 29347 | Clostridium scindens | 98.28 | 87 |
| FBI00090 | Eubacterium eligens | bacteria | firmicutes | 39485 | Eubacterium eligens | 98.71 | 90 |
| FBI00096 | Eggerthella lenta | bacteria | actinobacteria | 84112 | Eggerthella lenta | 98.76 | 96 |
| FBI00101 | Faecalibacterium prausnitzii | bacteria | firmicutes | 853 | Faecalibacterium prausnitzii | 97.97 | 101 |
| FBI00102 | Clostridium fessum | bacteria | firmicutes | 2126740 | Clostridium symbiosum | 94.31 | 102 |
| FBI00109 | Coprococcus comes | bacteria | firmicutes | 410072 | Coprococcus comes | 98.39 | 109 |
| FBI00111 | Bacteroides vulgatus | bacteria | bacteroidetes | 821 | Bacteroides vulgatus | 99.43 | 111 |
| FBI00112 | Bacteroides uniformis | bacteria | bacteroidetes | 820 | Bacteroides uniformis | 99.78 | 112 |
| FBI00113 | Parabacteroides merdae | bacteria | bacteroidetes | 46503 | Parabacteroides merdae | 99.79 | 113 |
| FBI00115 | Dorea formicigenerans | bacteria | firmicutes | 39486 | Dorea formicigenerans | 97.98 | 115 |
| FBI00120 | Hungatella effluvii | bacteria | firmicutes | 154046 | Hungatella hathewayi | 98.78 | 120 |
| FBI00122 | Bacteroides uniformis | bacteria | bacteroidetes | 820 | Bacteroides uniformis | 99.57 | 122 |
| FBI00125 | Bacteroides stercoris | bacteria | bacteroidetes | 46506 | Bacteroides stercoris | 99.64 | 125 |
| FBI00127 | Collinsella aerofaciens | bacteria | actinobacteria | 74426 | Collinsella aerofaciens | 98.81 | 127 |
| FBI00133 | Oxalobacter formigenes | bacteria | proteobacteria | 847 | Oxalobacter formigenes | 99.21 | 133 |
| FBI00135 | Bifidobacterium | bacteria | actinobacteria | 28026 | Bifidobacterium | 99.57 | 135 |
| | pseudocatenulatum | | | | pseudocatenulatum | | |
| FBI00137 | Bacteroides fragilis | bacteria | bacteroidetes | 817 | Bacteroides fragilis | 99.71 | 137 |
| FBI00139 | Bacteroides thetaiotaomicron | bacteria | bacteroidetes | 818 | Bacteroides thetaiotaomicron | 99.5 | 139 |
| FBI00142 | Clostridium fessum | bacteria | firmicutes | 2126740 | Clostridium symbiosum | 94.07 | 142 |
| FBI00143 | Parabacteroides merdae | bacteria | bacteroidetes | 46503 | Parabacteroides merdae | 99.07 | 143 |
| FBI00147 | Clostridium bolteae | bacteria | firmicutes | 208479 | Clostridium bolteae | 99.28 | 147 |
| FBI00162 | Bifidobacterium catenulatum | bacteria | actinobacteria | 1686 | Bifidobacterium catenulatum | 99.14 | 162 |
| FBI00164 | Bacteroides stercoris | bacteria | bacteroidetes | 46506 | Bacteroides stercoris | 98.56 | 164 |
| FBI00167 | Dorea longicatena | bacteria | firmicutes | 88431 | Dorea longicatena | 99.39 | 167 |
| FBI00168 | Collinsella aerofaciens | bacteria | actinobacteria | 74426 | Collinsella aerofaciens | 99.26 | 168 |
| FBI00170 | Eggerthella lenta | bacteria | actinobacteria | 84112 | Eggerthella lenta | 98.61 | 170 |
| FBI00171 | Bilophila wadsworthia | bacteria | proteobacteria | 35833 | Desulfovibrio desulfuricans | 91.45 | 171 |
| FBI00172 | Bifidobacterium longum | bacteria | actinobacteria | 216816 | Bifidobacterium longum | 99.05 | 172 |
| FBI00173 | Bacteroides vulgatus | bacteria | bacteroidetes | 821 | Bacteroides vulgatus | 100 | 173 |
| FBI00182 | Bacteroides coprocola | bacteria | bacteroidetes | 310298 | Bacteroides coprocola | 99.64 | 182 |
| FBI00197 | Bifidobacterium bifidum | bacteria | actinobacteria | 1681 | Bifidobacterium bifidum | 99.85 | 197 |
| FBI00199 | Clostridium bolteae | bacteria | firmicutes | 208479 | Clostridium bolteae | 99.28 | 199 |
| FBI00201 | Eggerthella lenta | bacteria | actinobacteria | 84112 | Eggerthella lenta | 98.83 | 201 |
| FBI00206 | Bacteroides xylanisolvens | bacteria | bacteroidetes | 371601 | Bacteroides xylanisolvens | 99.56 | 206 |
| FBI00208 | Anaerotruncus massiliensis | bacteria | firmicutes | 1673720 | Anaerotruncus colihominis | 96.52 | 208 |
| FBI00210 | Bifidobacterium bifidum | bacteria | actinobacteria | 1681 | Bifidobacterium bifidum | 99.93 | 210 |
| FBI00211 | Bacteroides vulgatus | bacteria | bacteroidetes | 821 | Bacteroides vulgatus | 99.78 | 211 |
| FBI00232 | Bacteroides stercoris | bacteria | bacteroidetes | 46506 | Bacteroides stercoris | 98.84 | 232 |
| FBI00238 | Alistipes sp. FBI00238 | bacteria | bacteroidetes | 239759 | Alistipes finegoldii | 95.84 | 238 |
| FBI00241 | Collinsella aerofaciens | bacteria | actinobacteria | 74426 | Collinsella aerofaciens | 99.34 | 241 |
| FBI00243 | Eubacterium siraeum | bacteria | firmicutes | 39492 | Eubacterium siraeum | 98.53 | 243 |
| FBI00244 | Faecalibacterium prausnitzii | bacteria | firmicutes | 853 | Faecalibacterium prausnitzii | 98.69 | 244 |
| FBI00246 | Bifidobacterium longum | bacteria | actinobacteria | 216816 | Bifidobacterium longum | 99.7 | 246 |
| FBI00251 | Bifidobacterium | bacteria | actinobacteria | 28026 | Bifidobacterium | 99.85 | 251 |
| | pseudocatenulatum | | | | pseudocatenulatum | | |
| FBI00254 | Eubacterium hallii | bacteria | firmicutes | 39488 | Eubacterium hallii | 99.08 | 254 |
| FBI00255 | Hungatella effluvii | bacteria | firmicutes | 1096246 | Hungatella hathewayi | 98.56 | 255 |
| FBI00257 | Eubacterium eligens | bacteria | firmicutes | 39485 | Eubacterium eligens | 99.28 | 257 |
| FBI00260 | Eubacterium rectale | bacteria | firmicutes | 39491 | Eubacterium rectale | 99.64 | 260 |
| FBI00265 | Bacteroides cellulosilyticus | bacteria | bacteroidetes | 246787 | Bacteroides cellulosilyticus | 99.21 | 265 |
| FBI00266 | Coprococcus eutactus | bacteria | firmicutes | 33043 | Coprococcus eutactus | 99.2 | 266 |
| FBI00269 | Alistipes putredinis | bacteria | bacteroidetes | 28117 | Alistipes putredinis | 100 | 269 |
| FBI00271 | Bacteroides xylanisolvens | bacteria | bacteroidetes | 371601 | Bacteroides xylanisolvens | 98.42 | 271 |
| FBI00278 | Eubacterium ventriosum | bacteria | firmicutes | 39496 | Eubacterium ventriosum | 94.14 | 278 |
| FBI00283 | Ruminococcus bromii | bacteria | firmicutes | 40518 | Ruminococcus bromii | 96.02 | 283 |
| FBI00288 | Blautia hydrogenotrophica | bacteria | firmicutes | 53443 | Blautia hydrogenotrophica | 99.57 | 288 |
| FBI00289 | Oxalobacter formigenes | bacteria | proteobacteria | 847 | Oxalobacter formigenes | 99.21 | 289 |

TABLE 19

| | | | | | | % | |
|---|---|---|---|---|---|---|---|
| | | | | NCBI | | Match | SEQ ID |
| Strain # | Species ID | Kingdom | Phylum | Taxonomy ID | Closest 16S Species | (16S) | NO: X |
| FBI00001 | Clostridium citroniae | bacteria | firmicutes | 358743 | Clostridium citroniae | 99.64 | 1 |
| FBI00002 | Bacteroides salyersiae | bacteria | bacteroidetes | 291644 | Bacteroides salyersiae | 99.5 | 2 |
| FBI00003 | Enterococcus faecalis | bacteria | firmicutes | 1351 | Enterococcus faecalis | 99.93 | 3 |
| FBI00004 | Neglecta timonensis | bacteria | firmicutes | 1776382 | Neglecta timonensis | 99.14 | 4 |
| FBI00005 | Enterococcus casseliflavus | bacteria | firmicutes | 37734 | Enterococcus gallinarum | 99.65 | 5 |
| FBI00006 | Enterobacter himalayensis | bacteria | proteobacteria | 547 | Enterobacter | 99 | 6 |
| | | | | | hormaechei | | |
| FBI00009 | Bifidobacterium adolescentis | bacteria | actinobacteria | 1680 | Bifidobacterium faecale | 98.6 | 9 |
| FBI00010 | Blautia obeum | bacteria | firmicutes | 40520 | Blautia obeum | 98.12 | 10 |
| FBI00011 | Bifidobacterium longum | bacteria | actinobacteria | 216816 | Bifidobacterium longum | 99.28 | 11 |
| FBI00012 | Alistipes onderdonkii | bacteria | bacteroidetes | 328813 | Alistipes onderdonkii | 99.71 | 12 |
| FBI00013 | Parabacteroides merdae | bacteria | bacteroidetes | 46503 | Parabacteroides merdae | 99.5 | 13 |
| FBI00015 | Bacteroides uniformis | bacteria | bacteroidetes | 820 | Bacteroides uniformis | 99.78 | 15 |
| FBI00016 | Bifidobacterium | bacteria | actinobacteria | 28026 | Bifidobacterium | 99.64 | 16 |
| | pseudocatenulatum | | | | pseudocatenulatum | | |
| FBI00017 | Blautia obeum | bacteria | firmicutes | 40520 | Blautia obeum | 98.34 | 17 |
| FBI00018 | Eubacterium rectale | bacteria | firmicutes | 39491 | Eubacterium rectale | 99.71 | 18 |
| FBI00019 | Alistipes timonensis | bacteria | bacteroidetes | 1465754 | Alistipes timonensis | 99.78 | 19 |
| FBI00020 | Bacteroides thetaiotaomicron | bacteria | bacteroidetes | 818 | Bacteroides | 99.57 | 20 |
| | | | | | thetaiotaomicron | | |
| FBI00021 | Bacteroides kribbi/ | bacteria | bacteroidetes | 816 | Bacteroides kribbi | 99.07 | 21 |
| | Bacteroides koreensis | | | | | | |
| | species cluster | | | | | | |
| FBI00022 | Alistipes putredinis | bacteria | bacteroidetes | 28117 | Alistipes putredinis | 99.93 | 22 |
| FBI00025 | Coprococcus comes | bacteria | firmicutes | 410072 | Coprococcus comes | 99.21 | 25 |
| FBI00027 | Fusicatenibacter saccharivorans | bacteria | firmicutes | 1150298 | Fusicatenibacter | 97.6 | 27 |
| | | | | | saccharivorans | | |
| FBI00029 | Parabacteroides distasonis | bacteria | bacteroidetes | 823 | Parabacteroides | 99.26 | 29 |
| | | | | | distasonis | | |
| FBI00030 | Eggerthella lenta | bacteria | firmicutes | 84112 | Eggerthella lenta | 98.47 | 30 |
| FBI00031 | Enterobacter hormaechei | bacteria | proteobacteria | 158836 | Enterobacter | 99.43 | 31 |
| | | | | | hormaechei | | |
| FBI00033 | Lachnospiraceae sp. FBI00033 | bacteria | firmicutes | 186803 | Clostridium | 93.56 | 33 |
| | | | | | amygdalinum | | |
| FBI00034 | Eubacterium eligens | bacteria | firmicutes | 39485 | Eubacterium eligens | 98.78 | 34 |
| FBI00036 | Blautia faecis | bacteria | firmicutes | 871665 | Blautia faecis | 99.53 | 36 |
| FBI00038 | Coprococcus eutactus | bacteria | firmicutes | 33043 | Coprococcus eutactus | 95.96 | 38 |
| FBI00040 | Bilophila wadsworthia | bacteria | proteobacteria | 35833 | Desulfovibrio | 91.38 | 40 |
| | | | | | desulfuricans | | |
| FBI00041 | Phascolarctobacterium faecium | bacteria | firmicutes | 33025 | Phascolarctobacterium | 99.23 | 41 |
| | | | | | faecium | | |
| FBI00043 | Bifidobacterium dentium | bacteria | actinobacteria | 1689 | Bifidobacterium | 99.35 | 43 |
| | | | | | dentium | | |
| FBI00044 | Blautia wexlerae | bacteria | firmicutes | 418240 | Blautia wexlerae | 98.69 | 44 |
| FBI00046 | Bacteroides caccae | bacteria | bacteroidetes | 47678 | Bacteroides caccae | 99.71 | 46 |
| FBI00047 | Eubacterium eligens | bacteria | firmicutes | 39485 | Eubacterium eligens | 98.79 | 47 |
| FBI00048 | Fusicatenibacter saccharivorans | bacteria | firmicutes | 1150298 | Fusicatenibacter | 97.95 | 48 |
| | | | | | saccharivorans | | |
| FBI00049 | Dialister succinatiphilus | bacteria | firmicutes | 487173 | Dialister succinatiphilus | 95.74 | 49 |
| FBI00050 | Bacteroides nordii | bacteria | bacteroidetes | 291645 | Bacteroides nordii | 98.63 | 50 |
| FBI00051 | Dorea formicigenerans | bacteria | firmicutes | 39486 | Dorea formicigenerans | 98.07 | 51 |
| FBI00052 | Bacteroides xylanisolvens | bacteria | bacteroidetes | 371601 | Bacteroides | 99.14 | 52 |
| | | | | | xylanisolvens | | |
| FBI00053 | Lactobacillus rogosae | bacteria | firmicutes | 706562 | Lachnospira | 97.36 | 53 |
| | | | | | pectinoschiza | | |
| FBI00055 | Bacteroides kribbi/ | bacteria | bacteroidetes | 816 | Bacteroides kribbi | 99.64 | 55 |
| | Bacteroides koreensis | | | | | | |
| | species cluster | | | | | | |
| FBI00056 | Clostridium citroniae | bacteria | firmicutes | 358743 | Clostridium citroniae | 99.2 | 56 |
| FBI00057 | Dorea longicatena | bacteria | firmicutes | 88431 | Dorea longicatena | 99.7 | 57 |
| FBI00058 | Eubacterium rectale | bacteria | firmicutes | 39491 | Eubacterium rectale | 99.71 | 58 |
| FBI00060 | Bifidobacterium longum | bacteria | actinobacteria | 216816 | Bifidobacterium longum | 99.49 | 60 |
| FBI00061 | Alistipes shahii | bacteria | bacteroidetes | 328814 | Alistipes shahii | 99.19 | 61 |
| FBI00062 | Collinsella aerofaciens | bacteria | actinobacteria | 74426 | Collinsella aerofaciens | 99.48 | 62 |
| FBI00066 | Parasutterella excrementihominis | bacteria | proteobacteria | 487175 | Parasutterella | 99.13 | 66 |
| | | | | | excrementihominis | | |
| FBI00067 | Oxalobacter formigenes | bacteria | proteobacteria | 847 | Oxalobacter formigenes | 98.84 | 67 |
| FBI00068 | Akkermansia muciniphila | bacteria | verrucomicrobia | 239935 | Akkermansia | 99.42 | 68 |
| | | | | | muciniphila | | |
| FBI00069 | Ruminococcus bromii | bacteria | firmicutes | 40518 | Ruminococcus bromii | 98.84 | 69 |
| FBI00070 | Bacteroides kribbi/ | bacteria | bacteroidetes | 816 | Bacteroides koreensis | 99.71 | 70 |
| | Bacteroides koreensis | | | | | | |
| | species cluster | | | | | | |
| FBI00073 | Parabacteroides distasonis | bacteria | bacteroidetes | 823 | Parabacteroides | 98.99 | 73 |
| | | | | | distasonis | | |
| FBI00075 | Paraprevotella clara | bacteria | bacteroidetes | 454154 | Paraprevotella clara | 98.85 | 75 |
| FBI00076 | Bacteroides thetaiotaomicron | bacteria | bacteroidetes | 818 | Bacteroides | 99.78 | 76 |
| | | | | | thetaiotaomicron | | |
| FBI00077 | Sutterella wadsworthensis | bacteria | proteobacteria | 40545 | Sutterella | 99.86 | 77 |
| | | | | | wadsworthensis | | |
| FBI00080 | Sutterella massiliensis | bacteria | proteobacteria | 1816689 | Sutterella massiliensis | 99.78 | 80 |
| FBI00085 | Ruminococcus bromii | bacteria | firmicutes | 40518 | Ruminococcus bromii | 98.62 | 85 |
| FBI00087 | Clostridium scindens | bacteria | firmicutes | 29347 | Clostridium scindens | 98.28 | 87 |
| FBI00092 | Monoglobus pectinilyticus | bacteria | firmicutes | 1981510 | Monoglobus | 99.5 | 92 |
| | | | | | pectinilyticus | | |
| FBI00093 | Roseburia hominis | bacteria | firmicutes | 301301 | Roseburia hominis | 99.71 | 93 |
| FBI00098 | Bacteroides dorei | bacteria | bacteroidetes | 357276 | Bacteroides dorei | 99.93 | 98 |
| FBI00101 | Faecalibacterium prausnitzii | bacteria | firmicutes | 853 | Faecalibacterium | 97.97 | 101 |
| | | | | | prausnitzii | | |
| FBI00102 | Clostridium fessum | bacteria | firmicutes | 2126740 | Clostridium symbiosum | 94.31 | 102 |
| FBI00104 | Blautia wexlerae | bacteria | firmicutes | 418240 | Blautia luti | 97.18 | 104 |
| FBI00110 | Lachnoclostridium pacaense | bacteria | firmicutes | 1917870 | Lachnoclostridium | 98.92 | 110 |
| | | | | | pacaense | | |
| FBI00111 | Bacteroides vulgatus | bacteria | bacteroidetes | 821 | Bacteroides vulgatus | 99.43 | 111 |
| FBI00116 | Ruminococcus faecis | bacteria | firmicutes | 592978 | Ruminococcus faecis | 99.57 | 116 |
| FBI00117 | Blautia faecis | bacteria | firmicutes | 871665 | Blautia faecis | 99.52 | 117 |
| FBI00132 | Gordonibacter pamelaeae | bacteria | actinobacteria | 471189 | Gordonibacter | 99.48 | 132 |
| | | | | | pamelaeae | | |
| FBI00133 | Oxalobacter formigenes | bacteria | proteobacteria | 847 | Oxalobacter formigenes | 99.21 | 133 |
| FBI00140 | Phascolarctobacterium faecium | bacteria | firmicutes | 33025 | Phascolarctobacterium | 99.58 | 140 |
| | | | | | faecium | | |
| FBI00141 | Phascolarctobacterium faecium | bacteria | firmicutes | 33025 | Phascolarctobacterium | 99.15 | 141 |
| | | | | | faecium | | |
| FBI00155 | Blautia obeum | bacteria | firmicutes | 40520 | Blautia obeum | 98.7 | 155 |
| FBI00289 | Oxalobacter formigenes | bacteria | proteobacteria | 847 | Oxalobacter formigenes | 99.21 | 289 |

TABLE 20

| | | | | | | % | |
|---|---|---|---|---|---|---|---|
| | | | | NCBI | | Match | SEQ ID |
| Strain # | Species ID | Kingdom | Phylum | Taxonomy ID | Closest 16S Species | (16S) | NO: X |
| FBI00001 | Clostridium citroniae | bacteria | firmicutes | 358743 | Clostridium citroniae | 99.64 | 1 |
| FBI00002 | Bacteroides salyersiae | bacteria | bacteroidetes | 291644 | Bacteroides salyersiae | 99.5 | 2 |
| FBI00004 | Neglecta timonensis | bacteria | firmicutes | 1776382 | Neglecta timonensis | 99.14 | 4 |
| FBI00009 | Bifidobacterium adolescentis | bacteria | actinobacteria | 1680 | Bifidobacterium faecale | 98.6 | 9 |
| FBI00010 | Blautia obeum | bacteria | firmicutes | 40520 | Blautia obeum | 98.12 | 10 |
| FBI00011 | Bifidobacterium longum | bacteria | actinobacteria | 216816 | Bifidobacterium longum | 99.28 | 11 |
| FBI00012 | Alistipes onderdonkii | bacteria | bacteroidetes | 328813 | Alistipes onderdonkii | 99.71 | 12 |
| FBI00013 | Parabacteroides merdae | bacteria | bacteroidetes | 46503 | Parabacteroides merdae | 99.5 | 13 |
| FBI00015 | Bacteroides uniformis | bacteria | bacteroidetes | 820 | Bacteroides uniformis | 99.78 | 15 |
| FBI00016 | Bifidobacterium | bacteria | actinobacteria | 28026 | Bifidobacterium | 99.64 | 16 |
| | pseudocatenulatum | | | | pseudocatenulatum | | |
| FBI00018 | Eubacterium rectale | bacteria | firmicutes | 39491 | Eubacterium rectale | 99.71 | 18 |
| FBI00019 | Alistipes timonensis | bacteria | bacteroidetes | 1465754 | Alistipes timonensis | 99.78 | 19 |
| FBI00020 | Bacteroides thetaiotaomicron | bacteria | bacteroidetes | 818 | Bacteroides | 99.57 | 20 |
| | | | | | thetaiotaomicron | | |
| FBI00021 | Bacteroides kribbi/ | bacteria | bacteroidetes | 816 | Bacteroides kribbi | 99.07 | 21 |
| | Bacteroides koreensis | | | | | | |
| | species cluster | | | | | | |
| FBI00022 | Alistipes putredinis | bacteria | bacteroidetes | 28117 | Alistipes putredinis | 99.93 | 22 |
| FBI00025 | Coprococcus comes | bacteria | firmicutes | 410072 | Coprococcus comes | 99.21 | 25 |
| FBI00027 | Fusicatenibacter saccharivorans | bacteria | firmicutes | 1150298 | Fusicatenibacter | 97.6 | 27 |
| | | | | | saccharivorans | | |
| FBI00029 | Parabacteroides distasonis | bacteria | bacteroidetes | 823 | Parabacteroides | 99.26 | 29 |
| | | | | | distasonis | | |
| FBI00030 | Eggerthella lenta | bacteria | firmicutes | 84112 | Eggerthella lenta | 98.47 | 30 |
| FBI00032 | Anaerostipes hadrus | bacteria | firmicutes | 649756 | Anaerostipes hadrus | 99.64 | 32 |
| FBI00033 | Lachnospiraceae sp. FBI00033 | bacteria | firmicutes | 186803 | Clostridium | 93.56 | 33 |
| | | | | | amygdalinum | | |
| FBI00034 | Eubacterium eligens | bacteria | firmicutes | 39485 | Eubacterium eligens | 98.78 | 34 |
| FBI00036 | Blautia faecis | bacteria | firmicutes | 871665 | Blautia faecis | 99.53 | 36 |
| FBI00038 | Coprococcus eutactus | bacteria | firmicutes | 33043 | Coprococcus eutactus | 95.96 | 38 |
| FBI00040 | Bilophila wadsworthia | bacteria | proteobacteria | 35833 | Desulfovibrio | 91.38 | 40 |
| | | | | | desulfuricans | | |
| FBI00043 | Bifidobacterium dentium | bacteria | actinobacteria | 1689 | Bifidobacterium dentium | 99.35 | 43 |
| FBI00044 | Blautia wexlerae | bacteria | firmicutes | 418240 | Blautia wexlerae | 98.69 | 44 |
| FBI00046 | Bacteroides caccae | bacteria | bacteroidetes | 47678 | Bacteroides caccae | 99.71 | 46 |
| FBI00047 | Eubacterium eligens | bacteria | firmicutes | 39485 | Eubacterium eligens | 98.79 | 47 |
| FBI00048 | Fusicatenibacter saccharivorans | bacteria | firmicutes | 1150298 | Fusicatenibacter | 97.95 | 48 |
| | | | | | saccharivorans | | |
| FBI00049 | Dialister succinatiphilus | bacteria | firmicutes | 487173 | Dialister succinatiphilus | 95.74 | 49 |
| FBI00050 | Bacteroides nordii | bacteria | bacteroidetes | 291645 | Bacteroides nordii | 98.63 | 50 |
| FBI00051 | Dorea formicigenerans | bacteria | firmicutes | 39486 | Dorea formicigenerans | 98.07 | 51 |
| FBI00052 | Bacteroides xylanisolvens | bacteria | bacteroidetes | 371601 | Bacteroides | 99.14 | 52 |
| | | | | | xylanisolvens | | |
| FBI00053 | Lactobacillus rogosae | bacteria | firmicutes | 706562 | Lachnospira | 97.36 | 53 |
| | | | | | pectinoschiza | | |
| FBI00055 | Bacteroides kribbi/ | bacteria | bacteroidetes | 816 | Bacteroides kribbi | 99.64 | 55 |
| | Bacteroides koreensis | | | | | | |
| | species cluster | | | | | | |
| FBI00056 | Clostridium citroniae | bacteria | firmicutes | 358743 | Clostridium citroniae | 99.2 | 56 |
| FBI00057 | Dorea longicatena | bacteria | firmicutes | 88431 | Dorea longicatena | 99.7 | 57 |
| FBI00059 | Bacteroides stercorirosoris | bacteria | bacteroidetes | 871324 | Bacteroides oleiciplenus | 98.81 | 59 |
| FBI00060 | Bifidobacterium longum | bacteria | actinobacteria | 216816 | Bifidobacterium longum | 99.49 | 60 |
| FBI00061 | Alistipes shahii | bacteria | bacteroidetes | 328814 | Alistipes shahii | 99.19 | 61 |
| FBI00062 | Collinsella aerofaciens | bacteria | actinobacteria | 74426 | Collinsella aerofaciens | 99.48 | 62 |
| FBI00066 | Parasutterella excrementihominis | bacteria | proteobacteria | 487175 | Parasutterella | 99.13 | 66 |
| | | | | | excrementihominis | | |
| FBI00067 | Oxalobacter formigenes | bacteria | proteobacteria | 847 | Oxalobacter formigenes | 98.84 | 67 |
| FBI00068 | Akkemiansia muciniphila | bacteria | verrucomicrobia | 239935 | Akkermansia | 99.42 | 68 |
| | | | | | muciniphila | | |
| FBI00069 | Ruminococcus bromii | bacteria | firmicutes | 40518 | Ruminococcus bromii | 98.84 | 69 |
| FBI00070 | Bacteroides kribbi/ | bacteria | bacteroidetes | 816 | Bacteroides koreensis | 99.71 | 70 |
| | Bacteroides koreensis | | | | | | |
| | species cluster | | | | | | |
| FBI00071 | Lachnospiraceae sp. FBI00071 | bacteria | firmicutes | 186803 | Roseburia faecis | 94.92 | 71 |
| FBI00072 | Coprococcus eutactus | bacteria | firmicutes | 33043 | Coprococcus eutactus | 96.17 | 72 |
| FBI00074 | Clostridium fessum | bacteria | firmicutes | 2126740 | Clostridium symbiosum | 94.03 | 74 |
| FBI00075 | Paraprevotella clara | bacteria | bacteroidetes | 454154 | Paraprevotella clara | 98.85 | 75 |
| FBI00076 | Bacteroides thetaiotaomicron | bacteria | bacteroidetes | 818 | Bacteroides | 99.78 | 76 |
| | | | | | thetaiotaomicron | | |
| FBI00077 | Sutterella wadsworthensis | bacteria | proteobacteria | 40545 | Sutterella | 99.86 | 77 |
| | | | | | wadsworthensis | | |
| FBI00078 | Blautia obeum | bacteria | firmicutes | 40520 | Blautia obeum | 98.34 | 78 |
| FBI00079 | Clostridium clostridioforme | bacteria | firmicutes | 1531 | Clostridium | 99.14 | 79 |
| | | | | | clostridioforme | | |
| FBI00080 | Sutterella massiliensis | bacteria | proteobacteria | 1816689 | Sutterella massiliensis | 99.78 | 80 |
| FBI00081 | Porphyromonas asaccharolytica | bacteria | bacteroidetes | 28123 | Porphyromonas | 99.35 | 81 |
| | | | | | asaccharolytica | | |
| FBI00082 | Ruminococcaceae sp. FBI00082 | bacteria | firmicutes | 541000 | Phocea massiliensis | 93.08 | 82 |
| | FBI00097 | | | | | | |
| FBI00085 | Ruminococcus bromii | bacteria | firmicutes | 40518 | Ruminococcus bromii | 98.62 | 85 |
| FBI00087 | Clostridium scindens | bacteria | firmicutes | 29347 | Clostridium scindens | 98.28 | 87 |
| FBI00092 | Monoglobus pectinilyticus | bacteria | firmicutes | 1981510 | Monoglobus | 99.5 | 92 |
| | | | | | pectinilyticus | | |
| FBI00093 | Roseburia hominis | bacteria | firmicutes | 301301 | Roseburia hominis | 99.71 | 93 |
| FBI00096 | Eggerthella lenta | bacteria | actinobacteria | 84112 | Eggerthella lenta | 98.76 | 96 |
| FBI00097 | Ruminococcaceae sp. FBI00082 | bacteria | firmicutes | 541000 | Phocea massiliensis | 93.07 | 97 |
| | FBI00097 | | | | | | |
| FBI00099 | Gordonibacter pamelaeae | bacteria | actinobacteria | 471189 | Gordonibacter | 99.56 | 99 |
| | | | | | pamelaeae | | |
| FBI00101 | Faecalibacterium prausnitzii | bacteria | firmicutes | 853 | Faecalibacterium | 97.97 | 101 |
| | | | | | prausnitzii | | |
| FBI00102 | Clostridium fessum | bacteria | firmicutes | 2126740 | Clostridium symbiosum | 94.31 | 102 |
| FBI00104 | Blautia wexlerae | bacteria | firmicutes | 418240 | Blautia luti | 97.18 | 104 |
| FBI00109 | Coprococcus comes | bacteria | firmicutes | 410072 | Coprococcus comes | 98.39 | 109 |
| FBI00110 | Lachnoclostridium pacacnse | bacteria | firmicutes | 1917870 | Lachnoclostridium | 98.92 | 110 |
| | | | | | pacaense | | |
| FBI00111 | Bacteroides vulgatus | bacteria | bacteroidetes | 821 | Bacteroides vulgatus | 99.43 | 111 |
| FBI00112 | Bacteroides uniformis | bacteria | bacteroidetes | 820 | Bacteroides uniformis | 99.78 | 112 |
| FBI00113 | Parabacteroides merdae | bacteria | bacteroidetes | 46503 | Parabacteroides merdae | 99.79 | 113 |
| FBI00115 | Dorea formicigenerans | bacteria | firmicutes | 39486 | Dorea formicigenerans | 97.98 | 115 |
| FBI00116 | Ruminococcus faecis | bacteria | firmicutes | 592978 | Ruminococcus faecis | 99.57 | 116 |
| FBI00117 | Blautia faecis | bacteria | firmicutes | 871665 | Blautia faecis | 99.52 | 117 |
| FBI00120 | Hungatella effluvii | bacteria | firmicutes | 154046 | Hungatella hathewayi | 98.78 | 120 |
| FBI00123 | Roseburia hominis | bacteria | firmicutes | 301301 | Roseburia hominis | 100 | 123 |
| FBI00124 | Anaerostipes hadrus | bacteria | firmicutes | 649756 | Anaerostipes hadrus | 99.86 | 124 |
| FBI00125 | Bacteroides stercoris | bacteria | bacteroidetes | 46506 | Bacteroides stercoris | 99.64 | 125 |
| FBI00126 | Bifidobacterium adolescentis | bacteria | actinobacteria | 1680 | Bifidobacterium | 98.98 | 126 |
| | | | | | adolescentis | | |
| FBI00127 | Collinsella aerofaciens | bacteria | actinobacteria | 74426 | Collinsella aerofaciens | 98.81 | 127 |
| FBI00128 | Hungatella effluvii | bacteria | firmicutes | 1096246 | Hungatella effluvii | 98.71 | 128 |
| FBI00132 | Gordonibacter pamelaeae | bacteria | actinobacteria | 471189 | Gordonibacter | 99.48 | 132 |
| | | | | | pamelaeae | | |
| FBI00133 | Oxalobacter formigenes | bacteria | proteobacteria | 847 | Oxalobacter formigenes | 99.21 | 133 |
| FBI00135 | Bifidobacterium | bacteria | actinobacteria | 28026 | Bifidobacterium | 99.57 | 135 |
| | pseudocatenulatum | | | | pseudocatenulatum | | |
| FBI00137 | Bacteroides fragilis | bacteria | bacteroidetes | 817 | Bacteroides fragilis | 99.71 | 137 |
| FBI00140 | Phascolarctobacterium faecium | bacteria | firmicutes | 33025 | Phascolarctobacterium | 99.58 | 140 |
| | | | | | faecium | | |
| FBI00141 | Phascolarctobacterium faecium | bacteria | firmicutes | 33025 | Phascolarctobacterium | 99.15 | 141 |
| | | | | | faecium | | |
| FBI00145 | Bifidobacterium adolescentis | bacteria | actinobacteria | 1680 | Bifidobacterium | 99.14 | 145 |
| | | | | | adolescentis | | |
| FBI00147 | Clostridium bolteae | bacteria | firmicutes | 208479 | Clostridium bolteae | 99.28 | 147 |
| FBI00149 | Monoglobus pectinilyticus | bacteria | firmicutes | 1981510 | Monoglobus | 99.5 | 149 |
| | | | | | pectinilyticus | | |
| FBI00151 | Clostridium aldenense | bacteria | firmicutes | 358742 | Clostridium aldenense | 98.55 | 151 |
| FBI00152 | Dialister invisus | bacteria | firmicutes | 218538 | Dialister invisus | 99.58 | 152 |
| FBI00159 | Eisenbergiella tayi | bacteria | firmicutes | 1432052 | Eisenbergiella tayi | 99.03 | 159 |
| FBI00162 | Bifidobacterium catenulatum | bacteria | actinobacteria | 1686 | Bifidobacterium | 99.14 | 162 |
| | | | | | catenulatum | | |
| FBI00165 | Bacteroides massiliensis | bacteria | bacteroidetes | 204516 | Bacteroides massiliensis | 99.71 | 165 |
| FBI00167 | Dorea longicatena | bacteria | firmicutes | 88431 | Dorea longicatena | 99.39 | 167 |
| FBI00170 | Eggerthella lenta | bacteria | actinobacteria | 84112 | Eggerthella lenta | 98.61 | 170 |
| FBI00171 | Bilophila wadsworthia | bacteria | proteobacteria | 35833 | Desulfovibrio | 91.45 | 171 |
| | | | | | desulfuricans | | |
| FBI00174 | Lactobacillus rogosae | bacteria | firmicutes | 706562 | Lachnospira | 97.92 | 174 |
| | | | | | pectinoschiza | | |
| FBI00175 | Holdemanella biformis | bacteria | firmicutes | 1735 | Holdemanella biformis | 98.19 | 175 |
| FBI00176 | Ruthenibacterium lactatiformans | bacteria | firmicutes | 1550024 | Ruthenibacterium | 99.71 | 176 |
| | | | | | lactatiformans | | |
| FBI00177 | Parasutterella excrementihominis | bacteria | proteobacteria | 487175 | Parasutterella | 99.71 | 177 |
| | | | | | excrementihominis | | |
| FBI00180 | Alistipes sp. FBI00180 | bacteria | bacteroidetes | 239759 | Alistipes senegalensis | 97.56 | 180 |
| FBI00182 | Bacteroides coprocola | bacteria | bacteroidetes | 310298 | Bacteroides coprocola | 99.64 | 182 |
| FBI00184 | Bacteroides faecis | bacteria | bacteroidetes | 674529 | Bacteroides faecis | 99.78 | 184 |
| FBI00189 | Bacteroides ovatus | bacteria | bacteroidetes | 28116 | Bacteroides koreensis | 99.93 | 189 |
| FBI00190 | Bacteroides finegoldii | bacteria | bacteroidetes | 338188 | Bacteroides finegoldii | 98.91 | 190 |
| FBI00191 | Clostridiaceae sp. FBI00191 | bacteria | firmicutes | 31979 | Clostridium | 96.24 | 191 |
| | | | | | swellfunianum | | |
| FBI00194 | Ruminococcus faecis | bacteria | firmicutes | 592978 | Ruminococcus faecis | 98.41 | 194 |
| FBI00197 | Bifidobacterium bifidum | bacteria | actinobacteria | 1681 | Bifidobacterium bifidum | 99.85 | 197 |
| FBI00198 | Lachnoclostridium pacaense | bacteria | firmicutes | 1917870 | Lachnoclostridium | 99.71 | 198 |
| | | | | | pacaense | | |
| FBI00199 | Clostridium bolteae | bacteria | firmicutes | 208479 | Clostridium bolteae | 99.28 | 199 |
| FBI00200 | Longicatena caecimuris | bacteria | firmicutes | 1796635 | Longicatena caecimuris | 99.71 | 200 |
| FBI00201 | Eggerthella lenta | bacteria | actinobacteria | 84112 | Eggerthella lenta | 98.83 | 201 |
| FBI00205 | Blautia massiliensis | bacteria | firmicutes | 1737424 | Blautia luti | 97.55 | 205 |
| FBI00206 | Bacteroides xylanisolvens | bacteria | bacteroidetes | 371601 | Bacteroides xylanisolvens | 99.56 | 206 |
| FBI00208 | Anaerotruncus massiliensis | bacteria | firmicutes | 1673720 | Anaerotruncus colihominis | 96.52 | 208 |
| FBI00210 | Bifidobacterium bifidum | bacteria | actinobacteria | 1681 | Bifidobacterium bifidum | 99.93 | 210 |
| FBI00211 | Bacteroides vulgatus | bacteria | bacteroidetes | 821 | Bacteroides vulgatus | 99.78 | 211 |
| FBI00212 | Clostridium aldenense | bacteria | firmicutes | 358742 | Clostridium aldenense | 99.1 | 212 |
| FBI00220 | Megasphaera massiliensis | bacteria | firmicutes | 1232428 | Megasphaera massiliensis | 98.8 | 220 |
| FBI00221 | Butyricimonas faecihominis | bacteria | bacteroidetes | 1472416 | Butyricimonas faecihominis | 98.61 | 221 |
| FBI00224 | Sutterella wadsworthensis | bacteria | proteobacteria | 40545 | Sutterella wadsworthensis | 99.71 | 224 |
| FBI00226 | Catabacter hongkongensis | bacteria | firmicutes | 270498 | Catabacter hongkongensis | 99.71 | 226 |
| FBI00229 | Alistipes senegalensis | bacteria | bacteroidetes | 1288121 | Alistipes senegalensis | 99.19 | 229 |
| FBI00232 | Bacteroides stercoris | bacteria | bacteroidetes | 46506 | Bacteroides stercoris | 98.84 | 232 |
| FBI00233 | Ruminococcaceae sp. FBI00233 | bacteria | firmicutes | 474960 | Anaerotruncus | 91.63 | 233 |
| | | | | | colihominis | | |
| FBI00234 | Faecalicatena contorta | bacteria | firmicutes | 39482 | Faecalicatena contorta | 99.21 | 234 |
| FBI00236 | Eisenbergiella tayi | bacteria | firmicutes | 1432052 | Eisenbergiella tayi | 99.41 | 236 |
| FBI00237 | Dielma fastidiosa | bacteria | firmicutes | 1034346 | Dielma fastidiosa | 99.78 | 237 |
| FBI00238 | Alistipes sp. FBI00238 | bacteria | bacteroidetes | 239759 | Alistipes finegoldii | 95.84 | 238 |
| FBI00239 | Lactonifactor longoviformis | bacteria | firmicutes | 341220 | Lactonifactor | 98.99 | 239 |
| | | | | | longoviformis | | |
| FBI00243 | Eubacterium siraeum | bacteria | firmicutes | 39492 | Eubacterium siraeum | 98.53 | 243 |
| FBI00244 | Faecalibacterium prausnitzii | bacteria | firmicutes | 853 | Faecalibacterium | 98.69 | 244 |
| | | | | | prausnitzii | | |
| FBI00245 | Acidaminococcus intestini | bacteria | firmicutes | 187327 | Acidaminococcus | 99.72 | 245 |
| | | | | | intestini | | |
| FBI00248 | Neglecta timonensis | bacteria | firmicutes | 1776382 | Emergencia timonensis | 99.64 | 248 |
| FBI00249 | Citrobacter portucalensis | bacteria | proteobacteria | 1639133 | Citrobacter freundii | 99.79 | 249 |
| FBI00251 | Bifidobacterium | bacteria | actinobacteria | 28026 | Bifidobacterium | 99.85 | 251 |
| | pseudocatenulatum | | | | pseudocatenulatum | | |
| FBI00254 | Eubacterium hallii | bacteria | firmicutes | 39488 | Eubacterium hallii | 99.08 | 254 |
| FBI00255 | Hungatella effluvii | bacteria | firmicutes | 1096246 | Hungatella hathewayi | 98.56 | 255 |
| FBI00258 | Turicibacter sanguinis | bacteria | firmicutes | 154288 | Turicibacter sanguinis | 99.93 | 258 |
| FBI00260 | Eubacterium rectale | bacteria | firmicutes | 39491 | Eubacterium rectale | 99.64 | 260 |
| FBI00263 | Bacteroides caccae | bacteria | bacteroidetes | 47678 | Bacteroides caccae | 99.56 | 263 |
| FBI00267 | Anaerofustis stercorihominis | bacteria | firmicutes | 214853 | Anaerofustis | 97.29 | 267 |
| | | | | | stercorihominis | | |
| FBI00269 | Alistipes putredinis | bacteria | bacteroidetes | 28117 | Alistipes putredinis | 100 | 269 |
| FBI00271 | Bacteroides xylanisolvens | bacteria | bacteroidetes | 371601 | Bacteroides | 98.42 | 271 |
| | | | | | xylanisolvens | | |
| FBI00273 | Barnesiella intestinihominis | bacteria | bacteroidetes | 487174 | Barnesiella | 99.43 | 273 |
| | | | | | intestinihominis | | |
| FBI00274 | Eubacterium xylanophilum | bacteria | firmicutes | 39497 | Eubacterium | 93.5 | 274 |
| | | | | | xylanophilum | | |
| FBI00275 | Holdemanella biformis | bacteria | firmicutes | 1735 | Holdemanella biformis | 98.99 | 275 |
| FBI00277 | Alistipes onderdonkii | bacteria | bacteroidetes | 328813 | Alistipes onderdonkii | 99.63 | 277 |
| FBI00278 | Eubacterium ventriosum | bacteria | firmicutes | 39496 | Eubacterium ventriosum | 94.14 | 278 |
| FBI00281 | Senegalimassilia anaerobia | bacteria | actinobacteria | 1473216 | Senegalimassilia anaerobia | 99.45 | 281 |
| FBI00282 | Porphyromonas asaccharolytica | bacteria | bacteroidetes | 28123 | Porphyromonas | 99.35 | 282 |
| | | | | | asaccharolytica | | |
| FBI00288 | Blautia hydrogenotrophica | bacteria | firmicutes | 53443 | Blautia hydrogenotrophica | 99.57 | 288 |
| FBI00289 | Oxalobacter formigenes | bacteria | proteobacteria | 847 | Oxalobacter formigenes | 99.21 | 289 |
| FBI00290 | Lachnospiraceae sp. FBI00290 | bacteria | firmicutes | 186803 | Eubacterium | 94.81 | 290 |
| | | | | | ruminantium | | |

### Example 12: In Vivo Oxalate Reduction by Candidate Consortia in a Germ-Free Mouse Model Fed a Low-Complexity Diet

A set of five candidate oxalate-eliminating microbial consortia, comprising active and supportive microbes isolated from human fecal samples as described in Example 1, were tested for the ability to control oxalate levels in vivo in germ-free mice fed a limited ingredient, low-complexity diet supplemented with oxalate (see Table 1).

One week prior to colonization, germ-free C57Bl/6NTac mice (n=4 per condition) were fed a refined diet rich in casein and simple sugars supplemented with oxalate to induce hyperoxaluria (see Table 2). One week later, the candidate consortia described in Example 11 (I to V) were introduced to the mice via oral gavage. One group of mice was mock-colonized with PBS alone as a negative control. Another group of mice was colonized with a previously-characterized microbial consortium as a positive control, which contains microbial strains sourced from depositories and was previously shown to reduce oxalate levels in vivo (see Examples 6 and 7; Table 8). Urine and fecal samples were collected each week for two weeks thereafter, with an endpoint at 14 days following colonization. Terminal urine (collected immediately following euthanasia) was processed by solid-phase extraction and oxalate levels were quantified by LC/MS as described in Example 4.

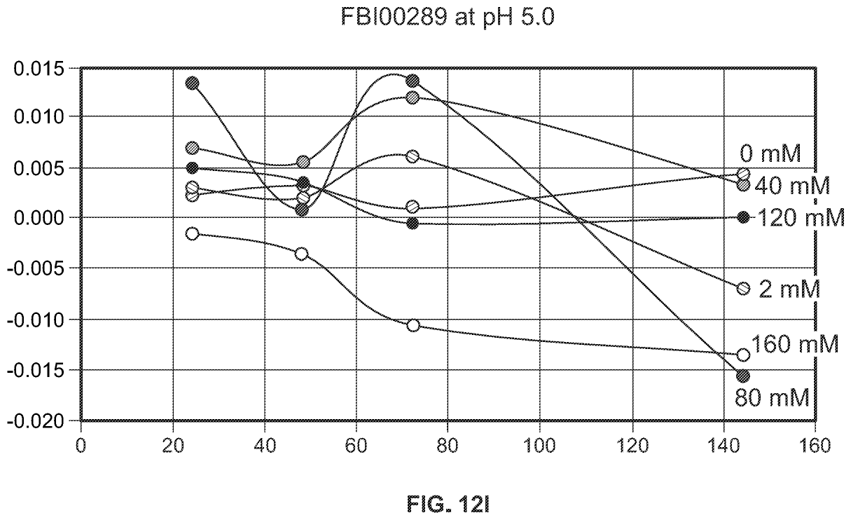

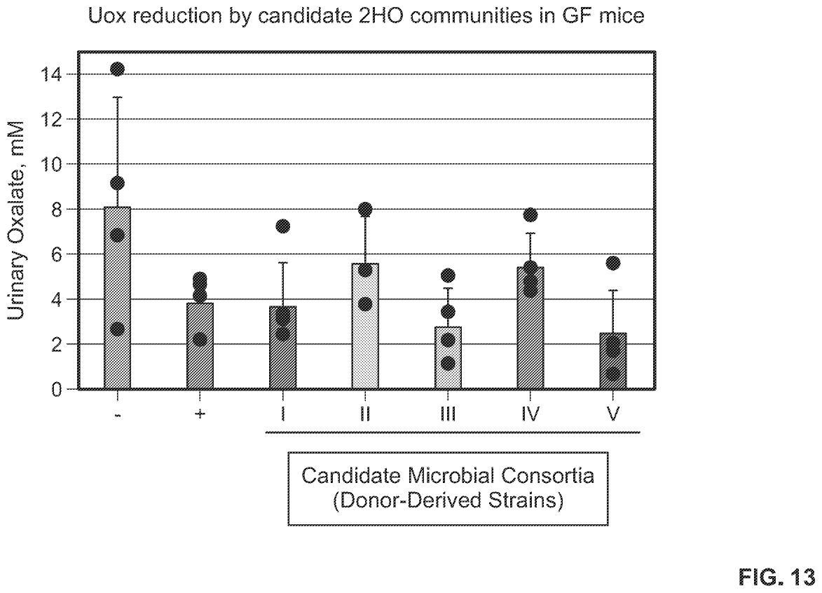

Average urinary oxalate concentrations for each study group at study endpoint are reported in FIG. 13. Mice colonized with the positive control proof-of-concept community containing commercially sourced strains of O. formigenes (+) exhibited a 53% average reduction in urinary oxalate relative to the uncolonized negative control (−). The five proprietary candidate communities (I-V), each of which comprised three internally isolated strains of O. formigenes, were found to reduce urinary oxalate by 32-70%, demonstrating efficacy on par with the positive control community. The reduction in urinary oxalate for all tested communities was statistically significant relative to the negative control.

### Example 13: In Vivo Oxalate Reduction by Candidate Consortia in a Germ Free Mouse Fed a High-Complexity Diet

The set of five candidate oxalate-eliminating microbial consortia described in Example 10 were further tested for the ability to control oxalate levels in vivo in germ free mice fed a complex, nutritionally complete diet.

One week prior to colonization, germ-free C57Bl/6NTac mice (n=4 per condition) were fed a complex, grain-based diet and given ad libitum drinking water supplemented with 0.875% oxalate to induce hyperoxaluria. One week later, the mice were colonized with the therapeutic communities via oral gavage. One group of mice was mock-colonized with PBS alone as a negative control. Another group of mice was colonized with a previously-characterized microbial consortium as a positive control, which contained microbial strains sourced from depositories and was previously shown to reduce oxalate levels in vivo (see Examples 6 and 7; Table 8). Urine and fecal samples were collected each week for two weeks thereafter, with a study endpoint at 8 days following colonization. Terminal urine (collected immediately following euthanasia) was processed by solid-phase extraction and oxalate levels were quantified using LC-MS as described in Example 4.

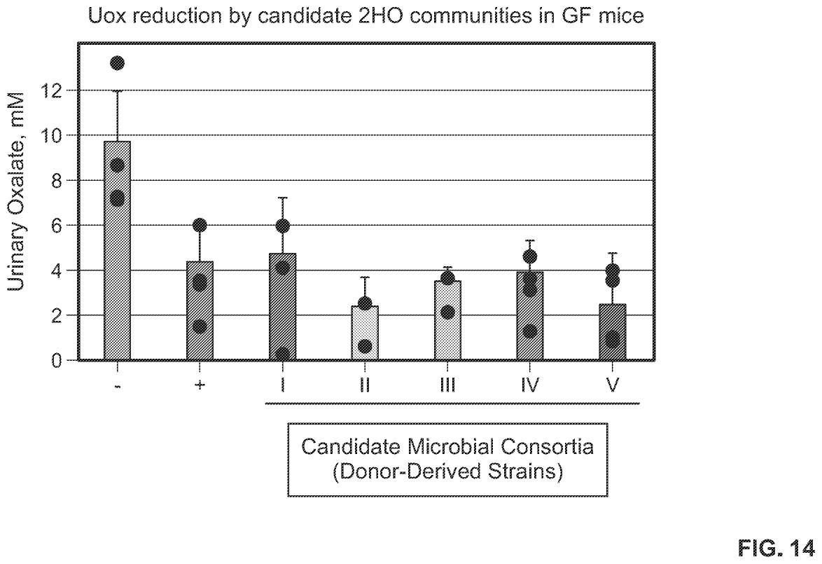

Average urinary oxalate concentrations for each study group at study endpoint are presented in FIG. 14. Mice colonized with the positive control community containing commercially sourced strains of O. formigenes (+) exhibited a 54% average reduction in urinary oxalate relative to the uncolonized negative control (−). The five proprietary candidate communities (I-V), each of which comprised three internally isolated strains of O. formigenes, were found to reduce urinary oxalate by 49-75%, demonstrating efficacy on par with the positive control community. The reduction in urinary oxalate for all tested communities was statistically significant relative to the negative control.

### Example 14: In Vivo Oxalate Reduction by Candidate Consortia in a Humanized Gnotobiotic Mouse

The set of five candidate oxalate-eliminating microbial consortia described in Example 11 were further tested for the ability to control oxalate levels in vivo in humanized, recolonized mice fed a complex, nutritionally complete diet.

Germ-free C57B1/6NTac mice (n=4 per condition) were humanized by introducing a previously characterized human donor fecal sample that lacks O. formigenes and does not appreciably degrade urinary oxalate. One week following colonization, the humanized mice were fed a complex, grain-based diet supplemented with oxalate to induce hyperoxaluria. One week later, the mice were given an antibiotic cocktail containing ampicillin (1 mg/ml) and enrofloxacin (0.575 mg/ml) ad libidum in drinking water for seven days, after which the antibiotic treatment was ended and the therapeutic communities (I-V) were introduced via oral gavage. One group of mice was mock-colonized with PBS alone as a negative control. Another group of mice was colonized with a previously-characterized microbial consortium as a positive control, which contained microbial strains sourced from depositories and was previously shown to reduce oxalate levels in vivo (see Examples 6 and 7; Table 8). A final group of mice was colonized with a set of strains (“Putative Oxalate Degraders”) that included three donor-derived strains of O. formigenes in addition to other donor-derived strains predicted to have oxalate-degrading activity. This set of strains is listed in Table 21.

TABLE 21

| | | SEQ ID |
|---|---|---|
| Strain # | Species ID | NO: X |
| FBI00001 | Clostridium citroniae | 1 |
| FBI00002 | Bacteroides salyersiae | 2 |
| FBI00004 | Neglecta timonensis | 4 |
| FBI00008 | Blautia luti | 8 |
| FBI00009 | Bifidobacterium adolescentis | 9 |
| FBI00011 | Bifidobacterium longum | 11 |
| FBI00016 | Bifidobacterium pseudocatenulatum | 16 |
| FBI00017 | Blautia obeum | 17 |
| FBI00020 | Bacteroides thetaiotaomicron | 20 |
| FBI00021 | Bacteroides kribbi/Bacteroides koreensis | 21 |
| | species cluster | |
| FBI00028 | Oscillibacter sp. FBI00028 | 28 |
| FBI00030 | Eggerthella lenta | 30 |
| FBI00033 | Lachnospiraceae sp. FBI00033 | 33 |
| FBI00041 | Phascolarctobacterium faecium | 41 |
| FBI00043 | Bifidobacterium dentium | 43 |
| FBI00045 | Bifidobacterium adolescentis | 45 |
| FBI00050 | Bacteroides nordii | 50 |
| FBI00052 | Bacteroides xylanisolvens | 52 |
| FBI00053 | Lactobacillus rogosae | 53 |
| FBI00056 | Clostridium citroniae | 56 |
| FBI00060 | Bifidobacterium longum | 60 |
| FBI00063 | Lachnospira sp. FBI00063 FBI00285 FBI00364 | 63 |
| FBI00064 | Dorea sp. FBI00064 | 64 |
| FBI00067 | Oxalobacter formigenes | 67 |
| FBI00069 | Ruminococcus bromii | 69 |
| FBI00070 | Bacteroides kribbi/Bacteroides koreensis | 70 |
| | species cluster | |
| FBI00133 | Oxalobacter formigenes | 133 |
| FBI00289 | Oxalobacter formigenes | 289 |

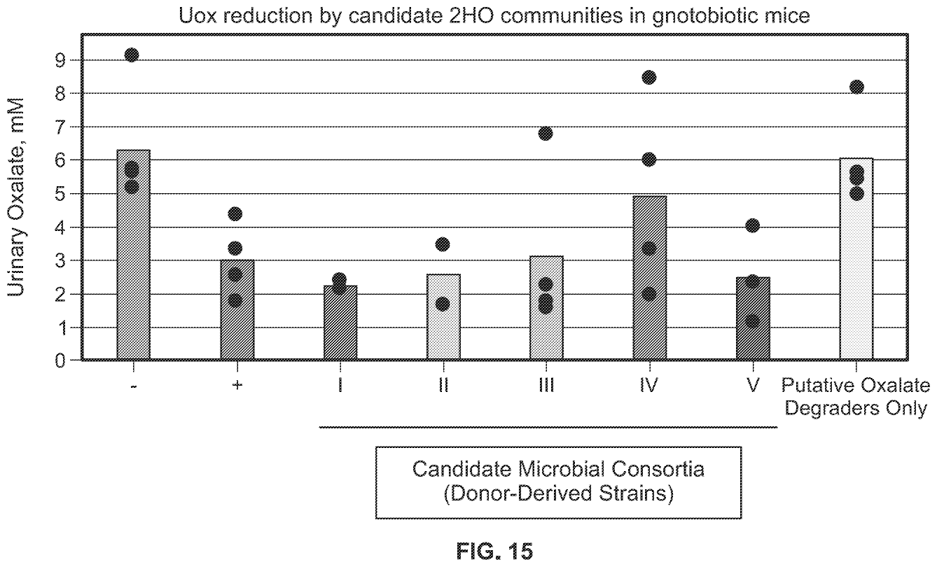

Mice were sampled each week for two weeks following recolonization to determine microbiome composition and urinary oxalate levels, with a study endpoint at 14 days following colonization with the experimental communities. Average urinary oxalate concentrations for each study group at study endpoint are presented in FIG. 15. Re-colonization with the positive control proof-of-concept community containing commercially sourced strains of O. formigenes (+) yielded a 52% average reduction in urinary oxalate relevant to the mock-treated negative control (−). The five proprietary candidate communities (I-V), each of which comprises three donor-derived strains of O. formigenes, were found to reduce urinary oxalate by 22-65%, demonstrating efficacy on par with the positive control community. The reduction in urinary oxalate for all tested communities was statistically significant for all but one community (IV), with no significant differences observed between the remaining colonized groups. Notably, recolonization with the set of putative oxalate-degrading microbes alone did not result in a reduction in urinary oxalate, demonstrating the enhanced effect of combining a plurality of active oxalate-degrading microbes with a rationally designed supportive community.

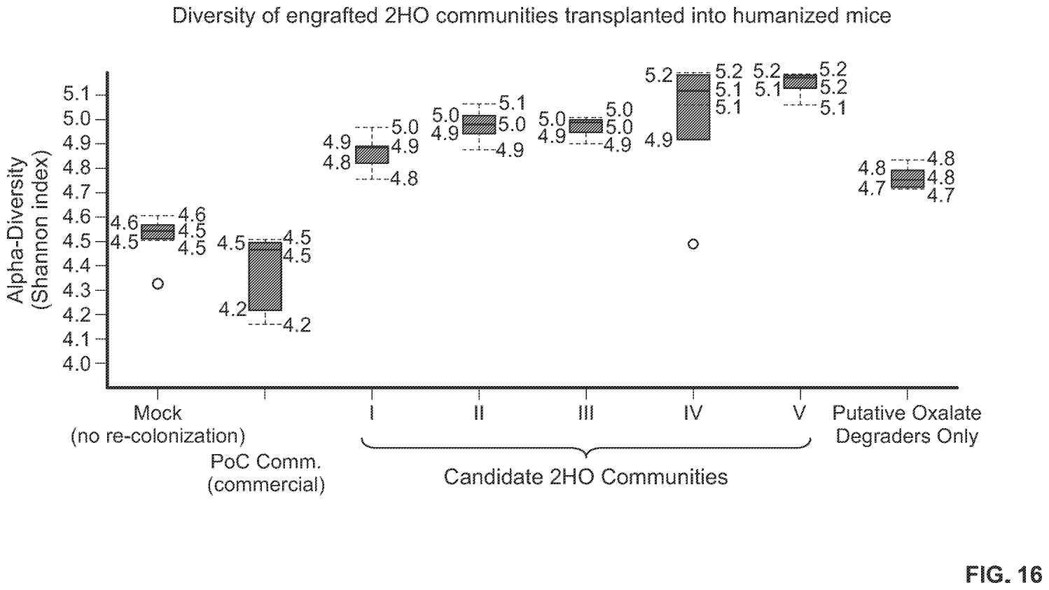

Mouse fecal samples were analyzed by metagenomic sequencing in order to determine the composition of the microbiome. Briefly, genomic DNA was extracted from mouse fecal pellets and sequenced using short-read (Illumina) sequencing. Individual reads were classified against a comprehensive reference database, containing genomes from species throughout the tree of life. The total reads classified to a species were summed and normalized by genome size to obtain estimates of relative abundance. The results of this analysis are summarized in FIG. 16. Re-colonization with one of the candidate microbial consortia (I-V) resulted in enhanced microbiome species diversity relative to both the proof-of-concept consortium and the collection of Putative Oxalate Degraders.

### Example 15: Effect of Candidate Supportive Communities on In Vivo Engraftment of O. Formigenes

The set of five candidate oxalate-eliminating microbial consortia described in Example 10 were further tested for the ability to support engraftment of the active oxalate-degrading microbe O. formigenes into germ-free mice.

Germ-free C57Bl/6NTac mice (n=4 per condition) were colonized with candidate microbial consortia (I to V) via oral gavage. One group of mice was colonized with only a supportive community of microbes as a negative control. At the conclusion of the experiment, fecal samples were analyzed via metagenomic sequencing to measure the relative and absolute abundance of O. formigenes in the microbiome. Briefly, genomic DNA was extracted from mouse fecal pellets and sequenced using short-read (Illumina) sequencing. Individual reads were classified against a comprehensive reference database, containing genomes from species throughout the tree of life. The total reads classified to a species were summed and normalized by genome size to obtain estimates of relative abundance. Absolute abundance estimates were obtained by injecting a known quantity of heterologous cells into the fecal sample prior to DNA extraction and sequencing.

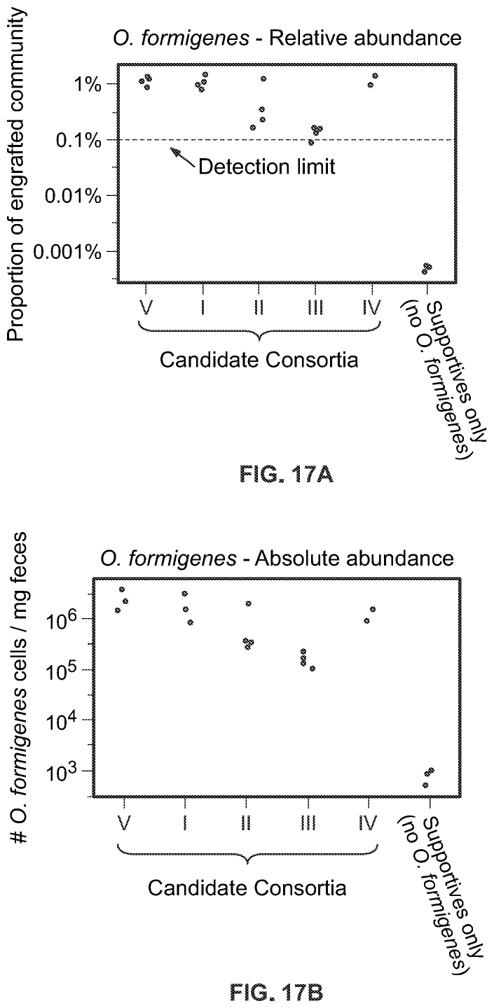

The results of this study are reported in FIG. 17. O. formigenes was detected in all mice colonized with one of the five candidate consortia, and treatment with candidate V resulted in the largest quantity of O. formigenes in the fecal sample.

### Example 16: Production of an Exemplary Therapeutic Oxalate-Degrading Consortium

This example describes the production of an exemplary microbial consortium intended for use in human subjects. In one embodiment of the invention, said exemplary consortium consists of the strains listed in Table 22, including three active oxalate-degrader strains of donor-derived O. formigenes. In another embodiment of the invention, said exemplary consortium consists of the strains listed in in Table 23. In another embodiment of the invention, said exemplary consortium consists of the strains listed in Table 24. All strains included in the exemplary consortium meet at least one of five criteria:
a. Has an experimentally confirmed ability to eliminate oxalate in vitro
b. Belongs to a species that is known to metabolize formate, a primary byproduct and potential inhibitor of oxalate metabolism in the gut
c. Belongs to a species known to contribute to metabolism of one or more nutrients typically found in the human diet
d. Belongs to species known to fulfill unique and potentially beneficial biological functions in the GI tract (e.g., bile salt hydrolase activity or butyrate production)
e. Belongs to a species found in the GI tract of one or more healthy human adults.

The final drug product consists of up to 7 drug substances, each comprising at least one characterized bacterial strain. Some drug substances are pure cultures, whereas others are from mixed-culture fermentation of anaerobic and facultative aerobic bacteria. The drug substance culture conditions are determined by one skilled in the art.

Cells are harvested and concentrated by a combination of microfiltration using 0.2-0.45 μm pore size membranes made of nonreactive polymers such as Polyvinylidene fluoride, Polysulfones, and/or nitrocellulose; and centrifugation (10,000-20,000 g force) to a final CFU concentration of 1×10⁶ to 1×10¹² CFU/ml. The concentrated biomass is mixed with sterilized cryoprotectant agent (CPA) at a volumetric ratio between 10:1 to 1:10.

The CPA is composed of a cryoprotectant/carbohydrate/bulking agent/nutrient such as glycerol (0 to 250 g/l), maltodextrin (0 to 100 g/l), sucrose (0 to 100 g/l), inulin (0 to 40 g/l), trehalose (0 to 50 g/l) and/or alginate (0 to 10 g/l). Additionally, antioxidants such as cysteine (0.25 to 0.50 g/l), ascorbic acid (0 to 5 g/l) and/or riboflavin (0 to 0.01 g/l) are added to CPA. The specific concentrations are determined by a person skilled in the art.

Finally, additional nutrients such as oxalate (0-100 mM) or formate (0-100 mM) are added to support robust revival of specific strains from the capsule, the specific concentrations being determined by one skilled in the art.

The cells are either stored frozen in a CPA or combination of CPAs, or are lyophilized to prepare various solid oral dosage forms (e.g., enteric coated capsules or enteric coated tablets). The formulated cells are lyophilized to yield a stable product. Primary drying is conducted below collapse temperature of the chosen formulation (typically below −20° C.), followed by secondary drying at higher temperature (5° C. or higher). Lyophilized powder is filled in “0” to “000” size capsules to accommodate various strengths. To prepare tablets, lyophilized powder is added to a binding agent (e.g. sucrose or starch) and pressed into tablets. The tablets are enteric coated to protect the drug product from the low pH gastric environment.

Composition of the drug product is defined by the Relative Abundance of the various intended strains. The relative abundance of microbial strains in the drug substance or drug product is determined as follows: total bacterial genomic DNA is extracted from a pelleted aliquot (e.g. 1 ml) of the drug substance/product and quantified, normalized by concentration, and prepared into an indexed library for whole-genome shotgun sequencing on an Illumina sequencer (e.g. NovaSeq). Following quality trimming, short paired-end Illumina reads (PE-150) are classified using a custom bioinformatics pipeline and taxonomically-structured database built from the genome sequences of strains in the drug product. The taxonomically-structured database links genome nucleotide sequences of a fixed length (k-mers) to a least common ancestor(s) (strain, species . . . phylum) that contain the same k-mer in the database. 150 base-pair sequencing reads are classified by retrieving the taxa for all k-mers in the read and assigning a classification based on the least common ancestor. Sequences that have no k-mers in the database are discarded. Reads that do not get classified to the strain level are distributed to the strain level using Bayes theorem to estimate true strain-level abundance. The relative abundance of a strain is calculated as the percentage of reads that are classified as that strain, divided by genome size. Absolute abundance is calculated by dividing the total bacterial cell number in the drug product (quantified by Beckman Coulter Counter) by the percent relative abundance.

A person of ordinary skill in the art shall be able to determine useful ratios of active and supportive microbes that constitute the exemplary consortium, and shall ensure that the relative abundance of supportive microbial strains is at least sufficient to enable function and stable engraftment of the plurality of active microbes.

TABLE 22

| | | | | NCBI | | % | SEQ |
|---|---|---|---|---|---|---|---|
| | | | | Taxonomy | | Match | ID NO: |
| Strain # | Species ID | Kingdom | Phylum | ID | Closest 16S Species | (16S) | X |
| FBI00001 | Clostridium citroniae | bacteria | firmicutes | 358743 | Clostridium citroniae | 99.64 | 1 |
| FBI00002 | Bacteroides salyersiae | bacteria | bacteroidetes | 291644 | Bacteroides salyersiae | 99.5 | 2 |
| FBI00004 | Neglecta timonensis | bacteria | firmicutes | 1776382 | Neglecta timonensis | 99.14 | 4 |
| FBI00009 | Bifidobacterium adolescentis | bacteria | actinobacteria | 1680 | Bifidobacterium faecale | 98.6 | 9 |
| FBI00010 | Blautia obeum | bacteria | firmicutes | 40520 | Blautia obeum | 98.12 | 10 |
| FBI00011 | Bifidobacterium longum | bacteria | actinobacteria | 216816 | Bifidobacterium longum | 99.28 | 11 |
| FBI00012 | Alistipes onderdonkii | bacteria | bacteroidetes | 328813 | Alistipes onderdonkii | 99.71 | 12 |
| FBI00013 | Parabacteroides merdae | bacteria | bacteroidetes | 46503 | Parabacteroides merdae | 99.5 | 13 |
| FBI00015 | Bacteroides uniformis | bacteria | bacteroidetes | 820 | Bacteroides uniformis | 99.78 | 15 |
| FBI00016 | Bifidobacterium pseudocatenulatum | bacteria | actinobacteria | 28026 | Bifidobacterium pseudocatenulatum | 99.64 | 16 |
| FBI00018 | Eubacterium rectale | bacteria | firmicutes | 39491 | Eubacterium rectale | 99.71 | 18 |
| FBI00019 | Alistipes timonensis | bacteria | bacteroidetes | 1465754 | Alistipes timonensis | 99.78 | 19 |
| FBI00020 | Bacteroides thetaiotaomicron | bacteria | bacteroidetes | 818 | Bacteroides thetaiotaomicron | 99.57 | 20 |
| FBI00021 | Bacteroides kribbi/ | bacteria | bacteroidetes | 816 | Bacteroides kribbi | 99.07 | 21 |
| | Bacteroides koreensis species | | | | | | |
| | cluster | | | | | | |
| FBI00022 | Alistipes putredinis | bacteria | bacteroidetes | 28117 | Alistipes putredinis | 99.93 | 22 |
| FBI00025 | Coprococcus comes | bacteria | firmicutes | 410072 | Coprococcus comes | 99.21 | 25 |
| FBI00027 | Fusicatenibacter saccharivorans | bacteria | firmicutes | 1150298 | Fusicatenibacter saccharivorans | 97.6 | 27 |
| FBI00029 | Parabacteroides distasonis | bacteria | bacteroidetes | 823 | Parabacteroides distasonis | 99.26 | 29 |
| FBI00030 | Eggerthella lenta | bacteria | firmicutes | 84112 | Eggerthella lenta | 98.47 | 30 |
| FBI00032 | Anaerostipes hadrus | bacteria | firmicutes | 649756 | Anaerostipes hadrus | 99.64 | 32 |
| FBI00033 | Lachnospiraceae sp. FBI00033 | bacteria | firmicutes | 186803 | Clostridium amygdalinum | 93.56 | 33 |
| FBI00034 | Eubacterium eligens | bacteria | firmicutes | 39485 | Eubacterium eligens | 98.78 | 34 |
| FBI00036 | Blautia faecis | bacteria | firmicutes | 871665 | Blautia faecis | 99.53 | 36 |
| FBI00038 | Coprococcus eutactus | bacteria | firmicutes | 33043 | Coprococcus eutactus | 95.96 | 38 |
| FBI00040 | Bilophila wadsworthia | bacteria | proteobacteria | 35833 | Desulfovibrio desulfuricans | 91.38 | 40 |
| FBI00043 | Bifidobacterium dentium | bacteria | actinobacteria | 1689 | Bifidobacterium dentium | 99.35 | 43 |
| FBI00044 | Blautia wexlerae | bacteria | firmicutes | 418240 | Blautia wexlerae | 98.69 | 44 |
| FBI00046 | Bacteroides caccae | bacteria | bacteroidetes | 47678 | Bacteroides caccae | 99.71 | 46 |
| FBI00047 | Eubacterium eligens | bacteria | firmicutes | 39485 | Eubacterium eligens | 98.79 | 47 |
| FBI00048 | Fusicatenibacter saccharivorans | bacteria | firmicutes | 1150298 | Fusicatenibacter saccharivorans | 97.95 | 48 |
| FBI00049 | Dialister succinatiphilus | bacteria | firmicutes | 487173 | Dialister succinatiphilus | 95.74 | 49 |
| FBI00050 | Bacteroides nordii | bacteria | bacteroidetes | 291645 | Bacteroides nordii | 98.63 | 50 |
| FBI00051 | Dorea formicigenerans | bacteria | firmicutes | 39486 | Dorea formicigenerans | 98.07 | 51 |
| FBI00052 | Bacteroides xylanisolvens | bacteria | bacteroidetes | 371601 | Bacteroides xylanisolvens | 99.14 | 52 |
| FBI00053 | Lactobacillus rogosae | bacteria | firmicutes | 706562 | Lachnospira pectinoschiza | 97.36 | 53 |
| FBI00055 | Bacteroides kribbi/ | bacteria | bacteroidetes | 816 | Bacteroides kribbi | 99.64 | 55 |
| | Bacteroides koreensis species | | | | | | |
| | cluster | | | | | | |
| FBI00056 | Clostridium citroniae | bacteria | firmicutes | 358743 | Clostridium citroniae | 99.2 | 56 |
| FBI00057 | Dorea longicatena | bacteria | firmicutes | 88431 | Dorea longicatena | 99.7 | 57 |
| FBI00059 | Bacteroides stercorirosoris | bacteria | bacteroidetes | 871324 | Bacteroides oleiciplenus | 98.81 | 59 |
| FBI00060 | Bifidobacterium longum | bacteria | actinobacteria | 216816 | Bifidobacterium longum | 99.49 | 60 |
| FBI00061 | Alistipes shahii | bacteria | bacteroidetes | 328814 | Alistipes shahii | 99.19 | 61 |
| FBI00062 | Collinsella aerofaciens | bacteria | actinobacteria | 74426 | Collinsella aerofaciens | 99.48 | 62 |
| FBI00066 | Parasutterella excrementihominis | bacteria | proteobacteria | 487175 | Parasutterella excrementihominis | 99.13 | 66 |
| FBI00067 | Oxalobacter formigenes | bacteria | proteobacteria | 847 | Oxalobacter formigenes | 98.84 | 67 |
| FBI00068 | Akkermansia muciniphila | bacteria | verruco- | 239935 | Akkermansia muciniphila | 99.42 | 68 |
| | | | microbia | | | | |
| FBI00069 | Ruminococcus bromii | bacteria | firmicutes | 40518 | Ruminococcus bromii | 98.84 | 69 |
| FBI00070 | Bacteroides kribbi/ | bacteria | bacteroidetes | 816 | Bacteroides koreensis | 99.71 | 70 |
| | Bacteroides koreensis species | | | | | | |
| | cluster | | | | | | |
| FBI00071 | Lachnospiraceae sp. FBI00071 | bacteria | firmicutes | 186803 | Roseburia faecis | 94.92 | 71 |
| FBI00072 | Coprococcus eutactus | bacteria | firmicutes | 33043 | Coprococcus eutactus | 96.17 | 72 |
| FBI00074 | Clostridium fessum | bacteria | firmicutes | 2126740 | Clostridium symbiosum | 94.03 | 74 |
| FBI00075 | Paraprevotella clara | bacteria | bacteroidetes | 454154 | Paraprevotella clara | 98.85 | 75 |
| FBI00076 | Bacteroides thetaiotaomicron | bacteria | bacteroidetes | 818 | Bacteroides thetaiotaomicron | 99.78 | 76 |
| FBI00077 | Sutterella wadsworthensis | bacteria | proteobacteria | 40545 | Sutterella wadsworthensis | 99.86 | 77 |
| FBI00078 | Blautia obeum | bacteria | firmicutes | 40520 | Blautia obeum | 98.34 | 78 |
| FBI00079 | Clostridium clostridioforme | bacteria | firmicutes | 1531 | Clostridium clostridioforme | 99.14 | 79 |
| FBI00080 | Sutterella massiliensis | bacteria | proteobacteria | 1816689 | Sutterella massiliensis | 99.78 | 80 |
| FBI00081 | Porphyromonas asaccharolytica | bacteria | bacteroidetes | 28123 | Porphyromonas asaccharolytica | 99.35 | 81 |
| FBI00082 | Ruminococcaceae sp. FBI00082 | bacteria | firmicutes | 541000 | Phocea massiliensis | 93.08 | 82 |
| | FBI00097 | | | | | | |
| FBI00085 | Ruminococcus bromii | bacteria | firmicutes | 40518 | Ruminococcus bromii | 98.62 | 85 |
| FBI00087 | Clostridium scindens | bacteria | firmicutes | 29347 | Clostridium scindens | 98.28 | 87 |
| FBI00092 | Monoglobus pectinilyticus | bacteria | firmicutes | 1981510 | Monoglobus pectinilyticus | 99.5 | 92 |
| FBI00093 | Roseburia hominis | bacteria | firmicutes | 301301 | Roseburia hominis | 99.71 | 93 |
| FBI00096 | Eggerthella lenta | bacteria | actinobacteria | 84112 | Eggerthella lenta | 98.76 | 96 |
| FBI00097 | Ruminococcaceae sp. FBI00082 | bacteria | firmicutes | 541000 | Phocea massiliensis | 93.07 | 97 |
| | FBI00097 | | | | | | |
| FBI00099 | Gordonibacter pamelaeae | bacteria | actinobacteria | 471189 | Gordonibacter pamelaeae | 99.56 | 99 |
| FBI00101 | Faecalibacterium prausnitzii | bacteria | firmicutes | 853 | Faecalibacterium prausnitzii | 97.97 | 101 |
| FBI00102 | Clostridium fessum | bacteria | firmicutes | 2126740 | Clostridium symbiosum | 94.31 | 102 |
| FBI00104 | Blautia wexlerae | bacteria | firmicutes | 418240 | Blautia luti | 97.18 | 104 |
| FBI00109 | Coprococcus comes | bacteria | firmicutes | 410072 | Coprococcus comes | 98.39 | 109 |
| FBI00110 | Lachnoclostridium pacaense | bacteria | firmicutes | 1917870 | Lachnoclostridium pacaense | 98.92 | 110 |
| FBI00111 | Bacteroides vulgatus | bacteria | bacteroidetes | 821 | Bacteroides vulgatus | 99.43 | 111 |
| FBI00112 | Bacteroides uniformis | bacteria | bacteroidetes | 820 | Bacteroides uniformis | 99.78 | 112 |
| FBI00113 | Parabacteroides merdae | bacteria | bacteroidetes | 46503 | Parabacteroides merdae | 99.79 | 113 |
| FBI00115 | Dorea formicigenerans | bacteria | firmicutes | 39486 | Dorea formicigenerans | 97.98 | 115 |
| FBI00116 | Ruminococcus faecis | bacteria | firmicutes | 592978 | Ruminococcus faecis | 99.57 | 116 |
| FBI00117 | Blautia faecis | bacteria | firmicutes | 871665 | Blautia faecis | 99.52 | 117 |
| FBI00120 | Hungatella effluvii | bacteria | firmicutes | 154046 | Hungatella hathewayi | 98.78 | 120 |
| FBI00123 | Roseburia hominis | bacteria | firmicutes | 301301 | Roseburia hominis | 100 | 123 |
| FBI00124 | Anaerostipes hadrus | bacteria | firmicutes | 649756 | Anaerostipes hadrus | 99.86 | 124 |
| FBI00125 | Bacteroides stercoris | bacteria | bacteroidetes | 46506 | Bacteroides stercoris | 99.64 | 125 |
| FBI00126 | Bifidobacterium adolescentis | bacteria | actinobacteria | 1680 | Bifidobacterium adolescentis | 98.98 | 126 |
| FBI00127 | Collinsella aerofaciens | bacteria | actinobacteria | 74426 | Collinsella aerofaciens | 98.81 | 127 |
| FBI00128 | Hungatella effluvii | bacteria | firmicutes | 1096246 | Hungatella effluvii | 98.71 | 128 |
| FBI00132 | Gordonibacter pamelaeae | bacteria | actinobacteria | 471189 | Gordonibacter pamelaeae | 99.48 | 132 |
| FBIOO133 | Oxalobacter formigenes | bacteria | proteobacteria | 847 | Oxalobacter formigenes | 99.21 | 133 |
| FBI00135 | Bifidobacterium pseudocatenulatum | bacteria | actinobacteria | 28026 | Bifidobacterium pseudocatenulatum | 99.57 | 135 |
| FBI00137 | Bacteroides fragilis | bacteria | bacteroidetes | 817 | Bacteroides fragilis | 99.71 | 137 |
| FBI00140 | Phascolarctobacterium faecium | bacteria | firmicutes | 33025 | Phascolarctobacterium faecium | 99.58 | 140 |
| FBI00141 | Phascolarctobacterium faecium | bacteria | firmicutes | 33025 | Phascolarctobacterium faecium | 99.15 | 141 |
| FBI00145 | Bifidobacterium adolescentis | bacteria | actinobacteria | 1680 | Bifidobacterium adolescentis | 99.14 | 145 |
| FBI00147 | Clostridium bolteae | bacteria | firmicutes | 208479 | Clostridium bolteae | 99.28 | 147 |
| FBI00149 | Monoglobus pectinilyticus | bacteria | firmicutes | 1981510 | Monoglobus pectinilyticus | 99.5 | 149 |
| FBI00151 | Clostridium aldenense | bacteria | firmicutes | 358742 | Clostridium aldenense | 98.55 | 151 |
| FBI00152 | Dialister invisus | bacteria | firmicutes | 218538 | Dialister invisus | 99.58 | 152 |
| FBI00159 | Eisenbergiella tayi | bacteria | firmicutes | 1432052 | Eisenbergiella tayi | 99.03 | 159 |
| FBI00162 | Bifidobacterium catenulatum | bacteria | actinobacteria | 1686 | Bifidobacterium catenulatum | 99.14 | 162 |
| FBI00165 | Bacteroides massiliensis | bacteria | bacteroidetes | 204516 | Bacteroides massiliensis | 99.71 | 165 |
| FBI00167 | Dorea longicatena | bacteria | firmicutes | 88431 | Dorea longicatena | 99.39 | 167 |
| FBI00170 | Eggerthella lenta | bacteria | actinobacteria | 84112 | Eggerthella lenta | 98.61 | 170 |
| FBI00171 | Bilophila wadsworthia | bacteria | proteobacteria | 35833 | Desulfovibrio desulfuricans | 91.45 | 171 |
| FBI00174 | Lactobacillus rogosae | bacteria | firmicutes | 706562 | Lachnospira pectinoschiza | 97.92 | 174 |
| FBI00175 | Holdemanella biformis | bacteria | firmicutes | 1735 | Holdemanella biformis | 98.19 | 175 |
| FBI00176 | Ruthenibacterium lactatiformans | bacteria | firmicutes | 1550024 | Ruthenibacterium lactatiformans | 99.71 | 176 |
| FBI00177 | Parasutterella excrementihominis | bacteria | proteobacteria | 487175 | Parasutterella excrementihominis | 99.71 | 177 |
| FBI00180 | Alistipes sp. FBI00180 | bacteria | bacteroidetes | 239759 | Alistipes senegalensis | 97.56 | 180 |
| FBI00182 | Bacteroides coprocola | bacteria | bacteroidetes | 310298 | Bacteroides coprocola | 99.64 | 182 |
| FBI00184 | Bacteroides faecis | bacteria | bacteroidetes | 674529 | Bacteroides faecis | 99.78 | 184 |
| FBI00189 | Bacteroides ovatus | bacteria | bacteroidetes | 28116 | Bacteroides koreensis | 99.93 | 189 |
| FBI00190 | Bacteroides finegoldii | bacteria | bacteroidetes | 338188 | Bacteroides finegoldii | 98.91 | 190 |
| FBI00191 | Clostridiaceae sp. FBI00191 | bacteria | firmicutes | 31979 | Clostridium swellfunianum | 96.24 | 191 |
| FBI00194 | Ruminococcus faecis | bacteria | firmicutes | 592978 | Ruminococcus faecis | 98.41 | 194 |
| FBI00197 | Bifidobacterium bifidum | bacteria | actinobacteria | 1681 | Bifidobacterium bifidum | 99.85 | 197 |
| FBI00198 | Lachnoclostridium pacaense | bacteria | firmicutes | 1917870 | Lachnoclostridium pacaense | 99.71 | 198 |
| FBI00199 | Clostridium bolteae | bacteria | firmicutes | 208479 | Clostridium bolteae | 99.28 | 199 |
| FBI00200 | Longicatena caecimuris | bacteria | firmicutes | 1796635 | Longicatena caecimuris | 99.71 | 200 |
| FBI00201 | Eggerthella lenta | bacteria | actinobacteria | 84112 | Eggerthella lenta | 98.83 | 201 |
| FBI00205 | Blautia massiliensis | bacteria | firmicutes | 1737424 | Blautia luti | 97.55 | 205 |
| FBI00206 | Bacteroides xylanisolvens | bacteria | bacteroidetes | 371601 | Bacteroides xylanisolvens | 99.56 | 206 |
| FBI00208 | Anaerotruncus massiliensis | bacteria | firmicutes | 1673720 | Anaerotruncus colihominis | 96.52 | 208 |
| FBI00210 | Bifidobacterium bifidum | bacteria | actinobacteria | 1681 | Bifidobacterium bifidum | 99.93 | 210 |
| FBI00211 | Bacteroides vulgatus | bacteria | bacteroidetes | 821 | Bacteroides vulgatus | 99.78 | 211 |
| FBI00212 | Clostridium aldenense | bacteria | firmicutes | 358742 | Clostridium aldenense | 99.1 | 212 |
| FBI00220 | Megasphaera massiliensis | bacteria | firmicutes | 1232428 | Megasphaera massiliensis | 98.8 | 220 |
| FBI00221 | Butyricimonas faecihominis | bacteria | bacteroidetes | 1472416 | Butyricimonas faecihominis | 98.61 | 221 |
| FBI00224 | Sutterella wadsworthensis | bacteria | proteobacteria | 40545 | Sutterella wadsworthensis | 99.71 | 224 |
| FBI00226 | Catabacter hongkongensis | bacteria | firmicutes | 270498 | Catabacter hongkongensis | 99.71 | 226 |
| FBI00229 | Alistipes senegalensis | bacteria | bacteroidetes | 1288121 | Alistipes senegalensis | 99.19 | 229 |
| FBI00231 | Parabacteroides distasonis | bacteria | bacteroidetes | 823 | Parabacteroides distasonis | 99.11 | 231 |
| FBI00232 | Bacteroides stercoris | bacteria | bacteroidetes | 46506 | Bacteroides stercoris | 98.84 | 232 |
| FBI00233 | Ruminococcaceae sp. FBI00233 | bacteria | firmicutes | 474960 | Anaerotruncus colihominis | 91.63 | 233 |
| FBI00235 | Alistipes shaliii | bacteria | bacteroidetes | 328814 | Alistipes shahii | 99.86 | 235 |
| FBI00236 | Eisenbergiella tayi | bacteria | firmicutes | 1432052 | Eisenbergiella tayi | 99.41 | 236 |
| FBI00237 | Dielma fastidiosa | bacteria | firmicutes | 1034346 | Dielma fastidiosa | 99.78 | 237 |
| FBI00238 | Alistipes sp. FBI00238 | bacteria | bacteroidetes | 239759 | Alistipes finegoldii | 95.84 | 238 |
| FBI00243 | Eubacterium siraeum | bacteria | firmicutes | 39492 | Eubacterium siraeum | 98.53 | 243 |
| FBI00244 | Faecalibacterium prausnitzii | bacteria | firmicutes | 853 | Faecalibacterium prausnitzii | 98.69 | 244 |
| FBI00245 | Acidaminococcus intestini | bacteria | firmicutes | 187327 | Acidaminococcus intestini | 99.72 | 245 |
| FBI00248 | Neglecta timonensis | bacteria | firmicutes | 1776382 | Emergencia timonensis | 99.64 | 248 |
| FBI00251 | Bifidobacterium pseudocatenulatum | bacteria | actinobacteria | 28026 | Bifidobacterium pseudocatenulatum | 99.85 | 251 |
| FBI00254 | Eubacterium hallii | bacteria | firmicutes | 39488 | Eubacterium hallii | 99.08 | 254 |
| FBI00255 | Hungatella effluvii | bacteria | firmicutes | 1096246 | Hungatella hathewayi | 98.56 | 255 |
| FBI00258 | Turicibacter sanguinis | bacteria | firmicutes | 154288 | Turicibacter sanguinis | 99.93 | 258 |
| FBI00260 | Eubacterium rectale | bacteria | firmicutes | 39491 | Eubacterium rectale | 99.64 | 260 |
| FBI00263 | Bacteroides caccae | bacteria | bacteroidetes | 47678 | Bacteroides caccae | 99.56 | 263 |
| FBI00267 | Anaerofustis stercorihominis | bacteria | firmicutes | 214853 | Anaerofustis stercorihominis | 97.29 | 267 |
| FBI00269 | Alistipes putredinis | bacteria | bacteroidetes | 28117 | Alistipes putredinis | 100 | 269 |
| FBI00270 | Methanobrevibacter smithii | archaea | euryarchaeota | 2173 | Methanobrevibacter smithii | 99.69 | 270 |
| FBI00271 | Bacteroides xylanisolvens | bacteria | bacteroidetes | 371601 | Bacteroides xylanisolvens | 98.42 | 271 |
| FBI00273 | Bamesiella intestinihominis | bacteria | bacteroidetes | 487174 | Bamesiella intestinihominis | 99.43 | 273 |
| FBI00274 | Eubacterium xylanophilum | bacteria | firmicutes | 39497 | Eubacterium xylanophilum | 93.5 | 274 |
| FBI00275 | Holdemanella biformis | bacteria | firmicutes | 1735 | Holdemanella biformis | 98.99 | 275 |
| FBI00277 | Alistipes onderdonkii | bacteria | bacteroidetes | 328813 | Alistipes onderdonkii | 99.63 | 277 |
| FBI00278 | Eubacterium ventriosum | bacteria | firmicutes | 39496 | Eubacterium ventriosum | 94.14 | 278 |
| FBI00281 | Senegalimassilia anaerobia | bacteria | actinobacteria | 1473216 | Senegalimassilia anaerobia | 99.45 | 281 |
| FBI00282 | Porphyromonas asaccharolytica | bacteria | bacteroidetes | 28123 | Porphyromonas asaccharolytica | 99.35 | 282 |
| FBI00288 | Blautia hydrogenotrophica | bacteria | firmicutes | 53443 | Blautia hydrogenotrophica | 99.57 | 288 |
| FBI00289 | Oxalobacter formigenes | bacteria | proteobacteria | 847 | Oxalobacter formigenes | 99.21 | 289 |
| FBI00290 | Lachnospiraceae sp. FBI00290 | bacteria | firmicutes | 186803 | Eubacterium ruminantium | 94.81 | 290 |
| FBI00292 | Methanobrevibacter smithii | archaea | euryarchaeota | 2173 | Methanobrevibacter smithii | 99.44 | 292 |
| FBI00377 | Clostridiales sp. FBI00377 | bacteria | firmicutes | 186802 | Christensenella massiliensis | 88.69 | 377 |

TABLE 23

| | | | | NCBI | | % | SEQ |
|---|---|---|---|---|---|---|---|
| | | | | Taxonomy | | Match | ID NO: |
| Strain # | Species ID | Kingdom | Phylum | ID | Closest 16S Species | (16S) | X |
| FBI00001 | Clostridium citroniae | bacteria | firmicutes | 358743 | Clostridium citroniae | 99.64 | 1 |
| FBI00002 | Bacteroides salyersiae | bacteria | bacteroidetes | 291644 | Bacteroides salyersiae | 99.5 | 2 |
| FBI00004 | Neglecta timonensis | bacteria | firmicutes | 1776382 | Neglecta timonensis | 99.14 | 4 |
| FBI00009 | Bifidobacterium adolescentis | bacteria | actinobacteria | 1680 | Bifidobacterium faecale | 98.6 | 9 |
| FBI00010 | Blautia obeum | bacteria | firmicutes | 40520 | Blautia obeum | 98.12 | 10 |
| FBI00011 | Bifidobacterium longum | bacteria | actinobacteria | 216816 | Bifidobacterium longum | 99.28 | 11 |
| FBI00012 | Alistipes onderdonkii | bacteria | bacteroidetes | 328813 | Alistipes onderdonkii | 99.71 | 12 |
| FBI00013 | Parabacteroides merdae | bacteria | bacteroidetes | 46503 | Parabacteroides merdae | 99.5 | 13 |
| FBI00015 | Bacteroides uniformis | bacteria | bacteroidetes | 820 | Bacteroides uniformis | 99.78 | 15 |
| FBI00016 | Bifidobacterium pseudocatenulatum | bacteria | actinobacteria | 28026 | Bifidobacterium pseudocatenulatum | 99.64 | 16 |
| FBI00018 | Eubacterium rectale | bacteria | firmicutes | 39491 | Eubacterium rectale | 99.71 | 18 |
| FBI00019 | Alistipes timonensis | bacteria | bacteroidetes | 1465754 | Alistipes timonensis | 99.78 | 19 |
| FBI00020 | Bacteroides thetaiotaomicron | bacteria | bacteroidetes | 818 | Bacteroides thetaiotaomicron | 99.57 | 20 |
| FBI00021 | Bacteroides kribbi/ | bacteria | bacteroidetes | 816 | Bacteroides kribbi | 99.07 | 21 |
| | Bacteroides koreensis species | | | | | | |
| | cluster | | | | | | |
| FBI00022 | Alistipes putredinis | bacteria | bacteroidetes | 28117 | Alistipes putredinis | 99.93 | 22 |
| FBI00025 | Coprococcus comes | bacteria | firmicutes | 410072 | Coprococcus comes | 99.21 | 25 |
| FBI00027 | Fusicatenibacter saccharivorans | bacteria | firmicutes | 1150298 | Fusicatenibacter saccharivorans | 97.6 | 27 |
| FBI00029 | Parabacteroides distasonis | bacteria | bacteroidetes | 823 | Parabacteroides distasonis | 99.26 | 29 |
| FBI00030 | Eggerthella lenta | bacteria | firmicutes | 84112 | Eggerthella lenta | 98.47 | 30 |
| FBI00032 | Anaerostipes hadrus | bacteria | firmicutes | 649756 | Anaerostipes hadrus | 99.64 | 32 |
| FBI00033 | Lachnospiraceae sp. FBI00033 | bacteria | firmicutes | 186803 | Clostridium amygdalinum | 93.56 | 33 |
| FBI00034 | Eubacterium eligens | bacteria | firmicutes | 39485 | Eubacterium eligens | 98.78 | 34 |
| FBI00036 | Blautia faecis | bacteria | firmicutes | 871665 | Blautia faecis | 99.53 | 36 |
| FBI00038 | Coprococcus eutactus | bacteria | firmicutes | 33043 | Coprococcus eutactus | 95.96 | 38 |
| FBI00040 | Bilophila wadsworthia | bacteria | proteobacteria | 35833 | Desulfovibrio desulfuricans | 91.38 | 40 |
| FBI00043 | Bifidobacterium dentium | bacteria | actinobacteria | 1689 | Bifidobacterium dentium | 99.35 | 43 |
| FBI00044 | Blautia wexlerae | bacteria | firmicutes | 418240 | Blautia wexlerae | 98.69 | 44 |
| FBI00046 | Bacteroides caccae | bacteria | bacteroidetes | 47678 | Bacteroides caccae | 99.71 | 46 |
| FBI00047 | Eubacterium eligens | bacteria | firmicutes | 39485 | Eubacterium eligens | 98.79 | 47 |
| FBI00048 | Fusicatenibacter saccharivorans | bacteria | firmicutes | 1150298 | Fusicatenibacter saccharivorans | 97.95 | 48 |
| FBI00049 | Dialister succinatiphilus | bacteria | firmicutes | 487173 | Dialister succinatiphilus | 95.74 | 49 |
| FBI00050 | Bacteroides nordii | bacteria | bacteroidetes | 291645 | Bacteroides nordii | 98.63 | 50 |
| FBI00051 | Dorea formicigenerans | bacteria | firmicutes | 39486 | Dorea formicigenerans | 98.07 | 51 |
| FBI00052 | Bacteroides xylanisolvens | bacteria | bacteroidetes | 371601 | Bacteroides xylanisolvens | 99.14 | 52 |
| FBI00053 | Lactobacillus rogosae | bacteria | firmicutes | 706562 | Lachnospira pectinoschiza | 97.36 | 53 |
| FBI00055 | Bacteroides kribbi/ | bacteria | bacteroidetes | 816 | Bacteroides kribbi | 99.64 | 55 |
| | Bacteroides koreensis species | | | | | | |
| | cluster | | | | | | |
| FBI00056 | Clostridium citroniae | bacteria | firmicutes | 358743 | Clostridium citroniae | 99.2 | 56 |
| FBI00057 | Dorea longicatena | bacteria | firmicutes | 88431 | Dorea longicatena | 99.7 | 57 |
| FBI00059 | Bacteroides stercorirosoris | bacteria | bacteroidetes | 871324 | Bacteroides oleiciplenus | 98.81 | 59 |
| FBI00060 | Bifidobacterium longum | bacteria | actinobacteria | 216816 | Bifidobacterium longum | 99.49 | 60 |
| FBI00061 | Alistipes shahii | bacteria | bacteroidetes | 328814 | Alistipes shahii | 99.19 | 61 |
| FBI00062 | Collinsella aerofaciens | bacteria | actinobacteria | 74426 | Collinsella aerofaciens | 99.48 | 62 |
| FBI00066 | Parasutterella excrementihominis | bacteria | proteobacteria | 487175 | Parasutterella excrementihominis | 99.13 | 66 |
| FBI00067 | Oxalobacter formigenes | bacteria | proteobacteria | 847 | Oxalobacter formigenes | 98.84 | 67 |
| FBI00068 | Akkermansia muciniphila | bacteria | verrucomicrobi a | 239935 | Akkermansia muciniphila | 99.42 | 68 |
| FBI00069 | Ruminococcus bromii | bacteria | firmicutes | 40518 | Ruminococcus bromii | 98.84 | 69 |
| FBI00070 | Bacteroides kribbi/ | bacteria | bacteroidetes | 816 | Bacteroides koreensis | 99.71 | 70 |
| | Bacteroides koreensis species | | | | | | |
| | cluster | | | | | | |
| FBI00071 | Lachnospiraceae sp. FBI00071 | bacteria | firmicutes | 186803 | Roseburia faecis | 94.92 | 71 |
| FBI00072 | Coprococcus eutactus | bacteria | firmicutes | 33043 | Coprococcus eutactus | 96.17 | 72 |
| FBI00074 | Clostridium fessum | bacteria | firmicutes | 2126740 | Clostridium symbiosum | 94.03 | 74 |
| FBI00075 | Paraprevotella clara | bacteria | bacteroidetes | 454154 | Paraprevotella clara | 98.85 | 75 |
| FBI00076 | Bacteroides thetaiotaomicron | bacteria | bacteroidetes | 818 | Bacteroides thetaiotaomicron | 99.78 | 76 |
| FBI00077 | Sutterella wadsworthensis | bacteria | proteobacteria | 40545 | Sutterella wadsworthensis | 99.86 | 77 |
| FBI00078 | Blautia obeum | bacteria | firmicutes | 40520 | Blautia obeum | 98.34 | 78 |
| FBI00079 | Clostridium clostridioforme | bacteria | firmicutes | 1531 | Clostridium clostridioforme | 99.14 | 79 |
| FBI00080 | Sutterella massiliensis | bacteria | proteobacteria | 1816689 | Sutterella massiliensis | 99.78 | 80 |
| FBI00081 | Porphyromonas asaccharolytica | bacteria | bacteroidetes | 28123 | Porphyromonas asaccharolytica | 99.35 | 81 |
| FBI00082 | Ruminococcaceae sp. FBI00082 | bacteria | firmicutes | 541000 | Phocea massiliensis | 93.08 | 82 |
| | FBI00097 | | | | | | |
| FBI00085 | Ruminococcus bromii | bacteria | firmicutes | 40518 | Ruminococcus bromii | 98.62 | 85 |
| FBI00087 | Clostridium scindens | bacteria | firmicutes | 29347 | Clostridium scindens | 98.28 | 87 |
| FBI00092 | Monoglobus pectinilyticus | bacteria | firmicutes | 1981510 | Monoglobus pectinilyticus | 99.5 | 92 |
| FBI00093 | Roseburia hominis | bacteria | firmicutes | 301301 | Roseburia hominis | 99.71 | 93 |
| FBI00096 | Eggerthella lenta | bacteria | actinobacteria | 84112 | Eggerthella lenta | 98.76 | 96 |
| FBI00097 | Ruminococcaceae sp. FBI00082 | bacteria | firmicutes | 541000 | Phocea massiliensis | 93.07 | 97 |
| | FBI00097 | | | | | | |
| FBI00099 | Gordonibacter pamelaeae | bacteria | actinobacteria | 471189 | Gordonibacter pamelaeae | 99.56 | 99 |
| FBI00101 | Faecalibacterium prausnitzii | bacteria | firmicutes | 853 | Faecalibacterium prausnitzii | 97.97 | 101 |
| FBI00102 | Clostridium fessum | bacteria | firmicutes | 2126740 | Clostridium symbiosum | 94.31 | 102 |
| FBI00104 | Blautia wexlerae | bacteria | firmicutes | 418240 | Blautia luti | 97.18 | 104 |
| FBI00109 | Coprococcus comes | bacteria | firmicutes | 410072 | Coprococcus comes | 98.39 | 109 |
| FBI00110 | Lachnoclostridium pacaense | bacteria | firmicutes | 1917870 | Lachnoclostridium pacaense | 98.92 | 110 |
| FBI00111 | Bacteroides vulgatus | bacteria | bacteroidetes | 821 | Bacteroides vulgatus | 99.43 | 111 |
| FBI00112 | Bacteroides uniformis | bacteria | bacteroidetes | 820 | Bacteroides uniformis | 99.78 | 112 |
| FBI00113 | Parabacteroides merdae | bacteria | bacteroidetes | 46503 | Parabacteroides merdae | 99.79 | 113 |
| FBI00115 | Dorea formicigenerans | bacteria | firmicutes | 39486 | Dorea formicigenerans | 97.98 | 115 |
| FBI00116 | Ruminococcus faecis | bacteria | firmicutes | 592978 | Ruminococcus faecis | 99.57 | 116 |
| FBI00117 | Blautia faecis | bacteria | firmicutes | 871665 | Blautia faecis | 99.52 | 117 |
| FBI00120 | Hungatella effluvii | bacteria | firmicutes | 154046 | Hungatella hathewayi | 98.78 | 120 |
| FBI00123 | Roseburia hominis | bacteria | firmicutes | 301301 | Roseburia hominis | 100 | 123 |
| FBI00124 | Anaerostipes hadrus | bacteria | firmicutes | 649756 | Anaerostipes hadrus | 99.86 | 124 |
| FBI00125 | Bacteroides stercoris | bacteria | bacteroidetes | 46506 | Bacteroides stercoris | 99.64 | 125 |
| FBI00126 | Bifidobacterium adolescentis | bacteria | actinobacteria | 1680 | Bifidobacterium adolescentis | 98.98 | 126 |
| FBI00127 | Collinsella aerofaciens | bacteria | actinobacteria | 74426 | Collinsella aerofaciens | 98.81 | 127 |
| FBI00128 | Hungatella effluvii | bacteria | firmicutes | 1096246 | Hungatella effluvii | 98.71 | 128 |
| FBI00132 | Gordonibacter pamelaeae | bacteria | actinobacteria | 471189 | Gordonibacter pamelaeae | 99.48 | 132 |
| FBI00133 | Oxalobacter formigenes | bacteria | proteobacteria | 847 | Oxalobacter formigenes | 99.21 | 133 |
| FBI00135 | Bifidobacterium pseudocatenulatum | bacteria | actinobacteria | 28026 | Bifidobacterium pseudocatenulatum | 99.57 | 135 |
| FBI00137 | Bacteroides fragilis | bacteria | bacteroidetes | 817 | Bacteroides fragilis | 99.71 | 137 |
| FBI00140 | Phascolarctobacterium faecium | bacteria | firmicutes | 33025 | Phascolarctobacterium faecium | 99.58 | 140 |
| FBI00141 | Phascolarctobacterium faecium | bacteria | firmicutes | 33025 | Phascolarctobacterium faecium | 99.15 | 141 |
| FBI00145 | Bifidobacterium adolescentis | bacteria | actinobacteria | 1680 | Bifidobacterium adolescentis | 99.14 | 145 |
| FBI00147 | Clostridium bolteae | bacteria | firmicutes | 208479 | Clostridium bolteae | 99.28 | 147 |
| FBI00149 | Monoglobus pectinilyticus | bacteria | firmicutes | 1981510 | Monoglobus pectinilyticus | 99.5 | 149 |
| FBI00151 | Clostridium aldenense | bacteria | firmicutes | 358742 | Clostridium aldenense | 98.55 | 151 |
| FBI00152 | Dialister invisus | bacteria | firmicutes | 218538 | Dialister invisus | 99.58 | 152 |
| FBI00159 | Eisenbergiella tayi | bacteria | firmicutes | 1432052 | Eisenbergiella tayi | 99.03 | 159 |
| FBI00162 | Bifidobacterium catenulatum | bacteria | actinobacteria | 1686 | Bifidobacterium catenulatum | 99.14 | 162 |
| FBI00165 | Bacteroides massiliensis | bacteria | bacteroidetes | 204516 | Bacteroides massiliensis | 99.71 | 165 |
| FBI00167 | Dorea longicatena | bacteria | firmicutes | 88431 | Dorea longicatena | 99.39 | 167 |
| FBI00170 | Eggerthella lenta | bacteria | actinobacteria | 84112 | Eggerthella lenta | 98.61 | 170 |
| FBI00171 | Bilophila wadsworthia | bacteria | proteobacteria | 35833 | Desulfovibrio desulfuricans | 91.45 | 171 |
| FBI00174 | Lactobacillus rogosae | bacteria | firmicutes | 706562 | Lachnospira pectinoschiza | 97.92 | 174 |
| FBI00175 | Holdemanella biformis | bacteria | firmicutes | 1735 | Holdemanella biformis | 98.19 | 175 |
| FBI00176 | Ruthenibacterium lactatiformans | bacteria | firmicutes | 1550024 | Ruthenibacterium lactatiformans | 99.71 | 176 |
| FBI00289 | Oxalobacter formigenes | bacteria | proteobacteria | 847 | Oxalobacter formigenes | 99.21 | 289 |

TABLE 24

| | | | | | NCBI | |
|---|---|---|---|---|---|---|
| | | % Match | | | Taxonomy | SEQ ID |
| Strain # | Species: 16S Best-BLAST | (16S) | Kingdom | Phylum | ID | NO: X |
| FBI00002 | Bacteroides salyersiae | 99.5 | bacteria | bacteroidetes | 291644 | 2 |
| FBI00004 | Neglecta timonensis | 99.14 | bacteria | firmicutes | 1776382 | 4 |
| FBI00009 | Bifidobacterium faecale | 98.6 | bacteria | actinobacteria | 1454229 | 9 |
| FBI00010 | Blautia obeum | 98.12 | bacteria | firmicutes | 40520 | 10 |
| FBI00012 | Alistipes onderdonkii | 99.71 | bacteria | bacteroidetes | 328813 | 12 |
| FBI00013 | Parabacteroides merdae | 99.5 | bacteria | bacteroidetes | 46503 | 13 |
| FBI00015 | Bacteroides uniformis | 99.78 | bacteria | bacteroidetes | 820 | 15 |
| FBI00016 | Bifidobacterium pseudocatenulatum | 99.64 | bacteria | actinobacteria | 28026 | 16 |
| FBI00018 | Eubacterium rectale | 99.71 | bacteria | firmicutes | 39491 | 18 |
| FBI00019 | Alistipes timonensis | 99.78 | bacteria | bacteroidetes | 1465754 | 19 |
| FBI00021 | Bacteroides kribbi | 99.07 | bacteria | bacteroidetes | 1912894 | 21 |
| FBI00022 | Alistipes putredinis | 99.93 | bacteria | bacteroidetes | 28117 | 22 |
| FBI00029 | Parabacteroides distasonis | 99.26 | bacteria | bacteroidetes | 823 | 29 |
| FBI00032 | Anaerostipes hadrus | 99.64 | bacteria | firmicutes | 649756 | 32 |
| FBI00033 | Clostridium amygdalinum | 93.56 | bacteria | firmicutes | 31979 | 33 |
| FBI00034 | Eubacterium eligens | 98.78 | bacteria | firmicutes | 39485 | 34 |
| FBI00038 | Coprococcus eutactus | 95.96 | bacteria | firmicutes | 33043 | 38 |
| FBI00043 | Bifidobacterium dentium | 99.35 | bacteria | actinobacteria | 1689 | 43 |
| FBI00044 | Blautia wexlerae | 98.69 | bacteria | firmicutes | 418240 | 44 |
| FBI00046 | Bacteroides caccae | 99.71 | bacteria | bacteroidetes | 47678 | 46 |
| FBI00048 | Fusicatenibacter saccharivorans | 97.95 | bacteria | firmicutes | 1150298 | 48 |
| FBI00049 | Dialister succinatiphilus | 95.74 | bacteria | firmicutes | 487173 | 49 |
| FBI00050 | Bacteroides nordii | 98.63 | bacteria | bacteroidetes | 291645 | 50 |
| FBI00051 | Dorea formicigenerans | 98.07 | bacteria | firmicutes | 39486 | 51 |
| FBI00052 | Bacteroides xylanisolvens | 99.14 | bacteria | bacteroidetes | 28116 | 52 |
| FBI00056 | Clostridium citroniae | 99.2 | bacteria | firmicutes | 358743 | 56 |
| FBI00057 | Dorea longicatena | 99.7 | bacteria | firmicutes | 88431 | 57 |
| FBI00059 | Bacteroides oleiciplenus | 98.81 | bacteria | bacteroidetes | 626931 | 59 |
| FBI00060 | Bifidobacterium longum | 99.49 | bacteria | actinobacteria | 216816 | 60 |
| FBI00061 | Alistipes shahii | 99.19 | bacteria | bacteroidetes | 328814 | 61 |
| FBI00067 | Oxalobacter formigenes | 98.84 | bacteria | proteobacteria | 847 | 67 |
| FBI00068 | Akkermansia muciniphila | 99.42 | bacteria | verrucomicrobia | 239935 | 68 |
| FBI00069 | Ruminococcus bromii | 98.84 | bacteria | firmicutes | 40518 | 69 |
| FBI00070 | Bacteroides koreensis | 99.71 | bacteria | bacteroidetes | 1912896 | 70 |
| FBI00071 | Roseburia faecis | 94.92 | bacteria | firmicutes | 1732 | 71 |
| FBI00075 | Paraprevotella clara | 98.85 | bacteria | bacteroidetes | 454154 | 75 |
| FBI00076 | Bacteroides thetaiotaomicron | 99.78 | bacteria | bacteroidetes | 818 | 76 |
| FBI00079 | Clostridium clostridioforme | 99.14 | bacteria | firmicutes | 1531 | 79 |
| FBI00080 | Sutterella massiliensis | 99.78 | bacteria | proteobacteria | 1816689 | 80 |
| FBI00087 | Clostridium scindens | 98.28 | bacteria | firmicutes | 29347 | 87 |
| FBI00093 | Roseburia hominis | 99.71 | bacteria | firmicutes | 301301 | 93 |
| FBI00097 | Phocea massiliensis | 93.07 | bacteria | firmicutes | 1841867 | 97 |
| FBI00101 | Faecalibacterium prausnitzii | 97.97 | bacteria | firmicutes | 853 | 101 |
| FBI00102 | Clostridium symbiosum | 94.31 | bacteria | firmicutes | 1512 | 102 |
| FBI00104 | Blautia luti | 97.18 | bacteria | firmicutes | 418240 | 104 |
| FBI00109 | Coprococcus comes | 98.39 | bacteria | firmicutes | 410072 | 109 |
| FBI00117 | Blautia faecis | 99.52 | bacteria | firmicutes | 871665 | 117 |
| FBI00120 | Hungatella hathewayi | 98.78 | bacteria | firmicutes | 154046 | 120 |
| FBI00125 | Bacteroides stercoris | 99.64 | bacteria | bacteroidetes | 46506 | 125 |
| FBI00127 | Collinsella aerofaciens | 98.81 | bacteria | actinobacteria | 74426 | 127 |
| FBI00128 | Hungatella effluvii | 98.71 | bacteria | firmicutes | 1096246 | 128 |
| FBI00132 | Gordonibacter pamelaeae | 99.48 | bacteria | actinobacteria | 471189 | 132 |
| FBI00133 | Oxalobacter formigenes | 99.21 | bacteria | proteobacteria | 847 | 133 |
| FBI00137 | Bacteroides fragilis | 99.71 | bacteria | bacteroidetes | 817 | 137 |
| FBI00141 | Phascolarctobacterium faecium | 99.15 | bacteria | firmicutes | 33025 | 141 |
| FBI00145 | Bifidobacterium adolescentis | 99.14 | bacteria | actinobacteria | 1680 | 145 |
| FBI00149 | Monoglobus pectinilyticus | 99.5 | bacteria | firmicutes | 1981510 | 149 |
| FBI00151 | Clostridium aldenense | 98.55 | bacteria | firmicutes | 358742 | 151 |
| FBI00152 | Dialister invisus | 99.58 | bacteria | firmicutes | 218538 | 152 |
| FBI00162 | Bifidobacterium catenulatum | 99.14 | bacteria | actinobacteria | 1686 | 162 |
| FBI00165 | Bacteroides massiliensis | 99.71 | bacteria | bacteroidetes | 204516 | 165 |
| FBI00171 | Desulfovibrio desulfuricans | 91.45 | bacteria | proteobacteria | 876 | 171 |
| FBI00174 | Lachnospira pectinoschiza | 97.92 | bacteria | firmicutes | 28052 | 174 |
| FBI00176 | Ruthenibacterium lactatiformans | 99.71 | bacteria | firmicutes | 1550024 | 176 |
| FBI00177 | Parasutterella excrementihominis | 99.71 | bacteria | proteobacteria | 487175 | 177 |
| FBI00180 | Alistipes senegalensis | 97.56 | bacteria | bacteroidetes | 1288121 | 180 |
| FBI00182 | Bacteroides coprocola | 99.64 | bacteria | bacteroidetes | 310298 | 182 |
| FBI00184 | Bacteroides faecis | 99.78 | bacteria | bacteroidetes | 674529 | 184 |
| FBI00190 | Bacteroides finegoldii | 98.91 | bacteria | bacteroidetes | 338188 | 190 |
| FBI00191 | Clostridium swellfunianum | 96.24 | bacteria | firmicutes | 1367462 | 191 |
| FBI00194 | Ruminococcus faecis | 98.41 | bacteria | firmicutes | 592978 | 194 |
| FBI00197 | Bifidobacterium bifidum | 99.85 | bacteria | actinobacteria | 1681 | 197 |
| FBI00198 | Lachnoclostridium pacaense | 99.71 | bacteria | firmicutes | 1917870 | 198 |
| FBI00199 | Clostridium bolteae | 99.28 | bacteria | firmicutes | 208479 | 199 |
| FBI00200 | Longicatena caecimuris | 99.71 | bacteria | firmicutes | 1796635 | 200 |
| FBI00201 | Eggerthella lenta | 98.83 | bacteria | actinobacteria | 84112 | 201 |
| FBI00205 | Blautia luti | 97.55 | bacteria | firmicutes | 89014 | 205 |
| FBI00208 | Anaerotruncus colihominis | 96.52 | bacteria | firmicutes | 169435 | 208 |
| FBI00211 | Bacteroides vulgatus | 99.78 | bacteria | bacteroidetes | 821 | 211 |
| FBI00220 | Megasphaera massiliensis | 98.8 | bacteria | firmicutes | 1232428 | 220 |
| FBI00221 | Butyricimonas faecihominis | 98.61 | bacteria | bacteroidetes | 1472416 | 221 |
| FBI00224 | Sutterella wadsworthensis | 99.71 | bacteria | proteobacteria | 40545 | 224 |
| FBI00226 | Catabacter hongkongensis | 99.71 | bacteria | firmicutes | 270498 | 226 |
| FBI00233 | Anaerotruncus colihominis | 91.63 | bacteria | firmicutes | 474960 | 233 |
| FBI00236 | Eisenbergiella tayi | 99.41 | bacteria | firmicutes | 1432052 | 236 |
| FBI00237 | Dielma fastidiosa | 99.78 | bacteria | firmicutes | 1034346 | 237 |
| FBI00238 | Alistipes finegoldii | 95.84 | bacteria | bacteroidetes | 214856 | 238 |
| FBI00243 | Eubacterium siraeum | 98.53 | bacteria | firmicutes | 39492 | 243 |
| FBI00245 | Acidaminococcus intestini | 99.72 | bacteria | firmicutes | 187327 | 245 |
| FBI00248 | Emergencia timonensis | 99.64 | bacteria | firmicutes | 1776384 | 248 |
| FBI00254 | Eubacterium hallii | 99.08 | bacteria | firmicutes | 39488 | 254 |
| FBI00258 | Turicibacter sanguinis | 99.93 | bacteria | firmicutes | 154288 | 258 |
| FBI00267 | Anaerofustis stercorihominis | 97.29 | bacteria | firmicutes | 214853 | 267 |
| FBI00273 | Bamesiella intestinihominis | 99.43 | bacteria | bacteroidetes | 487174 | 273 |
| FBI00274 | Eubacterium xylanophilum | 93.5 | bacteria | firmicutes | 39497 | 274 |
| FBI00275 | Holdemanella biformis | 98.99 | bacteria | firmicutes | 1735 | 275 |
| FBI00278 | Eubacterium ventriosum | 94.14 | bacteria | firmicutes | 39496 | 278 |
| FBI00281 | Senegalimassilia anaerobia | 99.45 | bacteria | actinobacteria | 1473216 | 281 |
| FBI00282 | Porphyromonas asaccharolytica | 99.35 | bacteria | bacteroidetes | 28123 | 282 |
| FBI00288 | Blautia hydrogenotrophica | 99.57 | bacteria | firmicutes | 53443 | 288 |
| FBI00289 | Oxalobacter formigenes | 99.21 | bacteria | proteobacteria | 847 | 289 |
| FBI00290 | Eubacterium ruminantium | 94.81 | bacteria | firmicutes | 42322 | 290 |
| FBI00292 | Methanobrevibacter smithii | 99.44 | archaea | euryarchaeota | 2173 | 292 |
| FBI00377 | Christensenella massiliensis | 88.69 | bacteria | firmicutes | 186802 | 377 |

### Example 17: In Vivo Oxalate Reduction by a Therapeutic Microbial Consortium in Healthy Humans Treated with a High Oxalate/Low Calcium Diet

This study evaluates the ability of a rationally designed oxalate-degrading microbial consortium to reduce urinary oxalate levels in vivo in human subjects.

Approximately 64 healthy subjects are enrolled for the study. Six days prior to administration of the consortium, subjects are placed on a high oxalate/low calcium (HOLC) diet in order to create a temporary hyperoxaluric state akin to what is seen in enteric hyperoxaluria (Langman et al., 2016, “A double-blind, placebo controlled, randomized phase 1 cross-over study with ALLN-177, an orally administered oxalate degrading enzyme,” Am J Nephrol. 44(2):150-8). When administered to healthy subjects over 7 days, this diet has been previously shown to increase urinary oxalate from 27.2±9.5 mg/day during screening to 80.8±24.1 mg/day. This is well above the generally accepted upper limit of normal (40 mg/day) and clearly within the range seen in enteric hyperoxaluria.

Some subjects are additionally pre-treated with a course of broad spectrum antibiotics (a combination of metronidazole and clarithromycin) in order to pre-clear bacteria from the gut and facilitate subsequent engraftment of the heterologous community. This combination is selected based on the complementary coverage of gram-positive as well as gram-negative bacteria, broad coverage of obligate anaerobes (which dominate the microbial population in the GI tract) as well as facultative anaerobes, including enteric pathobionts (i.e. human commensals with pathogenic potential), and the relatively favorable safety and tolerability profiles of the constituent drugs. The goal of antibiotic pretreatment is to reduce pre-existing gastrointestinal bacterial load in an attempt to suppress colonization resistance, a microbially-mediated phenomenon that could limit the engraftment of strains in the consortium.

On Day 6 of administration of the HOLC diet and (optionally) the antibiotic pretreatment, some subjects are additionally given a polyethylene glycol (PEG) bowel preparation treatment, an approach commonly used in fecal matter transplant administration and that will be familiar to one skilled in the art. This treatment is designed to clear remaining antibiotics from the gastrointestinal tract and further reduce remaining bacterial load from the host.

Six days following administration of the HOLC diet, subjects are administered the therapeutic microbial consortium. The duration of treatment with the consortium or the placebo is 10 days. Urine oxalate excretion is used as a biomarker for treatment efficacy, and is monitored by LC-MS as described in Example 4. Stool samples are collected at all stages of the trial (including 1 month post-treatment) and used to monitor the composition of the microbiome by metagenomic sequencing. This facilitates monitoring the level and duration of engraftment of consortium strains.

Approximately 64 healthy human subjects are randomly assigned to one of the following five regimens in a 1:1:1:1 ratio:
a. Antibiotic pretreatment followed by bowel preparation with PEG followed by the treatment with the consortium.
b. Antibiotic pretreatment followed by treatment with the consortium.
c. Antibiotic placebo treatment followed by bowel preparation with PEG followed by treatment with the consortium.
d. Antibiotic pretreatment followed by treatment with a placebo.

Subjects are kept in confinement for two periods, separated by an approximately 20 day washout. The first confinement period is approximately 18 days, which includes antibiotic/antibiotic placebo pretreatment, followed by either a bowel preparation with PEG or no bowel preparation, followed by 10-day course of a therapeutic consortium or a placebo. The second confinement period is approximately 6 days. The sample size of this study was chosen to distinguish an approximately 20% change in in urinary oxalate levels between cohorts. This study enables evaluation of the ability of a therapeutic consortium to reduce levels of urine oxalate in a human subject. This study further evaluates the efficacy of the described pretreatment methods (antibiotic pretreatment and PEG preparation).

### Example 18: In Vivo Oxalate Reduction by a Therapeutic Microbial Consortium in Humans Patients with Enteric Hyperoxaluria

Enteric hyperoxaluria is characterized by excess absorption or consumption of dietary oxalate leading to increased renal oxalate excretion (>40 mg/day), recurrent kidney stones, renal calcium deposition (nephrocalcinosis) and, in severe cases, progressive renal impairment and end-stage renal failure (Liu and Nazzal, 2019, “Enteric hyperoxaluria: role of microbiota and antibiotics,” Curr Opin Nephrol Hypertens. 28(4):352-359; Ermer et al., 2016, “Oxalate, inflammasome, and progression of kidney disease,” Curr Opin Nephrol Hypertens. 25(4):363-71). Roux-en-Y Gastric Bypass (RYGB) surgery is a common comorbidity associated with enteric hyperoxaluria (˜60% of RYGB patients). This study evaluates the ability of an oxalate-degrading microbial consortium to reduce urinary oxalate levels in vivo in a cohort of up to approximately 16 Roux-en-Y Gastric Bypass (RYGB) patients with enteric hyperoxaluria.

A cohort of up to approximately 16 subjects is given an antibiotic pretreatment, a PEG bowel preparation treatment, and a 10-day treatment with a therapeutic microbial consortium as described in Example 17. Urine and stool samples are collected at different stages of the treatment to monitor urine oxalate levels and engraftment of consortium strains as described in Example 17. Stool samples are further collected after 30, 60, and 90 days to evaluate long-term engraftment of consortium strains by metagenomic sequencing. This study will demonstrate the ability of the consortium to reduce urinary oxalate levels in the RYGB patients.

### Example 19: Screening Strains for In Vitro Bile Acid Compound Metabolic Activity

in vitro metabolic screening is necessary to definitively characterize the ability of a microbial strain to degrade bile acid compounds. Strains are screened against a panel of bile acid compounds and structural conversion of the bile acids are evaluated as described. Briefly, overnight microbial monocultures are harvested by anaerobic centrifugation and resuspended in fresh pre-reduced growth medium (e.g. Mega Medium) spiked with 100 μM of bile acid (e.g. TCA, TCDCA, GCA, GCDCA, CA, CDCA, 3oxoCA, 7oxoCA, 12oxoCA, UDCA, DCA, LCA, 3oxoLCA) and allowed to incubate at 37° C. for 24 h. Cultures are sampled for bile acid analysis at 0, 6 and 24 h post-bile acid spike. For bile acid analysis, 2 ml of culture are sampled and immediately acidified with 50 μl of 6 N HCl to stop all metabolic activity and protonate bile acids to make them more soluble in organic solvent. Acidified cultures are extracted for bile acids and analyzed by LCMS (UPLC-QTOF or UPLC-QQQ).

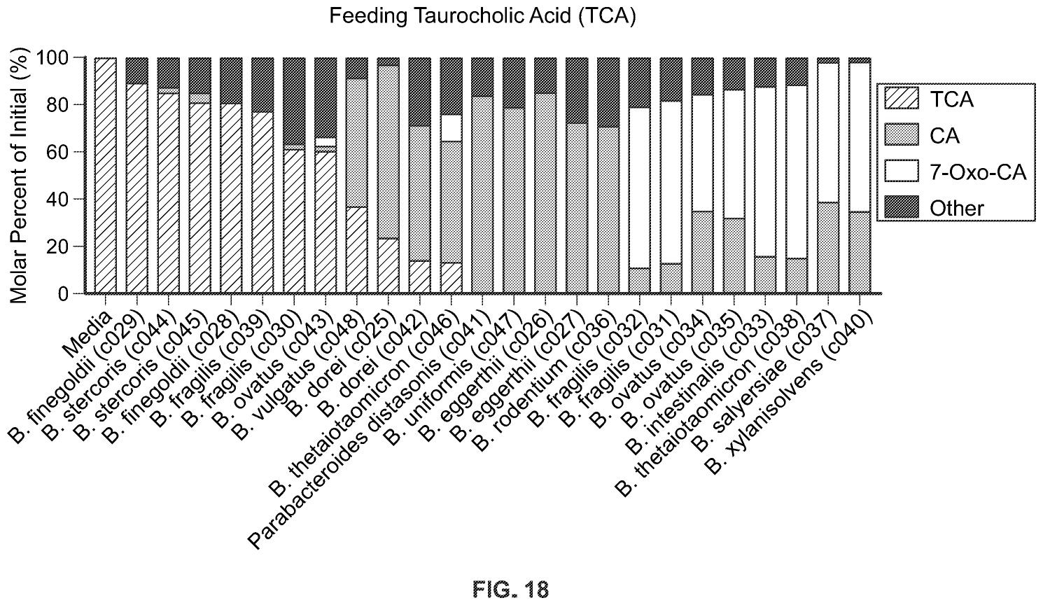

Preliminary screening of commercial strains using TCA as the feeder molecule were obtained using this protocol, and the results are illustrated in FIG. 18.

### Example 20: Screening Strains for Resistance to Bile Acids

To determine the effect of the presence of bile acid on microbial strain growth, microbial cultures are grown in their respective banking medium (e.g. Mega Media or Chopped Meat Media) to saturation and back-diluted into the same respective banking medium containing a variable concentration of bile acids. % growth inhibition is calculated by determining the ratio of background-subtracted optical density (O.D.) of a microbial strain grown in the presence of bile acid to the O.D. of the same microbial strain grown in the absence of bile acid.

### Example 21: Murine Model of Chemically-Induced Primary Sclerosing Cholangitis and Microbiome-Induced Shift in Bile Acid Composition

This example describes the establishment of a chemically-induced murine model of primary sclerosing cholangitis (PSC) and demonstrates that alterations to a microbiome can alter the composition of the bile acid pool and affect disease severity.

On Day 0 of the experiment, germ-free 7-9 week-old germ-free C57B/6N female mice are weighed and colonized by oral gavage with one of two rationally-designed microbial consortia. One cohort of mice is colonized with a full microbial consortium that comprises a plurality of microbes including species having 7α-dehydroxylation activity and species having bile salt hydrolase (BSH) activity. A second cohort of mice is colonized with a partial microbial consortium which is identical in composition to the full consortium except that it lacks species having 7α-dehydroxylation activity. A control cohort of mice is treated with sterile saline.

The mice are fed for two weeks on a standard laboratory diet while the microbiome stabilizes. Beginning on Day 14 and for the following 14 days, the standard diet is supplemented either with 1% (w/w) hepatotoxic secondary bile acid LCA to induce PSC, or with an equimolar concentration of the conjugated bile acid GCDCA or the primary bile acid CDCA. GCDCA can be metabolized into CDCA by a population of microbes having BSH activity, and CDCA can be metabolized into LCA by a population of microbes having 7α-dehydroxylation activity.

On Days 0, 7, 14, 21, and 28, mice are monitored for indicators of chemically-induced PSC (e.g. reduced body weight, reduced food consumption, elevated liver enzyme levels) and fecal samples are collected. Fecal samples are analyzed by both LC/MS to determine the composition of the bile acid pool and by metagenomic sequencing to monitor microbial strain engraftment. Mice are euthanized on or before Day 28 and terminal samples are collected to enable screening for additional PSC indicators (e.g. changes to GI physiology, cecum bile acid composition).

Mice fed a diet supplemented with hepatotoxic LCA are expected to have elevated levels of fecal LCA and are expected to exhibit signs of PSC, thereby establishing a murine model of the disease. Mice colonized with the full set of microbes and fed a diet supplemented with GCDCA or CDCA are likewise expected to have elevated LCA content, as the upstream substrates can be metabolized into LCA by the engrafted set of microbes. Mice implanted with the partial set of microbes and fed a diet supplemented with conjugated bile acid are expected to not have LCA in their bile acid pool because the implanted microbial population lacks the activity necessary to metabolize the upstream substrates into LCA; these mice are accordingly expected to exhibit less severe signs of PSC. Taken together, these results will demonstrate that alterations to the microbiome can drive shifts in the bile acid pool in an animal and affect disease severity.

### Example 22: In Vivo Reduction of Hepatotoxic Bile Acids in a Mouse Model of PSC by Treatment with a Microbial Consortium

This example evaluates the ability of a bile-acid-metabolizing microbial consortium, comprising a plurality of active microbes and a supportive community of microbes, to alter the bile acid pool of an animal and affect disease severity. Said microbial consortium comprises a plurality of active microbes and a supportive community of microbes, wherein said plurality of active microbes comprises strains experimentally verified to have 3α-HSDH and/or 3β-HSDH activity, and said supportive community of microbes comprises strains experimentally verified to have 7α-HSDH activity, 7β-HSDH activity, and/or bile salt hydrolase activity.

To test the in vivo activity of a bile-acid-metabolizing microbial consortium described herein, germ-free C57B/6N female mice are weighed on Day 0 and colonized by oral gavage with either a plurality of active microbes alone, a supportive community alone, or a complete microbial consortium (actives and supportives). The mice are fed for two weeks on a standard laboratory diet while the microbiome stabilizes. Beginning on Day 14 and for the following 14 days, the standard diet is supplemented with the hepatotoxic secondary bile acid LCA (1% w/w) to induce PSC. Body weight, food weight, and fecal bile acid composition are monitored over the course of two weeks. After the two-week period, mice are sacrificed and a variety of terminal samples are collected including the cecum, feces, and serum.

Mice treated with the complete microbial consortium (actives and supportives) are expected to have reduced levels of hepatotoxic LCA and are expected to exhibit less severe signs of PSC relative to an untreated control (no microbial implantation). The mice treated with the active microbes alone are also expected to have lowered LCA levels relative to the untreated mice, but less so than the mice treated with the full consortium. The mice implanted with the supportive community only are not expected to have substantially lower LCA levels than the untreated mice. Taken together, these results will demonstrate the ability of a bile-acid-metabolizing microbial consortium to alter the pool of bile acids in an animal and consequently alleviate PSC symptoms.

1. A microbial consortium for administration to an animal, comprising:
a plurality of active microbes and an effective amount of a supportive community of microbes, wherein
the plurality of active microbes metabolize a first metabolic substrate to produce one or more than one metabolite, wherein the first metabolic substrate causes or contributes to disease in an animal, and
the supportive community of microbes comprises between 1 and 300 microbial strains,
wherein for the supportive community of microbes, at least one of the following four conditions is met:
1) the supportive community of microbes metabolizes one or more than one metabolite produced by the plurality of active microbes, wherein the one or more than one metabolite inhibits metabolism of the first metabolic substrate by one or more of the plurality of active microbes,
2) the supportive community of microbes increases the flux of a precursor of the first metabolic substrate into a biochemical pathway that converts said precursor into a metabolite that is not the first metabolic substrate,
3) the supportive community of microbes enhances one or more than one characteristic of the plurality of active microbes when administered to an animal selected from the group consisting of: a) gastrointestinal engraftment, b) biomass, c) first metabolic substrate metabolism, and d) longitudinal stability as compared to administration of the plurality of active microbes in the absence of the supportive community of microbes, and
4) the supportive community of microbes catalyzes one or more than one reaction selected from the group consisting of: fermentation of polysaccharides to one or more than one of the group consisting of acetate, acetoin, 2-oxoglutarate, propionate, 1,3-propanediol, succinate, ethanol, lactate, butyrate, 2,3-butanediol, acetone, butanol, formate, H₂, and CO₂, fermentation of amino acids to one or more than one of the group consisting of acetate, propionate, butanoate, butyrate, isobutyrate, 2-methylbutyrate, isovalerate, isocaproate, 3-phenylpropanoate, phloretate, 3-(1H-indol-3-yl)propanoate, 5-aminopentanoate, H₂, H₂S, and CO₂, synthesis of one or more than one of the group consisting of methane from H₂ and CO₂, methane from formate and H₂, acetate from H₂ and CO₂, acetate from formate and H₂, acetate and sulfide from H₂, CO₂, and sulfate, propionate and CO₂ from succinate, succinate from H₂ and fumarate; synthesis of succinate from formate and fumarate, and butyrate, acetate, H₂, and CO₂ from lactate, deconjugation of conjugated bile acids to produce primary bile acids, conversion of cholic acid (CA) to 7-oxocholic acid, conversion of 7-oxocholic acid to 7-beta-cholic acid (7betaCA), conversion of chenodeoxycholic acid (CDCA) to 7-oxochenodeoxycholic acid, and conversion of 7-oxochenodeoxycholic acid to ursodeoxycholic acid (UDCA).

2.-12. (canceled)

13. The microbial consortium of claim 1, wherein at least one of the two following conditions is met:
the first metabolic substrate metabolizing activity of at least one of the plurality of active microbes is significantly different when measured in a standardized substrate metabolization assay at two pH values within a range of 4 to 8, and wherein the difference between the two pH values is at least one pH unit, and
the first metabolic substrate metabolizing activity of at least one of the plurality of active microbes is significantly different when measured in a standardized substrate metabolization assay at two first metabolic substrate concentrations within a 100 fold range, and wherein the difference between the two first metabolic substrate concentrations is at least 1.2-fold.

14. The microbial consortium of claim 1, wherein the supportive community of microbes comprises at least three phyla selected from the group consisting of Bacteroidetes, Firmicutes, Actinobacteria, Proteobacteria, Verrucomicrobia, and Euryarchaeota.

15.-24. (canceled)

25. The microbial consortium of claim 1, wherein:
the plurality of active microbes comprises Oxalobacter formigenes;
the first metabolic substrate is oxalate;
the metabolite is (i) formate or (ii) formate and carbon dioxide; and
the supportive community of microbes catalyzes the synthesis of methane from formate and H₂.

26.-27. (canceled)

28. The microbial consortium of claim 25, wherein the supportive community of microbes comprises a Bacteroidetes and a Euryarchaeota.

29.-30. (canceled)

31. The microbial consortium of claim 25, wherein the supportive community of microbes comprises between 20 and 200 microbial strains and comprises at least 4 phyla selected from the group consisting of Bacteroidetes, Firmicutes, Actinobacteria, and Proteobacteria.

32.-33. (canceled)

34. The microbial consortium of claim 31, wherein the supportive community comprises:
(i) Ruminococcus bromii, Clostridium citroniae, Bacteroides salyersiae, Neglecta timonensis, Bifidobacterium longum, Bifidobacterium pseudocatenulatum, Bacteroides thetaiotaomicron, Eggerthella lenta, Clostridiaceae sp., Bifidobacterium dentium, Parabacteroides merdae, Bilophila wadsworthia, Bacteroides caccae, Dorea longicatena, Collinsella aerofaciens, Clostridium scindens, Faecalibacterium prausnitzii, Clostridium symbiosum, and Bacteroides vulgatus;
(ii) Acidaminococcus intestine, Akkermansia muciniphila, Alistipes onderdonkii, Alistipes putredinis, Alistipes senegalensis, Alistipes shahii, Alistipes sp., Alistipes timonensis, Anaerofustis stercorihominis, Anaerostipes hadrus, Anaerotruncus massiliensis, Bacteroides caccae, Bacteroides coprocola, Bacteroides faecis, Bacteroides finegoldii, Bacteroides fragilis, Bacteroides kribbi, Bacteroides massiliensis, Bacteroides nordii, Bacteroides ovatus, Bacteroides salyersiae, Bacteroides stercorirosoris, Bacteroides stercoris, Bacteroides thetaiotaomicron, Bacteroides uniformis, Bacteroides vulgatus, Bacteroides xylanisolvens, Barnesiella intestinihominis, Bifidobacterium adolescentis, Bifidobacterium bifidum, Bifidobacterium catenulatum, Bifidobacterium dentium, Bifidobacterium longum, Bifidobacterium pseudocatenulatum, Bilophila wadsworthia, Blautia faecis, Blautia hydrogenotrophica, Blautia massiliensis, Blautia obeum, Blautia wexlerae, Butyricimonas faecihominis, Catabacter hongkongensis, Clostridiaceae sp., Clostridiales sp., Clostridium aldenense, Clostridium bolteae, Clostridium citroniae, Clostridium clostridioforme, Clostridium fessum, Clostridium scindens, Collinsella aerofaciens, Coprococcus comes, Coprococcus eutactus, Dialister invisus, Dialister succinatiphilus, Dielma fastidiosa, Dorea formicigenerans, Dorea longicatena, Eggerthella lenta, Eisenbergiella tayi, Eubacterium eligens, Eubacterium hallii, Eubacterium rectale, Eubacterium siraeum, Eubacterium ventriosum, Eubacterium xylanophilum, Faecalibacterium prausnitzii, Fusicatenibacter saccharivorans, Gordonibacter pamelaeae, Holdemanella biformis, Hungatella effluvia, Lachnoclostridium pacaense, Lachnospiraceae sp., Lactobacillus rogosae, Longicatena caecimuris, Megasphaera massiliensis, Methanobrevibacter smithii, Monoglobus pectinilyticus, Neglecta timonensis, Parabacteroides distasonis, Parabacteroides merdae, Paraprevotella clara, Parasutterella excrementihominis, Phascolarctobacterium faecium, Porphyromonas asaccharolytica, Roseburia hominis, Ruminococcaceae sp., Ruminococcus bromii, Ruminococcus faecis, Ruthenibacterium lactatiformans, Senegalimassilia anaerobia, Sutterella massiliensis, Sutterella wadsworthensis, and Turicibacter sanguinis; or
(iii) Akkermansia muciniphila, Alistipes onderdonkii, Alistipes putredinis, Alistipes shahii, Alistipes timonensis, Anaerostipes hadrus, Bacteroides caccae, Bacteroides fragilis, Bacteroides kribbi, Bacteroides koreensis, Bacteroides massiliensis, Bacteroides nordii, Bacteroides salyersiae, Bacteroides stercorirosoris, Bacteroides stercoris, Bacteroides thetaiotaomicron, Bacteroides uniformis, Bacteroides vulgatus, Bacteroides xylanisolvens, Bifidobacterium adolescentis, Bifidobacterium catenulatum, Bifidobacterium dentium, Bifidobacterium longum, Bifidobacterium pseudocatenulatum, Bilophila wadsworthia, Bilophila wadsworthia, Blautia faecis, Blautia obeum, Blautia wexlerae, Clostridium aldenense, Clostridium bolteae, Clostridium citroniae, Clostridium clostridioforme, Clostridium fessum, Clostridium scindens, Collinsella aerofaciens, Coprococcus comes, Coprococcus eutactus, Dialister invisus, Dialister succinatiphilus, Dorea formicigenerans, Dorea longicatena, Eggerthella lenta, Eisenbergiella tayi, Eubacterium eligens, Eubacterium rectale, Faecalibacterium prausnitzii, Fusicatenibacter saccharivorans, Gordonibacter pamelaeae, Holdemanella biformis, Hungatella effluvia, Lachnoclostridium pacaense, Lachnospiraceae sp., Lactobacillus rogosae, Monoglobus pectinilyticus, Neglecta timonensis, Parabacteroides distasonis, Parabacteroides merdae, Paraprevotella clara, Parasutterella excrementihominis, Phascolarctobacterium faecium, Porphyromonas asaccharolytica, Roseburia hominis, Ruminococcaceae sp., Ruminococcus bromii, Ruminococcus faecis, Ruthenibacterium lactatiformans, Sutterella massiliensis, and Sutterella wadsworthensis.

35.-45. (canceled)

46. The microbial consortium of claim 1, wherein the plurality of active microbes and the supportive community of microbes are selected from a group of microbes each comprising a 16S sequence at least 97% identical to any one of the microbes listed in Table 4, Table 22, Table 23, Table 20, Table 16, Table 17, Table 18 or Table 19.

47.-61. (canceled)

62. The microbial consortium of claim 1, wherein at least one of the plurality of active microbes has a higher first metabolic substrate metabolizing activity at
(A) a lower pH compared to:
(i) at least one other of the plurality of active microbes at the same lower pH; or
(ii) a first metabolic substrate metabolizing activity of the same active microbe at a higher pH,
wherein the lower pH is at 4.5±0.5, or
(B) a higher pH compared to:
(i) at least one other of the plurality of active microbes at the same higher pH; or
(ii) a first metabolic substrate activity of the same active microbe at a lower pH,
wherein the higher pH is at 7.5±0.5.

63.-67. (canceled)

68. The microbial consortium of claim 1, wherein at least one of the plurality of active microbes has a higher first metabolic substrate metabolizing activity at a lower pH and one of the plurality of active microbes has a higher first metabolic substrate metabolizing activity at a higher pH, wherein the difference between the two pH values is at least 1.5, 2.0, 2.5, 3.0, 3.5, or 4.0 pH units.

69. (canceled)

70. The microbial consortium of claim 1, wherein at least one of the plurality of active microbes has a higher first metabolic substrate metabolizing activity at
(A) a lower concentration of first metabolic substrate compared to:
(i) the first metabolic substrate activity of at least one other of the plurality of active microbes when measured in a standardized substrate metabolization assay under the same conditions; or
(ii) a first metabolic substrate metabolizing activity of the same active microbe at a higher concentration of first metabolic substrate, or
(B) a higher concentration of first metabolic substrate compared to:
(i) the first metabolic substrate activity of at least one other of the plurality of active microbes when measured in a standardized substrate metabolization assay under the same conditions; or
(ii) a first metabolic substrate metabolizing activity of the same active microbe at a lower concentration of first metabolic substrate.

71.-73. (canceled)

74. The microbial consortium of claim 1, wherein at least one of the plurality of active microbes has a higher first metabolic substrate metabolizing activity at a lower first metabolic substrate concentration and one of the plurality of active microbes has a higher first metabolic substrate metabolizing activity at a higher first metabolic substrate concentration, and wherein the difference between the two first metabolic substrate concentrations is at least 1.2 fold, 2.0 fold, 3.0 fold, 4.0 fold, 5.0 fold, 6.0 fold, 7.0 fold, 8.0 fold, 9.0 fold, 10 fold, 20 fold, 30 fold, 40 fold, 50 fold, 60 fold, 70 fold, 80 fold, 90 fold, or 100 fold.

75.-77. (canceled)

78. The microbial consortium of claim 25, wherein at least one of the plurality of active microbes has a higher oxalate metabolizing activity
(A) at 0.75 mM of oxalate compared to:
(i) the oxalate metabolizing activity of at least one other of the plurality of active microbes when measured in a standardized oxalate metabolization assay under the same conditions; or
(ii) an oxalate metabolizing activity of the same active microbe at a higher concentration of oxalate, or
(B) at 40 mM of oxalate compared to:
(i) the oxalate metabolizing activity of at least one other of the plurality of active microbes when measured in a standardized oxalate metabolization assay under the same conditions; or
(ii) an oxalate metabolizing activity of the same active microbe at a lower concentration of oxalate.

79.-82. (canceled)

83. The microbial consortium of claim 78, wherein the standardized substrate metabolization assay comprises using:
(A) a colorimetric enzyme assay that measures the activity of oxalate oxidase in a culture sample comprising the microbial consortium, wherein the culture sample comprises three or more microbial strains in an appropriate culture media incubated for 1 hour to 96 hours in the presence of oxalate at a concentration of 0.5 mM to 50 mM, at a pH of 3.5 to 8.0, and at a temperature of 35° C. to 40° C.; or
(B) liquid chromatography-tandem mass spectrometry (LC-MS/MS) to measure the amount of oxalate in a culture sample comprising the microbial consortium, wherein the culture sample comprises three or more microbial strains in an appropriate culture media incubated for 1 hour to 96 hours in the presence of oxalate at a concentration of 0.5 mM to 50 mM, at a pH of 3.5 to 8.0, and at a temperature of 35° C. to 40° C.

84. (canceled)

85. The microbial consortium of claim 25, wherein the consortium further comprises:
a fermenting microbe that metabolizes a fermentation substrate to one or more than one fermentation product; and
a synthesizing microbe that catalyzes a synthesis reaction that combines the one or more than one metabolite and the one or more than one fermentation product to generate one or more than one synthesis product,
wherein the fermentation substrate is
(A) a polysaccharide and the one or more than one fermentation product is selected from the group consisting of acetate, acetoin, 2-oxoglutarate, propionate, 1,3-propanediol, succinate, ethanol, lactate, butyrate, 2,3-butanediol, acetone, butanol, formate, hydrogen gas, and carbon dioxide, or
(B) an amino acid and the one or more than one fermentation product is selected from the group consisting of acetate, propionate, butanoate, butyrate, isobutyrate, 2-methylbutyrate, isovalerate, isocaproate, 3-phenylpropanoate, phloretate, 3-(1H-indol-3-yl)propanoate, 5-aminopentanoate, hydrogen gas, hydrogen sulfide, and carbon dioxide, and
wherein the reaction catalyzed by the synthesizing microbe is selected from the group consisting of: synthesis of methane from carbon dioxide and hydrogen gas; synthesis of methane from H₂ and CO₂, methane from formate and H₂, acetate from H₂ and CO₂, acetate from formate and H₂, acetate and sulfide from H₂, CO₂, and sulfate, propionate and CO₂ from succinate, succinate from H₂ and fumarate; synthesis of succinate from formate and fumarate, and butyrate, acetate, H₂, and CO₂ from lactate.

86. The microbial consortium of claim 85, wherein the one or more than one fermentation product is a second metabolic substrate for the plurality of active microbes or a third metabolic substrate for the synthesizing microbe.

87. The microbial consortium of claim 85, wherein the one or more than one synthesis product is a second metabolic substrate for the plurality of active microbes or a fourth metabolic substrate for the fermenting microbe.

88.-90. (canceled)

91. The microbial consortium of claim 85, wherein the microbial consortium, when administered to an animal on a high oxalate diet, significantly reduces oxalate concentration in a sample selected from the group consisting of blood, serum, stool, or urine, as compared to a sample collected from a corresponding control animal on a high oxalate diet that has not been administered with the microbial consortium.

92.-114. (canceled)

115. The microbial consortium of claim 1, wherein the first metabolic substrate is a primary bile acid selected from the group consisting of lithocholic acid (LCA), and deoxycholic acid (DCA), and
the one or more than one metabolite is selected from the group consisting of iso-lithocholic acid (iso-LCA), or iso-deoxycholic acid (iso-DCA).

116.-117. (canceled)

118. The microbial consortium claim 115, wherein the supportive community of microbes enhances the conversion of:
(i) one or more conjugated bile acids selected from the group consisting of taurochenodeoxycholic acid (TCDCA), glycochenodeoxycholic acid (GCDCA), taurocholic acid (TCA), and glycocholic acid (GCA), to cholic acid (CA) or chenodeoxycholic acid (CDCA);
(ii) CA to 7-beta-cholic acid (7betaCA); or
(iii) CDCA to ursodeoxycholic acid (UDCA).

119.-120. (canceled)

121. The microbial consortium of claim 115, wherein at least one of the plurality of active microbes has a higher bile acid metabolization activity
(A) at a bile acid concentration of 0.1 mM compared to:
(i) the bile acid metabolization activity of at least one other of the plurality of active microbes when measured in a standardized bile acid metabolization assay under the same conditions; or
(ii) a bile acid metabolizing activity of the same active microbe at a higher bile acid concentration, or
(B) at a bile acid concentration of 10 mM compared to:
(i) the bile acid metabolization activity of at least one other of the plurality of active microbes when measured in a standardized bile acid metabolization assay under the same conditions; or
(ii) a bile acid metabolizing activity of the same active microbe at a lower bile acid concentration.

122.-125. (canceled)

126. The microbial consortium of claim 115, wherein the standardized substrate metabolization assay comprises using liquid chromatography-mass spectrometry to determine the bile acid profile in a culture sample comprising the microbial consortium, wherein the culture sample comprises three or more microbial strains in an appropriate culture media incubated for 1 hour to 96 hours in the presence of bile acids at a concentration of 0.1 mM to 10 mM, at a pH of 3.5 to 8.0, and at a temperature of 35° C. to 40° C.

127. The microbial consortium of claim 115, wherein the plurality of active microbes comprises one or more microbial phyla selected from Firmicutes and Actinobacteria, one or more microbial strain selected from Eggerthella lenta and Clostridium scindens; and
the supportive community of microbes comprises 20 to 200 microbial strains.

128.-134. (canceled)

135. The microbial consortium of claim 1, wherein the microbial consortium, when administered to the animal, decreases a concentration of the first metabolic substrate in the animal.

136. (canceled)

137. A pharmaceutical composition comprising the microbial consortium of claim 1 and a pharmaceutically acceptable carrier or excipient.

138. A method of treating a subject diagnosed with or at risk for a metabolic disease or condition selected from the group consisting of primary hyperoxaluria, secondary hyperoxaluria, primary sclerosing cholangitis, primary biliary cholangitis, progressive familial intrahepatic cholestasis, nonalcoholic steatohepatitis, and multiple sclerosis, the method comprising administering to the subject, the pharmaceutical composition of claim 137,
wherein administration of the pharmaceutical composition reduces levels of the first metabolic substrate in the subject by at least 20% as compared to an untreated control subject or as compared to pre-administration levels of the first metabolic substrate in the subject.

139.-145. (canceled)

146. A supportive community of microbes comprising between 1 and 300 microbial strains, wherein at least one of the following four conditions is met:
1) the supportive community of microbes metabolizes one or more than one metabolite produced by a plurality of active microbes, wherein the one or more than one metabolite inhibits metabolism of a first metabolic substrate by one or more of the plurality of active microbes, wherein the first metabolic substrate causes or contributes to a disease in an animal,
2) the supportive community of microbes increases the flux of a precursor of the first metabolic substrate into a biochemical pathway that converts said precursor into a metabolite that is not the first metabolic substrate,
3) the supportive community of microbes enhances one or more than one characteristic of the plurality of active microbes when administered to an animal selected from the group consisting of: a) gastrointestinal engraftment, b) biomass, c) first metabolic substrate metabolism, and d) longitudinal stability as compared to administration of the plurality of active microbes in the absence of the supportive community of microbes, and
4) the supportive community of microbes catalyzes one or more than one reaction selected from the group consisting of: fermentation of polysaccharides to one or more than one of the group consisting of acetate, acetoin, 2-oxoglutarate, propionate, 1,3-propanediol, succinate, ethanol, lactate, butyrate, 2,3-butanediol, acetone, butanol, formate, H₂, and CO₂, fermentation of amino acids to one or more than one of the group consisting of acetate, propionate, butanoate, butyrate, isobutyrate, 2-methylbutyrate, isovalerate, isocaproate, 3-phenylpropanoate, phloretate, 3-(1H-indol-3-yl)propanoate, 5-aminopentanoate, H₂, H₂S, and CO₂, synthesis of one or more than one of the group consisting of methane from H₂ and CO₂, methane from formate and H₂, acetate from H₂ and CO₂, acetate from formate and H₂, acetate and sulfide from H₂, CO₂, and sulfate, propionate and CO₂ from succinate, succinate from H₂ and fumarate; synthesis of succinate from formate and fumarate, and butyrate, acetate, H₂, and CO₂ from lactate, deconjugation of conjugated bile acids to produce primary bile acids, conversion of cholic acid (CA) to 7-oxocholic acid, conversion of 7-oxocholic acid to 7-beta-cholic acid (7betaCA), conversion of chenodeoxycholic acid (CDCA) to 7-oxochenodeoxycholic acid, and conversion of 7-oxochenodeoxycholic acid to ursodeoxycholic acid (UDCA).

147. (canceled)

148. The supportive community of claim 146, wherein the supportive community comprises at least 4 phyla selected from the group consisting of Bacteroidetes, Firmicutes, Actinobacteria, and Proteobacteria.

149. (canceled)

150. The supportive community of claim 148, wherein the supportive community comprises:
(i) Ruminococcus bromii, Clostridium citroniae, Bacteroides salyersiae, Neglecta timonensis, Bifidobacterium longum, Bifidobacterium pseudocatenulatum, Bacteroides thetaiotaomicron, Eggerthella lenta, Clostridiaceae sp., Bifidobacterium dentium, Parabacteroides merdae, Bilophila wadsworthia, Bacteroides caccae, Dorea longicatena, Collinsella aerofaciens, Clostridium scindens, Faecalibacterium prausnitzii, Clostridium symbiosum, and Bacteroides vulgatus;
(ii) Acidaminococcus intestine, Akkermansia muciniphila, Alistipes onderdonkii, Alistipes putredinis, Alistipes senegalensis, Alistipes shahii, Alistipes sp., Alistipes timonensis, Anaerofustis stercorihominis, Anaerostipes hadrus, Anaerotruncus massiliensis, Bacteroides caccae, Bacteroides coprocola, Bacteroides faecis, Bacteroides finegoldii, Bacteroides fragilis, Bacteroides kribbi, Bacteroides massiliensis, Bacteroides nordii, Bacteroides ovatus, Bacteroides salyersiae, Bacteroides stercorirosoris, Bacteroides stercoris, Bacteroides thetaiotaomicron, Bacteroides uniformis, Bacteroides vulgatus, Bacteroides xylanisolvens, Barnesiella intestinihominis, Bifidobacterium adolescentis, Bifidobacterium bifidum, Bifidobacterium catenulatum, Bifidobacterium dentium, Bifidobacterium longum, Bifidobacterium pseudocatenulatum, Bilophila wadsworthia, Blautia faecis, Blautia hydrogenotrophica, Blautia massiliensis, Blautia obeum, Blautia wexlerae, Butyricimonas faecihominis, Catabacter hongkongensis, Clostridiaceae sp., Clostridiales sp., Clostridium aldenense, Clostridium bolteae, Clostridium citroniae, Clostridium clostridioforme, Clostridium fessum, Clostridium scindens, Collinsella aerofaciens, Coprococcus comes, Coprococcus eutactus, Dialister invisus, Dialister succinatiphilus, Dielma fastidiosa, Dorea formicigenerans, Dorea longicatena, Eggerthella lenta, Eisenbergiella tayi, Eubacterium eligens, Eubacterium hallii, Eubacterium rectale, Eubacterium siraeum, Eubacterium ventriosum, Eubacterium xylanophilum, Faecalibacterium prausnitzii, Fusicatenibacter saccharivorans, Gordonibacter pamelaeae, Holdemanella biformis, Hungatella effluvia, Lachnoclostridium pacaense, Lachnospiraceae sp., Lactobacillus rogosae, Longicatena caecimuris, Megasphaera massiliensis, Methanobrevibacter smithii, Monoglobus pectinilyticus, Neglecta timonensis, Parabacteroides distasonis, Parabacteroides merdae, Paraprevotella clara, Parasutterella excrementihominis, Phascolarctobacterium faecium, Porphyromonas asaccharolytica, Roseburia hominis, Ruminococcaceae sp., Ruminococcus bromii, Ruminococcus faecis, Ruthenibacterium lactatiformans, Senegalimassilia anaerobia, Sutterella massiliensis, Sutterella wadsworthensis, and Turicibacter sanguines; or
(iii) Akkermansia muciniphila, Alistipes onderdonkii, Alistipes putredinis, Alistipes shahii, Alistipes timonensis, Anaerostipes hadrus, Bacteroides caccae, Bacteroides Bacteroides kribbi, Bacteroides koreensis, Bacteroides massiliensis, Bacteroides nordii, Bacteroides salyersiae, Bacteroides stercorirosoris, Bacteroides stercoris, Bacteroides thetaiotaomicron, Bacteroides uniformis, Bacteroides vulgatus, Bacteroides xylanisolvens, Bifidobacterium adolescentis, Bifidobacterium catenulatum, Bifidobacterium dentium, Bifidobacterium longum, Bifidobacterium pseudocatenulatum, Bilophila wadsworthia, Bilophila wadsworthia, Blautia faecis, Blautia obeum, Blautia wexlerae, Clostridium aldenense, Clostridium bolteae, Clostridium citroniae, Clostridium clostridioforme, Clostridium fessum, Clostridium scindens, Collinsella aerofaciens, Coprococcus comes, Coprococcus eutactus, Dialister invisus, Dialister succinatiphilus, Dorea formicigenerans, Dorea longicatena, Eggerthella lenta, Eisenbergiella tayi, Eubacterium eligens, Eubacterium rectale, Faecalibacterium prausnitzii, Fusicatenibacter saccharivorans, Gordonibacter pamelaeae, Holdemanella biformis, Hungatella effluvia, Lachnoclostridium pacaense, Lachnospiraceae sp., Lactobacillus rogosae, Monoglobus pectinilyticus, Neglecta timonensis, Parabacteroides distasonis, Parabacteroides merdae, Paraprevotella clara, Parasutterella excrementihominis, Phascolarctobacterium faecium, Porphyromonas asaccharolytica, Roseburia hominis, Ruminococcaceae sp., Ruminococcus bromii, Ruminococcus faecis, Ruthenibacterium lactatiformans, Sutterella massiliensis, and Sutterella wadsworthensis.

151.-156. (canceled)
